(19) United States
(12) Patent Application Publication
(10) Pub. No.: US 2022/0213092 A1
(43) Pub. Date: Jul. 7, 2022

(54) BICYCLIC PYRAZOLE BRUTON'S TYROSINE KINASE INHIBITORS

(71) Applicant: AbbVie Inc., North Chicago, IL (US)

(72) Inventors: David A. DeGoey, Salem, WI (US); Felix DeAnda, Jr., Sunnyvale, CA (US); Amanda W. Dombrowski, Chicago, IL (US); Igor Dubovyk, San Jose, CA (US); Stephen N. Greszler, Vernon Hills, IL (US); Dimitri Khrakovsky, Oakland, CA (US); Chih-Hung Lee, San Carlos, CA (US); James N. Newton, Lake Bluff, IL (US); Elizabeth L. Noey, Franklin, WI (US); Akinori Okano, San Mateo, CA (US); Kevin B. Sippy, Antioch, IL (US); Andrew J. Souers, Libertyville, IL (US); Longcheng Wang, Los Altos, CA (US); Noel S. Wilson, Kenosha, WI (US); Jeffery A. Zablocki, Los Altos, CA (US)

(21) Appl. No.: 17/552,396

(22) Filed: Dec. 16, 2021

Related U.S. Application Data
(60) Provisional application No. 63/126,817, filed on Dec. 17, 2020.

(51) Int. Cl. C07D 471/04; C07D 519/00; C07D 487/04

(52) U.S. Cl. CPC C07D 471/04; C07D 487/04; C07D 519/00

(57) ABSTRACT

The present invention provides for compounds of formula (I)

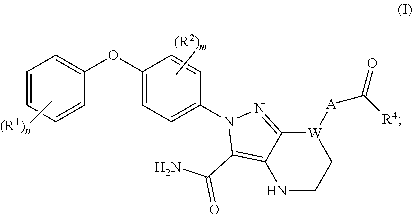

wherein A, R¹, R², R⁴, W, m, and n have any of the values defined herein, and pharmaceutically acceptable salts thereof, that are useful as agents in the treatment of CLL and/or SLL.

### CROSS-REFERENCE TO RELATED APPLICATION

The present application claims priority to U.S. Provisional Patent Application Ser. No. 63/126,817, filed Dec. 17, 2020. The disclosure of the foregoing reference is hereby incorporated by reference in its entirety.

### BRIEF DESCRIPTION OF THE SEQUENCE LISTING

Incorporated herein by reference in its entirety is a Sequence Listing entitled, “ABV12596USO1 sequence listing_ST25.txt”, comprising SEQ ID NO: 1 through SEQ ID NO: 4, which includes the amino acid sequence disclosed herein. The Sequence Listing has been submitted herewith in ASCII text format via EFS. The Sequence Listing was first created on Dec. 14, 2021 and is 1,180 bytes in size.

### BACKGROUND OF THE INVENTION

Bruton's tyrosine kinase (Btk) is a key signaling enzyme expressed in hematopoietic cell types. Btk plays an essential role in the B-cell signaling pathway linking cell surface B-cell receptor (BCR) stimulation to downstream intracellular responses.

Btk is a key regulator of B-cell development, activation, signaling, and survival (Kurosaki, Curr Op Imm, 2000, 276-281; Schaeffer and Schwartzberg, Curr Op Imm 2000, 282-288). In addition, Btk plays a role in a number of other hematopoietic cell signaling pathways, e.g., Toll like receptor (TLR) and cytokine receptor-mediated TNF-α production in macrophages, IgE receptor (FcepsilonRI) signaling in mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197:1603-1611; Iwaki et al. (2005), Journal of Biological Chemistry 280(48):40261-40270; Vassilev et al. (1999), Journal of Biological Chemistry 274(3):1646-1656, and Quek et al. (1998), Current Biology 8(20):1137-1140. Accordingly, there is an ongoing medical need to develop new BTK inhibitors.

### BRIEF SUMMARY OF THE INVENTION

In certain aspects, the present invention provides a compound of formula I,

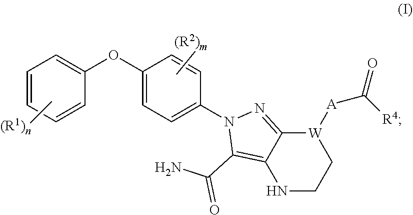

wherein

A is a 4-9 membered heterocycloalkylene substituted with —(R³)ₚ;

W is CH or N;

R¹ is independently selected from the group consisting of halo, C₁-C₄ alkyl, C₃-C₆ cycloalkyl, C₁-C₄ haloalkyl, —CN, —OH, and —OR^{1a};

R^{1a} is selected from the group consisting of C₁-C₄ alkyl and C₁-C₄ haloalkyl;

R² is independently selected from the group consisting of halo, C₁-C₄ alkyl, and OR^{2a};

R^{2a} is selected from the group consisting of C₁-C₄ alkyl and C₁-C₄ haloalkyl;

R³ is independently selected from the group consisting of —OH, C₁-C₄ alkyl, C₁-C₄ hydroxyalkyl, —CH₂CH₂—, and —CH₂CH₂CH₂—;

R⁴ is selected from the group consisting of C₁-C₄ haloalkyl, —CH═CHR^{4a}, and C₂-C₄ alkynyl;

R^{4a} is selected from the group consisting of hydrogen, halo, C₁-C₄ alkyl, —OR^{4b}, —CO₂R^{4b}, and —CO₂NH₂; wherein the R^{4a} C₁-C₄ alkyl may optionally be substituted with —OR^{4c} or —NR^{4c}R^{4d};

R^{4b}, R^{4c}, and R^{4d} are each independently C₁-C₄ alkyl;

m is 0, 1, 2, or 3;

n is 0, 1, 2, or 3; and

p is 0, 1, 2, or 3; or a pharmaceutically acceptable salt thereof.

In certain embodiments, A is a nitrogen-containing heterocycloalkylene, or a pharmaceutically acceptable salt thereof.

In certain embodiments, A is a 4-membered heterocycloalkylene, or a pharmaceutically acceptable salt thereof. In certain embodiments, A is a 4-membered nitrogen-containing heterocycloalkylene, or a pharmaceutically acceptable salt thereof.

In certain embodiments, A is a 5-membered heterocycloalkylene, or a pharmaceutically acceptable salt thereof. In certain embodiments, A is a 5-membered nitrogen-containing heterocycloalkylene, or a pharmaceutically acceptable salt thereof.

In certain embodiments, A is a 6-membered heterocycloalkylene, or a pharmaceutically acceptable salt thereof. In certain embodiments, A is a 6-membered nitrogen-containing heterocycloalkylene, or a pharmaceutically acceptable salt thereof.

In certain embodiments, A is a 7-membered heterocycloalkylene, or a pharmaceutically acceptable salt thereof. In certain embodiments, A is a 7-membered nitrogen-containing heterocycloalkylene, or a pharmaceutically acceptable salt thereof.

In certain embodiments, A is an 8-membered heterocycloalkylene, or a pharmaceutically acceptable salt thereof. In certain embodiments, A is an 8-membered nitrogen-containing heterocycloalkylene, or a pharmaceutically acceptable salt thereof.

In certain embodiments, A is a 9-membered heterocycloalkylene, or a pharmaceutically acceptable salt thereof. In certain embodiments, A is a 9-membered nitrogen-containing heterocycloalkylene, or a pharmaceutically acceptable salt thereof.

In certain embodiments, A is selected from the group consisting of piperidinediyl, piperazinediyl, pyrrolidinediyl, azetidinediyl, diazepanediyl, diazanonanediyl, diazaheptanediyl, diazaoctanediyl, diazaheptanediyl, diazaheptanediyl, and tetrahydropyrrolopyrrolediyl, or a pharmaceutically acceptable salt thereof. In certain embodiments, A is piperazinediyl, or a pharmaceutically acceptable salt thereof.

In certain embodiments, A is selected from the group consisting of

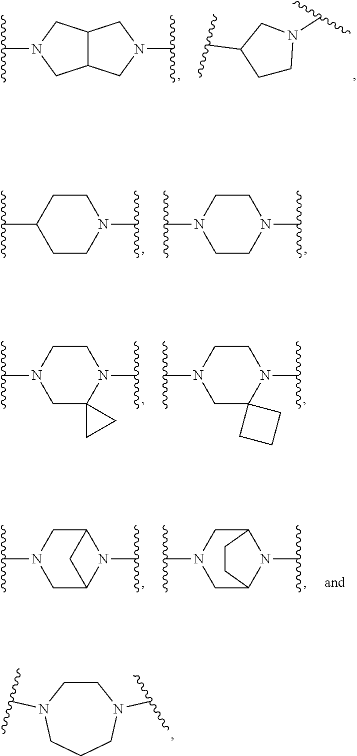

or a pharmaceutically acceptable salt thereof.

In certain embodiments, W is CH, or a pharmaceutically acceptable salt thereof. In certain embodiments, W is N, or a pharmaceutically acceptable salt thereof.

In certain embodiments, W is CH and A is a 6-membered heterocycloalkylene, or a pharmaceutically acceptable salt thereof. In certain embodiments, W is CH and A is a 6-membered nitrogen-containing heterocycloalkylene, or a pharmaceutically acceptable salt thereof. In certain embodiments, W is CH and A is piperazinediyl, or a pharmaceutically acceptable salt thereof. In certain embodiments, W is CH and A is piperidinediyl, or a pharmaceutically acceptable salt thereof. In certain embodiments, A is selected from the group consisting of piperidinediyl and piperazinediyl, or a pharmaceutically acceptable salt thereof. In certain embodiments, A is selected from the group consisting of piperidinediyl and piperazinediyl, and W is CH, or a pharmaceutically acceptable salt thereof.

In certain embodiments, W is N and A is 6-membered heterocycloalkylene, or a pharmaceutically acceptable salt thereof. In certain embodiments, W is N and A is a 4-membered heterocycloalkylene, or a pharmaceutically acceptable salt thereof. In certain embodiments, W is N and A is piperidinediyl, or a pharmaceutically acceptable salt thereof. In certain embodiments, W is N and A is azetidinediyl, or a pharmaceutically acceptable salt thereof. In certain embodiments, A is selected from the group consisting of piperidinediyl and azetidinediyl, or a pharmaceutically acceptable salt thereof. In certain embodiments, A is selected from the group consisting of piperidinediyl and azetidinediyl, and W is N, or a pharmaceutically acceptable salt thereof.

In certain embodiments, compounds of the present disclosure are represented by formula (II):

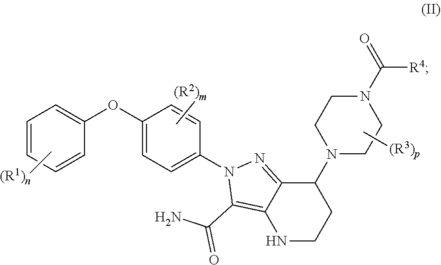

wherein R¹ is independently selected from the group consisting of halo, C₁-C₄ alkyl, C₃-C₆ cycloalkyl, C₁-C₄ haloalkyl, —CN, —OH, and —OR^{1a};

R^{1a} is selected from the group consisting of C₁-C₄ alkyl and C₁-C₄ haloalkyl;

R² is independently selected from the group consisting of halo, C₁-C₄ alkyl, and OR^{2a};

R^{2a} is selected from the group consisting of C₁-C₄ alkyl and C₁-C₄ haloalkyl;

R³ is independently selected from the group consisting of —OH, C₁-C₄ alkyl, C₁-C₄ hydroxyalkyl, —CH₂CH₂—, and —CH₂CH₂CH₂—;

R⁴ is selected from the group consisting of C₁-C₄ haloalkyl, —CH═CHR^{4a}, and C₂-C₄ alkynyl;

R^{4a} is selected from the group consisting of hydrogen, halo, C₁-C₄ alkyl, —OR^{4b}, —CO₂R^{4b}, and —CO₂NH₂; wherein the R^{4a} C₁-C₄ alkyl may optionally be substituted with —OR^{4c} or —NR^{4c}R^{4d};

R^{4b}, R^{4c}, and R^{4d} are each independently C₁-C₄ alkyl;

m is 0, 1, 2, or 3;

n is 0, 1, 2, or 3; and

p is 0, 1, 2, or 3; or a pharmaceutically acceptable salt thereof.

In certain embodiments, R¹ is independently selected from the group consisting of F, —CN, —OH, methyl, cyclopropyl, trifluoromethyl, methoxy, and trifluoromethoxy, or a pharmaceutically acceptable salt thereof.

In certain embodiments, R⁴ is selected from the group consisting of —CH═CHR^{4a} and C₂-C₄ alkynyl, or a pharmaceutically acceptable salt thereof. In certain embodiments, R⁴ is selected from the group consisting of CH═CH₂ and C≡CCH₃, or a pharmaceutically acceptable salt thereof. In certain embodiments, R⁴ is —CH═CH₂, or a pharmaceutically acceptable salt thereof. In certain embodiments, R⁴ is C≡CCH₃, or a pharmaceutically acceptable salt thereof.

In certain embodiments, n is 0, or a pharmaceutically acceptable salt thereof. In certain embodiments, n is 1, or a pharmaceutically acceptable salt thereof. In certain embodiments, n is 2, or a pharmaceutically acceptable salt thereof. In certain embodiments, n is 3, or a pharmaceutically acceptable salt thereof. In certain embodiments, n is selected from the group consisting of 0, 1, and 2, or a pharmaceutically acceptable salt thereof. In certain embodiments, n is selected from the group consisting of 0 and 1, or a pharmaceutically acceptable salt thereof.

In certain embodiments, m is 0, or a pharmaceutically acceptable salt thereof. In certain embodiments, m is 1, or a pharmaceutically acceptable salt thereof. In certain embodiments, m is 2, or a pharmaceutically acceptable salt thereof. In certain embodiments, m is 3, or a pharmaceutically acceptable salt thereof. In certain embodiments, m is selected from the group consisting of 0, 1, and 2, or a pharmaceutically acceptable salt thereof. In certain embodiments, m is selected from the group consisting of 0 and 1, or a pharmaceutically acceptable salt thereof.

In certain embodiments, p is 0, or a pharmaceutically acceptable salt thereof. In certain embodiments, p is 1, or a pharmaceutically acceptable salt thereof. In certain embodiments, p is 2, or a pharmaceutically acceptable salt thereof. In certain embodiments, p is 3, or a pharmaceutically acceptable salt thereof. In certain embodiments, p is selected from the group consisting of 0, 1, and 2, or a pharmaceutically acceptable salt thereof. In certain embodiments, p is selected from the group consisting of 0 and 1, or a pharmaceutically acceptable salt thereof.

In certain embodiments, n is 0 and m is 0, or a pharmaceutically acceptable salt thereof. In certain embodiments, n is 0 and p is 0, or a pharmaceutically acceptable salt thereof. In certain embodiments, m is 0 and p is 0, or a pharmaceutically acceptable salt thereof. In certain embodiments, n is 0, m is 0, and p is 0, or a pharmaceutically acceptable salt thereof.

In certain embodiments, n is 1 and R¹ is halo, or a pharmaceutically acceptable salt thereof. In certain embodiments, n is 1 and R¹ is F, or a pharmaceutically acceptable salt thereof. In certain embodiments, n is 1, R¹ is F, and A is 6-membered nitrogen-containing heterocycloalkylene, or a pharmaceutically acceptable salt thereof. In certain embodiments, n is 1, R¹ is F, and A is piperidinediyl, or a pharmaceutically acceptable salt thereof. In certain embodiments, n is 1, R¹ is F, A is piperidinediyl, and m is 0, or a pharmaceutically acceptable salt thereof. In certain embodiments, n is 1, R¹ is F, and A is piperazinediyl, or a pharmaceutically acceptable salt thereof. In certain embodiments, n is 1, R¹ is F, A is piperazinediyl, and m is 0, or a pharmaceutically acceptable salt thereof.

In certain embodiments, n is 2 and each R¹ is halo, or a pharmaceutically acceptable salt thereof. In certain embodiments, n is 2 and each R¹ is F, or a pharmaceutically acceptable salt thereof. In certain embodiments, n is 2, each R¹ is F, and A is 6-membered nitrogen-containing heterocycloalkylene, or a pharmaceutically acceptable salt thereof. In certain embodiments, n is 2, each R¹ is F, and A is piperidinediyl, or a pharmaceutically acceptable salt thereof. In certain embodiments, n is 2, each R¹ is F, A is piperidinediyl, and m is 0, or a pharmaceutically acceptable salt thereof. In certain embodiments, n is 2, each R¹ is F, and A is piperazinediyl, or a pharmaceutically acceptable salt thereof. In certain embodiments, n is 2, each R¹ is F, A is piperazinediyl, and m is 0, or a pharmaceutically acceptable salt thereof.

In certain embodiments, m is 1 and R² is OR^{2a}, or a pharmaceutically acceptable salt thereof. In certain embodiments, m is 1, R² is OR^{2a}, and R^{2a} is C₁-C₄ alkyl, or a pharmaceutically acceptable salt thereof. In certain embodiments, m is 1, R² is OR^{2a}, and R^{2a} is methyl, or a pharmaceutically acceptable salt thereof. In certain embodiments, m is 1, R² is OR^{2a}, and R^{2a} is methyl, and A is a 6-membered nitrogen-containing heterocycloalkylene, or a pharmaceutically acceptable salt thereof. In certain embodiments, m is 1, R² is OR^{2a}, and R^{2a} is methyl, and A is piperidinediyl, or a pharmaceutically acceptable salt thereof. In certain embodiments, m is 1, R² is OR^{2a}, R^{2a} is methyl, A is piperidinediyl, and n is 0, or a pharmaceutically acceptable salt thereof. In certain embodiments, m is 1, R² is OR^{2a}, and R^{2a} is methyl, and A is piperazinediyl, or a pharmaceutically acceptable salt thereof. In certain embodiments, m is 1, R² is OR^{2a}, R^{2a} is methyl, A is piperazinediyl, and n is 0, or a pharmaceutically acceptable salt thereof.

In certain embodiments, n is 1 and R¹ is OR^{1a}, or a pharmaceutically acceptable salt thereof. In certain embodiments, n is 1, R¹ is OR^{1a}, and R^{1a} is C₁-C₄ alkyl, or a pharmaceutically acceptable salt thereof. In certain embodiments, n is 1, R¹ is OR^{1a}, and R^{1a} is methyl, or a pharmaceutically acceptable salt thereof. In certain embodiments, n is 1, R¹ is OR^{1a}, and R^{1a} is methyl, and A is a 6-membered nitrogen-containing heterocycloalkylene, or a pharmaceutically acceptable salt thereof. In certain embodiments, n is 1, R¹ is OR^{1a}, and R^{1a} is methyl, and A is piperidinediyl, or a pharmaceutically acceptable salt thereof. In certain embodiments, n is 1, R¹ is OR^{1a}, R^{1a} is methyl, A is piperidinediyl, and m is 0, or a pharmaceutically acceptable salt thereof. In certain embodiments, n is 1, R¹ is OR^{1a}, and R^{1a} is methyl, and A is piperazinediyl, or a pharmaceutically acceptable salt thereof. In certain embodiments, n is 1, R¹ is OR^{1a}, R^{1a} is methyl, A is piperazinediyl, and m is 0, or a pharmaceutically acceptable salt thereof.

In certain embodiments, n is 1 and R¹ is C₁-C₄ haloalkyl, or a pharmaceutically acceptable salt thereof. In certain embodiments, n is 1 and R¹ is trifluoromethyl, or a pharmaceutically acceptable salt thereof. In certain embodiments, n is 1, R¹ is trifluoromethyl, and A is a 6-membered nitrogen-containing heterocycloalkylene, or a pharmaceutically acceptable salt thereof. In certain embodiments, n is 1, R¹ is trifluoromethyl, and A is piperazinediyl, or a pharmaceutically acceptable salt thereof. In certain embodiments, n is 1, R¹ trifluoromethyl, and A is piperidinediyl, or a pharmaceutically acceptable salt thereof.

In certain embodiments, n is 1, R¹ is —CN (cyanide), and A is a 6-membered nitrogen-containing heterocycloalkylene, or a pharmaceutically acceptable salt thereof. In certain embodiments, n is 1, R¹ is —CN, and A is piperazinediyl, or a pharmaceutically acceptable salt thereof. In certain embodiments, n is 1, R¹ is —CN, and A is piperidinediyl, or a pharmaceutically acceptable salt thereof.

In certain embodiments, n is 1 and R¹ is C₃-C₆ cycloalkyl, or a pharmaceutically acceptable salt thereof. In certain embodiments, n is 1, R¹ is cyclopropyl, and A is a 6-membered nitrogen-containing heterocycloalkylene, or a pharmaceutically acceptable salt thereof. In certain embodiments, n is 1, R¹ is cyclopropyl, and A is piperazinediyl, or a pharmaceutically acceptable salt thereof. In certain embodiments, n is 1, R¹ is cyclopropyl, and A is piperidinediyl, or a pharmaceutically acceptable salt thereof.

In certain embodiments, p is 2 and each R³ is independently C₁-C₄ alkyl, or a pharmaceutically acceptable salt thereof. In certain embodiments, p is 2 and each R³ is methyl, or a pharmaceutically acceptable salt thereof. In certain embodiments, p is 2, each R³ is methyl, and A is a 6-membered nitrogen-containing heterocycloalkylene, or a pharmaceutically acceptable salt thereof. In certain embodiments, p is 2, each R³ is methyl, and A is piperazinediyl, or a pharmaceutically acceptable salt thereof. In certain embodiments, p is 2, each R³ is methyl, and A is piperidinediyl, or a pharmaceutically acceptable salt thereof.

In certain embodiments, p is 1 and R³ is C₁-C₄ alkyl, or a pharmaceutically acceptable salt thereof. In certain embodiments, p is 1 and R³ is methyl, or a pharmaceutically acceptable salt thereof. In certain embodiments, p is 1, R³ is methyl, and A is a 6-membered nitrogen-containing heterocycloalkylene, or a pharmaceutically acceptable salt thereof. In certain embodiments, p is 1, R³ is methyl, and A is piperazinediyl, or a pharmaceutically acceptable salt thereof. In certain embodiments, p is 1, R³ is methyl, and A is piperidinediyl, or a pharmaceutically acceptable salt thereof.

In certain embodiments, R⁴ is —CH═CHR^{4a}, or a pharmaceutically acceptable salt thereof. In certain embodiments, R⁴ is —CH═CHR^{4a}, and R^{4a} is H, or a pharmaceutically acceptable salt thereof. In certain embodiments, R⁴ is —CH═CHR^{4a}, R^{4a} is H, and A is piperazinediyl, or a pharmaceutically acceptable salt thereof. In certain embodiments, R⁴ is —CH═CHR^{4a}, R^{4a} is H, and A is piperidinediyl, or a pharmaceutically acceptable salt thereof.

In certain embodiments, R⁴ is C₂-C₄ alkynyl, or a pharmaceutically acceptable salt thereof. In certain embodiments, R⁴ is C₃ alkynyl, or a pharmaceutically acceptable salt thereof. In certain embodiments, R⁴ is C₃ alkynyl and A is piperidinediyl, or a pharmaceutically acceptable salt thereof. In certain embodiments, R⁴ is C₃ alkynyl and A is piperazinediyl, or a pharmaceutically acceptable salt thereof.

In certain embodiments, R⁴ is —CH═CHR^{4a}, R^{4a} is C₁-C₄ alkyl, or a pharmaceutically acceptable salt thereof. In certain embodiments, R⁴ is —CH═CHR^{4a}, R^{4a} is C₁ alkyl, or a pharmaceutically acceptable salt thereof. In certain embodiments, R⁴ is —CH═CHR^{4a}, R^{4a} is C₁ alkyl, and R^{4a} is substituted with —NR^{4c}R^{4d} or a pharmaceutically acceptable salt thereof. In certain embodiments, R⁴ is —CH═CHR^{4a}, R^{4a} is C₁ alkyl, R^{4a} is substituted with —NR^{4c}R^{4d} and R^{4c} and R^{4d} are each methyl, or a pharmaceutically acceptable salt thereof.

In certain embodiments, R⁴ is —CH═CHR^{4a}, and R^{4a} is —CO₂NH₂, or a pharmaceutically acceptable salt thereof. In certain embodiments, R⁴ is C₁-C₄ haloalkyl, or a pharmaceutically acceptable salt thereof.

In certain embodiments, A is piperidinediyl, W is CH, and R⁴ is selected from the group consisting of —CH═CHR^{4a} and C₂-C₄ alkynyl, or a pharmaceutically acceptable salt thereof. In certain embodiments, A is piperidinediyl, W is CH, and R⁴ is selected from the group consisting of —CH═CH₂ and C≡CCH₃, or a pharmaceutically acceptable salt thereof. In certain embodiments, A is piperazinediyl, W is CH, and R⁴ is selected from the group consisting of —CH═CHR^{4a} and C₂-C₄ alkynyl, or a pharmaceutically acceptable salt thereof. In certain embodiments, A is piperazinediyl, W is CH, and R⁴ is selected from the group consisting of —CH═CH₂ and C≡CCH₃, or a pharmaceutically acceptable salt thereof.

In certain embodiments, A is piperidinediyl, W is CH, R⁴ is selected from the group consisting of —CH═CHR^{4a} and C₂-C₄ alkynyl, and m is 0, or a pharmaceutically acceptable salt thereof. In certain embodiments, A is piperazinediyl, W is CH, R⁴ is selected from the group consisting of —CH═CHR^{4a} and C₂-C₄ alkynyl, and m is 0, or a pharmaceutically acceptable salt thereof.

In certain embodiments, A is piperidinediyl; W is CH; R¹ is independently selected from the group consisting of halo, C₁-C₄ alkyl, C₁-C₄ haloalkyl, and —OR^{1a}; R⁴ is selected from the group consisting of —CH═CHR^{4a} and C₂-C₄ alkynyl; and m is 0, or a pharmaceutically acceptable salt thereof. In certain embodiments, A is piperazinediyl; W is CH; R¹ is independently selected from the group consisting of halo, C₁-C₄ alkyl, C₁-C₄ haloalkyl, and —OR^{1a}; R⁴ is selected from the group consisting of —CH═CHR^{4a} and C₂-C₄ alkynyl; and m is 0, or a pharmaceutically acceptable salt thereof.

In certain embodiments, A is piperidinediyl; W is CH; R¹ is independently selected from the group consisting of F, Cl, methyl, trifluoromethyl, and methoxy; R⁴ is selected from the group consisting of —CH═CHR^{4a} and C₂-C₄ alkynyl; and m is 0; or a pharmaceutically acceptable salt thereof. In certain embodiments, A is piperazinediyl; W is CH; R¹ is independently selected from the group consisting of F, Cl, methyl, trifluoromethyl, and methoxy; R⁴ is selected from the group consisting of —CH═CHR^{4a} and C₂-C₄ alkynyl; and m is 0; or a pharmaceutically acceptable salt thereof.

### Definition of Terms

As used in the specification and the appended claims, unless specified to the contrary, the following terms have the meaning indicated.

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds.

The term “alkenyl,” as used herein, refers to a straight or branched hydrocarbon chain radical containing at least one carbon-carbon double bond and having two, three, four, five, or six carbon atoms. In certain embodiments, the alkenyl has two carbon atoms. An example of an alkenyl is —CH═CH₂.

The term “alkyl,” as used herein, refers to a saturated, straight, or branched hydrocarbon chain radical having one, two, three, four, five, or six carbon atoms. Representative examples of alkyl include, but are not limited to, methyl, ethyl, n-propyl, isopropyl, and the like. In certain embodiments, the alkyl is methyl. In certain embodiments, the alkyl is isopropyl.

The term “alkynyl,” as used herein, refers to a straight or branched hydrocarbon chain radical containing at least one carbon-carbon triple bond and having two, three, four, five or six carbon atoms. In certain embodiments, the alkynyl has two, three, or four carbon atoms, i.e., is a C₂ C₄ alkynyl. An exemplary alkynyl is C≡CCH₃.

With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below.

The term “cycloalkyl,” as used herein, refers to a saturated hydrocarbon ring radical containing three, four, five, six, seven, or eight carbon ring atoms. In certain embodiments, the cycloalkyl is a monocyclic cycloalkyl. The monocyclic cycloalkyl is a carbocyclic ring system containing three to eight carbon atoms, zero heteroatoms and zero double bonds. In certain embodiments, the cycloalkyl has three, four, five, or six carbon atoms, i.e., is a C₃-C₆ cycloalkyl. Examples of monocyclic ring systems include cyclopropyl, cyclobutyl, and the like. In certain embodiments, the cycloalkyl is selected from the group consisting of cyclopropyl and cyclobutyl.

In a fused ring system, the two rings share one common bond.

The term “halo” or “halogen,” as used herein, means Cl, Br, I, and F. In certain embodiments, halo is selected from the group consisting of Cl or F. In certain embodiments, the halo is Cl. In certain embodiments, the halo is F.

The term “haloalkyl,” as used herein, refers to an alkyl group, as defined herein, in which one or more hydrogen atoms are replaced by halogen. In certain embodiments, one or more hydrogen atoms are replaced by fluorine. An example of a haloalkyl is trifluoromethyl.

The term “heteroatom,” as used herein, means a nitrogen, oxygen, or sulfur atom. In certain embodiments, the heteroatom is a nitrogen atom.

The term “heterocycloalkyl” refers to a stable, non-aromatic, saturated monocyclic or polycyclic heterocycloalkane radical having carbon atoms and 1 or more heteroatoms independently selected from S, N, or O. In certain embodiments, the heterocycloalkyl has 4 to 9 members. The heterocycloalkyl may be a monocyclic ring or a polycyclic ring (containing more than one ring). Examples of polycyclic heterocycloalkyls include bridged, fused, and spirocyclic heterocycloalkyls in which at least one ring is heterocycloalkyl and the other ring(s) are heterocycloalkyl, or cycloalkyl, rings.

The term “heterocycloalkylene,” refers to a stable, divalent group derived from a non-aromatic, saturated monocyclic or polycyclic heterocycloalkyl having carbon atoms and 1 or more heteroatoms independently selected from S, N or O. The heterocycloalkylene is connected to the parent molecular moiety through any substitutable carbon atoms or any substitutable nitrogen atoms contained within the rings. In certain embodiments, the heterocycloalkylene has 4-9 members. In certain embodiments, the heterocycloalkylene has 1 or more nitrogen atoms. In certain embodiments, the heterocycloalkylene has 1 nitrogen atom. In certain embodiments, the heterocycloalkylene has 2 nitrogen atoms. In certain embodiments, the heterocycloalkylene is monocyclic. In certain embodiments, the heterocycloalkylene is monocyclic and has 1 or more nitrogen atoms. In certain embodiments, the heterocycloalkylene is polycyclic. Examples of polycyclic heterocycloalkylenes include bridged, fused, and spirocyclic heterocycloalkylenes. In certain embodiments, the heterocycloalkylene is polycyclic and has 4-9 members. In certain embodiments, the polycyclic ring is bicyclo. In certain embodiments, the heterocycloalkylene contains 1 or more double bonds, as long as the double bonds do not render the heterocycloalkylene aromatic. In certain embodiments, the heterocycloalkylene does not contain any double bonds, i.e., is fully saturated. In certain embodiments, the heterocycloalkylene does not contain any double bonds and has 4-9 members. Examples of heterocycloalkylenes are piperidinediyl, piperazinediyl, pyrrolidinediyl, azetidinediyl, diazepanediyl, diazanonanediyl, diazaheptanediyl, diazaoctanediyl, tetrahydropyrrolopyrrolediyl, and the like.

In certain embodiments, the heterocycloalkylene is a stable, 4-membered, monocyclic ring having 3 carbon atoms and 1 heteroatom. In certain embodiments, the heterocycloalkylene is a stable, 4-membered, monocyclic ring having 3 carbon atoms and 1 nitrogen atom. An example of a 4-membered heterocycloalkylene is azetidinediyl.

In certain embodiments, the heterocycloalkylene is a stable, 5-membered monocyclic ring having 3 or 4 carbon atoms and 1 or 2 nitrogen atoms (i.e., 3 carbon atoms and two nitrogen atoms, or 4 carbon atoms and 1 nitrogen atom). An example of a 5-membered heterocycloalkylene is pyrrolidinediyl.

In certain embodiments, the heterocycloalkylene is a stable, 6-membered monocyclic ring having 4 or 5 carbon atoms and 1 or 2 nitrogen atoms (i.e., 4 carbon atoms and 2 nitrogen atoms, or 5 carbon atoms and 1 nitrogen atom). In certain embodiments, the 6-membered heterocycloalkylene is fully saturated, i.e., has no double bonds. Exemplary 6-membered heterocycloalkylenes include piperidinediyl, piperazinediyl, and the like.

In certain embodiments, the heterocycloalkylene is a stable, 7-membered ring having 1 or more heteroatoms. In certain embodiments, the 7-membered heterocycloalkylene has 1 or more nitrogen atoms. In certain embodiments, the 7-membered heterocycloalkylene has 2 nitrogen atoms. In certain embodiments, the 7-membered heterocycloalkylene is monocyclic. In certain embodiments, the 7-membered heterocycloalkylene is polycyclic. In certain embodiments, the 7-membered heterocycloalkylene is bridged. Examples of 7-membered heterocycloalkylenes are diazepanediyl and diazaheptanediyl.

In certain embodiments, the heterocycloalkylene is a stable, 8-membered polycyclic ring have 1 or more heteroatoms. In certain embodiments, the 8-membered heterocycloalkylene has 1 or more nitrogen atoms. In certain embodiments, the 8-membered heterocycloalkylene is bridged. In certain embodiments, the 8-membered heterocycloalkylene is a fused bicyclic ring. An example of an 8-membered heterocycloalkylene is tetrahydropyrrolopyrrolediyl.

In certain embodiments, the heterocycloalkylene is a stable, 9-membered polycyclic ring having 1 or more heteroatoms. In certain embodiments, the 9-membered heterocycloalkylene has 1 or more nitrogen atoms. In certain embodiments, the 9-membered heterocycloalkylene has 2 nitrogen atoms. In certain embodiments, the 9-membered heterocycloalkylene is a spirocyclic ring system. An example of a 9-membered heterocycloalkylene is diazanonanediyl.

The term “hydroxyalkyl,” as used herein, refers to a hydroxy group, as defined herein, appended to the parent molecular moiety through an alkyl group, as defined herein. The hydroxyalkyl group may have one, two, three, four, five, or six carbons. Representative examples of hydroxyalkyl include, but are not limited to, hydroxymethyl, 2-hydroxyethyl, 3-hydroxypropyl, and the like. In certain embodiments, the hydroxyalkyl is a C₁-C₄ hydroxyalkyl. In certain embodiments, the hydroxyalkyl is a C₁-C₆ hydroxyalkyl.

In some instances, the number of carbon atoms in a moiety is indicated by the prefix “Cₓ-C_{y}”, wherein x is the minimum and y is the maximum number of carbon atoms in the substituent. Thus, for example, “C₁-C₆ alkyl” means an alkyl substituent containing from 1 to 6 carbon atoms and “C₁-C₃ alkyl” means an alkyl substituent containing from 1 to 3 carbon atoms. Additionally, “C₁-C₄ alkyl” means an alkyl substituent containing from 1 to 4 carbon atoms.

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. To illustrate, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

The phrase “pharmaceutically acceptable salt” refers to those salts which are, within the scope of sound medical judgement, suitable for use in contact with the tissues of humans and lower animals without undue toxicity, irritation, allergic response and the like and are commensurate with a reasonable benefit/risk ratio. The term “subject,” as used herein, refers to a human. The terms “human,” “patient,” and “subject” are used interchangeably herein.

If a moiety is described as “substituted,” a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

The terms “treat,” “treating,” and “treatment,” as used herein, refer to a method of alleviating or abrogating a disease and/or its attendant symptoms. In certain embodiments, the compounds disclosed herein are useful in the treatment of chronic lymphocytic leukemia (CLL) and/or small lymphocytic lymphoma (SLL). In certain embodiments, a method of treating a human subject with CLL and/or SLL comprising administering to a patient a compound of formula (I) is provided. In one aspect, the invention provides a method for treating CLL and/or SLL comprising administering to a subject in need thereof a therapeutically effective amount of a compound of formula (I). The phrase “therapeutically effective amount” refers to an amount of a compound, or a pharmaceutically acceptable salt thereof, sufficient to prevent the development of or to alleviate to some extent one or more of the symptoms of the condition or disorder being treated when administered for treatment in a particular subject or subject population.

In another aspect, the invention relates to pharmaceutical compositions comprising a therapeutically effective amount of a compound of formula (I), or a pharmaceutically acceptable salt thereof, in combination with a pharmaceutically acceptable carrier.

In certain embodiments, the compound is:

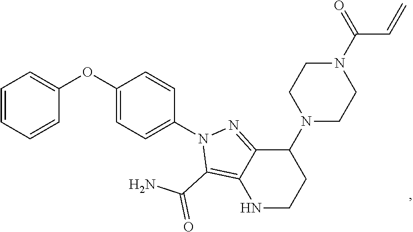 [SMILES: C=CC(=O)N1CCN(C2CCNc3c2nn(-c2ccc(Oc4ccccc4)cc2)c3C(N)=O)CC1]

or a pharmaceutically acceptable salt thereof.

In certain embodiments, the compound is:

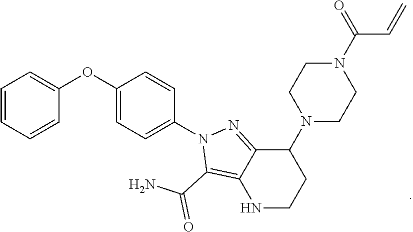 [SMILES: C=CC(=O)N1CCN(C2CCNc3c2nn(-c2ccc(Oc4ccccc4)cc2)c3C(N)=O)CC1]

In certain embodiments, the compound is the pharmaceutically acceptable salt of:

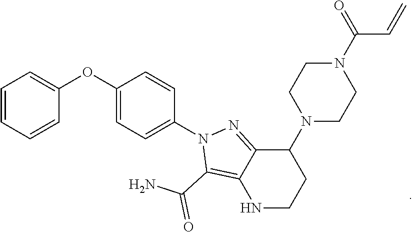 [SMILES: C=CC(=O)N1CCN(C2CCNc3c2nn(-c2ccc(Oc4ccccc4)cc2)c3C(N)=O)CC1]

In certain embodiments, the compound is:

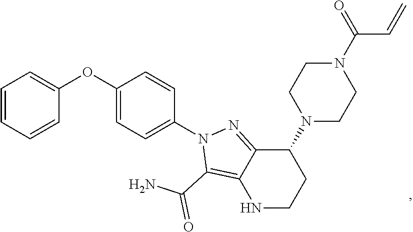 [SMILES: C=CC(=O)N1CCN([C@@H]2CCNc3c2nn(-c2ccc(Oc4ccccc4)cc2)c3C(N)=O)CC1]

or a pharmaceutically acceptable salt thereof.

In certain embodiments, the compound is:

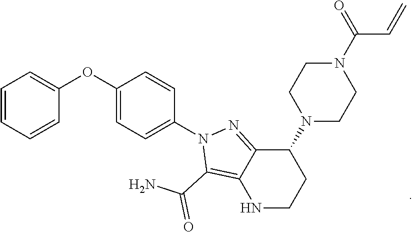 [SMILES: C=CC(=O)N1CCN([C@@H]2CCNc3c2nn(-c2ccc(Oc4ccccc4)cc2)c3C(N)=O)CC1]

In certain embodiments, the compound is the pharmaceutically acceptable salt of:

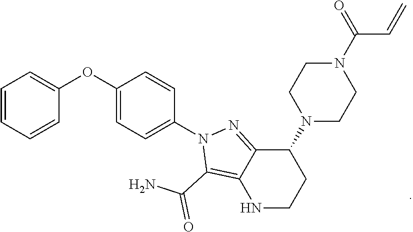 [SMILES: C=CC(=O)N1CCN([C@@H]2CCNc3c2nn(-c2ccc(Oc4ccccc4)cc2)c3C(N)=O)CC1]

In certain embodiments, the compound is 2-(4-phenoxyphenyl)-7-[4-(prop-2-enoyl)piperazin-1-yl]-4,5,6,7-tetrahydro-2H-pyrazolo[4,3-b]pyridine-3-carboxamide, or a pharmaceutically acceptable salt thereof. In certain embodiments, the compound is pharmaceutically acceptable salt of 2-(4-phenoxyphenyl)-7-[4-(prop-2-enoyl)piperazin-1-yl]-4,5,6,7-tetrahydro-2H-pyrazolo[4,3-b]pyridine-3-carboxamide. In certain embodiments, the compound is 2-(4-phenoxyphenyl)-7-[4-(prop-2-enoyl)piperazin-1-yl]-4,5,6,7-tetrahydro-2H-pyrazolo[4,3-b]pyridine-3-carboxamide.

In certain embodiments, the compound is (7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide, or a pharmaceutically acceptable salt thereof. In certain embodiments, the compound is the pharmaceutically acceptable salt of (7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide. In certain embodiments, the compound is (7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide.

In certain embodiments, the compound is selected from the group consisting of
2-(4-phenoxyphenyl)-7-[1-(prop-2-enoyl)piperidin-4-yl]-4,5,6,7-tetrahydro-2H-pyrazolo[4,3-b]pyridine-3-carboxamide;
7-[1-(but-2-ynoyl)piperidin-4-yl]-2-(4-phenoxyphenyl)-4,5,6,7-tetrahydro-2H-pyrazolo[4,3-b]pyridine-3-carboxamide;
2-(4-phenoxyphenyl)-7-[4-(prop-2-enoyl)piperazin-1-yl]-4,5,6,7-tetrahydro-2H-pyrazolo[4,3-b]pyridine-3-carboxamide;
(7R)-2-(4-phenoxyphenyl)-7-[1-(prop-2-enoyl)piperidin-4-yl]-4,5,6,7-tetrahydro-2H-pyrazolo[4,3-b]pyridine-3-carboxamide;
(7S)-2-(4-phenoxyphenyl)-7-[1-(prop-2-enoyl)piperidin-4-yl]-4,5,6,7-tetrahydro-2H-pyrazolo[4,3-b]pyridine-3-carboxamide;
(7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide;
(7S)-2-(4-phenoxyphenyl)-7-[4-(prop-2-enoyl)piperazin-1-yl]-4,5,6,7-tetrahydro-2H-pyrazolo[4,3-b]pyridine-3-carboxamide;
(7SR)-2-(4-phenoxyphenyl)-7-[(3RS)-1-(prop-2-enoyl)piperidin-3-yl]-4,5,6,7-tetrahydro-2H-pyrazolo[4,3-b]pyridine-3-carboxamide;
(7RS)-2-(4-phenoxyphenyl)-7-[(3RS)-1-(prop-2-enoyl)piperidin-3-yl]-4,5,6,7-tetrahydro-2H-pyrazolo[4,3-b]pyridine-3-carboxamide;
2-(4-phenoxyphenyl)-7-[1-(prop-2-enoyl)pyrrolidin-3-yl]-4,5,6,7-tetrahydro-2H-pyrazolo[4,3-b]pyridine-3-carboxamide;
2-[4-(4-fluorophenoxy)phenyl]-7-[1-(prop-2-enoyl)piperidin-4-yl]-4,5,6,7-tetrahydro-2H-pyrazolo[4,3-b]pyridine-3-carboxamide;
2-[4-(3-fluorophenoxy)phenyl]-7-[1-(prop-2-enoyl)piperidin-4-yl]-4,5,6,7-tetrahydro-2H-pyrazolo[4,3-b]pyridine-3-carboxamide;
2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide;
2-(2-methoxy-4-phenoxyphenyl)-7-[1-(prop-2-enoyl)piperidin-4-yl]-4,5,6,7-tetrahydro-2H-pyrazolo[4,3-b]pyridine-3-carboxamide;
2-[4-(3-methoxyphenoxy)phenyl]-7-[1-(prop-2-enoyl)piperidin-4-yl]-4,5,6,7-tetrahydro-2H-pyrazolo[4,3-b]pyridine-3-carboxamide;
2-(4-phenoxyphenyl)-7-[1-(prop-2-enoyl)piperidin-4-yl]-4,5,6,7-tetrahydro-2H-pyrazolo[3,4-b]pyrazine-3-carboxamide;
2-[4-(4-methoxyphenoxy)phenyl]-7-[1-(prop-2-enoyl)piperidin-4-yl]-4,5,6,7-tetrahydro-2H-pyrazolo[4,3-b]pyridine-3-carboxamide;
7-[(2S,5R)-2,5-dimethyl-4-(prop-2-enoyl)piperazin-1-yl]-2-(4-phenoxyphenyl)-4,5,6,7-tetrahydro-2H-pyrazolo[4,3-b]pyridine-3-carboxamide;
7-[1-(but-2-ynoyl)piperidin-4-yl]-2-(4-phenoxyphenyl)-4,5,6,7-tetrahydro-2H-pyrazolo[3,4-b]pyrazine-3-carboxamide;
2-(4-phenoxyphenyl)-7-[(3R)-3-(propan-2-yl)-4-(prop-2-enoyl)piperazin-1-yl]-4,5,6,7-tetrahydro-2H-pyrazolo[4,3-b]pyridine-3-carboxamide;
7-[(2R,5S)-2,5-dimethyl-4-(prop-2-enoyl)piperazin-1-yl]-2-(4-phenoxyphenyl)-4,5,6,7-tetrahydro-2H-pyrazolo[4,3-b]pyridine-3-carboxamide;
2-(4-phenoxyphenyl)-7-[1-(prop-2-enoyl)azetidin-3-yl]-4,5,6,7-tetrahydro-2H-pyrazolo[3,4-b]pyrazine-3-carboxamide;
7-[1-(prop-2-enoyl)piperidin-4-yl]-2-{4-[3-(trifluoromethyl)phenoxy]phenyl}-4,5,6,7-tetrahydro-2H-pyrazolo[4,3-b]pyridine-3-carboxamide;
2-[4-(3-methylphenoxy)phenyl]-7-[1-(prop-2-enoyl)piperidin-4-yl]-4,5,6,7-tetrahydro-2H-pyrazolo[4,3-b]pyridine-3-carboxamide;
2-(4-phenoxyphenyl)-7-[4-(prop-2-enoyl)-1,4-diazepan-1-yl]-4,5,6,7-tetrahydro-2H-pyrazolo[4,3-b]pyridine-3-carboxamide;
2-(4-phenoxyphenyl)-7-[5-(prop-2-enoyl)-5,8-diazaspiro[3.5]nonan-8-yl]-4,5,6,7-tetrahydro-2H-pyrazolo[4,3-b]pyridine-3-carboxamide;
7-[4-(but-2-ynoyl)piperazin-1-yl]-2-(4-phenoxyphenyl)-4,5,6,7-tetrahydro-2H-pyrazolo[4,3-b]pyridine-3-carboxamide;
2-[4-(4-chlorophenoxy)phenyl]-7-[1-(prop-2-enoyl)piperidin-4-yl]-4,5,6,7-tetrahydro-2H-pyrazolo[4,3-b]pyridine-3-carboxamide;
(7S)-2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide;
(7R)-2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide;
(7S)-7-[(2S)-2-methyl-4-(prop-2-enoyl)piperazin-1-yl]-2-(4-phenoxyphenyl)-4,5,6,7-tetrahydro-2H-pyrazolo[4,3-b]pyridine-3-carboxamide;
(7R)-7-[(2S)-2-methyl-4-(prop-2-enoyl)piperazin-1-yl]-2-(4-phenoxyphenyl)-4,5,6,7-tetrahydro-2H-pyrazolo[4,3-b]pyridine-3-carboxamide;
2-(4-phenoxyphenyl)-7-[(1R,4R)-5-(prop-2-enoyl)-2,5-diazabicyclo[2.2.1]heptan-2-yl]-4,5,6,7-tetrahydro-2H-pyrazolo[4,3-b]pyridine-3-carboxamide;
2-(4-phenoxyphenyl)-7-[(1S,4S)-5-(prop-2-enoyl)-2,5-diazabicyclo[2.2.1]heptan-2-yl]-4,5,6,7-tetrahydro-2H-pyrazolo[4,3-b]pyridine-3-carboxamide;
2-(4-phenoxyphenyl)-7-[(1R,5S)-8-(prop-2-enoyl)-3,8-diazabicyclo[3.2.1]octan-3-yl]-4,5,6,7-tetrahydro-2H-pyrazolo[4,3-b]pyridine-3-carboxamide;
2-[4-(2,4-difluorophenoxy)phenyl]-7-[4-(prop-2-enoyl)piperazin-1-yl]-4,5,6,7-tetrahydro-2H-pyrazolo[4,3-b]pyridine-3-carboxamide;
2-(2-fluoro-4-phenoxyphenyl)-7-[1-(prop-2-enoyl)piperidin-4-yl]-4,5,6,7-tetrahydro-2H-pyrazolo[4,3-b]pyridine-3-carboxamide;
2-[4-(2-fluorophenoxy)phenyl]-7-[4-(prop-2-enoyl)piperazin-1-yl]-4,5,6,7-tetrahydro-2H-pyrazolo[4,3-b]pyridine-3-carboxamide;
(7S)-7-[4-(but-2-ynoyl)piperazin-1-yl]-2-(4-phenoxyphenyl)-4,5,6,7-tetrahydro-2H-pyrazolo[4,3-b]pyridine-3-carboxamide;
(7R)-7-[4-(but-2-ynoyl)piperazin-1-yl]-2-(4-phenoxyphenyl)-4,5,6,7-tetrahydro-2H-pyrazolo[4,3-b]pyridine-3-carboxamide;
2-(4-phenoxyphenyl)-7-[3-(prop-2-enoyl)-3,6-diazabicyclo[3.1.1]heptan-6-yl]-4,5,6,7-tetrahydro-2H-pyrazolo[4,3-b]pyridine-3-carboxamide;
7-[(3R)-3-methyl-4-(prop-2-enoyl)piperazin-1-yl]-2-(4-phenoxyphenyl)-4,5,6,7-tetrahydro-2H-pyrazolo[4,3-b]pyridine-3-carboxamide;
7-[(3S)-3-methyl-4-(prop-2-enoyl)piperazin-1-yl]-2-(4-phenoxyphenyl)-4,5,6,7-tetrahydro-2H-pyrazolo[4,3-b]pyridine-3-carboxamide;
(7R)-2-[4-(4-hydroxyphenoxy)phenyl]-7-[4-(prop-2-enoyl)piperazin-1-yl]-4,5,6,7-tetrahydro-2H-pyrazolo[4,3-b]pyridine-3-carboxamide;
2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide;
(7R)-7-{4-[(2E)-4-(dimethylamino)but-2-enoyl]piperazin-1-yl}-2-(4-phenoxyphenyl)-4,5,6,7-tetrahydro-2H-pyrazolo[4,3-b]pyridine-3-carboxamide;
(7R)-7-{4-[(2E)-but-2-enoyl]piperazin-1-yl}-2-(4-phenoxyphenyl)-4,5,6,7-tetrahydro-2H-pyrazolo[4,3-b]pyridine-3-carboxamide;
(7R)-7-{4-[(2E)-4-amino-4-oxobut-2-enoyl]piperazin-1-yl}-2-(4-phenoxyphenyl)-4,5,6,7-tetrahydro-2H-pyrazolo[4,3-b]pyridine-3-carboxamide;
7-[4-(fluoroacetyl)piperazin-1-yl]-2-(4-phenoxyphenyl)-4,5,6,7-tetrahydro-2H-pyrazolo[4,3-b]pyridine-3-carboxamide;
(7R)-7-{4-[(2E)-3-ethoxyprop-2-enoyl]piperazin-1-yl}-2-(4-phenoxyphenyl)-4,5,6,7-tetrahydro-2H-pyrazolo[4,3-b]pyridine-3-carboxamide;
methyl (2E)-4-{4-[3-carbamoyl-2-(4-phenoxyphenyl)-4,5,6,7-tetrahydro-2H-pyrazolo[4,3-b]pyridin-7-yl]piperazin-1-yl}-4-oxobut-2-enoate;
2-(2-chloro-4-phenoxyphenyl)-7-[4-(prop-2-enoyl)piperazin-1-yl]-4,5,6,7-tetrahydro-2H-pyrazolo[4,3-b]pyridine-3-carboxamide;
2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide;
7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide;
2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide;
2-[4-(4-fluorophenoxy)phenyl]-7-[4-(prop-2-enoyl)piperazin-1-yl]-4,5,6,7-tetrahydro-2H-pyrazolo[4,3-b]pyridine-3-carboxamide;
2-[4-(3-fluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide;
(7S)-7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide;
2-[4-(3-fluorophenoxy)phenyl]-7-[1-(prop-2-enoyl)azetidin-3-yl]-4,5,6,7-tetrahydro-2H-pyrazolo[3,4-b]pyrazine-3-carboxamide;
2-[4-(3,4-difluorophenoxy)phenyl]-7-[1-(prop-2-enoyl)azetidin-3-yl]-4,5,6,7-tetrahydro-2H-pyrazolo[3,4-b]pyrazine-3-carboxamide;
(7R)-2-[4-(3-cyanophenoxy)phenyl]-7-[4-(prop-2-enoyl)piperazin-1-yl]-4,5,6,7-tetrahydro-2H-pyrazolo[4,3-b]pyridine-3-carboxamide;
(7R)-7-[4-(prop-2-enoyl)piperazin-1-yl]-2-{4-[3-(trifluoromethyl)phenoxy]phenyl}-4,5,6,7-tetrahydro-2H-pyrazolo[4,3-b]pyridine-3-carboxamide;
2-(4-phenoxyphenyl)-7-[rac-(3aR,6aS)-5-(prop-2-enoyl)hexahydropyrrolo[3,4-c]pyrrol-2(1H)-yl]-4,5,6,7-tetrahydro-2H-pyrazolo[4,3-b]pyridine-3-carboxamide;
(7R)-2-[4-(3-fluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2-cyclopropylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2-cyanophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2,4-difluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(4-fluorophenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide;
(7R)-2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide;
(7R)-2-[4-(2,5-difluorophenoxy)phenyl]-7-[4-(prop-2-enoyl)piperazin-1-yl]-4,5,6,7-tetrahydro-2H-pyrazolo[4,3-b]pyridine-3-carboxamide;
2-[4-(2-fluorophenoxy)phenyl]-7-[1-(prop-2-enoyl)azetidin-3-yl]-4,5,6,7-tetrahydro-2H-pyrazolo[3,4-b]pyrazine-3-carboxamide;
2-[4-(2,3-difluorophenoxy)phenyl]-7-[1-(prop-2-enoyl)azetidin-3-yl]-4,5,6,7-tetrahydro-2H-pyrazolo[3,4-b]pyrazine-3-carboxamide;
(7R)-2-[4-(2,3-difluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(3-cyclopropylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2-fluorophenoxy)phenyl]-7-[4-(prop-2-enoyl)piperazin-1-yl]-4,5,6,7-tetrahydro-2H-pyrazolo[4,3-b]pyridine-3-carboxamide;
2-[4-(2,5-difluorophenoxy)phenyl]-7-[1-(prop-2-enoyl)azetidin-3-yl]-4,5,6,7-tetrahydro-2H-pyrazolo[3,4-b]pyrazine-3-carboxamide;
2-[4-(2,4-difluorophenoxy)phenyl]-7-[1-(prop-2-enoyl)azetidin-3-yl]-4,5,6,7-tetrahydro-2H-pyrazolo[3,4-b]pyrazine-3-carboxamide;
(7R)-2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide;
(7S)-2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide;
7-[4-hydroxy-1-(prop-2-enoyl)piperidin-4-yl]-2-(4-phenoxyphenyl)-4,5,6,7-tetrahydro-2H-pyrazolo[4,3-b]pyridine-3-carboxamide;
(7R)-7-[4-(prop-2-enoyl)piperazin-1-yl]-2-{4-[4-(trifluoromethoxy)phenoxy]phenyl}-4,5,6,7-tetrahydro-2H-pyrazolo[4,3-b]pyridine-3-carboxamide;
(7R)-2-[4-(2-methylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(4-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(3-methylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-7-[4-(prop-2-enoyl)piperazin-1-yl]-2-{4-[3-(trifluoromethoxy)phenoxy]phenyl}-4,5,6,7-tetrahydro-2H-pyrazolo[4,3-b]pyridine-3-carboxamide;
(7R)-7-[4-(prop-2-enoyl)piperazin-1-yl]-2-{4-[2-(trifluoromethoxy)phenoxy]phenyl}-4,5,6,7-tetrahydro-2H-pyrazolo[4,3-b]pyridine-3-carboxamide;
(7R)-2-[4-(3-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(4-methylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(2-methylphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(3-methylphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(4-methylphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(2-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(3-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(4-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7S)-7-[4-(prop-2-enoyl)piperazin-1-yl]-2-{4-[2-(trifluoromethoxy)phenoxy]phenyl}-4,5,6,7-tetrahydro-2H-pyrazolo[4,3-b]pyridine-3-carboxamide;
(7S)-7-[4-(prop-2-enoyl)piperazin-1-yl]-2-{4-[3-(trifluoromethoxy)phenoxy]phenyl}-4,5,6,7-tetrahydro-2H-pyrazolo[4,3-b]pyridine-3-carboxamide; and
(7S)-7-[4-(prop-2-enoyl)piperazin-1-yl]-2-{4-[4-(trifluoromethoxy)phenoxy]phenyl}-4,5,6,7-tetrahydro-2H-pyrazolo[4,3-b]pyridine-3-carboxamide;
or a pharmaceutically acceptable salt thereof.

Exemplary compounds of formula (I) include, but are not limited to, the compounds shown in Table 1 below, and pharmaceutically acceptable salts thereof.

TABLE 1
Exemplary Compounds

| # | Structure |
|---|---|
| 1 | 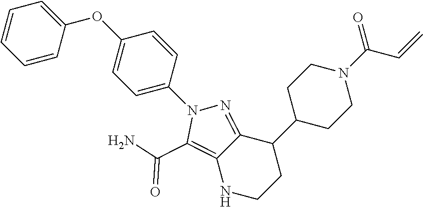 [SMILES: C=CC(=O)N1CCC(C2CCNc3c2nn(-c2ccc(Oc4ccccc4)cc2)c3C(N)=O)CC1] |
| 2 | 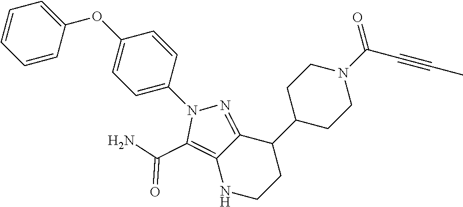 [SMILES: CC#CC(=O)N1CCC(C2CCNc3c2nn(-c2ccc(Oc4ccccc4)cc2)c3C(N)=O)CC1] |
| 3 | 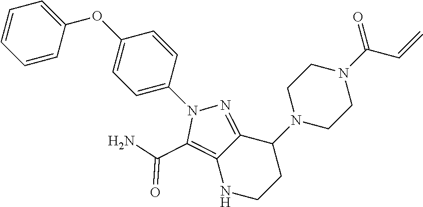 [SMILES: C=CC(=O)N1CCN(C2CCNc3c2nn(-c2ccc(Oc4ccccc4)cc2)c3C(N)=O)CC1] |
| 4 | 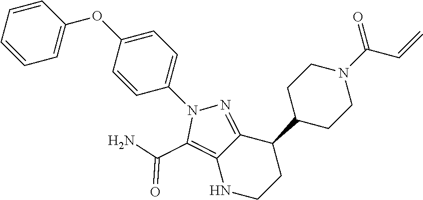 [SMILES: C=CC(=O)N1CCC([C@H]2CCNc3c2nn(-c2ccc(Oc4ccccc4)cc2)c3C(N)=O)CC1] |
| 5 | 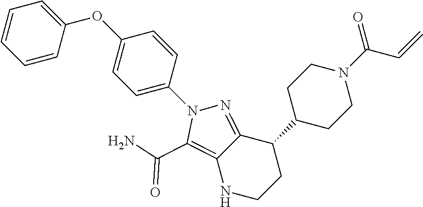 [SMILES: C=CC(=O)N1CCC([C@@H]2CCNc3c2nn(-c2ccc(Oc4ccccc4)cc2)c3C(N)=O)CC1] |
| 6 | 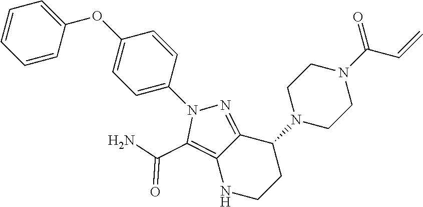 [SMILES: C=CC(=O)N1CCN([C@@H]2CCNc3c2nn(-c2ccc(Oc4ccccc4)cc2)c3C(N)=O)CC1] |
| 7 | 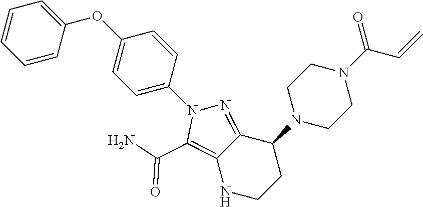 [SMILES: C=CC(=O)N1CCN([C@H]2CCNc3c2nn(-c2ccc(Oc4ccccc4)cc2)c3C(N)=O)CC1] |
| 8 | 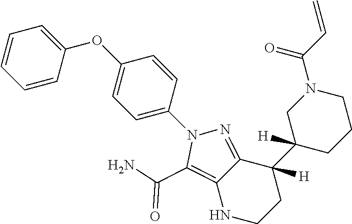 [SMILES: C=CC(=O)N1CCC[C@H]([C@@H]2CCNc3c2nn(-c2ccc(Oc4ccccc4)cc2)c3C(N)=O)C1] |
| 9 | 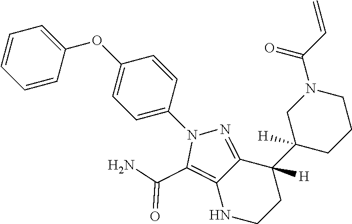 [SMILES: C=CC(=O)N1CCC[C@@H]([C@@H]2CCNc3c2nn(-c2ccc(Oc4ccccc4)cc2)c3C(N)=O)C1] |
| 10 | 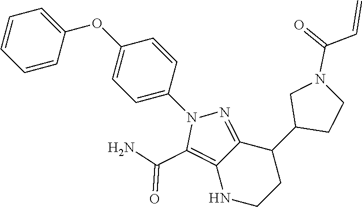 [SMILES: C=CC(=O)N1CCC(C2CCNc3c2nn(-c2ccc(Oc4ccccc4)cc2)c3C(N)=O)C1] |
| 11 | 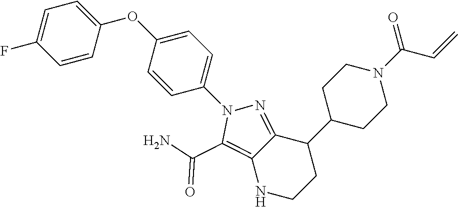 [SMILES: C=CC(=O)N1CCC(C2CCNc3c2nn(-c2ccc(Oc4ccc(F)cc4)cc2)c3C(N)=O)CC1] |
| 12 | 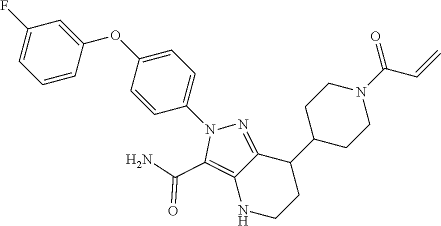 [SMILES: C=CC(=O)N1CCC(C2CCNc3c2nn(-c2ccc(Oc4cccc(F)c4)cc2)c3C(N)=O)CC1] |
| 13 | 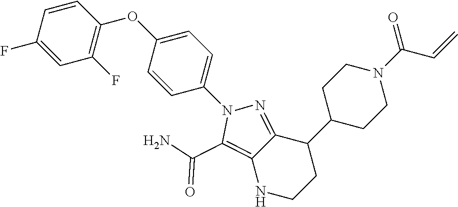 [SMILES: C=CC(=O)N1CCC(C2CCNc3c2nn(-c2ccc(Oc4ccc(F)cc4F)cc2)c3C(N)=O)CC1] |
| 14 | 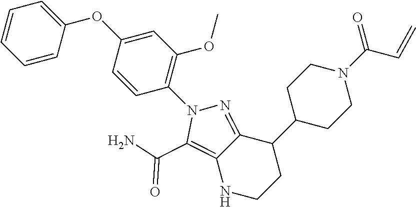 [SMILES: C=CC(=O)N1CCC(C2CCNc3c2nn(-c2ccc(Oc4ccccc4)cc2OC)c3C(N)=O)CC1] |
| 15 | 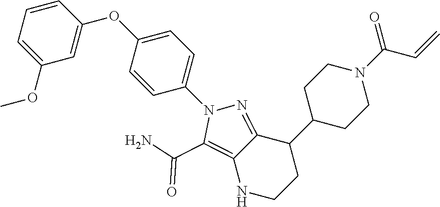 [SMILES: C=CC(=O)N1CCC(C2CCNc3c2nn(-c2ccc(Oc4cccc(OC)c4)cc2)c3C(N)=O)CC1] |
| 16 | 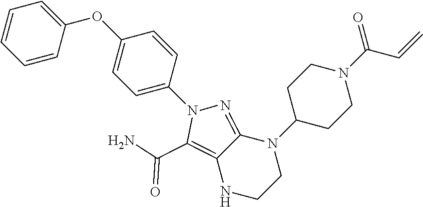 [SMILES: C=CC(=O)N1CCC(N2CCNc3c2nn(-c2ccc(Oc4ccccc4)cc2)c3C(N)=O)CC1] |
| 17 | 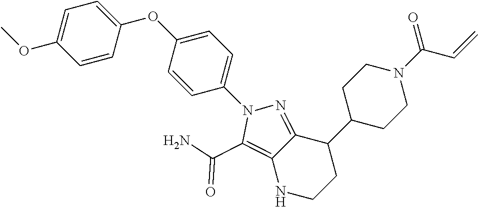 [SMILES: C=CC(=O)N1CCC(C2CCNc3c2nn(-c2ccc(Oc4ccc(OC)cc4)cc2)c3C(N)=O)CC1] |
| 18 | 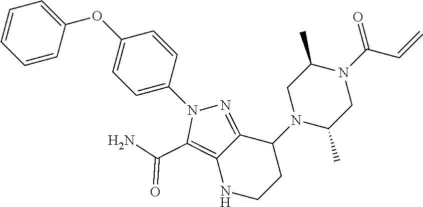 [SMILES: C=CC(=O)N1C[C@H](C)N(C2CCNc3c2nn(-c2ccc(Oc4ccccc4)cc2)c3C(N)=O)C[C@H]1C] |
| 19 | 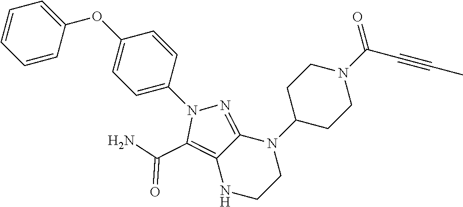 [SMILES: CC#CC(=O)N1CCC(N2CCNc3c2nn(-c2ccc(Oc4ccccc4)cc2)c3C(N)=O)CC1] |
| 20 | 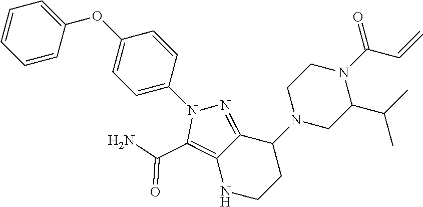 [SMILES: C=CC(=O)N1CCN(C2CCNc3c2nn(-c2ccc(Oc4ccccc4)cc2)c3C(N)=O)CC1C(C)C] |
| 21 | 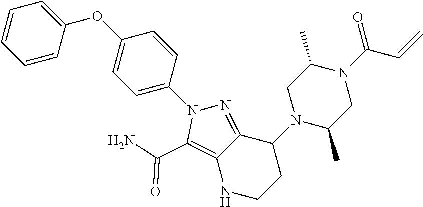 [SMILES: C=CC(=O)N1C[C@@H](C)N(C2CCNc3c2nn(-c2ccc(Oc4ccccc4)cc2)c3C(N)=O)C[C@@H]1C] |
| 22 | 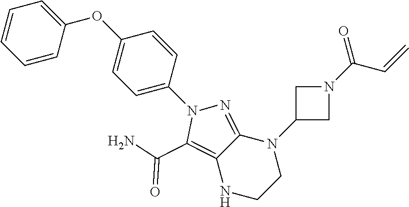 [SMILES: C=CC(=O)N1CC(N2CCNc3c2nn(-c2ccc(Oc4ccccc4)cc2)c3C(N)=O)C1] |
| 23 | 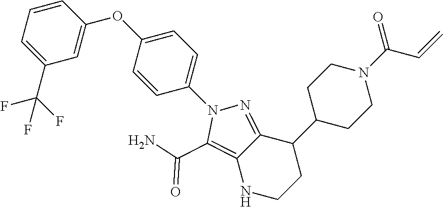 [SMILES: C=CC(=O)N1CCC(C2CCNc3c2nn(-c2ccc(Oc4cccc(C(F)(F)F)c4)cc2)c3C(N)=O)CC1] |
| 24 | 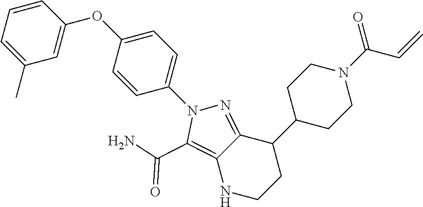 [SMILES: C=CC(=O)N1CCC(C2CCNc3c2nn(-c2ccc(Oc4cccc(C)c4)cc2)c3C(N)=O)CC1] |
| 25 | 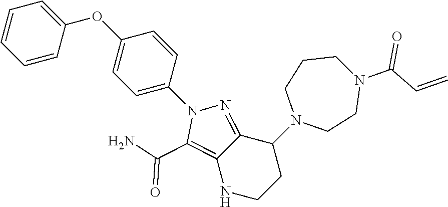 [SMILES: C=CC(=O)N1CCCN(C2CCNc3c2nn(-c2ccc(Oc4ccccc4)cc2)c3C(N)=O)CC1] |
| 26 | 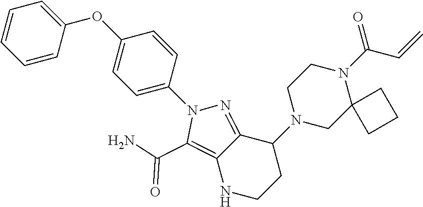 [SMILES: C=CC(=O)N1CCN(C2CCNc3c2nn(-c2ccc(Oc4ccccc4)cc2)c3C(N)=O)CC12CCC2] |
| 27 | 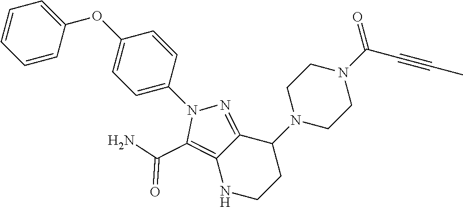 [SMILES: CC#CC(=O)N1CCN(C2CCNc3c2nn(-c2ccc(Oc4ccccc4)cc2)c3C(N)=O)CC1] |
| 28 | 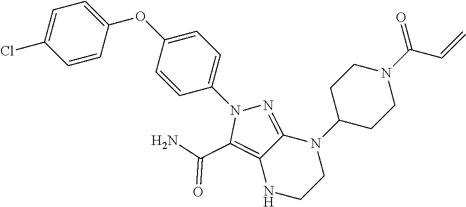 [SMILES: C=CC(=O)N1CCC(N2CCNc3c2nn(-c2ccc(Oc4ccc(Cl)cc4)cc2)c3C(N)=O)CC1] |
| 29 | 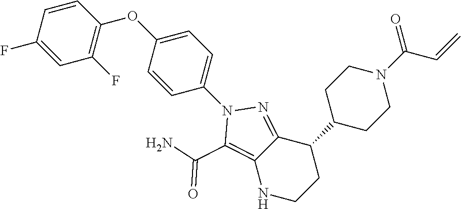 [SMILES: C=CC(=O)N1CCC([C@@H]2CCNc3c2nn(-c2ccc(Oc4ccc(F)cc4F)cc2)c3C(N)=O)CC1] |
| 30 | 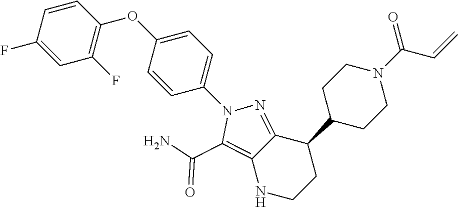 [SMILES: C=CC(=O)N1CCC([C@H]2CCNc3c2nn(-c2ccc(Oc4ccc(F)cc4F)cc2)c3C(N)=O)CC1] |
| 31 | 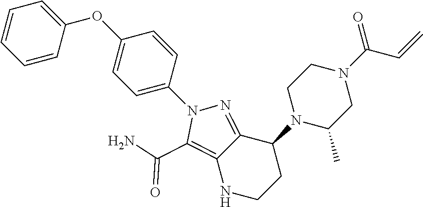 [SMILES: C=CC(=O)N1CCN([C@H]2CCNc3c2nn(-c2ccc(Oc4ccccc4)cc2)c3C(N)=O)[C@@H](C)C1] |
| 32 | 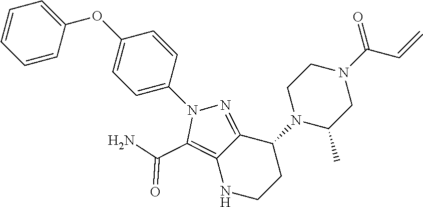 [SMILES: C=CC(=O)N1CCN([C@@H]2CCNc3c2nn(-c2ccc(Oc4ccccc4)cc2)c3C(N)=O)[C@@H](C)C1] |
| 33 | 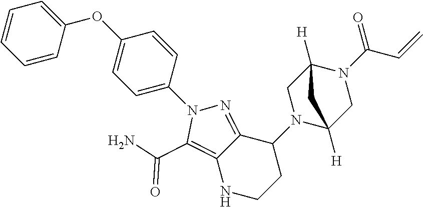 [SMILES: C=CC(=O)N1C[C@H]2C[C@@H]1CN2C1CCNc2c1nn(-c1ccc(Oc3ccccc3)cc1)c2C(N)=O] |
| 34 | 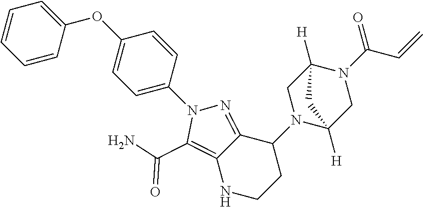 [SMILES: C=CC(=O)N1C[C@@H]2C[C@H]1CN2C1CCNc2c1nn(-c1ccc(Oc3ccccc3)cc1)c2C(N)=O] |
| 35 | 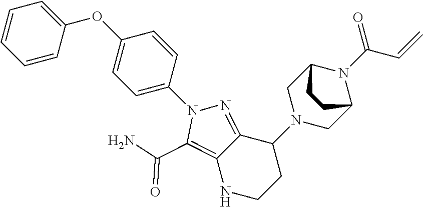 |
| 36 | 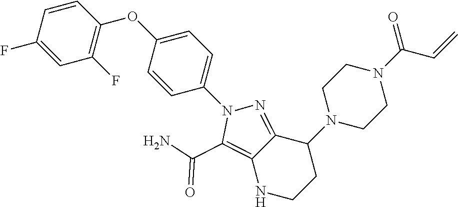 [SMILES: C=CC(=O)N1CCN(C2CCNc3c2nn(-c2ccc(Oc4ccc(F)cc4F)cc2)c3C(N)=O)CC1] |
| 37 | 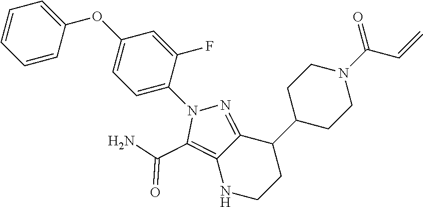 [SMILES: C=CC(=O)N1CCC(C2CCNc3c2nn(-c2ccc(Oc4ccccc4)cc2F)c3C(N)=O)CC1] |
| 38 | 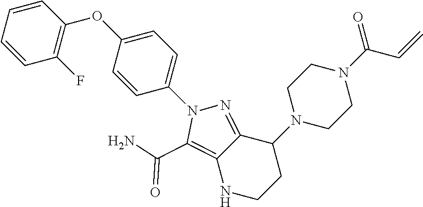 [SMILES: C=CC(=O)N1CCN(C2CCNc3c2nn(-c2ccc(Oc4ccccc4F)cc2)c3C(N)=O)CC1] |
| 39 | 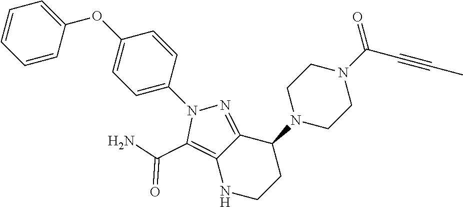 [SMILES: CC#CC(=O)N1CCN([C@H]2CCNc3c2nn(-c2ccc(Oc4ccccc4)cc2)c3C(N)=O)CC1] |
| 40 | 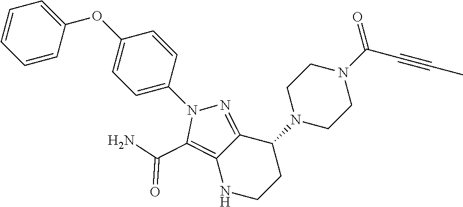 [SMILES: CC#CC(=O)N1CCN([C@@H]2CCNc3c2nn(-c2ccc(Oc4ccccc4)cc2)c3C(N)=O)CC1] |
| 41 | 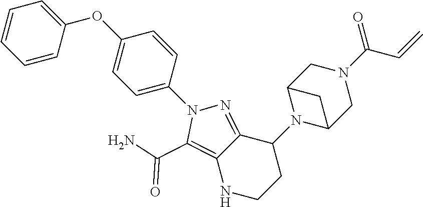 [SMILES: C=CC(=O)N1CC2CC(C1)N2C1CCNc2c1nn(-c1ccc(Oc3ccccc3)cc1)c2C(N)=O] |
| 42 | 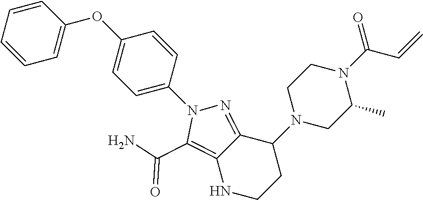 [SMILES: C=CC(=O)N1CCN(C2CCNc3c2nn(-c2ccc(Oc4ccccc4)cc2)c3C(N)=O)C[C@H]1C] |
| 43 | 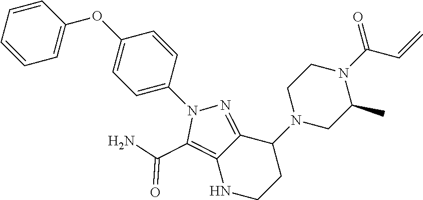 [SMILES: C=CC(=O)N1CCN(C2CCNc3c2nn(-c2ccc(Oc4ccccc4)cc2)c3C(N)=O)C[C@@H]1C] |
| 44 | 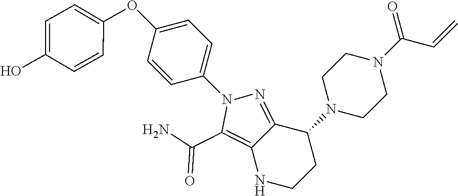 [SMILES: C=CC(=O)N1CCN([C@@H]2CCNc3c2nn(-c2ccc(Oc4ccc(O)cc4)cc2)c3C(N)=O)CC1] |
| 45 | 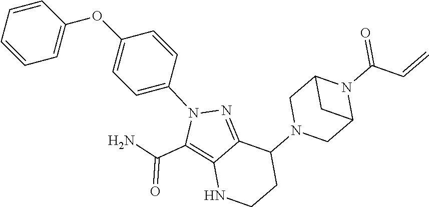 [SMILES: C=CC(=O)N1C2CC1CN(C1CCNc3c1nn(-c1ccc(Oc4ccccc4)cc1)c3C(N)=O)C2] |
| 46 | 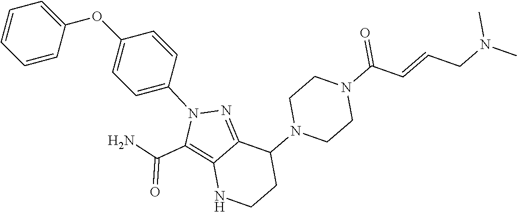 [SMILES: CN(C)C/C=C/C(=O)N1CCN(C2CCNc3c2nn(-c2ccc(Oc4ccccc4)cc2)c3C(N)=O)CC1] |
| 47 | 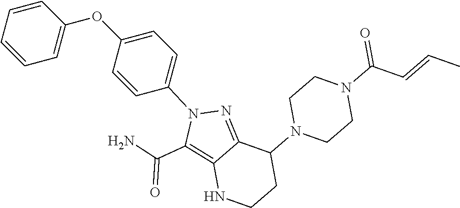 [SMILES: C/C=C/C(=O)N1CCN(C2CCNc3c2nn(-c2ccc(Oc4ccccc4)cc2)c3C(N)=O)CC1] |
| 48 | 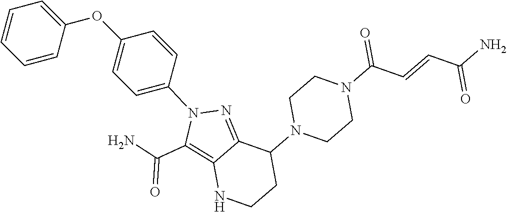 [SMILES: NC(=O)/C=C/C(=O)N1CCN(C2CCNc3c2nn(-c2ccc(Oc4ccccc4)cc2)c3C(N)=O)CC1] |
| 49 | 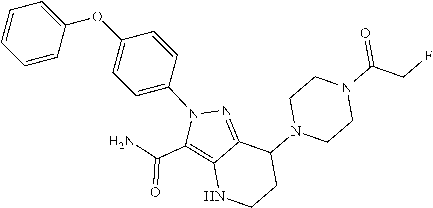 [SMILES: NC(=O)c1c2c(nn1-c1ccc(Oc3ccccc3)cc1)C(N1CCN(C(=O)CF)CC1)CCN2] |
| 50 | 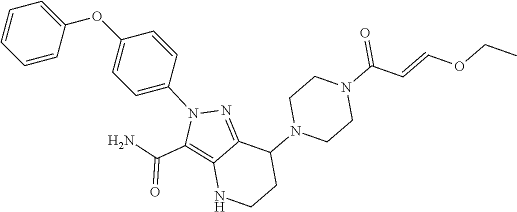 [SMILES: CCO/C=C/C(=O)N1CCN(C2CCNc3c2nn(-c2ccc(Oc4ccccc4)cc2)c3C(N)=O)CC1] |
| 51 | 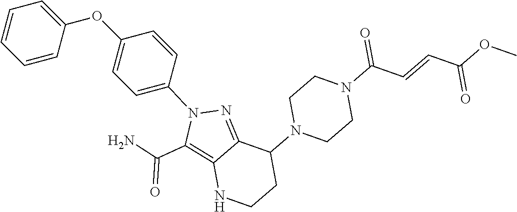 [SMILES: COC(=O)/C=C/C(=O)N1CCN(C2CCNc3c2nn(-c2ccc(Oc4ccccc4)cc2)c3C(N)=O)CC1] |
| 52 | 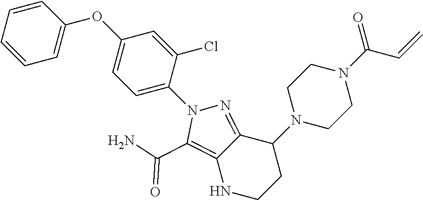 [SMILES: C=CC(=O)N1CCN(C2CCNc3c2nn(-c2ccc(Oc4ccccc4)cc2Cl)c3C(N)=O)CC1] |
| 53 | 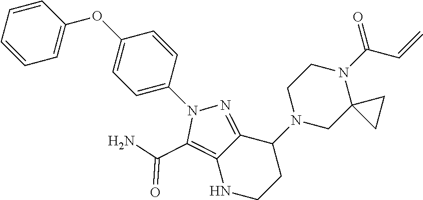 [SMILES: C=CC(=O)N1CCN(C2CCNc3c2nn(-c2ccc(Oc4ccccc4)cc2)c3C(N)=O)CC12CC2] |
| 54 | 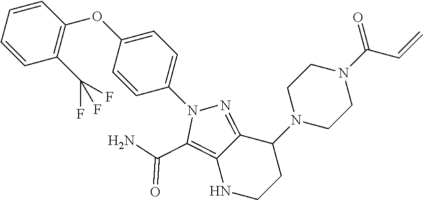 [SMILES: C=CC(=O)N1CCN(C2CCNc3c2nn(-c2ccc(Oc4ccccc4C(F)(F)F)cc2)c3C(N)=O)CC1] |
| 55 | 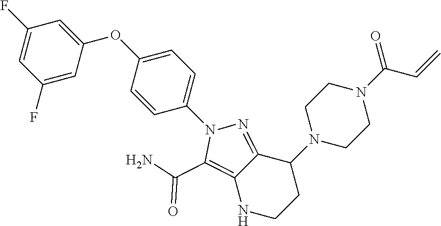 [SMILES: C=CC(=O)N1CCN(C2CCNc3c2nn(-c2ccc(Oc4cc(F)cc(F)c4)cc2)c3C(N)=O)CC1] |
| 56 | 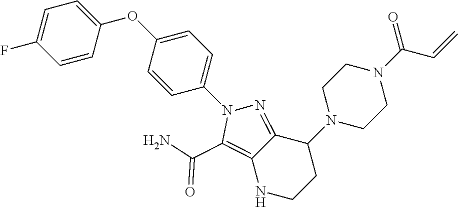 [SMILES: C=CC(=O)N1CCN(C2CCNc3c2nn(-c2ccc(Oc4ccc(F)cc4)cc2)c3C(N)=O)CC1] |
| 57 | 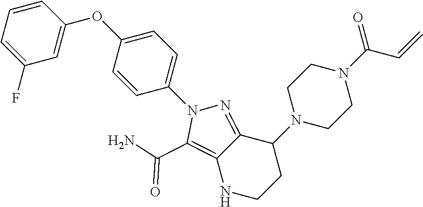 [SMILES: C=CC(=O)N1CCN(C2CCNc3c2nn(-c2ccc(Oc4cccc(F)c4)cc2)c3C(N)=O)CC1] |
| 59 | 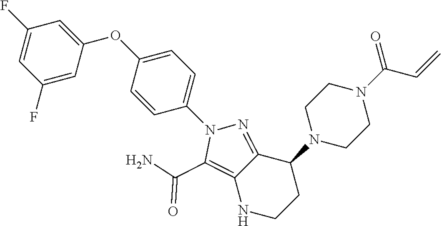 [SMILES: C=CC(=O)N1CCN([C@H]2CCNc3c2nn(-c2ccc(Oc4cc(F)cc(F)c4)cc2)c3C(N)=O)CC1] |
| 58 | 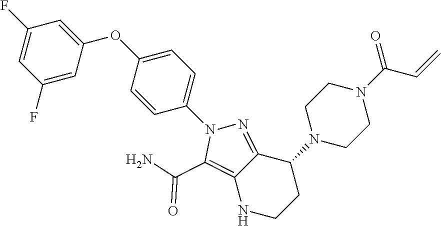 [SMILES: C=CC(=O)N1CCN([C@@H]2CCNc3c2nn(-c2ccc(Oc4cc(F)cc(F)c4)cc2)c3C(N)=O)CC1] |
| 60 | 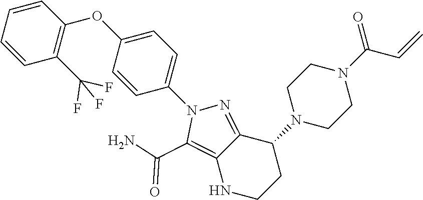 [SMILES: C=CC(=O)N1CCN([C@@H]2CCNc3c2nn(-c2ccc(Oc4ccccc4C(F)(F)F)cc2)c3C(N)=O)CC1] |
| 61 | 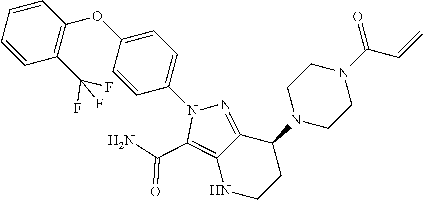 [SMILES: C=CC(=O)N1CCN([C@H]2CCNc3c2nn(-c2ccc(Oc4ccccc4C(F)(F)F)cc2)c3C(N)=O)CC1] |
| 62 | 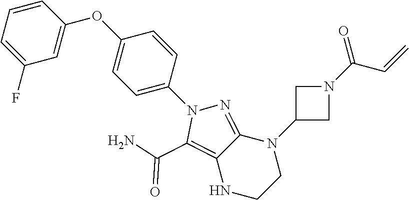 [SMILES: C=CC(=O)N1CC(N2CCNc3c2nn(-c2ccc(Oc4cccc(F)c4)cc2)c3C(N)=O)C1] |
| 63 | 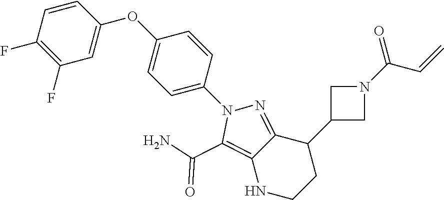 [SMILES: C=CC(=O)N1CC(C2CCNc3c2nn(-c2ccc(Oc4ccc(F)c(F)c4)cc2)c3C(N)=O)C1] |
| 64 | 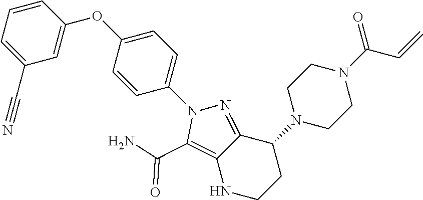 [SMILES: C=CC(=O)N1CCN([C@@H]2CCNc3c2nn(-c2ccc(Oc4cccc(C#N)c4)cc2)c3C(N)=O)CC1] |
| 65 | 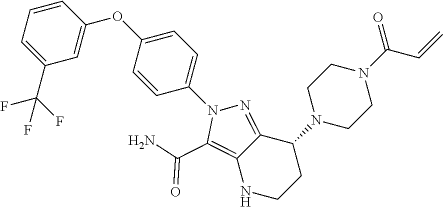 [SMILES: C=CC(=O)N1CCN([C@@H]2CCNc3c2nn(-c2ccc(Oc4cccc(C(F)(F)F)c4)cc2)c3C(N)=O)CC1] |
| 66 | 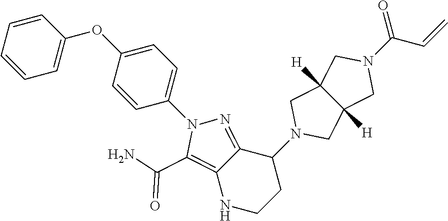 [SMILES: C=CC(=O)N1C[C@@H]2CN(C3CCNc4c3nn(-c3ccc(Oc5ccccc5)cc3)c4C(N)=O)C[C@@H]2C1] |
| 67 | 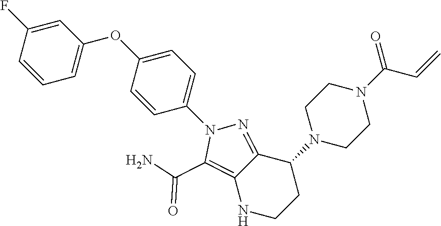 [SMILES: C=CC(=O)N1CCN([C@@H]2CCNc3c2nn(-c2ccc(Oc4cccc(F)c4)cc2)c3C(N)=O)CC1] |
| 68 | 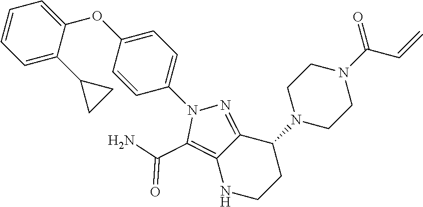 [SMILES: C=CC(=O)N1CCN([C@@H]2CCNc3c2nn(-c2ccc(Oc4ccccc4C4CC4)cc2)c3C(N)=O)CC1] |
| 69 | 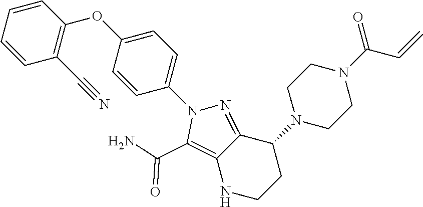 [SMILES: C=CC(=O)N1CCN([C@@H]2CCNc3c2nn(-c2ccc(Oc4ccccc4C#N)cc2)c3C(N)=O)CC1] |
| 70 | 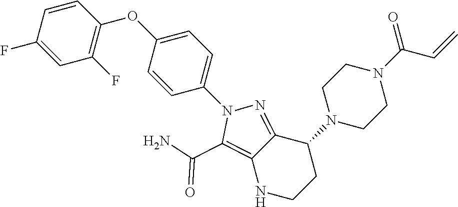 [SMILES: C=CC(=O)N1CCN([C@@H]2CCNc3c2nn(-c2ccc(Oc4ccc(F)cc4F)cc2)c3C(N)=O)CC1] |
| 71 | 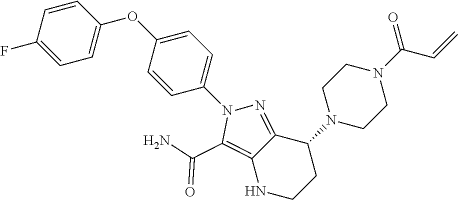 [SMILES: C=CC(=O)N1CCN([C@@H]2CCNc3c2nn(-c2ccc(Oc4ccc(F)cc4)cc2)c3C(N)=O)CC1] |
| 72 | 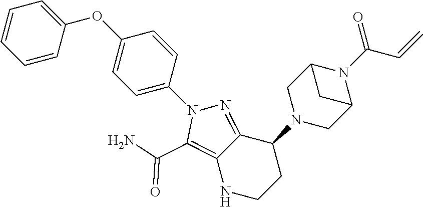 [SMILES: C=CC(=O)N1C2CC1CN([C@H]1CCNc3c1nn(-c1ccc(Oc4ccccc4)cc1)c3C(N)=O)C2] |
| 73 | 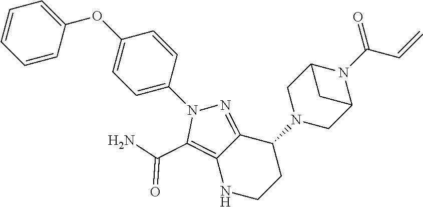 [SMILES: C=CC(=O)N1C2CC1CN([C@@H]1CCNc3c1nn(-c1ccc(Oc4ccccc4)cc1)c3C(N)=O)C2] |
| 74 | 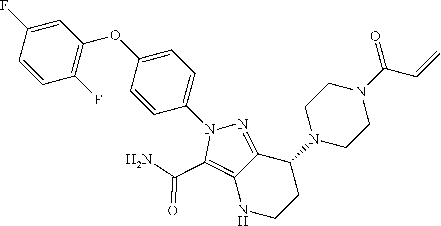 [SMILES: C=CC(=O)N1CCN([C@@H]2CCNc3c2nn(-c2ccc(Oc4cc(F)ccc4F)cc2)c3C(N)=O)CC1] |
| 75 | 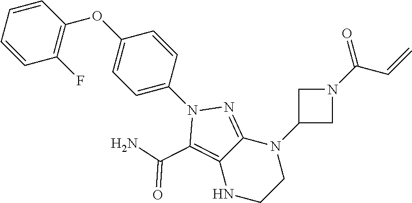 [SMILES: C=CC(=O)N1CC(N2CCNc3c2nn(-c2ccc(Oc4ccccc4F)cc2)c3C(N)=O)C1] |
| 76 | 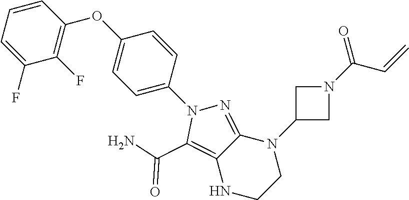 [SMILES: C=CC(=O)N1CC(N2CCNc3c2nn(-c2ccc(Oc4cccc(F)c4F)cc2)c3C(N)=O)C1] |
| 77 | 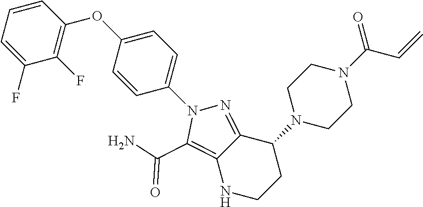 [SMILES: C=CC(=O)N1CCN([C@@H]2CCNc3c2nn(-c2ccc(Oc4cccc(F)c4F)cc2)c3C(N)=O)CC1] |
| 78 | 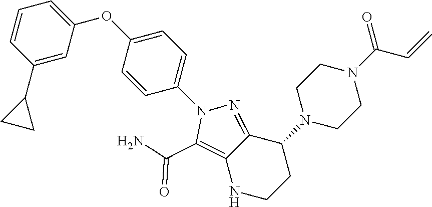 [SMILES: C=CC(=O)N1CCN([C@@H]2CCNc3c2nn(-c2ccc(Oc4cccc(C5CC5)c4)cc2)c3C(N)=O)CC1] |
| 79 | 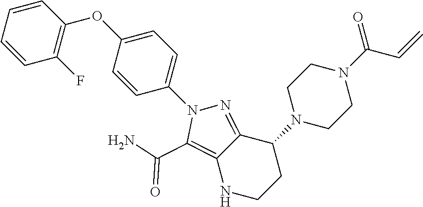 [SMILES: C=CC(=O)N1CCN([C@@H]2CCNc3c2nn(-c2ccc(Oc4ccccc4F)cc2)c3C(N)=O)CC1] |
| 80 | 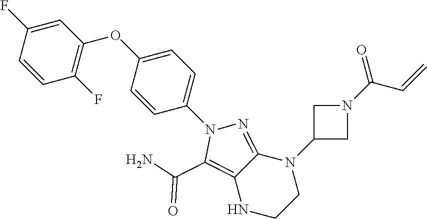 [SMILES: C=CC(=O)N1CC(N2CCNc3c2nn(-c2ccc(Oc4cc(F)ccc4F)cc2)c3C(N)=O)C1] |
| 81 | 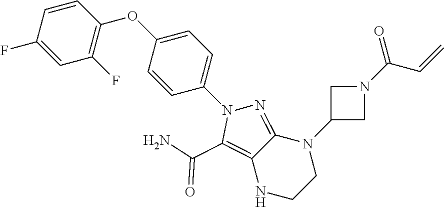 [SMILES: C=CC(=O)N1CC(N2CCNc3c2nn(-c2ccc(Oc4ccc(F)cc4F)cc2)c3C(N)=O)C1] |
| 82 | 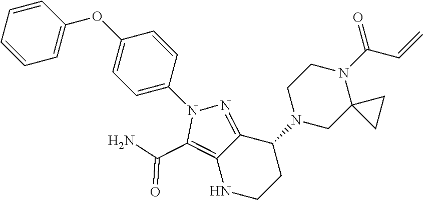 [SMILES: C=CC(=O)N1CCN([C@@H]2CCNc3c2nn(-c2ccc(Oc4ccccc4)cc2)c3C(N)=O)CC12CC2] |
| 83 | 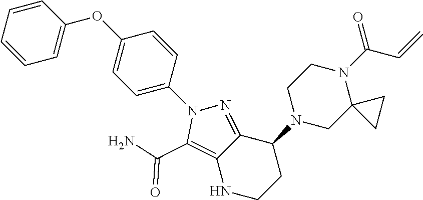 [SMILES: C=CC(=O)N1CCN([C@H]2CCNc3c2nn(-c2ccc(Oc4ccccc4)cc2)c3C(N)=O)CC12CC2] |
| 84 | 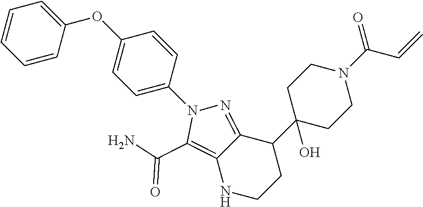 [SMILES: C=CC(=O)N1CCC(O)(C2CCNc3c2nn(-c2ccc(Oc4ccccc4)cc2)c3C(N)=O)CC1] |
| 85 | 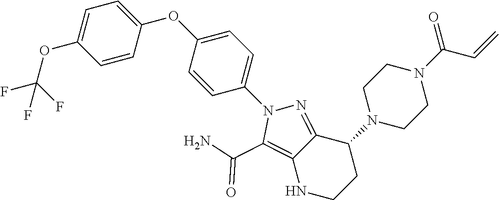 [SMILES: C=CC(=O)N1CCN([C@@H]2CCNc3c2nn(-c2ccc(Oc4ccc(OC(F)(F)F)cc4)cc2)c3C(N)=O)CC1] |
| 86 | 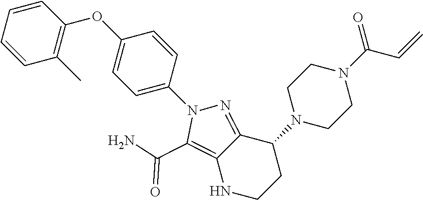 [SMILES: C=CC(=O)N1CCN([C@@H]2CCNc3c2nn(-c2ccc(Oc4ccccc4C)cc2)c3C(N)=O)CC1] |
| 87 | 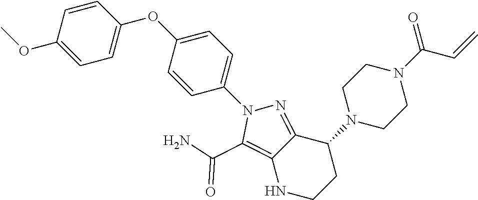 [SMILES: C=CC(=O)N1CCN([C@@H]2CCNc3c2nn(-c2ccc(Oc4ccc(OC)cc4)cc2)c3C(N)=O)CC1] |
| 88 | 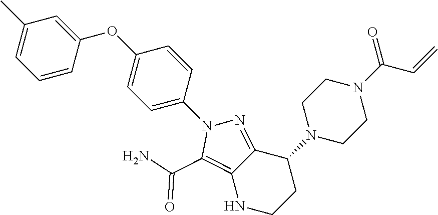 [SMILES: C=CC(=O)N1CCN([C@@H]2CCNc3c2nn(-c2ccc(Oc4cccc(C)c4)cc2)c3C(N)=O)CC1] |
| 89 | 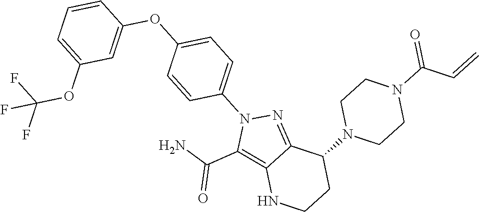 [SMILES: C=CC(=O)N1CCN([C@@H]2CCNc3c2nn(-c2ccc(Oc4cccc(OC(F)(F)F)c4)cc2)c3C(N)=O)CC1] |
| 90 | 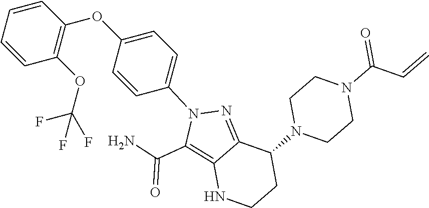 [SMILES: C=CC(=O)N1CCN([C@@H]2CCNc3c2nn(-c2ccc(Oc4ccccc4OC(F)(F)F)cc2)c3C(N)=O)CC1] |
| 91 | 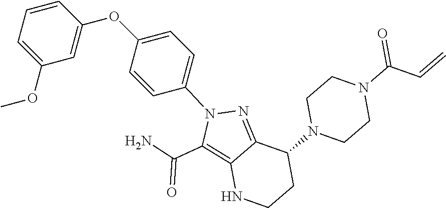 [SMILES: C=CC(=O)N1CCN([C@@H]2CCNc3c2nn(-c2ccc(Oc4cccc(OC)c4)cc2)c3C(N)=O)CC1] |
| 92 | 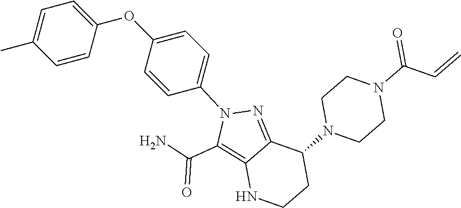 [SMILES: C=CC(=O)N1CCN([C@@H]2CCNc3c2nn(-c2ccc(Oc4ccc(C)cc4)cc2)c3C(N)=O)CC1] |
| 93 | 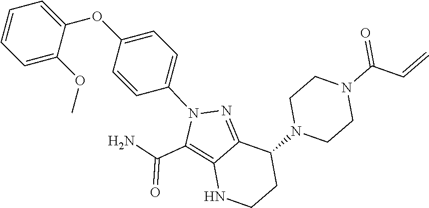 [SMILES: C=CC(=O)N1CCN([C@@H]2CCNc3c2nn(-c2ccc(Oc4ccccc4OC)cc2)c3C(N)=O)CC1] |
| 94 | 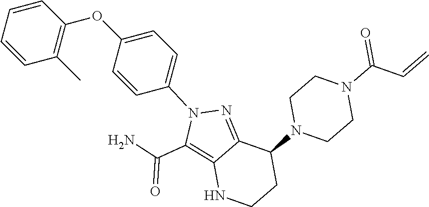 [SMILES: C=CC(=O)N1CCN([C@H]2CCNc3c2nn(-c2ccc(Oc4ccccc4C)cc2)c3C(N)=O)CC1] |
| 95 | 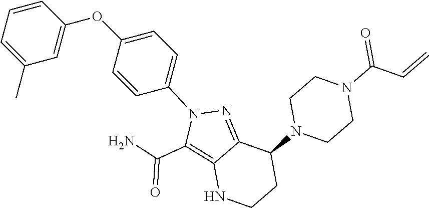 [SMILES: C=CC(=O)N1CCN([C@H]2CCNc3c2nn(-c2ccc(Oc4cccc(C)c4)cc2)c3C(N)=O)CC1] |
| 96 | 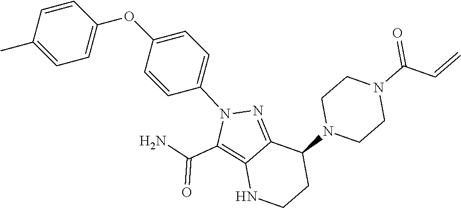 [SMILES: C=CC(=O)N1CCN([C@H]2CCNc3c2nn(-c2ccc(Oc4ccc(C)cc4)cc2)c3C(N)=O)CC1] |
| 97 | 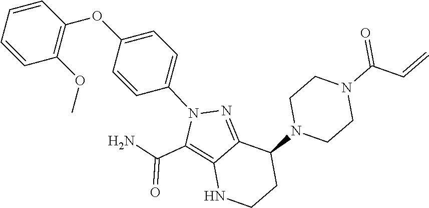 [SMILES: C=CC(=O)N1CCN([C@H]2CCNc3c2nn(-c2ccc(Oc4ccccc4OC)cc2)c3C(N)=O)CC1] |
| 98 | 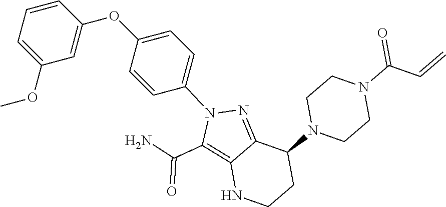 [SMILES: C=CC(=O)N1CCN([C@H]2CCNc3c2nn(-c2ccc(Oc4cccc(OC)c4)cc2)c3C(N)=O)CC1] |
| 99 | 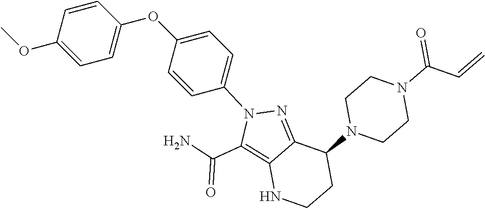 [SMILES: C=CC(=O)N1CCN([C@H]2CCNc3c2nn(-c2ccc(Oc4ccc(OC)cc4)cc2)c3C(N)=O)CC1] |
| 100 | 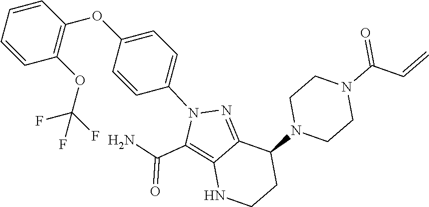 [SMILES: C=CC(=O)N1CCN([C@H]2CCNc3c2nn(-c2ccc(Oc4ccccc4OC(F)(F)F)cc2)c3C(N)=O)CC1] |
| 101 | 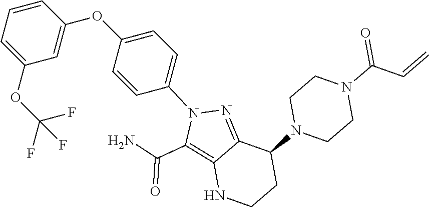 [SMILES: C=CC(=O)N1CCN([C@H]2CCNc3c2nn(-c2ccc(Oc4cccc(OC(F)(F)F)c4)cc2)c3C(N)=O)CC1] |
| 102 | 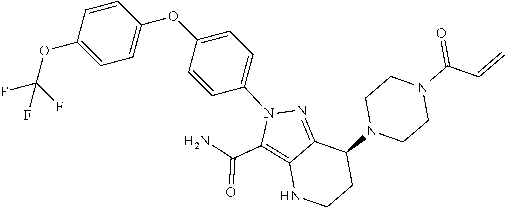 [SMILES: C=CC(=O)N1CCN([C@H]2CCNc3c2nn(-c2ccc(Oc4ccc(OC(F)(F)F)cc4)cc2)c3C(N)=O)CC1] |

Compound names are assigned by using Name 2019 by Advanced Chemical Development (ACD)/ChemSketch 2019.1.1 naming algorithm.

### General Synthesis

The compounds of the present disclosure can be better understood in connection with the following synthetic schemes and methods which illustrate a means by which the compounds can be prepared. Representative procedures are shown in, but are not limited to, Schemes 1-15. In Schemes 1-15, the variables R¹, R², R³, R⁴, m, n, and p, are as described in herein.

Starting materials useful for Schemes 1-15 are commercially available, may be prepared by the procedures described herein, by literature procedures, or by procedures that would be well known to one skilled in the art of organic chemistry. Further functionalization of any of the R groups in the Schemes 1-15 below (e.g. R¹, R²) may be performed, if desired, at any point in the reaction sequence using reactions known to one skilled in the art. In a second non-limiting example, an R group containing a halide may be reacted with an amine to give a substituted amine or an alcohol to give substituted ether. In a third non-limiting example, formation of ethers, carbamates, and esters may be prepared with a corresponding R group containing an alcohol. Deprotection of an R group to yield deprotected compounds may be performed using conditions known to one skilled in the art, and the deprotected compounds may then be reacted further as described above. Additionally, racemic and diastereomeric intermediates and examples may be separated into their corresponding isomers at any point in the reaction sequence using methods known to one skilled in the art or final compounds described herein (for example chiral supercritical fluid chromatography and classical chiral resolution).

Compounds of the present invention may be prepared according to the schemes set forth below. Methods for preparing tetrahydropyrazolopyridine-3-carboxamides 3-5 of the invention are illustrated in Schemes 1-3. As shown in Scheme 1, intermediates 1-7 may be prepared from compounds 1-1. Methoxymethyl protected 4-nitropyrazol-3-carboxlates 1-2, where R is alkyl, benzyl, or other suitable carboxylate protecting group, may be prepared from the corresponding 4-nitro-1H-pyrazole-3-carboxylates 1-2 using conditions such as those described for Intermediate A. Deprotonation of pyrazoles 1-2 with a lithium reagent such as lithium hexamethyldisilazide followed by addition of acetaldehyde affords secondary alcohols 1-3. Oxidation of 1-3 may be achieved using Dess-Martin periodinane or via other conditions to afford ketones 1-4. Reduction of nitropyrazoles 1-4 by Raney nickel provides intermediates 1-5. Aminopyrazoles 1-5 may be treated with methyl or ethyl formate and sodium methoxide to yield the pyrazolopyridines 1-6. Treatment of pyrazolopyridine alcohols 1-6 with trichlorophosphine affords arylchlorides 1-7.

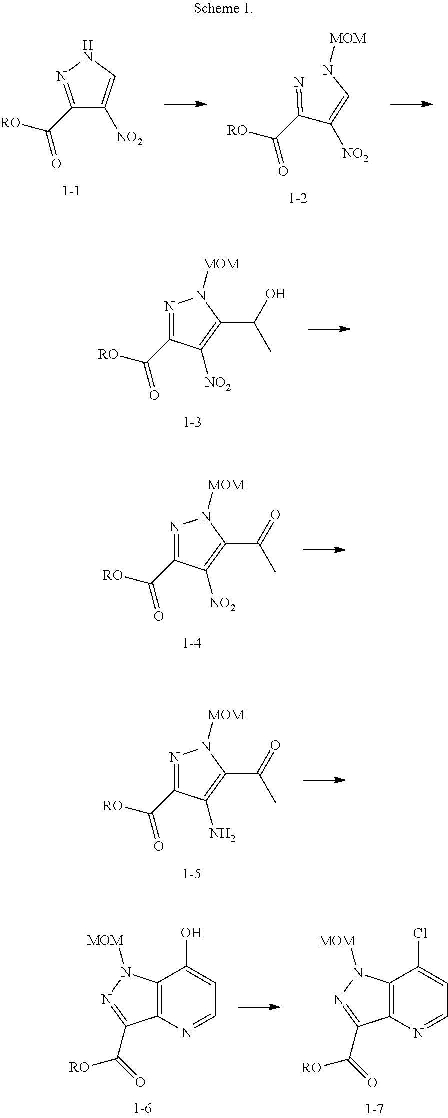

As shown in Scheme 2, compounds 2-4 may be prepared from compounds 1-7. Chloropyrazolopyridines 1-7 may undergo a variety of reactions including, but not limited to, a nucleophilic aromatic substitution reaction (for example, Step A.7) with an amine, such as tert-butyl piperazine-1-carboxylates, to give pyrazolopyridines 2-1. The Boc and MOM protecting groups of intermediates 2-1 may be simultaneously removed by methods such as those described in Step A.7 to give deprotected derivatives 2-2. Reprotection of piperazines 2-2 may be realized using di-tert-butyl dicarbonate in the presence of base. Catalytic hydrogenation of pyrazolopyridines 2-3 with a metal catalyst such as Pd/C affords tetrahydropyrazolopyridines 2-4.

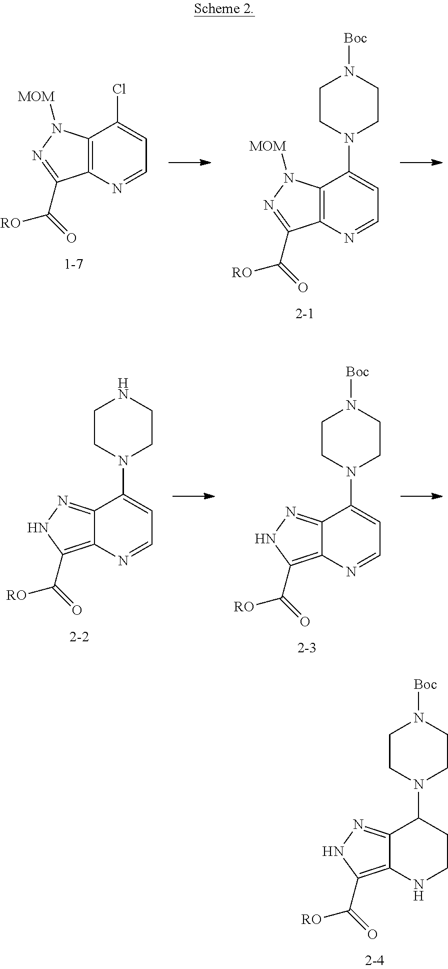

As shown in Scheme 3, compounds 3-5 may be prepared from compounds 1-7. (Phenoxyphenyl)boronic acids (boronic acids and/or boronic esters are commercially available, i.e., for example, Intermediate G and Intermediate Q) may be coupled with compounds 2-4 in the presence of cupric acetate (see for example, Step 55.1) to afford compounds of formula 3-1. Subsequent hydrolysis of esters 3-1 using, for example, Step 55.2, affords carboxylic acids 3-2. Carboxylic acids 3-2 may be converted to primary amides 3-3 as shown using conditions such as those described in Step 55.3. The Boc protecting group of 3-3 may be removed by methods such as those described in Step 12.3 to give phenylpyrazole derivatives 3-4. Acylation of the amines 3-4 with acids of formula R⁴CO₂H using reaction methods as described in Step 55.5 provides amides 3-5.

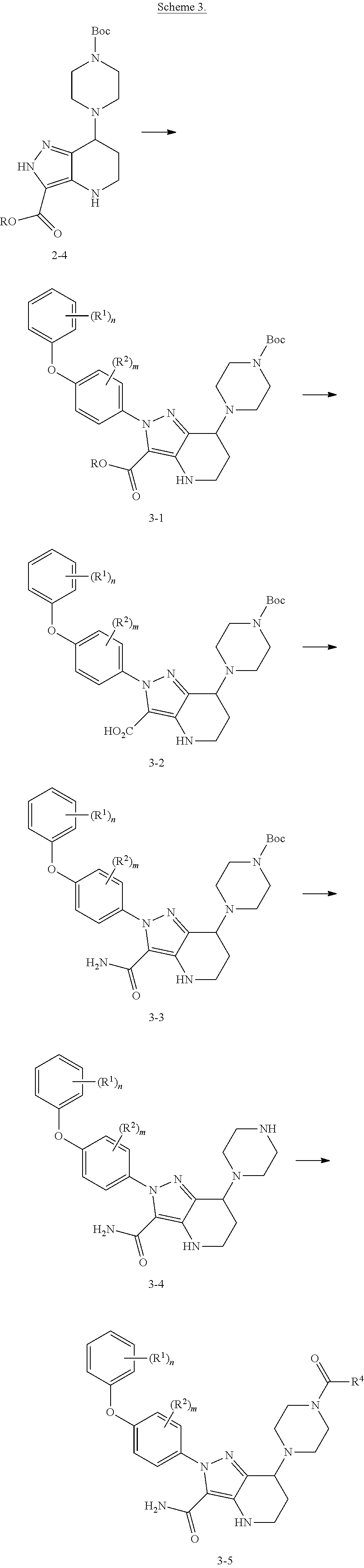

Methods for preparing tetrahydropyrazolopyridine-3-carboxamides 5-4 and 5-5 are illustrated in Schemes 4 and 5. Tetrahydropyran protected pyrazole diester 4-2 may be prepared from compound 4-1 where R is alkyl, benzyl, or other suitable carboxylate protecting group using conditions described for Step L.1 for example. Buchwald-Hartwig amination of bromopyrazole 4-2 with a 3-aminopropanoate affords aminopyrazoles 4-3 where R′ is alkyl, benzyl, or other suitable carboxylate protecting group. Subsequent cyclization of 4-3 to ketone 4-4 may be realized upon addition of lithium hexamethyldisilazide followed by addition of HCl. Condensation of the appropriate enantiopure Ellman's sulfinamides with ketones 4-4 yields the corresponding N-tert-butanesulfinyl ketimines 4-5. Reduction of N-tert-butanesulfinyl ketimines 4-5 using NaBH₄ affords the corresponding amine diastereomer 4-6. Deprotection of the tert-butanesulfinyl group followed by bisalkylation with (((2-nitrophenyl)sulfonyl)azanediyl)bis(ethane-2,1-diyl) bis(2-nitrobenzenesulfonate) provides the enantiomer enriched tetrahydropyrazolopyridines 4-7.

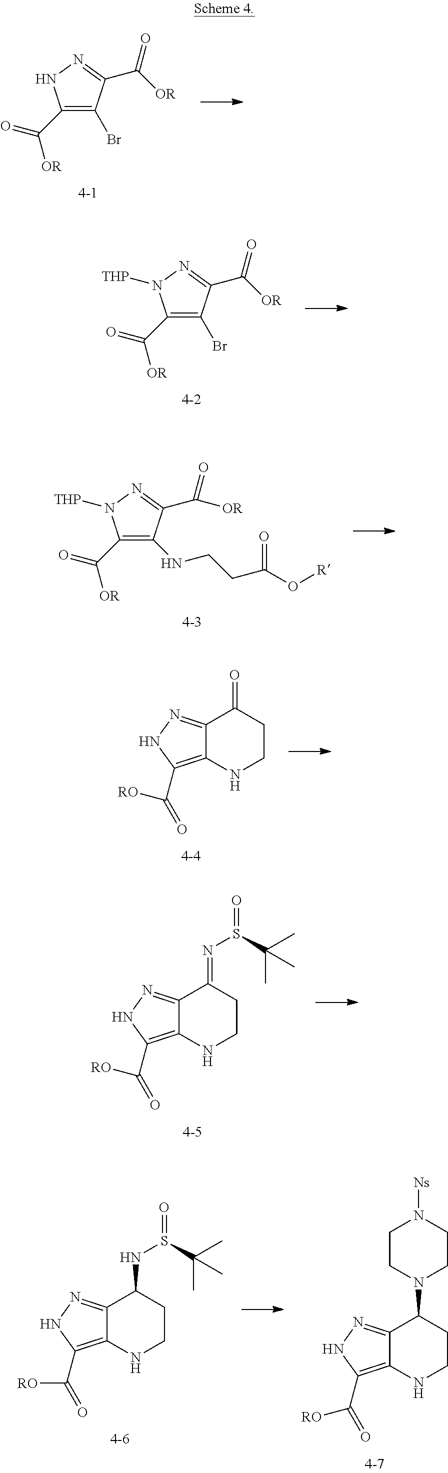

As shown in Scheme 5, compound 5-5 may be prepared from compound 4-7. The biphenyl ether moieties may be installed via a Chan-Lam coupling of substituted (4-phenoxyphenyl)boronic acids (boronic acids and/or boronic esters are commercially available or may be prepared by methods known to one skilled in the art) with compound 4-7 to afford compounds 5-1. Hydrolysis of esters 5-1 using conditions such as those described in Step 78.2 and subsequent amidation affords primary amides 5-2 as shown using conditions such as those described in Step 78.3 or Step 100.2. Deprotection of the nosyl (Ns) protecting group by methods such as those described in Step 69.4, Step 74.4, Step 100.3, or conditions known to one skilled in the art affords piperazine intermediates 5-3. Acylation of the piperazine with acids of formula R⁴CO₂H using reaction using methods as described in Step 55.5 provides amides 5-4. Late stage oxidation of amides 5-4 with liver microsomes (for example, Example 44) yields alcohols of formula 5-5.

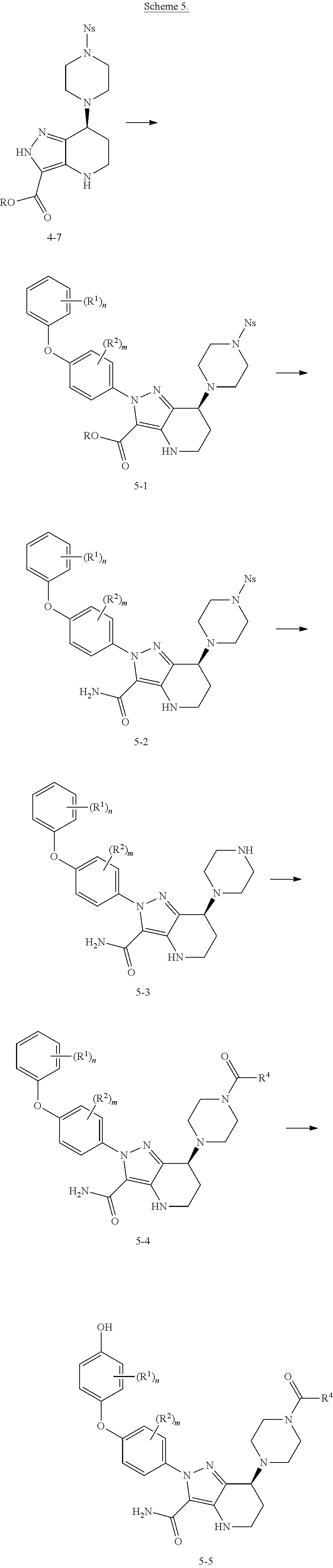

Methods for preparing 4-aminopyrazole-5-carboxamides 39 and 40 are illustrated in Schemes 6-8. α-Arylhydrazononitriles 6-2 may be synthesized from the coupling reaction of α-cyanocarbonyls with phenyl diazonium salts obtained commercially or prepared from arylamines 6-1 using conditions such as those described for Step B. Thorpe reaction of α-arylhydrazononitriles 6-2 with α-bromoester results in 4-aminopyrazoles 6-3, where R is alkyl, benzyl, or other suitable carboxylate protecting group. Cyclization of aminopyrazoles 6-3 with ethyl formate and NaH affords pyrazolopyridines 6-4. Treatment of pyrazolopyridine alcohols 6-4 with tribromophosphine affords bromides 6-5. Primary amides 6-7 may be obtained via hydrolysis of esters 6-5 to afford carboxylic acids 6-6 followed by the amidation using conditions for Steps B.5 and B.6. Nucleophilic aromatic substitution of bromide 6-7 with amine 6-8 where q and r are independently 0, 1, 2, or 3, including, for example tert-butyl piperazine-1-carboxylates (Step B.7), affords pyrazolopyridines 6-9. Catalytic hydrogenation (step B.8) of pyrazolopyridines 6-9 affords 6-10, which is deprotected using acetylchloride in methanol (step B.9), HCl (step 53.3) to afford 6-11.

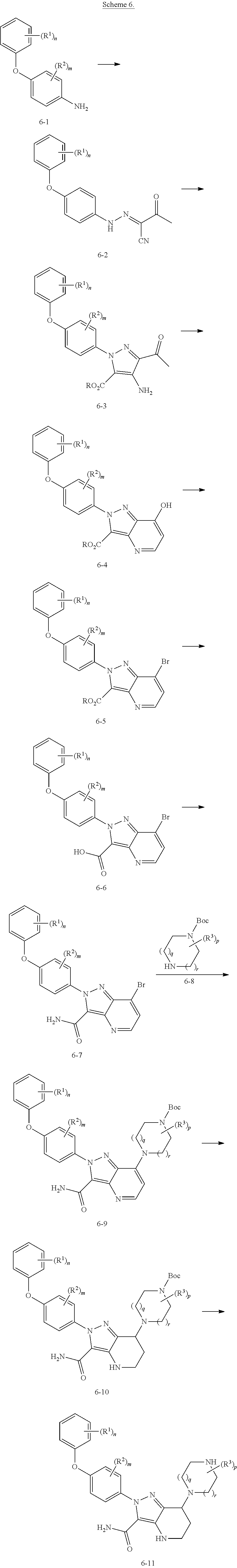

Alternatively, as shown in Scheme 7, compounds of formula 6-11 may be prepared from alcohols 6-4. Treatment of alcohols 6-4 with para-methoxybenzyl chloride (PMB-Cl) under basic conditions affords α,β-unsaturated ketones 7-1. Reduction of the double bond with lithium triethylborohydride affords ketones 7-2. Primary amides 7-4 may be prepared from esters 7-2 in two steps via 7-3 using methods for steps K.6 and K.7, or those described above. Reduction amination of the carbonyl group of derivatives 7-4 with various amines using conditions described for Step 43.1 affords tetrahydropyrazolopyridine-3-carboxamides 7-5. Deprotection of the PMB group under acidic conditions described for Step 43.2 affords compounds 6-11.

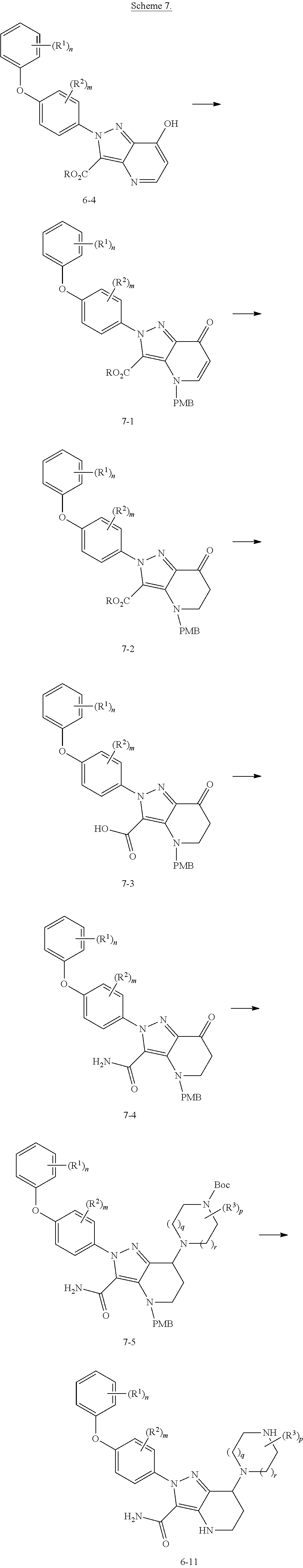

As shown in Scheme 8, acylation of 6-11 with acids of formula R⁴CO₂H using conditions known to one skilled in the art, Step 43.3, or those described above affords 8-1. Piperazines 6-11 may undergo classical resolution of the enantiomers using various chiral acids including, but not limited to (2R,3R)-2,3-bis((4-methylbenzoyl)oxy)succinic acid to afford the enriched chiral isomer 8-2. Single enantiomer 8-2 may be acylated with R⁴CO₂H using methods as those described above to afford 8-3.

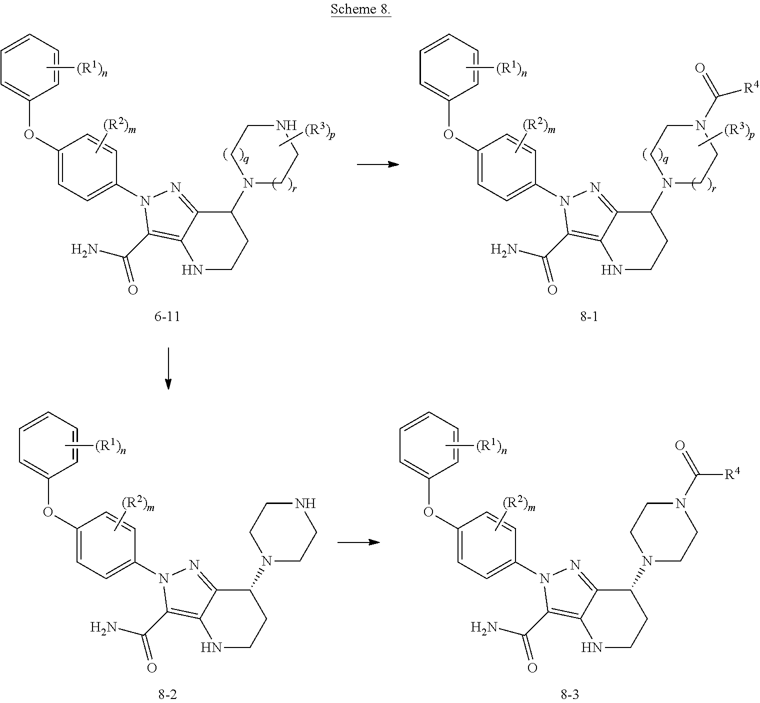

Methods for preparing tetrahydropyrazolopyridine-3-carboxamides 3-5 from amine 9-1 are illustrated in Scheme 9. Bromopyrazolopyridines 9-5 may be synthesized via intermediates 9-2, 9-3, and 9-4 in a similar manner as described in Scheme 6 for bromide 6-5, substituting various 4-bromoanilines 9-1 for arylamines 6-1. Subsequent hydrolysis of the ester 9-5 affords the acid 9-6. Conversion of 9-6 to the primary amide 9-7 followed by nucleophilic aromatic substitution using methods described in Steps K.5-K.7, methods as those described above provides aryl bromides 9-8. Copper-catalyzed diaryl ether formation from aryl bromides 9-8 and various phenols provides the Ullmann products 9-9 (Step 36.1). Derivatives 9-9 may be converted to tetrahydropyrazolopyridine-3-carboxamides 3-5 using methods illustrated in Example 36 or methods described above.

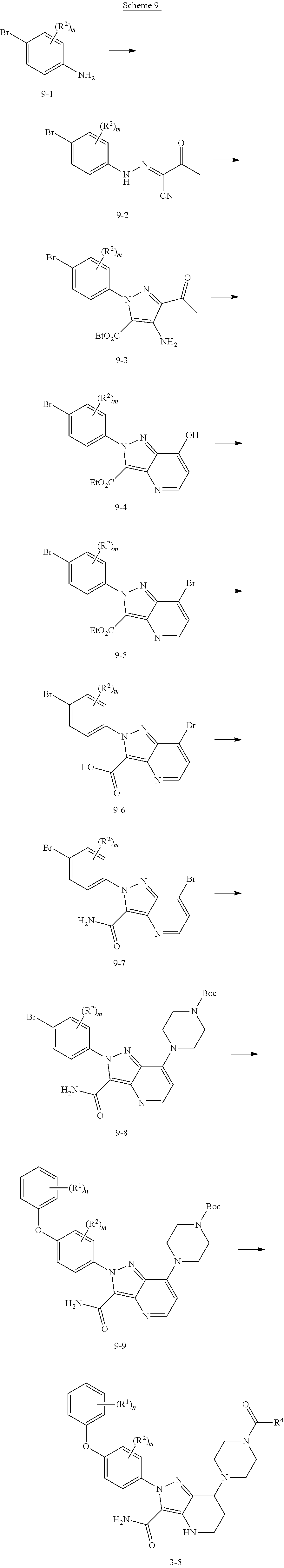

Alternatively, as shown in Scheme 10, nucleophilic aromatic substitution of bromopyrazolopyridines 9-5 with various amines using methods known to one skilled in the art provide intermediates 10-1. Treating aryl bromides 10-1 with [(2-di-tert-butylphosphino-3-methoxy-6-methyl-2′,4′,6′-triisopropyl-1,1′-biphenyl)-2-(2-aminobiphenyl)]palladium(II) methanesulfonate under basic conditions provides phenols 10-2 (Step 57.1). Pyrazolopyridines 10-2 undergo catalytic hydrogenation using Pd(OH)₂/C to provide tetrahydropyrazolopyridines 10-3. Diaryl ether formation from various aryl bromides and tetrahydropyrazolopyridines 10-3 affords Ullmann products 10-4 (Step 57.1) which may be further converted to tetrahydropyrazolopyridine-3-carboxamides 3-5 using methods illustrated in Example 57 or methods described above.

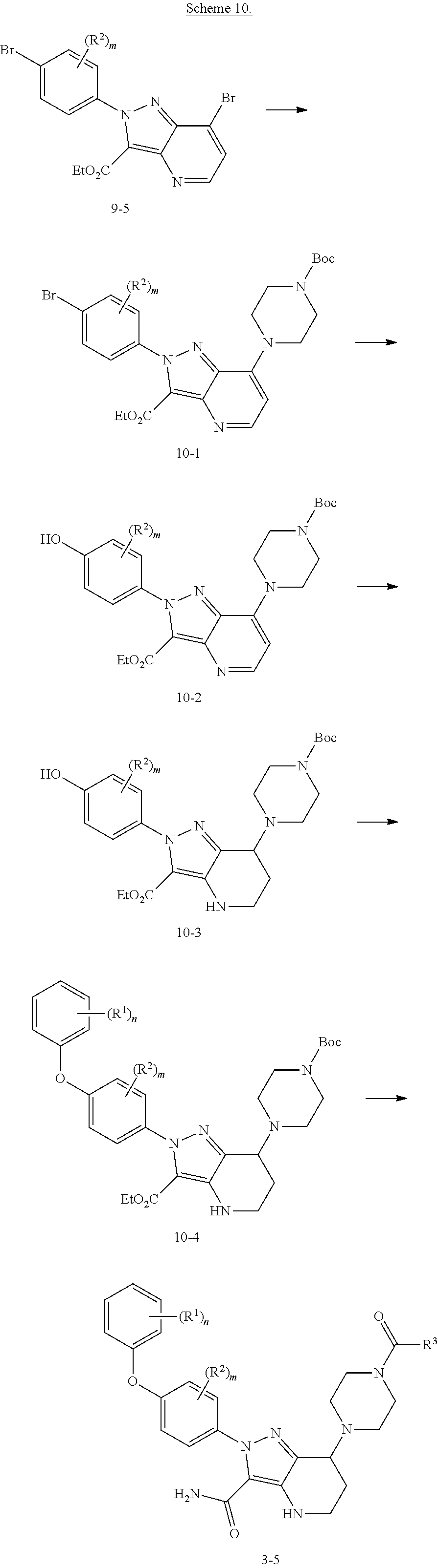

Methods for preparing tetrahydropyrazolopyridine-3-carboxamides 11-10 from 11-1 are illustrated in Scheme 11. Addition of deprotonated acetonitrile to esters 11-1 affords α-cyanoketones 11-2 (Step T1). Like Scheme 6 above, α-arylhydrazononitriles 11-3 may be synthesized from the coupling reaction of α-cyanocarbonyls 11-2 with phenyl diazonium salts obtained commercially or prepared from arylamines using conditions such as those described in Step C.2. The Thorpe reaction of α-arylhydrazononitriles 11-3 with an α-bromoester provides pyrazoles 11-4. Using methods outlined for Steps C.5-C.7 or above, 11-4 may be cyclized to 11-5, hydrolyzed to afford 11-6, and then amidated to afford key intermediates 11-7. Intermediate 11-7 may be subjected to hydrogenation conditions using catalytic Pd/C to afford tetrahydropyrazolopyridine-3-carboxamides 11-8. Deprotection of the Boc group under acidic conditions affords deprotected derivatives 11-9. Acylation of 11-9 with acids of formula R⁴CO₂H using reaction methods as described in Step 11.6 affords the product 11-10. When X¹ of 11-7 is a halogen such as, a bromide, Ullman products may be obtained in one of two methods. The first method provides the Ullmann products 11-11 by reacting aryl bromides 11-7 with various phenols (Step 12.1). Alternatively, primary amides 11-7 may undergo dehydration with trifluoroacetic acid to afford the corresponding nitriles 11-12 which when treated under Ullman conditions as described in Example 11.2 to afford Ullman products 11-13. Hydrolysis of nitriles 11-13 with hydrogen peroxide gives primary amides 11-11 (for example, using Example 11, Step 11.3). Primary amides 11-1 may be further diversified as described above.

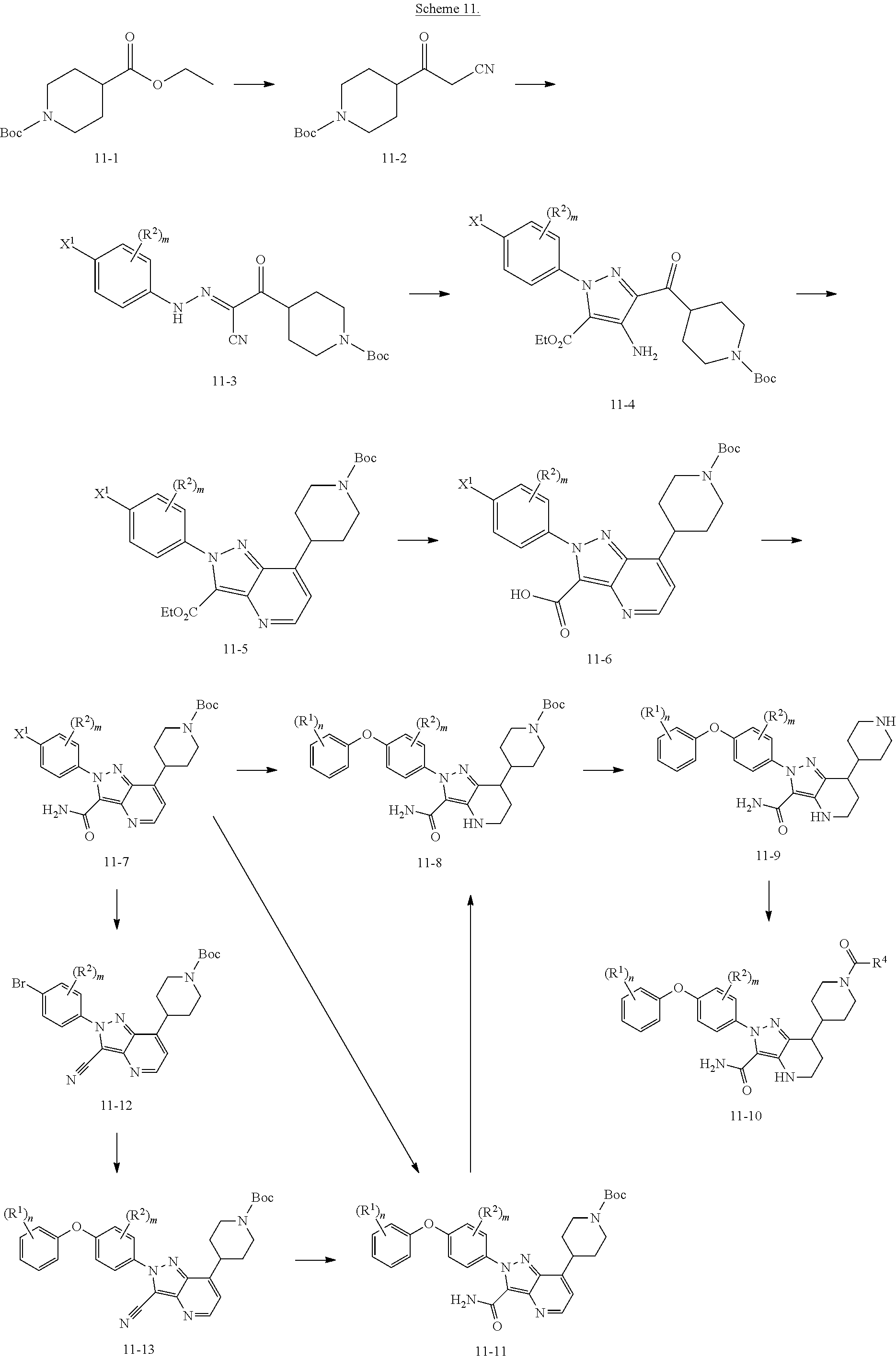

Methods for preparing tetrahydropyrazolopyridine-3-carboxamides 12-7 from alcohols 6-4 are illustrated in Scheme 12. Alcohol 6-4 may be converted to the corresponding trifate 12-1 using triflic anhydride in the presence of base. Compounds of formula 12-2 may be prepared from triflates 12-1 via a Suzuki coupling with an heterocycloalkylene or heterocycloalkenylene boronic acid or boronate and base such as potassium phosphate. Tertiary alcohol 12-3 may be prepared by reacting the double bond of 12-2 with tris(2,2,6,6-tetramethyl-3,5-heptanedionato)manganese(III) in the presence of phenyl silane. Hydrogenation of 12-3 using Pd(OH)₂/C affords tetrahydropyrazolopyridine 12-4. Conversion of the ester 12-4 using a one pot, two step reaction described in Step 66.2, to afford the primary amide 12-5. The Boc protecting group of 12-5 may be removed using HCl to give deprotected tetrahydropyrazolopyridine-3-carboxamides 12-6. Acylation of 12-6 with acids of formula R⁴CO₂H as described in Step 66.4 provides amides 12-7.

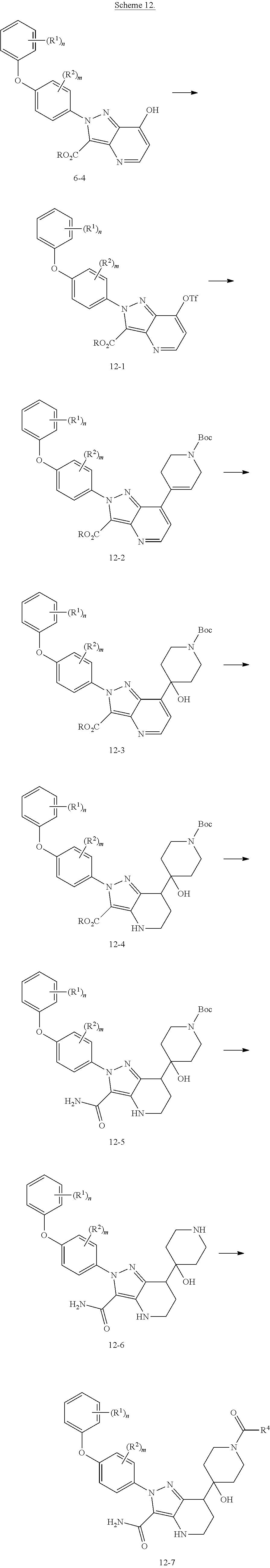

Methods for preparing chiral tetrahydropyrazolopyridine-3-carboxamides 5-4 and 13-5 are illustrated in Scheme 13. Direct amination of ester 10-4 with ammonia under pressure provides primary amide 13-1. Catalytic hydrogenation using Pd(OH)₂/C followed by chiral supercritical fluid chromatography separation affords both tetrahydropyrazolopyridine-3-carboxamide enantiomers 13-2 and 13-3. Independent deprotection of the Boc group of 13-2 and 13-3 using acetyl chloride in methanol (Step 7.8) affords the deprotected enantiomers 5-3 and 13-4, respectively. Acylation of the piperidine moiety of each of 5-3 and 13-4 independently with R⁴CO₂H or using reaction using methods as described in Step 7.9 affords tetrahydropyrazolopyridine-3-carboxamides 5-4 and 13-5, respectively.

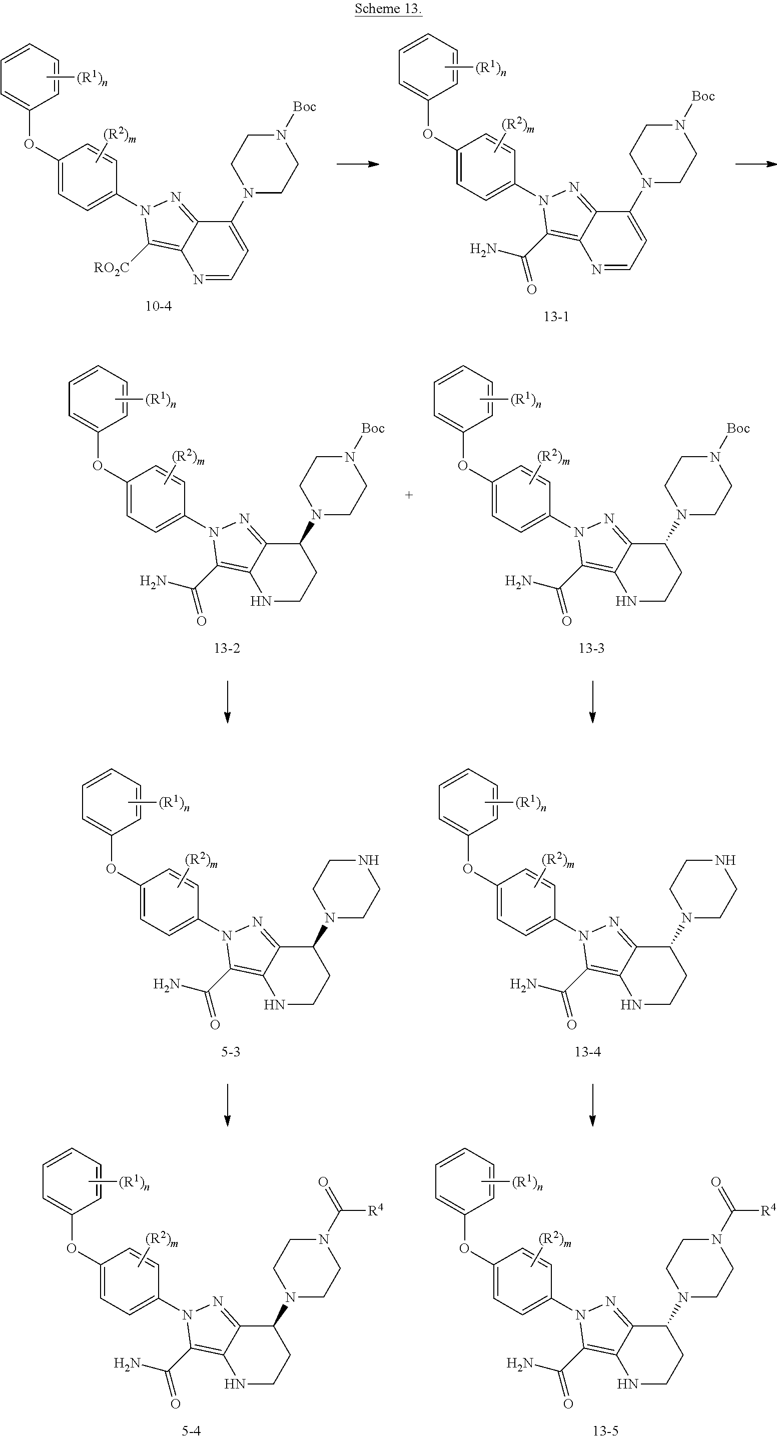

Methods for preparing tetrahydropyrazolopyridine-3-carboxamides 14-4 from bromide 6-7 are illustrated in Scheme 14. Suzuki coupling of aryl bromide 6-7 with an heterocycloalkylene or heterocycloalkenylene boronic acid or boronate and base such as potassium phosphate provides coupled products 14-1 where s is 1 or 2. Hydrogenation of 14-1 using Pd/C affords tetrahydropyrazolopyridines 14-2 (for example see, Step S.2). Removal of the Boc group of 14-2 using acidic conditions described for Step S.3 affords 14-3. Acylation of 14-3 with R⁴CO₂H using reaction using methods as illustrated in Example 8 provides amides 14-4.

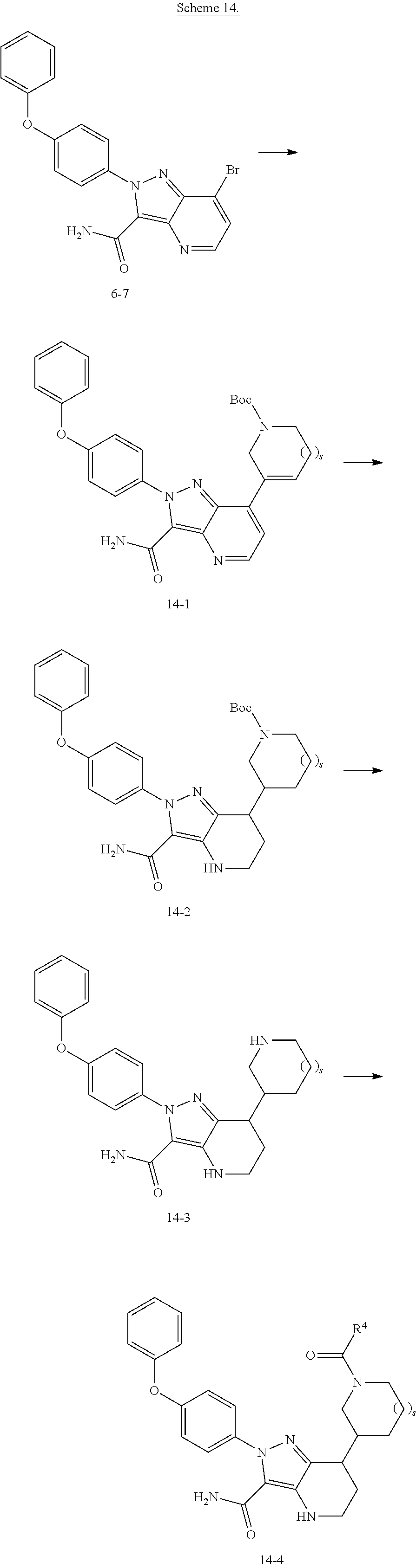

Methods for preparing tetrahydropyrazolepyrazine-3-carboxamides 15-12 are illustrated in Scheme 15. Commercially available 3,5-dibromo-4-nitro-1H-pyrazole, 15-1, may be protected with para-methoxybenzyl chloride and K₂CO₃ to afford 15-2. Bromo-4-nitropyrazoles 15-2 may undergo nucleophilic aromatic substitution with various mono-protected diamines using conditions such as those described in Step J.2 to afford compounds 15-3. Deprotonation of 15-3 with potassium hexamethyldisilazide followed by addition of allyl bromide provides N-alkylated products 15-4. Cyanation of 15-4 may be achieved via a range of conditions such as using a copper salt such as copper cyanide as illustrated in Step J.4 to afford nitriles 15-5. Subsequent hydrolysis of nitriles 15-5 with hydrogen peroxide affords primary amides 15-6 using methods such as for example, Step J.5. Treatment of intermediates 15-6 with trifluoroacetic acid and heat cleaves both the Boc and PMB protecting groups, which may be followed by reprotection with the Boc group using Boc anhydride and diisopropylethylamine (step J.6) to afford 15-7. The biphenyl ether moieties may be installed via a Chan-Lam coupling of substituted (4-phenoxyphenyl)boronic acids (boronic acids and/or boronic esters are either commercially available, or as described herein) with pyrazoles 15-7 in the presence of cupric acetate (for example, Step J.7) to afford 15-8. Alkenes 15-8 may be treated with osmium tetroxide to provide the corresponding vicinal diols which are treated with sodium periodate to provide aldehydes 15-9. Reduction of the nitro group in aldehyde intermediates 15-9 with palladium on carbon, Raney-Ni, or rhodium on carbon followed by an intramolecular ring closure affords tetrahydropyrazolepyrazine-3-carboxamides 15-10 (see examples Step J.9, Step 16.6, or Step 81.3). Removal of the Boc group of 15-10 using acidic conditions, such as acetyl chloride in methanol (described Step 16.7) affords deprotected intermediates 15-11. Acylation of amines 15-11 with acids of formula R⁴CO₂H using methods as described above or as illustrated in Step 16.8, affords tetrahydropyrazolepyrazines 15-12.

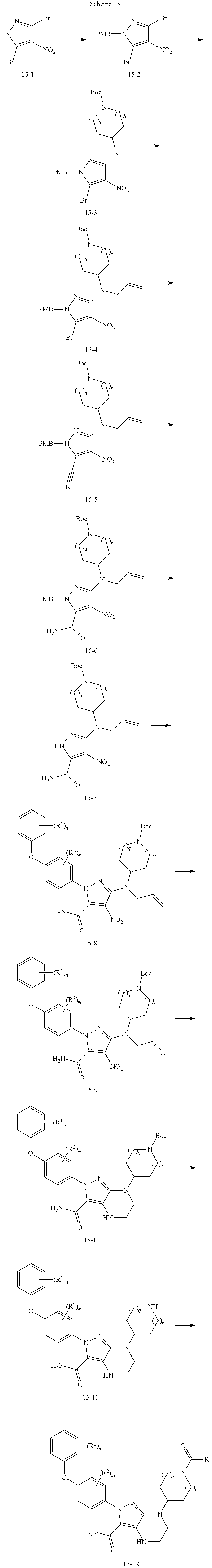

It can be appreciated that the synthetic schemes and specific examples as illustrated in the Examples section are illustrative and are not to be read as limiting the scope of the present disclosure as it is defined in the appended claims. All alternatives, modifications, and equivalents of the synthetic methods and specific examples are included within the scope of the claims.

The following Examples may be used for illustrative purposes and should not be deemed to narrow the scope of the present disclosure.

### EXAMPLES

### General

All reagents were of commercial grade and were used as received without further purification, unless otherwise stated. Commercially available anhydrous solvents were used for reactions conducted under inert atmosphere. Reagent grade solvents were used in all other cases, unless otherwise specified. Chemical shifts (δ) for ¹H NMR spectra were reported in parts per million (ppm) relative to tetramethylsilane (δ 0.00) or the appropriate residual solvent peak, i.e. CHCl₃ (δ 7.27), as internal reference.

Common abbreviations well known to those skilled in the art which are used throughout include those in Table 2.

TABLE 2
Abbreviations

| Abbreviation | Definition |
|---|---|
| NMR | nuclear magnetic resonance |
| s | singlet |
| br s | broad singlet |
| d | duplet or doublet |
| m | multiplet |
| t | triplet |
| q | quartet |
| min | minute |
| h | hour |
| mL | milliliter |
| μL | microliter |
| L | liter |
| g | gram |
| mg | milligram |
| mmol | millimoles |
| M | molarity (moles/liter) |
| μM | micromolar |
| N | normality (equivalent/liter) |
| ppm | parts per million |
| psi | pounds per square inch |
| rt | ambient temperature |
| HPLC | high pressure liquid chromatography |
| UPLC ® or UHPLC | ultra high performance liquid chromatography |
| LC/MS or LCMS | liquid chromatography- mass spectrometry |
| MS | mass spectrometry |
| APCI | atmospheric pressure chemical ionization |
| DCI | desorption chemical ionization |
| ESI | electrospray ionization |
| SFC | supercritical fluid chromatography |
| ATP | adenosine triphosphate |
| BSA | bovine serum albumin |
| EDTA | ethylenediaminetetraacetic acid |
| DTT | dithiothreitol |
| FRET | fluorescence energy transfer |
| HEPES | (4-(2-hydroxyethyl)-1- |
| | piperazineethanesulfonic acid) |
| IC₅₀ | half maximal inhibitory concentration |
| Boc | tert-butoxycarboxyl |
| MOM | methoxymethyl |
| Ns or Nosyl | nitrobenzenesulfonyl |
| PMB | para-methoxybenzyl |
| THP | tetrahydropyranyl |
| XPhos | 2-dicyclohexylphosphino-2,4,6-triisopropylbiphenyl |
| Xantphos | 4,5-bis(diphenylphosphino)-9,9-dimethylxanthene |
| XPhos Pd G2 | chloro(2-dicyclohexylphosphino- |
| | 2′,4′,6′-triisopropyl-1,1′-biphenyl) |
| | [2-(2′-amino-1,1′-biphenyl)]palladium(II) |

### Intermediate A

### ethyl 7-[4-(tert-butoxycarbonyl)piperazin-1-yl]-2H-pyrazolo[4,3-b]pyridine-3-carboxylate

### Step A.1

### methyl 1-(methoxymethyl)-4-nitro-1H-pyrazole-3-carboxylate

A solution of methyl-4-nitro-1H-pyrazole-3-carboxylate (49.0 g, 286 mmol) in tetrahydrofuran (490 mL) was cooled to about 5° C. in an ice bath and treated with N-ethyl-N-(propan-2-yl)propan-2-amine (75 mL, 430 mmol) in one portion. Chloromethyl methyl ether (24.0 mL, 315 mmol) was added over about 3 minutes, and the resulting suspension was stirred in the ice bath for 5 minutes. The reaction was removed from the ice bath and stirred at ambient temperature for 1 hour, diluted with tert-butyl methyl ether (500 mL), stirred for 5 minutes, and the precipitate was collected by filtration and washed with tert-butyl methyl ether. The combined filtrates (about 1.4 L) were washed with 1 M HCl (250 mL), saturated aqueous NaHCO₃ solution (100 mL), brine (100 mL), and dried over MgSO₄, filtered, and concentrated to a crude oil. The oil was treated with cyclopentyl methyl ether (250 mL) while stirring to induce precipitation of the title compound from the mixture of isomers. The mixture was stirred at ambient temperature for 10 minutes, then stirred in an ice bath for about 30 minutes. The precipitate was collected by filtration, washed with cold cyclopentyl methyl ether (50 mL), and dried under vacuum at 40° C. for 2 hours to provide the title compound (19.2 g, 31.2%). ¹H NMR (600 MHz, CDCl₃) δ ppm 8.33 (s, 1H), 5.46 (s, 2H), 4.01 (s, 3H), 3.44 (s, 3H).

### Step A.2

### methyl 5-(1-hydroxyethyl)-1-(methoxymethyl)-4-nitro-1H-pyrazole-3-carboxylate

Methyl 1-(methoxymethyl)-4-nitro-1H-pyrazole-3-carboxylate (20 g, 93 mmol, Step A.1) was dissolved in tetrahydrofuran (266 mL) at ambient temperature with stirring until complete dissolution occurred. The resulting light yellow solution was cooled in an acetone-dry ice bath to approximately −70° C. and lithium hexamethyldisilazide (1 M in tetrahydrofuran, 112 mL, 112 mmol) was added down the side of the flask, maintaining an internal temperature below −65° C. The reaction was stirred for 15 minutes at the same temperature, and then ice-cold acetaldehyde (20.8 mL, 372 mmol) was added by pouring directly into the reaction flask. The reaction was stirred for another 45 minutes at the same temperature, quenched with acetic acid (6.39 mL, 112 mmol) at the same temperature, then warmed to 0° C. before diluting with ethyl acetate (200 mL) and 50% saturated aqueous ammonium chloride (200 mL). The mixture was stirred until two clear layers formed, and the layers were separated. The organic layer was washed with 50% saturated ammonium chloride (2×), then saturated sodium bicarbonate (3×), and brine, dried over sodium sulfate, and concentrated under reduced pressure to afford the title compound (25 g). ¹H NMR (600 MHz, CDCl₃) δ ppm 5.71 (d, J=10.6 Hz, 1H), 5.40 (d, J=10.7 Hz, 1H), 5.35 (q, J=6.8 Hz, 1H), 3.83 (s, 3H), 3.28 (s, 3H), 1.52 (d, J=6.8 Hz, 3H). MS (APCI) m/z: 259.9 [M+H]⁺.

### Step A.3

### methyl 5-acetyl-1-(methoxymethyl)-4-nitro-1H-pyrazole-3-carboxylate

Methyl 5-(1-hydroxyethyl)-1-(methoxymethyl)-4-nitro-1H-pyrazole-3-carboxylate (22 g, 85 mmol, Step A.2) was dissolved in dichloromethane (220 mL), and the solution was placed into a ambient temperature water bath. Dess-Martin's Periodinane (1,1,1-tris(acetyloxy)-1,1-dihydro-1,2-benzodioxol-3-(1H)-one) (39.6 g, 93 mmol) was added in portions over 10 minutes. Once addition of the oxidant was complete, the reaction mixture was stirred for 1 hour at ambient temperature. The reaction mixture was then diluted with ethyl acetate (220 mL), saturated sodium bicarbonate (220 mL), and saturated with sodium thiosulfate (220 mL). The biphasic mixture was stirred for 30 minutes at ambient temperature, giving two layers and a clear organic layer. The layers were separated, and the organic layer was washed with an additional 1:1 saturated sodium thiosulfate:saturated bicarbonate (2×), then brine, dried over sodium sulfate and concentrated under reduced pressure to afford the title compound (22 g). ¹H NMR (600 MHz, CDCl₃) δ ppm 5.66 (d, J=0.5 Hz, 2H), 3.98 (s, 3H), 3.39 (d, J=0.5 Hz, 3H), 2.56 (d, J=0.5 Hz, 3H). MS (APCI) m/z: 257.9 [M+H]⁺.

### Step A.4

### methyl 5-acetyl-4-amino-1-(methoxymethyl)-1H-pyrazole-3-carboxylate

Methyl 5-acetyl-1-(methoxymethyl)-4-nitro-1H-pyrazole-3-carboxylate (22 g, 86 mmol, Step A.3) and tetrahydrofuran (220 mL) were added to Ra—Ni (water slurry, 7 g, 53.7 mmol) in a 600 mL stainless steel reactor, and the suspension was stirred for 21 hours at 60 psi H₂ at ambient temperature. The solid catalyst was removed via filtration, and the filtrate was concentrated under reduced pressure to afford a crude oil that was slurried in tert-butyl methyl ether (100 mL) at ambient temperature for 30 minutes and at 0° C. for 30 minutes. The resulting precipitate was isolated via filtration and dried to constant weight under an air stream to afford the title compound (14.4 g, 74% over 3 steps). ¹H NMR (600 MHz, CDCl₃) δ ppm 5.97 (s, 2H), 5.70 (s, 2H), 3.96 (s, 3H), 3.32 (s, 3H), 2.63 (s, 3H). MS (APCI) m/z: 228.3 [M+H]⁺.

### Step A.5

### ethyl 7-hydroxy-1-(methoxymethyl)-1H-pyrazolo[4,3-b]pyridine-3-carboxylate

Methyl 5-acetyl-4-amino-1-(methoxymethyl)-1H-pyrazole-3-carboxylate (14.5 g, 63.8 mmol, Step A.4) was dissolved in tetrahydrofuran (213 mL). Methyl formate (19.6 mL, 319 mmol) was added, and the solution was cooled in an ice-water bath to around 10° C. Sodium methoxide (25% w/w in methanol, 43.8 mL, 191 mmol) was added dropwise via syringe. The flask was removed from the bath and stirred at ambient temperature for 2 hours. The flask was re-cooled in an ice-water bath, and 2 M aqueous hydrogen chloride (223 mL, 447 mmol, 7.0 equivalents) was added slowly. The reaction flask was stirred at ambient temperature for 14 hours, and then dichoromethane (200 mL) was added. The flask was cooled in an ice-water bath to <10° C. before adjusting the pH to 4-5 with 6 M NaOH. The layers were separated, and the aqueous layer was extracted three times with a mixture of dichloromethane (60 mL), tetrahydrofuran (40 mL), and methanol (10 mL). The combined organic extracts were dried over sodium sulfate and concentrated under reduced pressure to afford a crude residue that was slurried in tert-butyl methyl ether at 0° C. for 30 minutes. The precipitate was collected, washed with tert-butyl methyl ether, and dried under reduced pressure to afford the title compound (12.1 g, 80%). ¹H NMR (500 MHz, CDCl₃) δ ppm 8.70 (d, J=4.8 Hz, 1H), 7.47 (d, J=4.9 Hz, 1H), 6.05 (s, 2H), 4.60 (q, J=7.1 Hz, 2H), 3.38 (s, 3H), 1.50 (t, J=7.1 Hz, 3H). MS (APCI) m/z: 252.0 [M+H]⁺.

### Step A.6

### ethyl 7-chloro-1-(methoxymethyl)-1H-pyrazolo[4,3-b]pyridine-3-carboxylate

Ethyl 7-hydroxy-1-(methoxymethyl)-1H-pyrazolo[4,3-b]pyridine-3-carboxylate (8.4 g, 33.4 mmol, Step A.5) was dissolved in N,N-dimethylformamide (130 mL), and the solution was cooled to <5° C. before addition of trichlorophosphine (2.79 mL, 33.4 mmol) dropwise. The reaction was stirred for 30 minutes at the same temperature, and then the reaction mixture was poured over ice-cold saturated sodium bicarbonate and extracted dichloromethane (3×100 mL). The combined extracts were washed with water and brine, dried over sodium sulfate, and concentrated under reduced pressure to afford a crude dark oil that was purified via flash chromatography, eluting on a silica gel column (220 g) with ethyl acetate:heptanes (0:100 to 100:0) over 15 minutes to afford the title compound (5.7 g, 51% over 2 steps). ¹H NMR (500 MHz, CDCl₃) δ ppm 8.63 (d, J=4.8 Hz, 1H), 7.39 (d, J=4.8 Hz, 1H), 5.97 (s, 2H), 4.52 (q, J=7.1 Hz, 2H), 3.30 (s, 3H), 1.42 (t, J=7.1 Hz, 3H). MS (APCI) m/z: 269.9 [M+H]⁺.

### Step A.7

### ethyl 7-[4-(tert-butoxycarbonyl)piperazin-1-yl]-1-(methoxymethyl)-1H-pyrazolo[4,3-b]pyridine-3-carboxylate

Ethyl 7-chloro-1-(methoxymethyl)-1H-pyrazolo[4,3-b]pyridine-3-carboxylate (5.7 g, 21.1 mmol, Step A.6) and tert-butyl piperazine-1-carboxylate (7.09 g, 38.0 mmol) were dissolved in N,N-dimethylacetamide (60 mL), and triethylamine (5.89 mL, 42.3 mmol) was added. The reaction was heated to 120° C. for 20 hours using a heating mantle, and then cooled to ambient temperature. The reaction was diluted with water (100 mL) and extracted with dichloromethane (3×50 mL). The combined organics were washed with water (3×50 mL), brine (50 mL), dried over sodium sulfate, and concentrated under reduced pressure to afford the crude product that was used in the next step. MS (APCI) m/z: 420.3 [M+H]⁺.

### Step A.8

### ethyl 7-(piperazin-1-yl)-2H-pyrazolo[4,3-b]pyridine-3-carboxylate-hydrogen chloride (1/2)

Ethyl 7-[4-(tert-butoxycarbonyl)piperazin-1-yl]-1-(methoxymethyl)-1H-pyrazolo[4,3-b]pyridine-3-carboxylate (8.87 g, 21.2 mmol, Step A.7) was dissolved in ethanol (3 mL), and the solution was treated with 4 M HCl in 1,4-dioxane (37.0 mL, 148 mmol). The reaction was heated to 50° C. in a heating block for 4 hours and then allowed to cool to ambient temperature overnight. The reaction mixture was concentrated under reduced pressure to afford a residue that was used directly in the subsequent reaction. MS (APCI) m/z: 276.0 [M+H]⁺.

### Step A.9

### ethyl 7-[4-(tert-butoxycarbonyl)piperazin-1-yl]-2H-pyrazolo[4,3-b]pyridine-3-carboxylate

Ethyl 7-(piperazin-1-yl)-2H-pyrazolo[4,3-b]pyridine-3-carboxylate hydrogen chloride (1/2) (7.36 g, 21.1 mmol, Step A.8) was suspended in tetrahydrofuran (70 mL), and the solution was treated with N-ethyl-N-(propan-2-yl)propan-2-amine (11.1 mL, 63.4 mmol) and di-tert-butyl dicarbonate (5.89 mL, 25.4 mmol). The reaction was stirred at ambient temperature for 3 hours, diluted with ethyl acetate, washed with water (3×), washed with brine, dried over sodium sulfate, and concentrated in vacuo. The resulting residue was slurried in tert-butyl methyl ether, isolated by filtration through a fritted funnel, and dried to constant weight. The crude material was loaded onto a silica gel column (120 g) and eluted with ethyl acetate:methanol (100:0 to 80:20) to afford the title compound (3.3 g, 42% over 3 steps). ¹H NMR (400 MHz, CDCl₃) δ ppm 8.12 (br s, 1H), 6.36 (d, J=6.2 Hz, 1H), 4.44 (q, J=7.1 Hz, 2H), 3.95 (br s, 4H), 3.64 (t, J=5.3 Hz, 4H), 1.48 (s, 9H), 1.35 (t, J=7.1 Hz, 3H). MS (APCI) m/z: 376.1 [M+H]⁺.

### Intermediate B

### 2-(4-phenoxyphenyl)-7-(piperazin-1-yl)-4,5,6,7-tetrahydro-2H-pyrazolo[4,3-b]pyridine-3-carboxamide

### Step B.1

### (2E)-3-oxo-2-[2-(4-phenoxyphenyl)hydrazinylidene]butanenitrile

4-Phenoxyaniline (17.7 g, 92 mmol) was suspended in ice-cold water (293 mL) and concentrated aqueous hydrogen chloride (76.4 mL, 924 mmol) was added, followed by dropwise addition of a solution of sodium nitrite (6.38 g, 92 mmol) in water (100 mL). The flask was warmed to ambient temperature for 1 hour. Insoluble material was removed by filtration through a fritted funnel to afford an aqueous solution of azide. A separate 3 L round-bottomed-flask was charged with sodium acetate (227 g, 2.77 mol), water (587 mL), 3-oxobutanenitrile (12 g, 139 mmol), and ethanol (440 mL), and the resulting solution was cooled to <5° C. in an ice-water bath. The azide solution was then added dropwise via addition funnel over 10 minutes, during which time a bright yellow color and precipitate occurred. Once the addition was complete, the flask was removed from the bath, and the resulting suspension was stirred at ambient temperature for another 5 minutes. The resulting precipitate was isolated via filtration through a fritted funnel then washed with water and slurried in tert-butyl methyl ether and isolated via filtration, drying to constant weight in a vacuum oven at 60° C. to afford the title compound (25.8 g). ¹H NMR (400 MHz, CDCl₃) δ ppm 14.88 (s, 1H), 7.42-7.30 (m, 4H), 7.20-6.98 (m, 5H), 2.49 (d, J=4.3 Hz, 3H). MS (DCI) m/z: 280.1 [M+H]⁺.

### Step B.2

### ethyl 3-acetyl-4-amino-1-(4-phenoxyphenyl)-1H-pyrazole-5-carboxylate

(2E)-3-Oxo-2-[2-(4-phenoxyphenyl)hydrazinylidene]butanenitrile (25.8 g, 92 mmol, Step B.1) was dissolved in 1,4-dioxane (257 mL) and N,N-diisopropylethylamine (161 mL, 924 mmol) was added, followed by ethyl 2-bromoacetate (30.7 mL, 277 mmol). The reaction was heated to 100° C. for 5 hours, cooled to ambient, diluted with ethyl acetate (400 mL), washed with water (3×150 mL) and brine (100 mL), dried over sodium sulfate, and concentrated under reduced pressure to afford a crude residue, which was triturated with tert-butyl methyl ether and heptanes. The mixture was slurried at ambient temperature and the resulting precipitate was isolated via filtration, washed with heptanes, and dried to constant weight in the funnel, to afford 18.65 g of the title compound. The mother liquor was purified by flash chromatography (silica gel column, 120 g silica), eluting with ethyl acetate:heptanes (0:100 to 40:60) over 20 minutes to afford an additional 3.2 g of the title compound. The lots were combined to afford the title compound (21.9 g, 65%). ¹H NMR (400 MHz, CDCl₃) δ ppm 7.42-7.32 (m, 4H), 7.20-7.01 (m, 5H), 5.78 (br s, 2H), 4.24 (q, J=7.1 Hz, 2H), 2.58 (s, 3H), 1.21 (t, J=7.1 Hz, 3H). MS (ESI) m/z: 366.2 [M+H]⁺.

### Step B.3

### ethyl 7-hydroxy-2-(4-phenoxyphenyl)-2H-pyrazolo[4,3-b]pyridine-3-carboxylate-hydrogen chloride (1/1)

Ethyl 3-acetyl-4-amino-1-(4-phenoxyphenyl)-1H-pyrazole-5-carboxylate (24.0 g, 65.5 mmol, Step B.2) was dissolved in tetrahydrofuran (468 mL), and the solution was treated portion wise at about 10° C. with sodium hydride (7.86 g, 197 mmol). After the addition was complete, the reaction mixture was warmed to ambient temperature for 10 minutes, and ethyl formate (27.5 mL, 328 mmol) was added. The reaction was stirred for 16 hours at ambient. 1 M HCl (450 mL, 450 mmol) was added, and stirring was continued at ambient temperature for 6 hours, at which point a light brown precipitate had formed, which was isolated via filtration through a fritted funnel. The solid was washed with tert-butyl methyl ether (2×) in the fritted funnel and dried to constant weight to afford the title compound (20.4 g, 83%). ¹H NMR (500 MHz, CDCl₃) δ ppm 7.71 (d, J=7.8 Hz, 1H), 7.51-7.45 (m, 2H), 7.39 (tt, J=8.7, 2.2 Hz, 2H), 7.18 (td, J=8.0, 7.4, 1.1 Hz, 1H), 7.11-7.05 (m, 4H), 6.38 (d, J=7.7 Hz, 1H), 4.39 (q, J=7.0 Hz, 2H), 1.31 (t, J=7.0 Hz, 3H). MS (ESI) m/z: 376.3 [M+H]⁺.

### Step B.4

### ethyl 7-bromo-2-(4-phenoxyphenyl)-2H-pyrazolo[4,3-b]pyridine-3-carboxylate

Ethyl 7-hydroxy-2-(4-phenoxyphenyl)-2H-pyrazolo[4,3-b]pyridine-3-carboxylate hydrogen chloride (1/1) (9.75 g, 23.7 mmol, Sep B.3) was dissolved in N,N-dimethyl formamide (95 mL), and the flask was placed into a ambient temperature water bath before PBr₃ (4.47 mL, 47.3 mmol) was added dropwise at ambient temperature. After 5 minutes, the reaction was poured over ice-cold saturated sodium bicarbonate, and then extracted with ethyl acetate (3×). The combined organic extracts were washed with brine (50 mL), dried over sodium sulfate, and concentrated under reduced pressure to afford a residue that was slurried in heptanes (100 mL), isolated via filtration, and dried to constant weight in a vacuum oven at 50° C. to afford the title compound (10.1 g, 97%) in combination with about 20% of ethyl 7-chloro-2-(4-phenoxyphenyl)-2H-pyrazolo[4,3-b]pyridine-3-carboxylate. Major Component: ¹H NMR (500 MHz, CDCl₃) δ ppm 8.63 (d, J=4.6 Hz, 1H), 7.63 (d, J=4.6 Hz, 1H), 7.55-7.50 (m, 2H), 7.45-7.38 (m, 2H), 7.23-7.17 (m, 1H), 7.16-7.10 (m, 4H), 4.47 (q, J=7.1 Hz, 2H), 1.35 (t, J=7.1 Hz, 3H). MS (ESI) m/z: 438.2 [M+H]⁺.

### Step B.5

### 7-bromo-2-(4-phenoxyphenyl)-2H-pyrazolo[4,3-b]pyridine-3-carboxylic acid

Ethyl 7-bromo-2-(4-phenoxyphenyl)-2H-pyrazolo[4,3-b]pyridine-3-carboxylate (10.0 g, 22.9 mmol, Step B.4) was suspended in tetrahydrofuran (53.4 mL) and lithium hydroxide hydrate (2.88 g, 68.6 mmol). Water (26.7 mL), and methanol (26.7 mL) were added. The reaction mixture was stirred at ambient temperature for 60 minutes. The reaction was then acidified with 1 M HCl to pH 3-4, and then stirred for 30 minutes. The resulting precipitate was isolated via filtration, washed with water and tert-butyl methyl ether, then dried under reduced pressure to constant weight to afford a mixture of the title compound and about 20% 7-chloro-2-(4-phenoxyphenyl)-2H-pyrazolo[4,3-b]pyridine-3-carboxylic acid (9.38 g, 100%). Major component: ¹H NMR (400 MHz, CDCl₃) δ ppm 8.51 (d, J=4.7 Hz, 1H), 7.73 (d, J=4.7 Hz, 1H), 7.68-7.59 (m, 2H), 7.41 (dd, J=8.6, 7.4 Hz, 2H), 7.19 (t, J=7.4 Hz, 1H), 7.16-7.08 (m, 4H). MS (ESI) m/z: 412.1 [M+H]⁺.

### Step B.6

### 7-bromo-2-(4-phenoxyphenyl)-2H-pyrazolo[4,3-b]pyridine-3-carboxamide

7-Bromo-2-(4-phenoxyphenyl)-2H-pyrazolo[4,3-b]pyridine-3-carboxylic acid (9.38 g, 22.9 mmol, Step B.5) was suspended in tetrahydrofuran (152 mL) and triethylamine (9.56 mL, 68.6 mmol) was added, which solubilized the material. Isobutyl chloroformate (6.01 mL, 45.7 mmol) was added, and immediately after the addition some solid precipitation occurred. Ammonia (0.5M in 1,4-dioxane, 320 mL, 160 mmol) was added, and the reaction was stirred at ambient temperature for 30 minutes. The reaction mixture was diluted with ethyl acetate and water, and the mixture was stirred for 5 minutes at ambient temperature. The layers were separated, and the organic layer was washed with brine, dried over sodium sulfate, and concentrated to about 100 mL under reduced pressure. The precipitate was removed via filtration using a fritted funnel, washed with heptanes (50 mL), and then dried to constant weight in a vacuum oven at 60° C. to afford the title compound mixed with approximately 20% of 7-chloro-2-(4-phenoxyphenyl)-2H-pyrazolo[4,3-b]pyridine-3-carboxamide (9.60 g, 23.5 mmol). Major component: ¹H NMR (400 MHz, dimethyl sulfoxide-d6) δ ppm 8.58 (d, J=4.6 Hz, 1H), 8.36 (s, 1H), 8.13 (s, 1H), 7.89 (d, J=4.6 Hz, 1H), 7.68-7.60 (m, 2H), 7.48 (dd, J=8.6, 7.3 Hz, 2H), 7.28-7.18 (m, 1H), 7.18-7.09 (m, 4H). MS (ESI) m/z: 409.1 [M+H]⁺.

### Step B.7

### tert-butyl 4-[3-carbamoyl-2-(4-phenoxyphenyl)-2H-pyrazolo[4,3-b]pyridin-7-yl]piperazine-1-carboxylate

A 250-mL round-bottomed flask was charged with 7-bromo-2-(4-phenoxyphenyl)-2H-pyrazolo[4,3-b]pyridine-3-carboxamide (14.8 g, 36.3 mmol, Step B.6). N,N-Dimethylacetamide (96 mL) was added, followed by tert-butyl piperazine-1-carboxylate (10.7 g, 57.6 mmol) and the resulting solution was heated to 100° C. for 16 hours. The reaction was cooled to about 50° C., and water was added dropwise via addition funnel over about 5 minutes. The reaction flask was cooled to ambient temperature, and the precipitate was isolated via filtration through a fritted funnel. The resulting precipitate was washed with tert-butyl methyl ether (2×) and dried under reduced pressure afford the title compound (14.8 g, 79%). ¹H NMR (500 MHz, CDCl₃) δ ppm 9.33 (s, 1H), 8.32 (d, J=5.4 Hz, 1H), 7.55-7.49 (m, 2H), 7.43-7.35 (m, 2H), 7.23-7.06 (m, 5H), 6.37 (d, J=5.4 Hz, 1H), 5.68-5.64 (m, 1H), 3.96 (s, 1H), 3.69-3.61 (m, 4H), 1.58 (br s, 4H), 1.48 (s, 9H). MS (APCI) m/z: 515.4 [M+H]⁺.

### Step B.8

### tert-butyl 4-[3-carbamoyl-2-(4-phenoxyphenyl)-4,5,6,7-tetrahydro-2H-pyrazolo[4,3-b]pyridin-7-yl]piperazine-1-carboxylate

tert-Butyl 4-[3-carbamoyl-2-(4-phenoxyphenyl)-2H-pyrazolo[4,3-b]pyridin-7-yl]piperazine-1-carboxylate (11.8 g, 22.9 mmol, Step B.7) and methanol (240 mL) were added to 10% Pd(OH)₂/C, wet basis (11.8 g, 41.9 mmol) in a 600 mL stainless steel reactor, and the reaction was stirred for 20 hours under 60 psi H₂ and heated to 50° C. for 43 hours. The reaction mixture was filtered through diatomaceous earth and washed with methanol (100 mL). The filtrate was concentrated under reduced pressure and azeotroped once with tert-butyl methyl ether. The crude material (11.9 g) was then triturated from acetonitrile via dropwise addition of water at ambient temperature. Stirring was continued for 16 hours, and the resulting precipitate was isolated via filtration and washed with cold 1:1 acetonitrile:water to afford the title compound (9.78 g, 82%). ¹H NMR (400 MHz, CDCl₃) δ ppm 7.45-7.35 (m, 4H), 7.22-7.13 (m, 1H), 7.11-7.02 (m, 4H), 5.38 (br s, 2H), 5.11 (br s, 1H), 3.82 (dd, J=6.5, 4.9 Hz, 1H), 3.53-3.36 (m, 5H), 3.27 (d, J=4.4 Hz, 1H), 2.72-2.57 (m, 4H), 2.18 (dtd, J=13.9, 7.1, 3.1 Hz, 1H), 1.94 (dddd, J=13.6, 8.4, 5.1, 3.3 Hz, 1H), 1.45 (s, 9H). MS (APCI) m/z: 519.2 [M+H]⁺.

### Step B.9

### 2-(4-phenoxyphenyl)-7-(piperazin-1-yl)-4,5,6,7-tetrahydro-2H-pyrazolo[4,3-b]pyridine-3-carboxamide

Acetyl chloride (3.56 mL, 50.1 mmol) was added dropwise to a cold (<10° C.) flask containing methanol (125 mL). The resulting solution of HCl-methanol was poured over tert-butyl 4-[3-carbamoyl-2-(4-phenoxyphenyl)-4,5,6,7-tetrahydro-2H-pyrazolo[4,3-b]pyridin-7-yl]piperazine-1-carboxylate (6.5 g, 12.5 mmol, B.8) and heated to 50° C. for 90 minutes, at which point complete deprotection had occurred. The reaction mixture was directly concentrated under reduced pressure to afford a crude residue, which was treated with NaOH in dichloromethane to afford the title compound (5.2 g, 99%). ¹H NMR (400 MHz, CDCl₃) δ ppm 7.44-7.32 (m, 4H), 7.21-7.11 (m, 1H), 7.11-7.02 (m, 4H), 5.55 (s, 1H), 5.10 (s, 1H), 3.76 (dd, J=6.4, 5.0 Hz, 1H), 3.43 (ddd, J=11.6, 8.5, 3.1 Hz, 1H), 3.28 (ddd, J=11.3, 7.7, 3.3 Hz, 1H), 2.90 (dt, J=5.5, 3.5 Hz, 4H), 2.69 (m, 4H), 2.22 (dtd, J=13.8, 7.0, 3.1 Hz, 1H), 1.93 (dddd, J=13.6, 8.4, 5.0, 3.3 Hz, 1H), 1.80 (s, 1H). MS (APCI) m/z: 419.2 [M+H]⁺.

### Intermediate C

### 2-(4-phenoxyphenyl)-7-(piperidin-4-yl)-4,5,6,7-tetrahydro-2H-pyrazolo[4,3-b]pyridine-3-carboxamide-hydrogen chloride (1/3)

### Step C.1

### tert-butyl 4-(cyanoacetyl)piperidine-1-carboxylate

1-tert-Butyl 4-ethyl piperidine-1,4-dicarboxylate (25.0 g, 94.0 mmol) was dissolved in tetrahydrofuran (471 mL) and acetonitrile (24.6 mL, 471 mmol) was added. The resulting solution was cooled to <10° C. in an ice-water bath prior to addition of a solution of potassium tert-butoxide (1 M in tetrahydrofuran, 188 mL, 188 mmol) via syringe over about 5 minutes, maintaining the internal temperature below 15° C. Once the addition was complete, the reaction mixture was warmed to ambient temperature for 1 hour. The reaction was quenched with saturated ammonium chloride and diluted with ethyl acetate. The layers were separated, and the aqueous layer was extracted with additional ethyl acetate (2×). The combined extracts were washed with brine, dried over sodium sulfate, and concentrated in vacuo. Heptanes were added, and the resulting mixture was slurried for 5 minutes. The precipitate was isolated via filtration through a fritted funnel, washed with heptanes and concentrated under reduced pressure to afford the title compound (23.0 g, 97%). MS (APCI) m/z: 253.2 [M+H]⁺.

### Step C.2

### tert-butyl 4-{(2E)-2-cyano-2-[2-(4-phenoxyphenyl)hydrazinylidene]acetyl}piperidine-1-carboxylate

4-Phenoxyaniline (5.00 g, 27.0 mmol) was suspended in ice-cold water (100 mL) and concentrated HCl (8.20 mL, 270 mmol) was added, followed by dropwise addition of sodium nitrite (1.86 g, 27.0 mmol) in water (27 mL). The reaction was stirred at the same temperature for 15 minutes, then warmed to ambient temperature for 30 minutes to afford a light brown solution of the azide. A separate 1 L round-bottomed flask was charged with sodium acetate trihydrate (110 g, 810 mmol) and tert-butyl 4-(cyanoacetyl)piperidine-1-carboxylate (10.2 g, 40.5 mmol, Step C.1) in ethanol (200 mL) and water (100 mL). The resulting solution was cooled to <5° C. in an ice-water bath. The azide solution was then added dropwise via addition funnel over 5 minutes (exotherms maintained below 10° C.). Once the addition was complete, the flask was removed from the bath, and the resulting suspension stirred at ambient temperature for another 5 minutes. The reaction mixture was diluted with additional water and the resulting precipitate was isolated via filtration through a fritted funnel and washed with water. The residue was slurried in tert-butyl methyl ether, isolated via filtration through a fritted funnel, washed with heptanes, and dried to constant weight at 50° C. in a vacuum oven to afford the title compound as a 4:1 ratio of isomers (11.0 g, 91%). Major isomer: ¹H NMR (400 MHz, CDCl₃) δ ppm 15.02 (s, 1H), 7.40-7.33 (m, 4H), 7.18-7.12 (m, 1H), 7.10-6.99 (m, 4H), 4.20 (s, 2H), 3.11 (ddd, J=11.4, 7.8, 3.6 Hz, 1H), 2.83 (d, J=14.1 Hz, 2H), 1.97-1.79 (m, 2H), 1.75-1.62 (m, 2H), 1.47 (d, J=3.0 Hz, 9H). MS (APCI) m/z: 434.2 [M+H]⁺.

### Step C.3

### tert-butyl 4-[4-amino-5-(ethoxycarbonyl)-1-(4-phenoxyphenyl)-1H-pyrazole-3-carbonyl]piperidine-1-carboxylate

tert-Butyl 4-{(2E)-2-cyano-2-[2-(4-phenoxyphenyl)hydrazinylidene]acetyl}piperidine-1-carboxylate (11.4 g, 25.4 mmol, Step C.2) was dissolved in a mixture of 1,4-dioxane (114 mL) and N-ethyl-N-isopropylpropan-2-amine (45.2 mL, 254 mmol), and the reaction mixture was treated with ethyl 2-bromoacetate (8.46 mL, 76 mmol) and heated to 100° C. with a heating mantle for 15 hours. The reaction was cooled to ambient temperature, diluted with ethyl acetate, washed with water (3×) and brine, dried over sodium sulfate, and concentrated under reduced pressure to afford a crude residue. The residue was slurried with heptanes (130 mL), filtered, and dried to constant weight to afford the title compound (10.8 g, 79%). ¹H NMR (400 MHz, CDCl₃) δ ppm 7.43-7.30 (m, 4H), 7.20-7.14 (m, 1H), 7.14-7.00 (m, 4H), 5.81 (br s, 2H), 4.25 (q, J=7.1 Hz, 2H), 4.22-4.05 (m, 2H), 3.59 (tt, J=11.6, 3.8 Hz, 1H), 2.84 (t, J=13.0 Hz, 2H), 1.88 (d, J=13.1 Hz, 2H), 1.78-1.63 (m, 2H), 1.46 (s, 9H), 1.21 (t, J=7.1 Hz, 3H). MS (APCI) m/z: 535.4 [M+H]⁺.

### Step C.4

### (1E)-N-tert-butylethanimine

A 3-neck 100-mL round bottom flask equipped with an addition funnel was charged with tert-butylamine (43.5 mL, 410 mmol) and the flask was cooled to <5° C. in an ice-water bath. Acetaldehyde (23.2 mL, 410 mmol) was added dropwise via addition funnel over 20 minutes, maintaining an internal temperature around 10° C. Once the addition was complete, the reaction mixture was stirred at the same temperature for 3 hours, and then powdered potassium hydroxide (4.10 g, 73.1 mmol) was added. The flask was placed in a fridge for 16 hours, and then the layers were separated. The organic layer was distilled at 1 atmosphere pressure (bp 66-70° C.) to afford the title compound (35.0 g, 86%). ¹H NMR (400 MHz, CDCl₃) δ ppm 7.69 (q, J=4.8 Hz, 1H), 1.96 (d, J=4.8 Hz, 3H), 1.17 (s, 9H).

### Step C.5

### ethyl 7-[1-(tert-butoxycarbonyl)piperidin-4-yl]-2-(4-phenoxyphenyl)-2H-pyrazolo[4,3-b]pyridine-3-carboxylate

Lithium diisopropylamide was prepared by addition of diisopropylamine (4.22 mL, 29.9 mmol) to a solution of n-butyllithium (11.6 mL, 29.0 mmol) and tetrahydrofuran (40 mL) at −78° C. (1E)-N-tert-butylethanimine (4.07 mL, 29.9 mmol, Step C.4) was added dropwise via syringe, maintaining the internal temperature below −60° C., and the solution was stirred for 20 minutes before addition of tert-butyl 4-[4-amino-5-(ethoxycarbonyl)-1-(4-phenoxyphenyl)-1H-pyrazole-3-carbonyl]piperidine-1-carboxylate (5.00 g, 9.35 mmol, Step C.3) as a thin slurry in tetrahydrofuran (22.3 mL) over 5 minutes, maintaining the internal temperature below −60° C. The reaction was stirred for another 15 minutes at the same temperature, and the resulting solution was quenched with 1 M HCl (50 mL) and warmed to ambient temperature. The reaction mixture was diluted with ethyl acetate, and the layers were separated. The aqueous layer was extracted with additional ethyl acetate (2×). The combined organic extracts were dried over sodium sulfate, concentrated under reduced pressure to afford a residue which was purified via flash chromatography, and eluted on a silica gel column with 30:70 to 100:0 tert-butyl methyl ether:heptanes to afford the title compound (2.54 g, 50%). ¹H NMR (400 MHz, CDCl₃) δ ppm 8.78 (d, J=4.4 Hz, 1H), 7.55-7.45 (m, 2H), 7.45-7.36 (m, 2H), 7.19 (t, J=7.4 Hz, 1H), 7.16-7.08 (m, 5H), 4.45 (q, J=7.1 Hz, 2H), 4.28 (s, 2H), 3.49 (tt, J=12.2, 3.5 Hz, 1H), 2.92 (t, J=12.7 Hz, 2H), 2.11-1.99 (m, 2H), 1.82 (qd, J=12.7, 4.3 Hz, 2H), 1.48 (s, 9H), 1.33 (t, J=7.1 Hz, 3H). MS (APCI) m/z: 543.4 [M+H]⁺.

### Step C.6

### 7-[1-(tert-butoxycarbonyl)piperidin-4-yl]-2-(4-phenoxyphenyl)-2H-pyrazolo[4,3-b]pyridine-3-carboxylic acid

Ethyl 7-[1-(tert-butoxycarbonyl)piperidin-4-yl]-2-(4-phenoxyphenyl)-2H-pyrazolo[4,3-b]pyridine-3-carboxylate (4.60 g, 8.48 mmol, Step C.5) was suspended in a mixture of tetrahydrofuran (34 mL), water (17 mL), and methanol (6 mL); and lithium hydroxide hydrate (1.78 g, 42.4 mmol) was added. The reaction was stirred at ambient temperature for 30 minutes, at which point complete saponification had occurred. The reaction mixture was acidified with HCl (42.4 mL, 42.4 mmol) to pH 2, and then extracted with ethyl acetate (3×). The organic extracts were washed with water, then brine, dried over sodium sulfate, and concentrated under reduced pressure to afford the title compound (4.36 g). ¹H NMR (400 MHz, dimethyl sulfoxide-d₆) δ ppm 8.70 (d, J=4.4 Hz, 1H), 7.67-7.59 (m, 2H), 7.50-7.42 (m, 2H), 7.36-7.32 (m, 1H), 7.26-7.19 (m, 1H), 7.19-7.05 (m, 4H), 4.11 (m, 1H), 3.39 (ddd, J=11.9, 8.5, 3.4 Hz, 2H), 2.90 (s, 2H), 1.97 (d, J=12.2 Hz, 2H), 1.85-1.71 (m, 2H), 1.41 (s, 9H).

### Step C.7

### tert-butyl 4-[3-carbamoyl-2-(4-phenoxyphenyl)-2H-pyrazolo[4,3-b]pyridin-7-yl]piperidine-1-carboxylate

7-[1-(tert-butoxycarbonyl)piperidin-4-yl]-2-(4-phenoxyphenyl)-2H-pyrazolo[4,3-b]pyridine-3-carboxylic acid (4.36 g, 8.47 mmol, Step C.6) was suspended in tetrahydrofuran (56.5 mL) and triethylamine (3.54 mL, 25.4 mmol) was added. The flask was cooled in an ice-water bath to <5° C. Isobutyl chloroformate (1.67 mL, 12.7 mmol) was added dropwise. After 5 minutes, ammonia (0.5 M in 1,4-dioxane, 85 mL, 42.4 mmol) was added quickly via syringe, and the resulting mixture was stirred for 5 minutes. The reaction was diluted with ethyl acetate, water was added, and the resulting biphasic mixture was stirred for 30 minutes at ambient temperature. The layers were separated, and the organic layer was concentrated under reduced pressure to afford the title compound (4.35 g, 100%). ¹H NMR (500 MHz, CDCl₃) δ ppm 9.03 (s, 1H), 8.63 (d, J=4.4 Hz, 1H), 7.58-7.51 (m, 2H), 7.44-7.34 (m, 2H), 7.22-7.07 (m, 6H), 5.70 (d, J=3.4 Hz, 1H), 4.29 (s, 2H), 3.51 (tt, J=12.2, 3.5 Hz, 1H), 2.92 (s, 2H), 2.15-2.02 (m, 2H), 1.88-1.71 (m, 2H), 1.49 (s, 9H). MS (APCI) m/z: 514.3 [M+H]⁺.

### Step C.8

### tert-butyl 4-[3-carbamoyl-2-(4-phenoxyphenyl)-4,5,6,7-tetrahydro-2H-pyrazolo[4,3-b]pyridin-7-yl]piperidine-1-carboxylate

tert-Butyl 4-[3-carbamoyl-2-(4-phenoxyphenyl)-2H-pyrazolo[4,3-b]pyridin-7-yl]piperidine-1-carboxylate (4.30 g, 8.37 mmol, Step C.7) and Pd—C(10 weight % metal basis, 2.67 g, 2.51 mmol) were weighed into a round-bottomed flask, and the flask was purged with N2. Tetrahydrofuran (84 mL) was added, and the flask was purged with a balloon of H₂ and then stirred under 1 atmosphere pressure of H₂ for 16 hours. The reaction flask was purged with N2 and then filtered through a pad of diatomaceous earth, which was washed with additional tetrahydrofuran (30 mL). The filtrate was concentrated under reduced pressure to afford the title compound (3.80 g, 88%). ¹H NMR (400 MHz, CDCl₃) δ ppm 7.43-7.31 (m, 4H), 7.21-7.14 (m, 1H), 7.11-7.00 (m, 4H), 5.37-5.12 (br s, 2H), 4.14 (br s, 2H), 3.32 (dq, J=30.3, 6.3 Hz, 2H), 2.84 (q, J=6.5 Hz, 1H), 2.68 (s, 2H), 2.10-1.75 (m, 4H), 1.67 (d, J=13.3 Hz, 2H), 1.45 (s, 9H), 1.33 (tdd, J=22.5, 11.5, 3.1 Hz, 2H). MS (APCI) m/z: 518.4 [M+H]⁺.

### Step C.9

### 2-(4-phenoxyphenyl)-7-(piperidin-4-yl)-4,5,6,7-tetrahydro-2H-pyrazolo[4,3-b]pyridine-3-carboxamide-hydrogen chloride (1/3)

Acetyl chloride (2.54 mL, 35.7 mmol) was added to cold methanol (36 mL) and the solution was added to a flask containing the starting material tert-butyl 4-[3-carbamoyl-2-(4-phenoxyphenyl)-4,5,6,7-tetrahydro-2H-pyrazolo[4,3-b]pyridin-7-yl]piperidine-1-carboxylate (3.70 g, 7.15 mmol, Step C.8). The flask was heated to 45° C. for 0.5 hours, at which point the reaction was complete. The flask was cooled to ambient temperature and concentrated under reduced pressure to afford the title compound (3.77 g). ¹H NMR (501 MHz, dimethyl sulfoxide-d₆) δ ppm 9.05 (d, J=10.8 Hz, 1H), 8.84 (d, J=11.5 Hz, 1H), 8.30 (s, 1H), 8.11 (s, 1H), 7.51-7.39 (m, 4H), 7.26-7.15 (m, 1H), 7.15-7.05 (m, 4H), 3.55 (ddd, J=12.8, 6.1, 2.6 Hz, 1H), 3.36-3.21 (m, 3H), 2.97-2.75 (m, 3H), 2.04 (td, J=14.7, 9.0 Hz, 3H), 1.89 (q, J=11.4 Hz, 1H), 1.78-1.53 (m, 3H). MS (APCI) m/z: 418.5 [M+H]⁺.

### Intermediate D

### tert-butyl 4-[2-(4-bromo-2-fluorophenyl)-3-carbamoyl-2H-pyrazolo[4,3-b]pyridin-7-yl]piperazine-1-carboxylate

### Step D.1

### (2E)-2-[2-(4-bromo-2-fluorophenyl)hydrazinylidene]-3-oxobutanenitrile

4-Bromo-2-fluoroaniline (6.00 g, 30.9 mmol) was suspended in ice-cold water (98 mL). Concentrated HCl (9.40 mL, 309 mmol) was added, followed by dropwise addition of a solution of sodium nitrite (2.20 g, 30.9 mmol) in water (15 mL). The reaction was stirred at the same temperature for 30 minutes, which resulted in a light brown solution of azide. A separate 1 L round-bottomed flask was charged with sodium acetate (76 g, 928 mmol), water (196 mL), 3-oxobutanenitrile (3.86 g, 46.4 mmol) and ethanol (147 mL), and the resulting solution was cooled to <5° C. in an ice-water bath. The azide solution was then added dropwise via an addition funnel over 10 minutes (exotherms maintained below 8° C.), during which time precipitation occurred. Once the addition was complete, the flask was removed from the bath, and the resulting suspension was stirred at ambient temperature for another 5 minutes. The precipitate was isolated via filtration, washed with water, slurried in tert-butyl methyl ether, isolated via filtration through a fritted funnel, and dried under reduced pressure at 60° C. to afford the title compound (7.20 g, 82%), which was used without additional purification. ¹H NMR (500 MHz, CDCl₃) δ ppm 14.68 (s, 1H), 7.65 (t, J=8.6 Hz, 1H), 7.40-7.32 (m, 2H), 2.54 (s, 3H).

### Step D.2

### ethyl 3-acetyl-4-amino-1-(4-bromo-2-fluorophenyl)-1H-pyrazole-5-carboxylate

(2E)-2-[2-(4-Bromo-2-fluorophenyl)hydrazinylidene]-3-oxobutanenitrile (7.20 g, 25.3 mmol, Step D.1) was dissolved in 1,4-dioxane (70.4 mL) and N,N-diisopropylethylamine (44.3 mL, 253 mmol) was added, followed by ethyl 2-bromoacetate (8.43 mL, 76 mmol). The reaction was heated to 100° C. for 3 hours, at which point LC-MS indicated complete consumption of the starting material. The reaction was then cooled to ambient temperature and diluted with ethyl acetate, washed with water (3×), brine, dried over sodium sulfate, and concentrated under reduced pressure. The crude residue was purified by flash column chromatography, eluting with ethyl acetate:heptanes (0:100 to 40:60) over 20 minutes on a 220 g silica gel column to afford the title compound (8.60 g, 92%). ¹H NMR (500 MHz, CDCl₃) δ ppm 7.45-7.38 (m, 2H), 7.33 (dd, J=8.4, 7.6 Hz, 1H), 5.73 (s, 2H), 4.24 (q, J=7.1 Hz, 2H), 2.57 (s, 3H), 1.20 (t, J=7.1 Hz, 3H). MS (APCI) m/z: 372.0 [M+H]⁺.

### Step D.3

### ethyl 2-(4-bromo-2-fluorophenyl)-7-hydroxy-2H-pyrazolo[4,3-b]pyridine-3-carboxylate-hydrogen chloride (1/1)

Ethyl 3-acetyl-4-amino-1-(4-bromo-2-fluorophenyl)-1H-pyrazole-5-carboxylate (8.60 g, 23.2 mmol, Step D.2) was dissolved in tetrahydrofuran (166 mL) and the solution was treated at about 10° C. with sodium hydride (2.79 g, 69.7 mmol) portion wise. After the addition was complete, the reaction mixture was warmed to ambient temperature for 10 minutes, and ethyl formate (9.75 mL, 116 mmol) was added. The reaction was stirred at ambient temperature for 20 hours. 1 M HCl (200 mL) was added, and stirring was continued at ambient temperature for 16 hours. The resulting precipitate was isolated via filtration through a fritted funnel, slurried with tert-butyl methyl ether in the fritted funnel, washed with tert-butyl methyl ether and dried to constant weight to afford the title compound (4.37 g, 50%). ¹H NMR (500 MHz, CDCl₃) δ ppm 7.81 (br s, 1H), 7.55-7.39 (m, 5H), 6.48 (br s, 1H), 4.41 (q, J=7.0 Hz, 2H), 1.30 (t, J=7.1 Hz, 3H). MS (APCI) m/z: 380.0 [M+H]⁺.

### Step D.4

### ethyl 7-bromo-2-(4-bromo-2-fluorophenyl)-2H-pyrazolo[4,3-b]pyridine-3-carboxylate

Ethyl 2-(4-bromo-2-fluorophenyl)-7-hydroxy-2H-pyrazolo[4,3-b]pyridine-3-carboxylate-hydrogen chloride (1/1) (4.37 g, 10.5 mmol, Step D.3) was dissolved in N,N-dimethylformamide (46.0 mL) and PBr₃ (2.17 mL, 23.0 mmol) was added dropwise at ambient temperature (placed into an ambient temperature water bath to control exotherm). The internal temperature reached 35° C. during the addition. Immediately after the addition, the reaction was complete as indicated by LC-MS. The reaction mixture was poured over ice-cold saturated sodium bicarbonate then extracted with ethyl acetate (3×). The combined extracts were washed with brine, dried over sodium sulfate, and concentrated under reduced pressure, then dried to constant weight in a vacuum oven at 50° C. to afford the title compound (5.09 g) mixed with about 20% of ethyl 2-(4-bromo-2-fluorophenyl)-7-chloro-2H-pyrazolo[4,3-b]pyridine-3-carboxylate. This product was used without additional purification. Major component: ¹H NMR (500 MHz, CDCl₃) δ ppm 8.65 (d, J=4.6 Hz, 1H), 7.64 (d, J=4.6 Hz, 1H), 7.54-7.49 (m, 2H), 7.49-7.45 (m, 1H), 4.47 (q, J=7.1 Hz, 2H), 1.35 (t, J=7.1 Hz, 3H). MS (APCI) m/z: 444.0 [M+H]⁺.

### Step D.5

### 7-bromo-2-(4-bromo-2-fluorophenyl)-2H-pyrazolo[4,3-b]pyridine-3-carboxylic acid

Ethyl 7-bromo-2-(4-bromo-2-fluorophenyl)-2H-pyrazolo[4,3-b]pyridine-3-carboxylate (5.09 g, 11.5 mmol, Step D.4) was suspended in tetrahydrofuran (26.8 mL) and lithium hydroxide hydrate (1.45 g, 34.5 mmol), water (13.4 mL), and methanol (13.4 mL) were added. The reaction was stirred at ambient temperature for 60 minutes. The reaction was acidified with 1 M HCl to pH 3-4, then stirred for 30 minutes. The resulting precipitate was isolated via filtration, washed with water and tert-butyl methyl ether, and dried under reduced pressure to constant weight to afford the title compound (4.77 g) that contained about 20% of 2-(4-bromo-2-fluorophenyl)-7-chloro-2H-pyrazolo[4,3-b]pyridine-3-carboxylic acid. ¹H NMR (500 MHz, CDCl₃) δ ppm 8.55 (d, J=4.4 Hz, 1H), 7.75 (d, J=4.6 Hz, 1H), 7.58-7.43 (m, 3H). MS (APCI) m/z: 416.0 [M+H]⁺.

### Step D.6

### 7-bromo-2-(4-bromo-2-fluorophenyl)-2H-pyrazolo[4,3-b]pyridine-3-carboxamide

7-Bromo-2-(4-bromo-2-fluorophenyl)-2H-pyrazolo[4,3-b]pyridine-3-carboxylic acid (4.77 g, 11.5 mmol, Step D.5) was suspended in tetrahydrofuran (77.0 mL) and triethylamine (4.81 mL, 34.5 mmol) was added, which solubilized the material. The reaction flask was cooled in an ice-water bath to an internal temperature <5° C. Isobutyl chloroformate (3.02 mL, 23.0 mmol) was added, resulting in immediate precipitation. After 5 minutes, ammonia (0.5 M in 1,4-dioxane, 161 mL, 80 mmol) was added and the reaction was stirred at ambient temperature for 60 minutes. The reaction mixture was diluted with ethyl acetate and water, and the biphasic mixture was stirred for 5 minutes at ambient temperature. The layers were separated, and the organic layer was washed with brine, dried over sodium sulfate, and concentrated under reduced pressure to a volume of about 100 mL. Heptanes were added, and the resulting precipitate was isolated via filtration through a fritted funnel, washed with additional heptanes (100 mL), and dried to afford the title compound which contained about 20% of 2-(4-bromo-2-fluorophenyl)-7-chloro-2H-pyrazolo[4,3-b]pyridine-3-carboxamide (4.76 g). Major component: ¹H NMR (400 MHz, CDCl₃) δ ppm 8.68 (s, 1H), 8.50 (d, J=4.6 Hz, 1H), 7.67 (d, J=4.6 Hz, 1H), 7.54-7.37 (m, 3H), 5.78 (s, 1H). MS (APCI) m/z: 415.0 [M+H]⁺.

### Step D.7

### tert-butyl 4-[2-(4-bromo-2-fluorophenyl)-3-carbamoyl-2H-pyrazolo[4,3-b]pyridin-7-yl]piperazine-1-carboxylate

7-Bromo-2-(4-bromo-2-fluorophenyl)-2H-pyrazolo[4,3-b]pyridine-3-carboxamide (4.76 g, 11.50 mmol, Step D.6) and tert-butyl piperazine-1-carboxylate (6.62 g, 34.5 mmol) were suspended in N,N-dimethylacetamide (84 mL) and heated to 100° C. for 4 hours. The reaction mixture was cooled to ambient temperature and precipitated. Water (70 mL) was added dropwise, and the resulting slurry was stirred for 60 minutes at ambient temperature. The resulting precipitate was isolated via filtration, washed with tert-butyl methyl ether, and dried to constant weight in a vacuum oven at 50° C. to afford the title compound (4.87 g, 82%). ¹H NMR (400 MHz, CDCl₃) δ ppm 9.20-9.00 (m, 1H), 8.33 (d, J=5.3 Hz, 1H), 7.53-7.30 (m, 3H), 6.36 (d, J=5.4 Hz, 1H), 5.74 (d, J=3.7 Hz, 1H), 3.94 (s, 4H), 3.70-3.49 (m, 4H), 1.48 (s, 9H). MS (APCI) m/z: 521.0 [M+H]⁺.

### Intermediate E

### tert-butyl 4-[2-(4-bromophenyl)-3-carbamoyl-2H-pyrazolo[4,3-b]pyridin-7-yl]piperazine-1-carboxylate

### Step E.1

### (2E)-2-[2-(4-bromophenyl)hydrazinylidene]-3-oxobutanenitrile

4-Bromoaniline (15 g, 85 mmol) was suspended in ice-cold water (269 mL) and concentrated hydrogen chloride (25.7 mL, 846 mmol) was added, followed by dropwise addition of sodium nitrite (5.84 g, 85 mmol) in water (80 mL). The reaction was stirred at ambient temperature for 30 minutes to afford a solution of azide. A separate 3 L round-bottomed flask was charged with sodium acetate (208 g, 2.54 mol), water (537 mL), 3-oxobutanenitrile (11.0 g, 127 mmol), and ethanol (403 mL). The resulting solution was cooled to <5° C. in an ice-water bath. The azide solution was then added dropwise via addition funnel over 10 minutes (exotherms maintained below 8° C.), during which time a precipitate formed. Once the addition was complete, the flask was removed from the bath, and the resulting suspension was stirred at ambient temperature for another 5 minutes. The resulting precipitate was isolated via filtration through a fritted funnel, washed with water, slurried in tert-butyl methyl ether in the funnel, and dried in a vacuum oven at 60° C. until constant weight to afford the title compound (21.8 g, 97%) as a mixture of isomers. Major isomer: ¹H NMR (500 MHz, CDCl₃) δ ppm 14.70 (s, 1H), 7.55-7.52 (m, 2H), 7.30-7.27 (m, 2H), 2.52 (s, 3H). MS (APCI) m/z: 267.9 [M+H]⁺.

### Step E.2

### ethyl 3-acetyl-4-amino-1-(4-bromophenyl)-1H-pyrazole-5-carboxylate

(2E)-2-[2-(4-Bromophenyl)hydrazinylidene]-3-oxobutanenitrile (43.5 g, 163 mmol, Step E.1) was dissolved in 1,4-dioxane (454 mL) and N,N-diisopropylethylamine (286 mL, 1.64 mol) was added, followed by ethyl 2-bromoacetate (54.4 mL, 490 mmol). The reaction was heated to 100° C. for 4 hours using a heating mantle. The flask was cooled to ambient temperature, diluted with ethyl acetate, washed with water (3×), washed with brine, dried over sodium sulfate and concentrated under reduced pressure to afford a crude residue, which was triturated with tert-butyl methyl ether and heptanes. The precipitate was isolated via filtration through a fritted funnel and dried to constant weight to afford the title compound (41.5 g, 72%). H NMR (400 MHz, CDCl₃) δ ppm 7.64-7.53 (m, 2H), 7.35-7.27 (m, 2H), 5.78 (s, 2H), 4.24 (q, J=7.1 Hz, 2H), 2.57 (s, 3H), 1.21 (t, J=7.1 Hz, 3H). MS (APCI) m/z: 352.0 [M+H]⁺.

### Step E.3

### ethyl 2-(4-bromophenyl)-7-hydroxy-2H-pyrazolo[4,3-b]pyridine-3-carboxylate-hydrogen chloride (1/1)

Ethyl 3-acetyl-4-amino-1-(4-bromophenyl)-1H-pyrazole-5-carboxylate (39.5 g, 112 mmol, Step E.2) was dissolved in tetrahydrofuran (800 mL), and the solution was treated at about 10° C. with portion wise addition of sodium hydride (13.4 g, 336 mmol). After the addition was complete, the reaction mixture was warmed to ambient temperature for 10 minutes, and ethyl formate (47.0 mL, 560 mmol) was added. The reaction was stirred overnight at ambient temperature. 1 M HCl (500 mL) was added and stirring was continued at ambient temperature for 40 hours. The resulting precipitate was isolated via filtration through a fritted funnel, and slurried with tert-butyl methyl ether in the fritted funnel. The precipitate was transferred to a 1 L round-bottomed flask and slurried in tert-butyl methyl ether for 16 hours. The resulting product was isolated via filtration through a fritted funnel, washed with additional tert-butyl methyl ether, and dried to constant weight to afford the title compound (37.0 g, 91%). MS (APCI) m/z: 364.0 [M+H]⁺.

### Step E.4

### ethyl 7-bromo-2-(4-bromophenyl)-2H-pyrazolo[4,3-b]pyridine-3-carboxylate

Ethyl 2-(4-bromophenyl)-7-hydroxy-2H-pyrazolo[4,3-b]pyridine-3-carboxylate-hydrogen chloride (1/1) (9.50 g, 23.8 mmol, Step E.3) was dissolved in N,N-dimethylformamide (95 mL), and the flask was placed into an ambient temperature water bath. PBr₃ (4.50 mL, 47.7 mmol) was added dropwise at ambient temperature. After 5 minutes, the reaction mixture was poured over ice-cold saturated sodium bicarbonate, and then extracted with ethyl acetate (3×). The combined extracts were washed with brine, dried over sodium sulfate, and concentrated under reduced pressure. The resulting residue was slurried in heptanes, isolated via filtration through a fritted funnel, and dried in a vacuum oven at 50° C. to constant weight, to afford the title compound that contained approximately 25% ethyl 2-(4-bromophenyl)-7-chloro-2H-pyrazolo[4,3-b]pyridine-3-carboxylate (7.45 g, 73.5%). Major component: ¹H NMR (500 MHz, CDCl₃) δ ppm 8.62 (d, J=4.6 Hz, 1H), 7.72-7.65 (m, 2H), 7.62 (d, J=4.5 Hz, 1H), 7.49-7.42 (m, 2H), 4.48-4.39 (m, 2H), 1.37-1.30 (m, 3H). MS (APCI) m/z: 426.1 [M+H]⁺.

### Step E.5

### 7-bromo-2-(4-bromophenyl)-2H-pyrazolo[4,3-b]pyridine-3-carboxylic acid

Ethyl 7-bromo-2-(4-bromophenyl)-2H-pyrazolo[4,3-b]pyridine-3-carboxylate (7.45 g, 17.5 mmol, Step E.4) was suspended in tetrahydrofuran (40.9 mL) and lithium hydroxide hydrate (2.21 g, 52.6 mmol), water (20.5 mL), and methanol (20.5 mL) were added. The reaction was stirred at ambient temperature for 60 minutes. The reaction was then acidified with 1 M HCl to pH 3-4, and then stirred for 30 minutes. The resulting solid was isolated via filtration through a fritted funnel and washed with water and tert-butyl methyl ether. Thereafter, it was dried under reduced pressure to constant weight to afford the title compound that contained about 25% of 2-(4-bromophenyl)-7-chloro-2H-pyrazolo[4,3-b]pyridine-3-carboxylic acid (7 g). Major component: MS (APCI) m/z: 397.9 [M+H]⁺.

### Step E.6

### 7-bromo-2-(4-bromophenyl)-2H-pyrazolo[4,3-b]pyridine-3-carboxamide

7-Bromo-2-(4-bromophenyl)-2H-pyrazolo[4,3-b]pyridine-3-carboxylic acid (6.96 g, 17.5 mmol, Step E.5) was suspended in tetrahydrofuran (117 mL) and triethylamine (7.33 mL, 52.6 mmol) was added, which solubilized the material. The reaction was cooled in an ice-water bath to <5° C. Isobutyl chloroformate (4.60 mL, 35.1 mmol) was added, and immediately after the addition some solid precipitation occurred. The reaction was stirred for an additional 5 minutes, at which point ammonia (0.5 M in 1,4-dioxane, 245 mL, 123 mmol) solution was added, and the reaction was stirred at ambient temperature for 30 minutes. The reaction mixture was diluted with ethyl acetate and water, then stirred for 5 minutes at ambient temperature. The layers were separated, and the organic layer was washed with brine, dried over sodium sulfate, and the filtrate concentrated to about 100 mL. Heptanes were added, and the resulting precipitate was isolated via filtration through a fritted funnel, washed with heptanes, and dried to constant weight in a vacuum oven at 60° C. to afford the title compound containing approximately 25% of 2-(4-bromophenyl)-7-chloro-2H-pyrazolo[4,3-b]pyridine-3-carboxamide (6.94 g). Major component: ¹H NMR (500 MHz, CDCl₃) δ ppm 8.85 (s, 1H), 8.48 (d, J=4.6 Hz, 1H), 7.66 (d, J=3.2 Hz, 2H), 7.60 (d, J=4.6 Hz, 1H), 7.49 (d, J=8.6 Hz, 2H), 5.89 (s, 1H). MS (APCI) m/z: 396.9 [M+H]⁺.

### Step E.7

### tert-butyl 4-[2-(4-bromophenyl)-3-carbamoyl-2H-pyrazolo[4,3-b]pyridin-7-yl]piperazine-1-carboxylate

7-Bromo-2-(4-bromophenyl)-2H-pyrazolo[4,3-b]pyridine-3-carboxamide (6.94 g, 17.5 mmol, Step E.6) and tert-butyl piperazine-1-carboxylate (10.1 g, 52.6 mmol) were suspended in N,N-dimethylacetamide (128 mL) and heated to 100° C. for 4 hours with a heating mantle. The flask was cooled to ambient temperature, and water (80 mL) was added dropwise. The resulting slurry was stirred for 60 minutes at ambient temperature. The product was isolated via filtration through a fritted funnel, washed with tert-butyl methyl ether, and dried to constant weight in vacuum oven at 60° C., to afford the title compound (6.43 g, 73.2%). ¹H NMR (500 MHz, CDCl₃) δ ppm 9.30 (d, J=3.5 Hz, 1H), 8.32 (d, J=5.4 Hz, 1H), 7.63 (d, J=8.6 Hz, 2H), 7.46 (d, J=8.7 Hz, 2H), 6.36 (d, J=5.5 Hz, 1H), 5.89 (d, J=3.4 Hz, 1H), 3.95 (s, 4H), 3.63 (dd, J=6.5, 4.0 Hz, 4H), 1.48 (s, 9H). MS (APCI) m/z: 503.3 [M+H]⁺.

### Intermediate F

### tert-butyl 4-[2-(4-bromo-2-methoxyphenyl)-3-carbamoyl-2H-pyrazolo[4,3-b]pyridin-7-yl]piperidine-1-carboxylate

### Step F.1

### tert-butyl 4-{(2E)-2-[2-(4-bromo-2-methoxyphenyl)hydrazinylidene]-2-cyanoacetyl}piperidine-1-carboxylate

4-Bromo-2-methoxyaniline (2.45 g, 11.9 mmol) was suspended in ice-cold water (37.7 mL) and concentrated HCl (3.61 mL, 119 mmol) was added, followed by dropwise addition of a solution of sodium nitrite (0.846 g, 11.89 mmol) in water (15.0 mL). The reaction was stirred at the same temperature for 30 minutes to afford a solution of the azide. A separate 1 L round-bottomed flask was charged with sodium acetate (29.3 g, 357 mmol), water (75 mL), tert-butyl 4-(2-cyanoacetyl)piperidine-1-carboxylate (3.90 g, 15.5 mmol, Step C.1), and ethanol (56.6 mL), and the resulting solution was cooled to <5° C. in an ice-water bath. The azide solution was then added dropwise via addition funnel over 10 minutes (exotherms maintained below 8° C.), during which time precipitation occurred. Once the addition was complete, the flask was removed from the bath and the resulting suspension was stirred at ambient temperature for another 5 minutes. The resulting product was isolated via filtration through a fritted funnel, washed with water, slurried in tert-butyl methyl ether in the funnel, and dried in a vacuum oven at 60° C. until constant weight to afford the title compound (5.7 g) that was used without additional purification. ¹H NMR showed a 3:1 ratio of isomers. Major isomer: ¹H NMR (400 MHz, CDCl₃) δ ppm 14.82 (s, 1H), 7.59 (d, J=8.6 Hz, 1H), 7.17 (dd, J=8.6, 1.9 Hz, 1H), 7.09 (d, J=1.9 Hz, 1H), 4.17 (br m, 2H), 3.97 (s, 3H), 3.13 (tt, J=11.4, 3.6 Hz, 1H), 2.96-2.72 (m, 2H), 1.90 (d, J=13.1 Hz, 2H), 1.71-1.55 (m, 2H), 1.47 (s, 9H). MS (APCI) m/z: 482.2 [M+H]⁺.

### Step F.2

### tert-butyl 4-[4-amino-1-(4-bromo-2-methoxyphenyl)-5-(ethoxycarbonyl)-1H-pyrazole-3-carbonyl]piperidine-1-carboxylate

A 500 mL round-bottomed flask was charged with tert-butyl 4-{(2E)-2-[2-(4-bromo-2-methoxyphenyl)hydrazinylidene]-2-cyanoacetyl}piperidine-1-carboxylate (5.53 g, 11.0 mmol, Step F.1) and 1,2-dioxane (33.0 mL). N,N-Diisopropylethylamine (20.8 mL, 119 mmol) was added, followed by addition of ethyl 2-bromoacetate (4.00 mL, 35.7 mmol). The reaction was heated to 100° C. for 3 hours using a heating mantle. The reaction flask was cooled to ambient temperature and diluted with ethyl acetate (150 mL). The reaction mixture was poured into a separatory funnel, and the layers were separated. The organic layer was washed with water (100 mL), and the combined aqueous extracts were extracted with additional ethyl acetate (2×). The combined organic extracts were washed with brine, dried over sodium sulfate, and concentrated under reduced pressure. The crude product was loaded onto a silica gel column and eluted with 0:100 to 40:60 ethyl acetate:heptanes to afford the title compound (5.10 g, 78%). ¹H NMR (400 MHz, CDCl₃) δ ppm 7.23-7.16 (m, 2H), 7.14 (d, J=1.8 Hz, 1H), 5.71 (br s, 2H), 4.26-4.07 (q, J=7.1 Hz, 2H), 3.77 (s, 3H), 3.56 (tt, J=11.4, 3.8 Hz, 1H), 3.01-2.69 (m, 4H), 1.86 (s, 2H), 1.71 (td, J=12.4, 4.3 Hz, 2H), 1.46 (s, 9H), 1.15 (t, J=7.1 Hz, 3H). MS (APCI) m/z: 452.9 [M+H]⁺.

### Step F.3

### ethyl 2-(4-bromo-2-methoxyphenyl)-7-[1-(tert-butoxycarbonyl)piperidin-4-yl]-2H-pyrazolo[4,3-b]pyridine-3-carboxylate

n-Butyllithium (11.5 mL, 28.7 mmol) was added slowly via syringe at −78° C. to a solution of N,N-diisopropylamine (4.15 mL, 29.6 mmol) dissolved in tetrahydrofuran (42.8 mL). (E)-N-ethylidene-2-methylpropan-2-amine (4.02 mL, 29.6 mmol, Step C.4) was added dropwise via syringe at the same temperature, and the solution was stirred for 20 minutes before addition of tert-butyl 4-[4-amino-1-(4-bromo-2-methoxyphenyl)-5-(ethoxycarbonyl)-1H-pyrazole-3-carbonyl]piperidine-1-carboxylate (5.1 g, 9.25 mmol, Step F.2) as a solution in tetrahydrofuran (25.7 mL). The reaction was stirred for another 15 minutes at the same temperature, quenched with 1 M aqueous HCl (50 mL), and warmed to ambient temperature. The mixture was diluted with ethyl acetate, stirred 5 minutes, and then the layers were separated. The organic layer was washed with 1 M aqueous HCl (2×), saturated sodium bicarbonate, brine, dried over sodium sulfate, and concentrated under reduced pressure. The crude product was purified by flash chromatography, eluting with 0:100 to 50:50 ethyl acetate:heptanes over 20 minutes on a silica gel column (120 g) to afford the title compound (1.23 g, 24%). ¹H NMR (400 MHz, CDCl₃) δ ppm 8.77 (d, J=4.4 Hz, 1H), 7.38 (d, J=8.3 Hz, 1H), 7.29 (dd, J=8.3, 1.9 Hz, 1H), 7.19 (d, J=1.9 Hz, 1H), 7.11 (dd, J=4.4, 0.7 Hz, 1H), 4.42 (q, J=7.1 Hz, 2H), 4.25 (s, 2H), 3.76 (s, 3H), 3.49 (tt, J=12.2, 3.5 Hz, 1H), 2.99-2.82 (m, 2H), 2.09-2.05 (m, 2H), 1.80 (qd, J=12.6, 4.3 Hz, 2H), 1.48 (s, 9H), 1.31 (t, J=7.1 Hz, 3H). MS (APCI) m/z: 559.1 [M+H]⁺.

### Step F.4

### 2-(4-bromo-2-methoxyphenyl)-7-[1-(tert-butoxycarbonyl)piperidin-4-yl]-2H-pyrazolo[4,3-b]pyridine-3-carboxylic acid

Ethyl 2-(4-bromo-2-methoxyphenyl)-7-[1-(tert-butoxycarbonyl)piperidin-4-yl]-2H-pyrazolo[4,3-b]pyridine-3-carboxylate (1.81 g, 3.24 mmol, Step F.3) was suspended in a mixture of tetrahydrofuran (10.8 mL), water (5.39 mL), and methanol (5.39 mL). Lithium hydroxide hydrate (0.679 g, 16.2 mmol) was added, and the reaction was stirred at ambient temperature for 60 minutes. Complete conversion was achieved as indicated by LC-MS. The reaction was acidified with 1 M HCl to pH 3-4, then stirred for 30 minutes at ambient temperature. The mixture was concentrated to approximately 10 mL volume in vacuo, and the organic material was extracted into ethyl acetate (3×). The combined organic extracts were washed with water, brine, dried over sodium sulfate, and concentrated under reduced pressure to afford the title compound (1.67 g, 97%). ¹H NMR (500 MHz, CDCl₃) δ ppm 9.95 (br s, 1H), 9.44 (d, J=5.2 Hz, 1H), 7.48 (d, J=5.2 Hz, 1H), 7.36 (d, J=8.3 Hz, 1H), 7.32-7.16 (m, 2H), 4.32 (br s, 2H), 3.81 (s, 3H), 3.63 (tt, J=12.0, 3.4 Hz, 1H), 2.93 (br d, J=15.3 Hz, 2H), 2.15-2.04 (m, 2H), 1.88 (qd, J=12.6, 4.3 Hz, 2H), 1.49 (s, 9H). MS (APCI) m/z: 531.3 [M+H]⁺.

### Step F.5

### tert-butyl 4-[2-(4-bromo-2-methoxyphenyl)-3-carbamoyl-2H-pyrazolo[4,3-b]pyridin-7-yl]piperidine-1-carboxylate

2-(4-bromo-2-methoxyphenyl)-7-[1-(tert-butoxycarbonyl)piperidin-4-yl]-2H-pyrazolo[4,3-b]pyridine-3-carboxylic acid (1.67 g, 3.14 mmol, Step F.4) was suspended in tetrahydrofuran (21.0 mL) in a 100-mL round-bottomed flask, and triethylamine (1.31 mL, 9.43 mmol) was added which solubilized the material. The flask was cooled in an ice-water bath to <5° C., and isobutyl chloroformate (0.825 mL, 6.29 mmol) was added, which resulted immediately in precipitation. The reaction was stirred for 5 minutes at ambient temperature, and ammonia (0.5 M in 1,4-dioxane, 44.0 mL, 22.00 mmol) was added. The reaction was stirred at ambient temperature for 30 minutes. The reaction mixture was diluted with ethyl acetate and water, and then stirred for 5 minutes at ambient temperature. The layers were separated. The organic layer was washed with brine, dried over sodium sulfate, and concentrated under reduced pressure. The residue was purified by flash chromatography, eluting with 0:100 to 50:50 ethyl acetate:heptanes over 20 minutes on a silica gel column (80 g) to afford the title compound (1.54 g, 92%). ¹H NMR (500 MHz, CDCl₃) δ ppm 8.78 (d, J=3.2 Hz, 1H), 8.62 (d, J=4.5 Hz, 1H), 7.29 (s, 1H), 7.28-7.25 (m, 1H), 7.20 (d, J=1.9 Hz, 1H), 7.14 (dd, J=4.5, 0.8 Hz, 1H), 5.88-5.80 (m, 1H), 4.44-4.17 (m, 2H), 3.77 (s, 3H), 3.51 (tt, J=12.2, 3.5 Hz, 1H), 2.91 (s, 2H), 2.07 (d, J=13.2 Hz, 2H), 1.85-1.75 (m, 2H), 1.48 (s, 9H). MS (APCI) m/z: 530.4 [M+H]⁺.

### Intermediate G

### [4-(2,5-difluorophenoxy)phenyl]boronic acid

### Step G.1

### 2,5-difluoro-1-(4-nitrophenoxy)benzene

To a stirred solution of 1-fluoro-4-nitrobenzene (20 g, 142 mmol) and 2,5-difluorophenol (20.28 g, 156 mmol) in dimethylformamide (100 mL) under nitrogen was added Cs₂CO₃ (69.3 g, 213 mmol). The mixture was stirred at 120° C. for 1 hour, cooled to room temperature and quenched with brine. The mixture was extracted with dichloromethane (3×). The combined organic layers were washed with brine, dried over Na₂SO₄, and concentrated under reduced pressure. The crude residue was purified by flash chromatography on silica gel eluting with 0 to 10% ethyl acetate in petroleum ether) to afford the title compound (33.8 g, 95%). ¹H NMR (400 MHz, dimethyl sulfoxide-d₆) δ ppm 7.22 (d, J=9.17 Hz, 3H) 7.44 (m, 1H) 7.51-7.60 (m, 1H) 8.24-8.29 (m, 2H).

### Step G.2

### 4-(2,5-difluorophenoxy)aniline

To the solution of 2,5-difluoro-1-(4-nitrophenoxy)benzene (3.5 g, 12.54 mmol, Step G.2) in ethanol (35 mL) and water (17.5 mL) was added ammonium chloride (1.01 g, 18.8 mmol) and iron (3.50 g, 62.7 mmol). The mixture was stirred at 70° C. for 2 hours then cooled to room temperature. The solids were filtered out and the filtrate was concentrated under reduced pressure. The crude residue was purified by flash chromatography on silica gel eluting with petroleum ether/ethyl acetate to afford the title compound (2.5 g, 77%). ¹H NMR (400 MHz, dimethyl sulfoxide-d₆) δ ppm 5.05 (s, 2H) 6.61 (s, 3H) 6.81 (d, J=8.80 Hz, 2H) 6.89 (br d, J=0.98 Hz, 1H) 7.36 (br d, J=5.26 Hz, 1H)

### Step G.3

### [4-(2,5-difluorophenoxy)phenyl]boronic acid

To a solution of 4-(2,5-difluorophenoxy)aniline (35 g, 160 mmol, prepared using Step G.2) and 12 N aqueous HCl (57.7 mL) in 4:1 methanol/water (250 mL) was added sodium nitrite (10.9 g, 158 mmol) at 0° C. The resulting solution was warmed to ambient temperature and stirred for 1 hour. Potassium acetate (46.6 g, 475 mmol) and tetrahydroxydiboron (42.6 g, 475 mmol) were added to the mixture. The reaction mixture was stirred at ambient temperature for 1 hour. The reaction was quenched with saturated aqueous NaHCO₃, and the resulting solution was extracted with dichloromethane (3×). The combined organic layers were washed with brine, dried over Na₂SO₄, and concentrated under reduced pressure to afford the crude residue, which was purified by preparative HPLC (35-75% acetonitrile in water with 0.2% formic acid over 20 minutes; Column: Kromasil® C18 150 mm×30 mm, 5 μm particle size; flow rate: 20 mL/min; detection wavelength: 220 nm and 254 nm) to provide the title compound (8.1 g, 32 mmol, 68%). ¹H NMR (400 MHz, dimethyl sulfoxide-d₆) δ ppm 8.02 (s, 2H), 7.87 (d, J=8.0 Hz, 1H), 7.79 (dd, J=8.8, 2.2 Hz, 1H), 7.51-7.40 (m, 1H), 7.17-7.04 (m, 2H), 7.01 (d, J=8.3 Hz, 1H), 6.95 (d, J=8.5 Hz, 1H).

### Intermediate H

### [4-(2-cyanophenoxy)phenyl]boronic acid

### Step H.1

### 2-(4-nitrophenoxy)benzonitrile

To a stirred solution of 1-fluoro-4-nitrobenzene (14.2 g, 101 mmol) and 2-hydroxybenzonitrile (12.0 g, 101 mmol) in N,N-dimethylformamide (120 mL) was added K₂CO₃ (13.9 g, 101 mmol). The resulting solution was warmed to 60° C. and stirred for 2 hours. The reaction mixture was cooled to 25° C. and quenched with brine. The resulting solution was extracted with dichloromethane (3×). The combined organic layers were washed with brine, dried over Na₂SO₄, and concentrated under reduced pressure to provide the title compound (21 g, 87%). MS (ESI) m/z: 241.1 [M+H]⁺.

### Step H.2

### 2-(4-aminophenoxy)benzonitrile

To a round bottom flask equipped with a stir bar was added 10% Pd/C (9.3 g, 8.8 mmol). The flask was sealed with a septum and purged with nitrogen (3×). Methanol (10 mL) and 2-(4-nitrophenoxy)benzonitrile (21 g, 88 mmol, Step H.1) were added. The vessel was backfilled with H₂ (3×) and stirred at ambient temperature for 6 hours. The resulting solution was filtered over diatomaceous earth. The filtrate was concentrated under reduced pressure to provide the title compound (18 g, 98%). MS (ESI) m/z: 211.2 [M+H]⁺.

### Step H.3

### [4-(2-cyanophenoxy)phenyl]boronic acid

Step H.3 was prepared according to the procedure for Intermediate G, substituting 2-(4-aminophenoxy)benzonitrile (Step H.2) for 4-(2,5-difluorophenoxy)aniline. MS (ESI) m/z: 283.9 [M+COOH⁻]⁻.

### Intermediate I

### [4-(2,4-difluorophenoxy)phenyl]boronic acid

### Step I.1

### 2,4-difluoro-1-(4-nitrophenoxy)benzene

Step I.1 was prepared according to the procedure for Step H.1, substituting 2,4-difluorophenol for 2-hydroxybenzonitrile. ¹H NMR (400 MHz, dimethyl sulfoxide-d₆) δ ppm 8.27-8.18 (m, 2H), 7.60-7.43 (m, 2H), 7.25-7.17 (m, 1H), 7.16-7.08 (m, 2H).

### Step I.2

### 4-(2,4-difluorophenoxy)aniline

Step I.2 was prepared according to the procedure for Step H.2, substituting 2,4-difluoro-1-(4-nitrophenoxy)benzene (Step I.1) for 2-(4-nitrophenoxy)benzonitrile. ¹H NMR (400 MHz, dimethyl sulfoxide-d₆) δ ppm 7.41-7.30 (m, 1H), 7.03-6.88 (m, 2H), 6.76-6.68 (m, 2H), 6.59-6.51 (m, 2H), 4.99 (s, 2H).

### Step I.3

### [4-(2,4-difluorophenoxy)phenyl]boronic acid

Step I.3 was prepared according to the procedure for Intermediate G substituting 4-(2,4-difluorophenoxy)aniline (Step I.2) for 4-(2,5-difluorophenoxy)aniline. ¹H NMR (400 MHz, CDCl₃) δ ppm 8.15 (d, J=8.5 Hz, 2H), 7.20-7.07 (m, 1H), 7.05-6.86 (m, 4H).

### Intermediate J

### trifluoroacetic acid-tert-butyl 3-[3-carbamoyl-2-(4-phenoxyphenyl)-2,4,5,6-tetrahydro-7H-pyrazolo[3,4-b]pyrazin-7-yl]azetidine-1-carboxylate (1/1)

### Step J.1

### 3,5-dibromo-1-[(4-methoxyphenyl)methyl]-4-nitro-1H-pyrazole

To a mixture of 3,5-dibromo-4-nitro-1H-pyrazole (5 g, 18.5 mmol) and potassium carbonate (7.65 g, 55.4 mmol) in acetonitrile (50 mL) was added 1-(chloromethyl)-4-methoxybenzene (3.47 g, 22.1 mmol) dropwise with stirring at 25° C. The reaction mixture was heated to 80° C. and stirred for 12 hours. Three additional reactions were set up as described above. After cooling to ambient temperature, all four reaction mixtures were combined and quenched by addition of water (200 mL). The mixture was then extracted with ethyl acetate (3×). The combined organic layers were washed with brine, dried over Na₂SO₄, and filtered. The filtrate was concentrated under reduced pressure to afford a crude residue, which was purified by silica gel column (eluted with 20:1 petroleum ether:ethyl acetate to 100% ethyl acetate) to afford the title compound (20 g, 65.8%). ¹H NMR (400 MHz, CDCl₃) δ ppm 3.77 (s, 3H) 5.31 (s, 2H) 6.86 (d, J=8.60 Hz, 2H) 7.24 (d, J=8.16 Hz, 2H)

### Step J.2

### tert-butyl 3-({3-bromo-1-[(4-methoxyphenyl)methyl]-4-nitro-1H-pyrazol-5-yl}amino)azetidine-1-carboxylate

To a solution of 3,5-dibromo-1-[(4-methoxyphenyl)methyl]-4-nitro-1H-pyrazole (6 g, 15.3 mmol, Step J.1) and N,N-diisopropylethylamine (8.04 mL, 46.0 mmol) in acetonitrile (60 mL) was added tert-butyl 3-aminoazetidine-1-carboxylate (2.91 g, 16.9 mmol) dropwise with stirring at 25° C. The reaction mixture was heated to 80° C. and stirred for 3 days. A second reaction was set up as described above. After cooling to ambient temperature, both reaction mixtures were combined and quenched by the addition of water. The mixture was then extracted with ethyl acetate (3×). The combined organic layers were washed with brine, dried over Na₂SO₄, and filtered. The filtrate was concentrated under reduced pressure to afford a crude residue, which was purified by silica gel column (eluted with 20:1 petroleum ether:ethyl acetate to 100% ethyl acetate) to afford the title compound (10 g, 64.2%). ¹H NMR (400 MHz, CDCl₃) δ ppm 1.38-1.49 (m, 9H) 1.58 (s, 1H) 3.76-3.87 (m, 5H) 4.11 (dd, J=8.99, 7.52 Hz, 2H) 4.18-4.29 (m, 1H) 5.14 (s, 2H) 6.89-6.94 (m, 2H) 7.06 (d, J=8.80 Hz, 2H) 7.25 (br d, J=8.44 Hz, 1H).

### Step J.3

### tert-butyl 3-[{3-bromo-1-[(4-methoxyphenyl)methyl]-4-nitro-1H-pyrazol-5-yl}(prop-2-en-1-yl)amino]azetidine-1-carboxylate

To a solution of tert-butyl 3-({3-bromo-1-[(4-methoxyphenyl)methyl]-4-nitro-1H-pyrazol-5-yl}amino)azetidine-1-carboxylate (5 g, 10.4 mmol, Step J.2) in N,N-dimethyl formamide (50 mL) was added potassium hexamethyldisilazide (2.48 g, 12.4 mmol) and allyl bromide (4.49 mL, 51.8 mmol) dropwise. The reaction mixture was stirred at 25° C. for 12 hours. A second reaction was set up as described above. Both reaction mixtures were combined and quenched by addition of water at ambient temperature, and the mixture was extracted with ethyl acetate (3×). The combined organic layers were washed with brine (100 mL), dried over Na₂SO₄, and filtered. The filtrate was concentrated under reduced pressure to afford a crude residue, which was purified by silica gel column (eluted with 5:1 petroleum ether:ethyl acetate to 100% ethyl acetate) to afford the title compound (7.7 g, yield 67.5%). ¹H NMR (400 MHz, CDCl₃) δ ppm 1.43 (s, 9H) 3.66 (br d, J=7.02 Hz, 3H) 3.80 (s, 4H) 3.86-3.99 (m, 2H) 4.15 (br d, J=5.70 Hz, 1H) 5.09-5.18 (m, 2H) 5.23 (s, 2H) 5.66 (ddt, J=16.88, 9.76, 7.07, 7.07 Hz, 1H) 6.88 (d, J=8.77 Hz, 2H) 7.25 (d, J=8.77 Hz, 2H).

### Step J.4

### tert-butyl 3-[{3-cyano-1-[(4-methoxyphenyl)methyl]-4-nitro-1H-pyrazol-5-yl}(prop-2-en-1-yl)amino]azetidine-1-carboxylate

To a solution of tert-butyl 3-[{3-bromo-1-[(4-methoxyphenyl)methyl]-4-nitro-1H-pyrazol-5-yl}(prop-2-en-1-yl)amino]azetidine-1-carboxylate (7.7 g, 14.7 mmol, Step J.3) in N,N-dimethyl formamide (70 mL) was added cyanocopper (6.60 g, 73.7 mmol) portion wise. The reaction mixture was heated to 100° C. and stirred for 12 hours, and then quenched with water. The mixture was filtered, and the filtrate was extracted with ethyl acetate (3×). The combined organic layers were washed with brine, dried over Na₂SO₄, and filtered. The filtrate was concentrated under reduced pressure to afford a crude residue which was purified by silica gel column (eluted with 5:1 petroleum ether:ethyl acetate to 100% ethyl acetate) to afford the title compound (4.5 g, 58.6%). ¹H NMR (400 MHz, CDCl₃) δ ppm 1.43 (s, 9H) 3.66 (br d, J=7.02 Hz, 3H) 3.80 (s, 4H) 3.92 (br d, J=5.26 Hz, 2H) 4.13-4.21 (m, 1H) 5.08-5.18 (m, 2H) 5.23 (s, 2H) 5.66 (ddt, J=16.88, 9.76, 7.07, 7.07 Hz, 1H) 6.88 (d, J=8.33 Hz, 2H) 7.25 (d, J=8.33 Hz, 2H).

### Step J.5

### tert-butyl 3-[{3-carbamoyl-1-[(4-methoxyphenyl)methyl]-4-nitro-1H-pyrazol-5-yl}(prop-2-en-1-yl)amino]azetidine-1-carboxylate

To a solution of tert-butyl 3-[{3-cyano-1-[(4-methoxyphenyl)methyl]-4-nitro-1H-pyrazol-5-yl}(prop-2-en-1-yl)amino]azetidine-1-carboxylate (2.2 g, 4.70 mmol, Step J.4) in dimethyl sulfoxide (20 mL) was added K₂CO₃ (1.29 g, 9.39 mmol) and H₂O₂ (2.39 mL, 23.5 mmol) dropwise. The reaction mixture was stirred at 25° C. for 12 hours. The reaction mixture was quenched by addition of saturated aqueous sodium sulfite solution at ambient temperature and then stirred for 10 minutes. The mixture was extracted with ethyl acetate (3×). The organic phase was washed with brine, dried over Na₂SO₄, filtered, and the filtrate was concentrated under reduced pressure to afford the title compound (2 g, 83%). ¹H NMR (400 MHz, CDCl₃) δ ppm 1.43 (s, 9H) 3.63 (br d, J=6.97 Hz, 4H) 3.80 (s, 3H) 3.92 (br t, J=8.19 Hz, 2H) 4.04-4.17 (m, 1H) 5.07-5.18 (m, 2H) 5.27 (s, 2H) 5.58-5.71 (m, 1H) 5.80 (br s, 1H) 6.88 (d, J=8.44 Hz, 2H) 6.97 (br s, 1H) 7.22 (br d, J=8.44 Hz, 2H).

### Step J.6

### tert-butyl 3-[(3-carbamoyl-4-nitro-1H-pyrazol-5-yl)(prop-2-en-1-yl)amino]azetidine-1-carboxylate

To a flask containing tert-butyl 3-[{3-carbamoyl-1-[(4-methoxyphenyl)methyl]-4-nitro-1H-pyrazol-5-yl}(prop-2-en-1-yl)amino]azetidine-1-carboxylate (500 mg, 1.03 mmol, Step J.5) was added trifluoroacetic acid (5 mL, 64.9 mmol). The reaction mixture was stirred at 80° C. for 1 hour. The reaction mixture was concentrated under reduced pressure to afford a crude oil. The crude oil was diluted with dichloromethane (2 mL) and N,N-diisopropylethylamine (0.215 mL, 1.23 mmol) was added dropwise until the pH was >7. Di-tert-butyl dicarbonate (0.286 mL, 1.23 mmol) was added into the mixture. The resulting mixture was stirred at 25° C. for 1 hour. Five additional reactions were set up as described above. All six reaction mixtures were combined and quenched by the addition of water at ambient temperature. The resulting mixture was extracted with dichloromethane (3×30 mL). The combined organic layers were washed with brine (15 mL), dried over Na₂SO₄, and filtered. The filtrate was concentrated under reduced pressure to afford a residue, which was purified by silica gel column (eluted with 5:1 petroleum ether:ethyl acetate to 100% ethyl acetate) to afford the title compound (1 g, 42.0%). ¹H NMR (400 MHz, dimethyl sulfoxide-d₆) δ ppm 1.36 (s, 9H) 3.60-3.77 (m, 2H) 3.78-4.10 (m, 4H) 4.18-4.40 (m, 1H) 5.05-5.20 (m, 2H) 5.66-5.81 (m, 1H) 7.41-8.40 (m, 2H) 12.99-14.08 (m, 1H).

### Step J.7

### tert-butyl 3-{[5-carbamoyl-4-nitro-1-(4-phenoxyphenyl)-1H-pyrazol-3-yl](prop-2-en-1-yl)amino}azetidine-1-carboxylate

To a solution of tert-butyl 3-[(3-carbamoyl-4-nitro-1H-pyrazol-5-yl)(prop-2-en-1-yl)amino]azetidine-1-carboxylate (1.23 g, 3.36 mmol, Step J.6) in dichloromethane (15 mL) was added pyridine (0.326 mL, 4.03 mmol), (4-phenoxyphenyl)boronic acid (1.44 g, 6.71 mmol), 4 Å molecular sieves (100 mg), and copper (II) acetate (0.671 g, 3.69 mmol). The reaction mixture was stirred at 25° C. for 12 hours under oxygen atmosphere. The mixture was quenched with water, and the resulting mixture was extracted with ethyl acetate (3×). The organic phase was washed with brine, dried over Na₂SO₄, and filtered. The filtrate was concentrated under reduced pressure to afford a crude residue which was purified by silica gel column (eluted with 10:1 petroleum ether:ethyl acetate to 100% ethyl acetate) to afford the title compound (700 mg, 37.1%). ¹H NMR (400 MHz, dimethyl sulfoxide-d₆) δ ppm 1.36 (s, 9H) 3.79 (br dd, J=8.38, 5.73 Hz, 2H) 3.90-4.02 (m, 4H) 4.25-4.35 (m, 1H) 5.12-5.24 (m, 2H) 5.77-5.88 (m, 1H) 7.11 (dd, J=15.44, 8.38 Hz, 4H) 7.18-7.26 (m, 1H) 7.41-7.49 (m, 2H) 7.56 (d, J=9.04 Hz, 2H) 8.20 (s, 1H) 8.44 (s, 1H).

### Step J.8

### tert-butyl 3-{[5-carbamoyl-4-nitro-1-(4-phenoxyphenyl)-1H-pyrazol-3-yl](2-oxoethyl)amino}azetidine-1-carboxylate

tert-Butyl 3-{[5-carbamoyl-4-nitro-1-(4-phenoxyphenyl)-1H-pyrazol-3-yl](prop-2-en-1-yl)amino}azetidine-1-carboxylate (100 mg, 0.187 mmol, Step J.7) was dissolved in a mixture solvent of tetrahydrofuran (1 mL) and water (0.05 mL). The solution was treated with osmium tetroxide (5.0 mL, 1.967 mmol) and 4-methylmorpholine N-oxide (43.8 mg, 0.374 mmol). The reaction mixture was stirred at 25° C. for 14 hours. Aqueous sodium bisulfate solution (1.87 mL, 1.871 mmol) was added to the solution and the biphasic mixture was stirred for 10 minutes at ambient temperature. The mixture was diluted with brine (20 mL) and extracted with ethyl acetate (3×20 mL). The combined organic phase was dried over sodium sulfate, filtered, and the filtrate was concentrated under reduced pressure to afford a crude residue, which was used without additional purification. Sodium periodate (80 mg, 0.374 mmol) was dissolved in water (0.75 mL) and added at 25° C. into a solution of the above crude residue in acetone (1.0 mL). The resulting mixture was stirred at 25° C. for 2 hours. Four reactions were set up as described above. All five reaction mixtures were combined and diluted with brine and extracted with ethyl acetate (3×). The organic layers were combined, washed with brine, dried over sodium sulfate, and filtered. The filtrate was concentrated under reduced pressure to afford a residue which was purified by silica gel column (eluted with 100:1 petroleum ether:ethyl acetate to 100% ethyl acetate) to afford the title compound (400 mg, 76%). ¹H NMR (400 MHz, dimethyl sulfoxide-d₆) δ ppm 1.37 (s, 9H) 3.78-3.88 (m, 2H) 4.03-4.09 (m, 2H) 4.28 (s, 2H) 4.50-4.60 (m, 1H) 7.07-7.17 (m, 4H) 7.19-7.25 (m, 1H) 7.41-7.48 (m, 2H) 7.56 (d, i=8.82 Hz, 2H) 8.20 (s, 1H) 8.44 (s, 1H) 9.65 (s, 1H).

### Step J.9 trifluoroacetic acid-tert-butyl 3-[3-carbamoyl-2-(4-phenoxyphenyl)-2,4,5,6-tetrahydro-7H-pyrazolo[3,4-b]pyrazin-7-yl]azetidine-1-carboxylate (1/1)

To a solution of tert-butyl 3-{[5-carbamoyl-4-nitro-1-(4-phenoxyphenyl)-1H-pyrazol-3-yl](2-oxoethyl)amino}azetidine-1-carboxylate (400 mg, 0.746 mmol, Step J.8) in tetrahydrofuran (20 mL) was added palladium on carbon (79 mg) under N2 atmosphere. The mixture was stirred at 70° C. for 2 hours under H₂ (15 psi). Three additional reactions were set up as described above. After cooling to ambient temperature, all four reaction mixtures were combined and filtered through a diatomaceous earth pad. The filtrate was diluted with water and the mixture was extracted with ethyl acetate (3×). The organic phases were combined, washed with brine, dried over Na₂SO₄, and filtered. The filtrate was concentrated under reduced pressure to afford a residue which was purified by preparative HPLC (Welch Ultimate AQ-C18 150×30 mm×5 μm, 5 μm particle size; Mobile phase: (A) 0.075% v/v CF₃COOH/H₂O and (B) Acetonitrile, gradient: B %=48-78% over 12 minutes; flow rate: 25 mL/min; detection wavelength: 220 nm and 254 nm) to afford the title compound (300 mg, 64.8%). ¹H NMR (400 MHz, CDCl₃) δ ppm 1.45 (s, 9H) 3.26-3.40 (m, 2H) 3.48-3.60 (m, 2H) 4.15 (d, J=6.84 Hz, 4H) 4.44 (t, J=6.50 Hz, 1H) 6.99-7.12 (m, 4H) 7.15-7.23 (m, 1H) 7.29-7.43 (m, 4H).

### Intermediate K

### 4-[(4-methoxyphenyl)methyl]-7-oxo-2-(4-phenoxyphenyl)-4,5,6,7-tetrahydro-2H-pyrazolo[4,3-b]pyridine-3-carboxamide

### Step K.1

### (2E)-3-oxo-2-[2-(4-phenoxyphenyl)hydrazinylidene]butanenitrile

To a round-bottomed flask charged with a suspension of 4-phenoxyaniline (11.80 g, 63.7 mmol) in water (79 mL) was added 1 M aqueous hydrogen chloride (53 mL, 53.0 mmol), followed by a dropwise addition of sodium nitrite (4.40 g, 63.7.0 mmol) in water (67 mL) at 0° C. The reaction mixture was warmed to ambient temperature and stirred for another 1 hour. The reaction mixture was filtered to remove the insoluble material. The crude 4-phenoxybenzenediazonium product was used in the following step without purification. MS m/z: 198.07 [M+H]⁺.

To a round-bottomed flask charged with sodium acetate (157 g, 1911 mmol), 3-oxobutanenitrile (7.94 g, 96 mmol), ethanol (250 mL) and water (333 mL) at 0° C. was added 4-phenoxybenzenediazonium (12.56 g, 63.7 mmol) aqueous solution via addition funnel over 10 minutes while keeping the internal temperature below 8° C. After addition, the reaction mixture was warmed to ambient temperature and stirred for another 5 minutes. The precipitate was collected by filtration, washed with water (3×) and dried under vacuum to afford the title compound (15 g, 84%). MS m/z: 280.1 [M+H]⁺.

### Step K.2

### ethyl 3-acetyl-4-amino-1-(4-phenoxyphenyl)-1H-pyrazole-5-carboxylate

To a 500 mL round-bottomed flask charged with (2E)-3-oxo-2-[2-(4-phenoxyphenyl)hydrazinylidene]butanenitrile (14 g, 50.1 mmol, Step K.1), N-ethyl-N-isopropylpropan-2-amine (64.8 g, 501 mmol) and 1,4-dioxane (140 mL) was added ethyl 2-bromoacetate (25.1 g, 150 mmol), and the resulting mixture was heated at 100° C. for 5 hours. The reaction mixture was diluted with ethyl acetate, washed with water (3×), dried over Na₂SO₄, and concentrated to remove the solvent. The residue was diluted with tert-butyl methyl ether and petroleum ether forming a slurry. The precipitate was collected by filtration to afford the title compound (10.5 g, 57.3%). MS (ESI) m/z: 366.14 [M+H]⁺.

### Step K.3

### ethyl 7-hydroxy-2-(4-phenoxyphenyl)-2H-pyrazolo[4,3-b]pyridine-3-carboxylate hydrochloride

To a 1 L round-bottomed flask charged with ethyl 3-acetyl-4-amino-1-(4-phenoxyphenyl)-1H-pyrazole-5-carboxylate (10 g, 27.4 mmol, Step K.2) and tetrahydrofuran (200 mL) cooled to 10° C., was added NaH (1.97 g, 82 mmol) portion wise. After the addition, the reaction mixture was warmed to ambient temperature and stirred for 10 minutes. Ethyl formate (10.14 g, 137 mmol) was added, and the resulting mixture was stirred for 16 hours at ambient temperature. 1 M aqueous hydrogen chloride (290 mL, 290 mmol) was then added, and the mixture was heated to 45° C. and stirred for another 16 hours. The precipitate was collected by filtration, washed with water (3×) and dried under vacuum to afford the title compound (8.0 g, 78%). MS (ESI) m/z: 376.23 [M+H]⁺.

### Step K.4

### ethyl 4-[(4-methoxyphenyl)methyl]-7-oxo-2-(4-phenoxyphenyl)-4,7-dihydro-2H-pyrazolo[4,3-b]pyridine-3-carboxylate

To a 50 mL round-bottomed flask charged with ethyl 7-hydroxy-2-(4-phenoxyphenyl)-2H-pyrazolo[4,3-b]pyridine-3-carboxylate hydrochloride (1 g, 2.66 mmol, Step K.3), 1,8-diazabicyclo[5.4.0]undec-7-ene (0.803 mL, 5.33 mmol) and N,N-dimethyl formamide (20 mL), was added 1-(chloromethyl)-4-methoxybenzene (0.417 g, 2.66 mmol), and the mixture was heated at 90° C. for 16 hours. The mixture was diluted with ethyl acetate and the organic layer was separated. The aqueous layer was extracted with ethyl acetate. The organic extracts were combined, washed with brine and water, and dried over anhydrous Na₂SO₄. The crude residue was purified by flash column chromatography on silica gel (100% ethyl acetate) to afford the title compound (535 mg, 40.5%). MS (ESI) m/z: 496.18 [M+H]⁺.

### Step K.5

### ethyl 4-[(4-methoxyphenyl)methyl]-7-oxo-2-(4-phenoxyphenyl)-4,5,6,7-tetrahydro-2H-pyrazolo[4,3-b]pyridine-3-carboxylate

To a round-bottomed flask charged with ethyl 4-[(4-methoxyphenyl)methyl]-7-oxo-2-(4-phenoxyphenyl)-4,7-dihydro-2H-pyrazolo[4,3-b]pyridine-3-carboxylate (205 mg, 0.414 mmol) and tetrahydrofuran (2 mL) cooled to 0° C., was added lithium triethylborohydride (1 M solution in tetrahydrofuran, 0.83 mL, 0.830 mmol) dropwise. After the addition, the reaction mixture was stirred for 1 hour. The mixture was quenched with the saturated aqueous NH₄Cl solution then extracted with ethyl acetate (2×). The organic extracts were combined, washed with brine, dried over Na₂SO₄ and concentrated to a residue, which was purified by flash column chromatography on silica gel (100% ethyl acetate) to afford the title compound (100 mg, 48.6%). MS (ESI) m/z: 498.20 [M+H]⁺.

### Step K.6

### 4-[(4-methoxyphenyl)methyl]-7-oxo-2-(4-phenoxyphenyl)-4,5,6,7-tetrahydro-2H-pyrazolo[4,3-b]pyridine-3-carboxylic acid

To a round-bottomed flask charged with ethyl 4-[(4-methoxyphenyl)methyl]-7-oxo-2-(4-phenoxyphenyl)-4,5,6,7-tetrahydro-2H-pyrazolo[4,3-b]pyridine-3-carboxylate (320 mg, 0.643 mmol, Step K.5) was added lithium hydroxide hydrate (81 mg, 1.929 mmol), tetrahydrofuran (15 mL), methanol (15 mL) and water (7.5 mL), and the reaction mixture was stirred for 1 hour at ambient temperature. The mixture was diluted with water and extracted with ethyl acetate (2×). The organic extracts were washed with brine and water once, dried over Na₂SO₄, and concentrated to afford the title compound (300 mg, 99%). MS (ESI) m/z: 470.16 [M+H]⁺.

### Step K.7

### 4-[(4-methoxyphenyl)methyl]-7-oxo-2-(4-phenoxyphenyl)-4,5,6,7-tetrahydro-2H-pyrazolo[4,3-b]pyridine-3-carboxamide

To a round-bottomed flask charged with ammonium chloride (171 mg, 3.19 mmol), triethylamine (0.445 mL, 3.19 mmol), 4-[(4-methoxyphenyl)methyl]-7-oxo-2-(4-phenoxyphenyl)-4,5,6,7-tetrahydro-2H-pyrazolo[4,3-b]pyridine-3-carboxylic acid (300 mg, 0.639 mmol, Step K.6) and dichloromethane (30 mL) was added 1-[bis(dimethylamino)methylene]-1H-1,2,3-triazolo[4,5-b]pyridinium 3-oxid-hexafluoro phosphate (486 mg, 1.278 mmol), and the mixture was stirred for 2 hours at ambient temperature. The mixture was diluted with dichloromethane, and the organic layer was separated and washed with water and brine. The organic layer was dried over Na₂SO₄ and concentrated to afford the title compound (300 mg, 100%). MS (ESI) m/z: 469.18 [M+H]⁺.

### Intermediate L

### ethyl (7S)-7-[4-(2-nitrobenzene-1-sulfonyl)piperazin-1-yl]-4,5,6,7-tetrahydro-2H-pyrazolo[4,3-b]pyridine-3-carboxylate

### Step L.1

### diethyl 4-bromo-1-(oxan-2-yl)-1H-pyrazole-3,5-dicarboxylate

To a solution of diethyl 4-bromo-1H-pyrazole-3,5-dicarboxylate (146.2 g, 502 mmol) in tetrahydrofuran (1 L) was added 3,4-dihydro-2H-pyran (63.4 g, 753 mmol) and pyridinium p-toluenesulfonate (25.2 g, 100 mmol) at ambient temperature. The mixture was heated to 70° C. and stirred for 15 hours. The reaction mixture was cooled to ambient temperature, diluted with water, and extracted with ethyl acetate (350 mL). The combined organic layers were washed with brine, dried over Na₂SO₄, filtered, and concentrated under reduced pressure. The crude residue was purified by column chromatography (0% to 50% ethyl acetate in heptanes) to afford the title compound (139 g, 74%). MS (ESI) m/z: 397.1 [M+Na]⁺.

### Step L.2

### diethyl 4-[(3-tert-butoxy-3-oxopropyl)amino]-1-(oxan-2-yl)-1H-pyrazole-3,5-dicarboxylate

To a solution of diethyl 4-bromo-1-(oxan-2-yl)-1H-pyrazole-3,5-dicarboxylate (75.0 g, 200 mmol, Step L.1) and tert-butyl 3-aminopropanoate (43.5 g, 300 mmol) in 1,4-dioxane (2 L) was added tris(dibenzylideneacetone)dipalladium(0) (Pd₂(dba)₃) (18.3 g, 20.0 mmol) and 4,5-bis(diphenylphosphino)-9,9-dimethylxanthene (XantPhos) (17.4 g, 30.0 mmol) and Cs₂CO₃ (261 g, 800 mmol) at ambient temperature. The mixture was heated to 100° C. and stirred for 26 hours under N₂ atmosphere. The reaction mixture was cooled to ambient temperature, filtered, and concentrated under reduced pressure. The crude residue was purified by column chromatography (0% to 10% ethyl acetate in heptanes) to afford the title compound (63.3 g, 72%). MS (ESI) m/z: 440.5 [M+H]⁺.

### Step L.3

### ethyl 7-oxo-4,5,6,7-tetrahydro-2H-pyrazolo[4,3-b]pyridine-3-carboxylate

To a solution of diethyl 4-[(3-tert-butoxy-3-oxopropyl)amino]-1-(oxan-2-yl)-1H-pyrazole-3,5-dicarboxylate (44.0 g, 100 mmol, Step L.2) in tetrahydrofuran (1 L) was added lithium hexamethyldisilazide (58.6 g, 350 mmol) at −78° C. under a nitrogen atmosphere for 3 hours. The reaction was quenched with ice water and extracted with dichloromethane (3×). The combined organic layers were washed with brine, dried over Na₂SO₄, filtered, and concentrated under reduced pressure to afford the crude residue (47.0 g). To a solution of the crude residue (47.0 g) in tetrahydrofuran (600 mL) was added 6 M aqueous HCl (50 mL) at 0° C. The reaction mixture was heated to 60° C. and stirred for 2 hours. The reaction mixture was cooled to ambient temperature and diluted with saturated NaHCO₃ and extracted with dichloromethane (3×). The combined organic layers were washed with brine, dried over Na₂SO₄, filtered, and concentrated under reduced pressure. The crude residue was purified by column chromatography (0% to 50% ethyl acetate in heptanes) to afford the title compound (14.3 g, 52%). MS (ESI) m/z: 210.2 [M+H]⁺.

### Step L.4

### ethyl (7E)-7-{[(S)-2-methylpropane-2-sulfinyl]imino}-4,5,6,7-tetrahydro-2H-pyrazolo[4,3-b]pyridine-3-carboxylate

To a solution of ethyl 7-oxo-4,5,6,7-tetrahydro-2H-pyrazolo[4,3-b]pyridine-3-carboxylate (12.9 g, 61.7 mmol, Step L.3) in toluene (350 mL) was added (S)-2-methylpropane-2-sulfinamide (22.4 g, 185 mmol) and tetraethoxytitanium (42.2 g, 185 mmol) at ambient temperature. The reaction mixture heated to 75° C. and stirred for 6 hours. The reaction mixture was cooled to ambient temperature, diluted with water, and extracted with ethyl acetate (3×). The combined organic layers were washed with brine, dried over Na₂SO₄, filtered, and concentrated under reduced pressure. The crude residue was purified by column chromatography (0% to 60% ethyl acetate in heptanes) to afford the title compound (16.1 g, 84%). MS (ESI) m/z: 313.2 [M+H]⁺.

### Step L.5

### ethyl (7S)-7-{[(S)-2-methylpropane-2-sulfinyl]amino}-4,5,6,7-tetrahydro-2H-pyrazolo[4,3-b]pyridine-3-carboxylate

To a solution of ethyl (7E)-7-{[(S)-2-methylpropane-2-sulfinyl]imino}-4,5,6,7-tetrahydro-2H-pyrazolo[4,3-b]pyridine-3-carboxylate (12.2 g, 38.9 mmol, Step L.4) in tetrahydrofuran (150 mL) and water (2 mL) was added NaBH₄ (5.15 g, 136 mmol) at 0° C. The reaction mixture was warmed to ambient temperature and stirred for 8 hours. The reaction mixture was cooled to ambient temperature and diluted with saturated NH₄Cl and extracted with dichloromethane (3×). The combined organic layers were washed with brine, dried over Na₂SO₄, filtered, and concentrated under reduced pressure. The crude residue was purified by column chromatography (0% to 60% ethyl acetate in heptanes) to afford the title compound (10.2 g, 83%). MS (ESI) m/z: 315.2 [M+H]⁺.

### Step L.6

### [(2-nitrobenzene-1-sulfonyl)azanediyl]di(ethane-2,1-diyl) bis(2-nitrobenzene-1-sulfonate)

To a solution of 2,2′-azanediyldiethanol (20.0 g, 190 mmol) and 2-nitrobenzene-1-sulfonyl chloride (131 g, 590 mmol) in dichloromethane (1.27 L) was added 1,4-diazabicyclo[2.2.2]octane (66.1 g, 590 mmol) at 0° C. The mixture was warmed to ambient temperature and stirred for 1 hour. The reaction mixture was diluted with water and extracted with dichloromethane (2×). The combined organic layers were washed with brine, dried over Na₂SO₄, filtered, and concentrated under reduced pressure. The crude residue was purified by column chromatography (1% to 25% ethyl acetate in dichloromethane) to afford the title compound (80.0 g, 64%). ¹H NMR (400 MHz, CDCl₃) δ ppm 8.49-8.42 (m, 4H), 8.06-7.90 (m, 8H), 3.95-4.02 (m, 4H). δ ppm MS (ESI) m/z: 661.0 [M+H]⁺.

### Step L.7

### ethyl (7S)-7-[4-(2-nitrobenzene-1-sulfonyl)piperazin-1-yl]-4,5,6,7-tetrahydro-2H-pyrazolo[4,3-b]pyridine-3-carboxylate

To a solution of ethyl (7S)-7-{[(S)-2-methylpropane-2-sulfinyl]amino}-4,5,6,7-tetrahydro-2H-pyrazolo[4,3-b]pyridine-3-carboxylate (10.2 g, 32.4 mmol, Step L.5) in methanol (60 mL) was added HCl (4 M in 1,4-dioxane, 10 mL) at 0° C. The reaction mixture stirred at 0° C. for 3 hours, and then concentrated under reduced pressure to afford a crude residue (12.1 g). To a solution of the crude residue (6.81 g) in acetonitrile (150 mL) was added [(2-nitrobenzene-1-sulfonyl)azanediyl]di(ethane-2,1-diyl) bis(2-nitrobenzene-1-sulfonate) (32.1 g, 48.6 mmol, Step L.6) and N,N-diisopropylethylamine (16.8 g, 130 mmol). The reaction mixture was heated to 40° C. and stirred for 8 hours. The reaction mixture was cooled to ambient temperature, diluted with water, and extracted with dichloromethane (3×). The combined organic layers were washed with brine, dried over Na₂SO₄, filtered, and concentrated under reduced pressure. The crude residue was purified by column chromatography (0% to 80% ethyl acetate in heptanes) to afford the title compound (9.33 g, 62%). ¹H NMR (400 MHz, dimethyl sulfoxide-d₆) δ ppm 12.7 (s, 1H), 8.00-7.96 (m, 2H), 7.92 (ddd, J=7.6, 1.3, 1.3 Hz, 1H), 7.89-7.84 (m, 1H), 5.06 (s, 1H), 4.27-4.18 (m, 2H), 3.60 (t, J=5.4 Hz, 1H), 3.23-3.18 (m, 2H), 3.11-3.06 (m, 2H), 3.14-3.11 (m, 2H), 2.71-2.59 (m, 4H), 1.98-1.92 (m, 1H), 1.70-1.62 (m, 1H), 1.27 (t, J=7.1 Hz, 3H). MS (ESI) m/z: 465.1 [M+H]⁺.

### Intermediate M

### [4-(3-fluorophenoxy)phenyl]boronic acid

### Step M.1

### 2-[4-(3-fluorophenoxy)phenyl]-4,4,5,5-tetramethyl-1,3,2-dioxaborolane

To a solution of 4-(4,4,5,5-tetramethyl-1,3,2-dioxaborolan-2-yl)phenol (1 g, 4.54 mmol) in dichloromethane (50 mL) was added (3-fluorophenyl) boronic acid (0.69 g, 5.00 mmol), copper (II) acetate (1.07 g, 5.91 mmol) and triethylamine (2.76 g, 27.3 mmol). The reaction mixture was stirred for 12 hours at 25° C. under oxygen (15 psi), quenched with water (10 mL) and extracted with dichloromethane (3×). The organic phases were combined and washed with brine, dried over Na₂SO₄, and filtered. The filtrate was concentrated under reduced pressure to afford a crude product which was purified by column chromatography on silica gel (100:1 to 19:1 petroleum ether:ethyl acetate) to afford the title compound (0.5 g, 31.5%). ¹H NMR (400 MHz, CD3Cl) δ ppm 1.36 (s, 12H) 6.69-6.77 (m, 1H) 6.78-6.86 (m, 2H) 7.02 (d, J=8.38 Hz, 2H) 7.23-7.33 (m, 1H) 7.82 (d, J=8.60 Hz, 2H).

### Step M.2

### [4-(3-fluorophenoxy)phenyl]boronic acid

To a solution of 2-[4-(3-fluorophenoxy)phenyl]-4,4,5,5-tetramethyl-1,3,2-dioxaborolane (450 mg, 1.43 mmol, Step M.1) in a mixture of acetone (5 mL) and water (2.5 mL) was added sodium periodate (919 mg, 4.30 mmol) and ammonium acetate (331 mg, 4.30 mmol), and then the reaction mixture was stirred for 2 hours at 20° C. The reaction mixture was quenched with water, and the resulting mixture was extracted with dichloromethane (3×). The organic phases were combined, washed with brine, dried over Na₂SO₄, and filtered. The filtrate was concentrated under reduced pressure to afford a crude residue which was purified by column chromatography on silica gel (30:1 to 0:1 petroleum ether:ethyl acetate) to afford the title compound (200 mg, 54.2%). ¹H NMR (400 MHz, dimethyl sulfoxide-d₆) δ ppm 6.79-6.93 (m, 3H) 6.96-7.03 (m, 3H) 7.07 (br d, J=8.44 Hz, 2H) 7.37-7.48 (m, 2H) 7.83 (d, J=8.44 Hz, 2H) 7.92 (br d, J=8.19 Hz, 1H) 8.01 (br s, 2H).

### Intermediate N

### [4-(3,4-difluorophenoxy)phenyl]boronic acid

### Step N.1

### 2-[4-(3,4-difluorophenoxy)phenyl]-4,4,5,5-tetramethyl-1,3,2-dioxaborolane

Step N.1 was prepared according to the procedure for Step M.1, substituting (3,4-difluorophenyl)boronic acid for (3-fluorophenyl) boronic acid. ¹H NMR (400 MHz, CDCl₃) δ ppm 1.30-1.40 (m, 12H) 6.70-6.79 (m, 1H) 6.86 (ddd, J=11.19, 6.67, 2.87 Hz, 1H) 6.98 (d, J=8.38 Hz, 2H) 7.13 (d, J=9.92 Hz, 1H) 7.81 (d, J=8.60 Hz, 2H).

### Step N.2

### [4-(3,4-difluorophenoxy)phenyl]boronic acid

Step N.2 was prepared according to the procedure for Step M.2, substituting 2-[4-(3,4-difluorophenoxy)phenyl]-4,4,5,5-tetramethyl-1,3,2-dioxaborolane (Step N.1) for 2-[4-(3-fluorophenoxy)phenyl]-4,4,5,5-tetramethyl-1,3,2-dioxaborolane. ¹H NMR (400 MHz, dimethyl sulfoxide-d₆) δ ppm 6.87 (dt, J=9.04, 1.43 Hz, 1H) 6.97 (d, J=8.60 Hz, 1H) 7.17-7.27 (m, 1H) 7.38-7.53 (m, 1H) 7.81 (d, J=8.60 Hz, 1H).

### Intermediate O

### [4-(2,3-difluorophenoxy)phenyl]boronic acid

### Step O.1

### 1,2-difluoro-3-(4-nitrophenoxy)benzene

To a stirred solution of 1-fluoro-4-nitrobenzene (20.0 g, 142 mmol) and 2,3-difluorophenol (20.3 g, 156 mmol) in N,N-dimethylformamide (100 mL) was added Cs₂CO₃ (69.3 g, 213 mmol). The resulting solution was heated to 120° C. and stirred for 1 hour. The reaction mixture was cooled to 25° C. and quenched with brine. The resulting solution was extracted with dichloromethane (3×). The combined organic layers were washed with brine, dried over Na₂SO₄, and concentrated under reduced pressure. The crude product was purified by silica gel chromatography (0-10% ethyl acetate in petroleum ether) to provide the title compound (30 g, 84%). MS (ESI) m/z: 251.67 [M+H]⁺.

### Step O.2

### 4-(2,3-difluorophenoxy)aniline

To a round bottom flask equipped with a stir bar was added 10% Pd/C (1.27 g, 1.19 mmol). The flask was sealed with a septum and purged with N₂ (3×). Methanol (20 mL) and 1,2-difluoro-3-(4-nitrophenoxy)benzene (30 g, 120 mmol, Step O.1) were added. The vessel was backfilled with H₂ (3×) and stirred at ambient temperature for 6 hours. The resulting solution was filtered over diatomaceous earth. The filtrate was concentrated under reduced pressure at 50° C. to provide the title compound (26 g, 98%). MS (ESI) m/z: 222.33 [M+H]⁺.

### Step O.3

### [4-(2,3-difluorophenoxy)phenyl]boronic acid

To a solution of 4-(2,3-difluorophenoxy)aniline (26 g, 120 mmol, Step O.2) and HCl (10.71 mL, 12 N aqueous) in 4:1 methanol/water (250 mL) was added NaNO₂ (12.2 g, 176 mmol) at 0° C. The resulting solution was heated to ambient temperature and stirred for 1 hour. Potassium acetate (34.6 g, 353 mmol) and tetrahydroxydiboron (31.6 g, 353 mmol) were added to the mixture. The reaction mixture was stirred at ambient temperature for 1 hour. The reaction was quenched with saturated aqueous NaHCO₃ and extracted with dichloromethane (3×). The combined organic layers were washed with brine, dried over Na₂SO₄ and concentrated under reduced pressure to afford the crude residue, which was purified by preparative HPLC (35-85% acetonitrile in water with 0.2% formic acid over 20 minutes; Column: Kromasil® C18 150 mm×30 mm, 5 μm particle size; flow rate: 20 mL/min; detection wavelength: 220 nm and 254 nm) to provide the title compound (20 g, 68%). MS (ESI) m/z: 248.96 [M+H]⁺.

### Intermediate P

### ethyl 7-[1-(tert-butoxycarbonyl)-1,2,3,6-tetrahydropyridin-4-yl]-2-(4-phenoxyphenyl)-2H-pyrazolo[4,3-b]pyridine-3-carboxylate

### Step P.1

### ethyl 2-(4-phenoxyphenyl)-7-[(trifluoromethanesulfonyl)oxy]-2H-pyrazolo[4,3-b]pyridine-3-carboxylate

To a cooled suspension of ethyl 7-hydroxy-2-(4-phenoxyphenyl)-2H-pyrazolo[4,3-b]pyridine-3-carboxylate (810 mg, 2.158 mmol, Step K.3) and triethylamine (1.770 mL, 12.95 mmol) in dichloromethane at −50° C. was added trifluoromethanesulfonic anhydride (1.094 mL, 6.47 mmol) dropwise via syringe. The reaction was quenched with slow addition of saturated sodium carbonate (10 mL). The reaction was warmed to ambient temperature, water was added, and the mixture was stirred for 10 minutes. The mixture was extracted with dichloromethane (3×), and the combined organic layers were concentrated under reduced pressure. The residue was purified by column chromatography on silica gel (0-60% tert-butyl methyl ether/heptanes) to afford the title compound (0.92 g, 84%). ¹H NMR (400 MHz, CDCl₃) δ ppm 8.88 (d, J=4.8 Hz, 1H), 7.56-7.47 (m, 2H), 7.46-7.38 (m, 2H), 7.32 (d, J=4.9 Hz, 1H), 7.23-7.17 (m, 1H), 7.16-7.06 (m, 4H), 4.46 (q, J=7.1 Hz, 2H), 1.34 (t, J=7.1 Hz, 3H).

### Step P.2

### ethyl 7-[1-(tert-butoxycarbonyl)-1,2,3,6-tetrahydropyridin-4-yl]-2-(4-phenoxyphenyl)-2H-pyrazolo[4,3-b]pyridine-3-carboxylate

Ethyl 2-(4-phenoxyphenyl)-7-[(trifluoromethanesulfonyl)oxy]-2H-pyrazolo[4,3-b]pyridine-3-carboxylate (910 mg, 1.793 mmol, Step. P.1), tert-butyl 4-(4,4,5,5-tetramethyl-1,3,2-dioxaborolan-2-yl)-5,6-dihydropyridine-1(2H)-carboxylate (721 mg, 2.331 mmol), potassium phosphate (761 mg, 3.59 mmol), and bis(triphenylphosphine)palladium (II) dichloride (189 mg, 0.269 mmol) were weighed into a 200-mL round-bottomed flask. The flask was purged with an N₂ stream for 10 minutes before addition of 1,4-dioxane (20 mL) and water (4 mL). The reaction was heated to 90° C. for 10 minutes, then cooled to ambient temperature, and diluted with ethyl acetate. The organic layer was washed with water and brine, concentrated under reduced pressure, and purified by column chromatography on a silica gel column (0-100% tert-butyl methyl ether/heptanes) to afford the title compound (0.87 g, 90%). ¹H NMR (400 MHz, CDCl₃) δ ppm 8.80 (d, J=4.6 Hz, 1H), 7.57-7.45 (m, 3H), 7.45-7.33 (m, 2H), 7.19 (td, J=7.5, 6.8, 2.6 Hz, 2H), 7.15-7.06 (m, 4H), 4.45 (q, J=7.1 Hz, 2H), 4.22 (q, J=3.0 Hz, 2H), 3.71 (t, J=5.7 Hz, 2H), 2.72 (dt, J=6.3, 3.3 Hz, 2H), 1.49 (s, 9H), 1.33 (t, J=7.1 Hz, 3H).

### Intermediate Q

### [4-(2-cyclopropylphenoxy)phenyl]boronic acid

### Step Q.1

### 1-bromo-2-(4-nitrophenoxy)benzene

To a solution of 2-bromophenol (7.00 g, 40.5 mmol) and 1-fluoro-4-nitrobenzene (5.71 g, 40.5 mmol) in dimethyl sulfoxide (135 mL) was added K₂CO₃ (11.18 g, 81 mmol), and the mixture was heated to 120° C. After 16 hours, the mixture was diluted with water and allowed to cool while stirring vigorously. Once cool, the resulting precipitate was collected via filtration, dissolved in ethyl acetate, washed with brine, dried over Na₂SO₄, filtered, and concentrated under reduced pressure to afford a residue that was triturated with tert-butyl methyl ether to afford the title compound (10.63 g, 89%). ¹H NMR (500 MHz, dimethyl sulfoxide-d₆) 6 ppm 8.29-8.23 (m, 2H), 7.82 (dd, J=8.0, 1.6 Hz, 1H), 7.52 (ddd, J=8.1, 7.4, 1.5 Hz, 1H), 7.36 (dd, J=8.1, 1.5 Hz, 1H), 7.31 (ddd, J=8.0, 7.4, 1.5 Hz, 1H), 7.11-7.03 (m, 2H).

### Step Q.2

### 1-cyclopropyl-2-(4-nitrophenoxy)benzene

1-Bromo-2-(4-nitrophenoxy)benzene (10.62 g, 36.1 mmol, Step Q.1), K₃PO₄ (26.8 g, 126.0 mmol), tricyclohexylphosphine (1.01 g, 3.61 mmol), cyclopropylboronic acid (6.20 g, 72.2 mmol), and palladium(II) acetate (0.405 g, 1.80 mmol) were dissolved in toluene (172 mL) and water (9 mL) and sparged with N₂ for 5 minutes. The flask was fitted with a condenser and heated to 100° C. After 3 hours, the reaction mixture was cooled to ambient temperature. The contents were transferred to a separatory funnel and the flask was rinsed with ethyl acetate (3×) then diluted with water and extracted with ethyl acetate (3×). The combined organic layers were washed with water, brine, dried over Na₂SO₄, filtered, and concentrated under reduced pressure. The crude residue was filtered through a silica plug with 10% ethyl acetate in heptanes then concentrated to afford the title compound that was used without further purification. ¹H NMR (400 MHz, dimethyl sulfoxide-d₆) δ ppm 8.27-8.21 (m, 2H), 7.26 (pd, J=7.4, 1.7 Hz, 2H), 7.14-7.00 (m, 4H), 1.84 (tt, J=8.4, 5.2 Hz, 1H), 0.86-0.78 (m, 2H), 0.69-0.63 (m, 2H).

### Step Q.3

### 4-(2-cyclopropylphenoxy)aniline

To a suspension of crude 1-cyclopropyl-2-(4-nitrophenoxy)benzene (9.21 g, 36.1 mmol, Step Q.2) in acetone (150 mL) and water (30 mL) at 0° C. was added NH₄Cl (28.9 g, 541 mmol) and Zn dust (35.4 g, 541 mmol). The flask warmed to ambient temperature. After 40 minutes, the reaction mixture was diluted with ethyl acetate, filtered, and transferred to a separatory funnel. The organic layer was washed with brine, dried over Na₂SO₄, filtered, and concentrated under reduced pressure to afford the title compound that was used without further purification. MS (ESI) m/z: 226.5 [M+H]⁺.

### Step Q.4

### [4-(2-cyclopropylphenoxy)phenyl]boronic acid

To a solution of crude 4-(2-cyclopropylphenoxy)aniline (8.13 g, 36.1 mmol, Step Q.3) in methanol (72 mL) and 1 N HCl (108 mL) at 0° C. was added NaNO₂ (2 M in water, 18.1 mL, 36.1 mmol) via syringe pump over 30 minutes. After stirring for 10 minutes, a slurry of tetrahydroxydiboron (9.71 g, 108 mmol) in methanol (140 mL) was added, and the flask was warmed to ambient temperature. After 16 hours the reaction mixture was diluted with water, extracted with dichloromethane (3×), dried over Na₂SO₄, filtered, and concentrated under reduced pressure. The crude residue was purified by column chromatography (0% to 30% ethyl acetate in heptanes) to afford the title compound (4.78 g, 52% over 3 steps). ¹H NMR (600 MHz, dimethyl sulfoxide-d₆) δ ppm 7.92 (s, 2H), 7.76 (d, J=8.7 Hz, 2H), 7.19 (ddd, J=8.0, 7.3, 1.7 Hz, 1H), 7.13 (tdd, J=7.3, 1.4, 0.5 Hz, 1H), 6.99 (dd, J=7.8, 1.7 Hz, 1H), 6.94 (dd, J=8.0, 1.3 Hz, 1H), 6.83 (d, J=8.7 Hz, 2H), 1.95 (tt, J=8.5, 5.2 Hz, 1H), 0.87-0.81 (m, 2H), 0.68-0.64 (m, 2H).

### Intermediate R

### ethyl (7R)-7-[4-(2-nitrobenzene-1-sulfonyl)piperazin-1-yl]-4,5,6,7-tetrahydro-2H-pyrazolo[4,3-b]pyridine-3-carboxylate

### Step R.1

### ethyl (7E)-7-{[(R)-2-methylpropane-2-sulfinyl]imino}-4,5,6,7-tetrahydro-2H-pyrazolo[4,3-b]pyridine-3-carboxylate

A mixture of ethyl 7-oxo-4,5,6,7-tetrahydro-2H-pyrazolo[4,3-b]pyridine-3-carboxylate (10.4 g, 49.7 mmol, Step L.3), (R)-2-methylpropane-2-sulfinamide (18.1 g, 149 mmol) and tetraethoxytitanium (35 mL, 149 mmol) in toluene (200 mL) was stirred at 75° C. for 14 hours. The reaction was cooled to ambient temperature, filtered, and concentrated under reduced pressure. Water was added, and the mixture was filtered. The filtrate was extracted with ethyl acetate (3×). The combined organic phases were dried over Na₂SO₄, filtered, and concentrated under reduced pressure. The residue was purified by column chromatography on silica gel (0-45% ethyl acetate in petroleum ether) to afford the title compound (13.9 g, 90%). MS (ESI) m/z: 313.18 [M+H]⁺.

### Step R.2

### ethyl (7R)-7-{[(R)-2-methylpropane-2-sulfinyl]amino}-4,5,6,7-tetrahydro-2H-pyrazolo[4,3-b]pyridine-3-carboxylate

Ethyl (7E)-7-{[(R)-2-methylpropane-2-sulfinyl]imino}-4,5,6,7-tetrahydro-2H-pyrazolo[4,3-b]pyridine-3-carboxylate (5.1 g, 16.33 mmol, Step R.1) was dissolved in a mixture of tetrahydrofuran (150 mL) and water (5 mL), stirred at −20° C. for 30 minutes, and NaBH₄ (2.162 g, 57.1 mmol) was added slowly. The resulting mixture was stirred at −20° C. for 8 hours. A NH₄Cl solution was added to quench the reaction, and the mixture was extracted with dichloromethane (3×). The organic layers were washed with brine, dried over Na₂SO₄, and concentrated under reduced pressure. The residue was purified by column chromatography on silica gel (0-60% ethyl acetate in petroleum ether) to afford the title compound (5.0 g, 97%). MS (ESI) m/z: 315.24 [M+H]⁺.

### Step R.3

### ethyl (7R)-7-amino-4,5,6,7-tetrahydro-2H-pyrazolo[4,3-b]pyridine-3-carboxylate

4M HCl in 1,4-dioxane (6 mL) was added slowly to a solution of ethyl (7R)-7-{[(S)-2-methylpropane-2-sulfinyl]amino}-4,5,6,7-tetrahydro-2H-pyrazolo[4,3-b]pyridine-3-carboxylate (1.5 g, 4.77 mmol, Step R.2) in methanol (20 mL) at 0° C. The resulting mixture was stirred at 0° C. for 30 minutes. The reaction was warmed to ambient temperature and concentrated under reduced pressure to afford the title compound (0.80 g, 80%). MS (ESI) m/z: 194.0 [M-NH₃]⁺.

### Step R.4

### ethyl (7R)-7-[4-(2-nitrobenzene-1-sulfonyl)piperazin-1-yl]-4,5,6,7-tetrahydro-2H-pyrazolo[4,3-b]pyridine-3-carboxylate

A mixture of ethyl (7R)-7-amino-4,5,6,7-tetrahydro-2H-pyrazolo[4,3-b]pyridine-3-carboxylate (2.3 g, 10.94 mmol, Step R.3), [(2-nitrobenzene-1-sulfonyl)azanediyl]di(ethane-2,1-diyl) bis(2-nitrobenzene-1-sulfonate) (10.84 g, 16.41 mmol, Step L.6) and N-ethyl-N-isopropylpropan-2-amine (5.66 g, 43.8 mmol) in acetonitrile (50 mL) was stirred at 40° C. for 8 hours. The reaction was extracted with dichloromethane (3×). The organic layers were dried over Na₂SO₄ and concentrated under reduced pressure. The residue was purified by column chromatography on silica gel, (0-80% ethyl acetate in petroleum ether) to afford the title compound (4.4 g, 87%). MS (ESI) m/z: 465.13 [M+H]⁺.

### Intermediate S

### 2-(4-phenoxyphenyl)-7-(piperidin-3-yl)-4,5,6,7-tetrahydro-2H-pyrazolo[4,3-b]pyridine-3-carboxamide

### Step S.1

### tert-butyl 5-[3-carbamoyl-2-(4-phenoxyphenyl)-2H-pyrazolo[4,3-b]pyridin-7-yl]-3,6-dihydropyridine-1(2H)-carboxylate

A mixture of 7-bromo-2-(4-phenoxyphenyl)-2H-pyrazolo[4,3-b]pyridine-3-carboxamide and 7-bromo-2-(4-phenoxyphenyl)-2H-pyrazolo[4,3-b]pyridine-3-carboxamide (0.299 g, 0.774 mmol, Step B.6), K₃PO₄ (0.443 g, 2.087 mmol), 2-dicyclohexylphosphino-2′,4′,6′-triisopropylbiphenyl (0.055 g, 0.116 mmol), tris(dibenzylideneacetone)dipalladium(0) (Pd₂(dba)₃) (0.035 g, 0.039 mmol), and tert-butyl 4-(4,4,5,5-tetramethyl-1,3,2-dioxaborolan-2-yl)-3,6-dihydropyridine-1(2H)-carboxylate (0.359 g, 1.160 mmol) in N₂ sparged 1,4-dioxane (10.3 mL) and water (5.2 mL) was heated to 70° C. After 16 hours, the reaction mixture was cooled to ambient temperature, diluted with water and brine, and extracted with ethyl acetate (3×). The combined organic layers were washed with brine, dried over Na₂SO₄, filtered, and concentrated under reduced pressure. The crude residue was purified by column chromatography (0% to 40% ethyl acetate in heptanes) to afford the title compound (0.328 g, 0.641 mmol, 83%). MS (ESI) m/z: 512.0 [M+H]⁺.

### Step S.2

### tert-butyl 3-[3-carbamoyl-2-(4-phenoxyphenyl)-4,5,6,7-tetrahydro-2H-pyrazolo[4,3-b]pyridin-7-yl]piperidine-1-carboxylate

To a solution of tert-butyl 5-[3-carbamoyl-2-(4-phenoxyphenyl)-2H-pyrazolo[4,3-b]pyridin-7-yl]-3,6-dihydropyridine-1(2H)-carboxylate (0.321 g, 0.627 mmol, Step S.1) in tetrahydrofuran (6.3 mL) was added 10% Pd/C (0.20 g). The mixture was sparged with H₂ for 5 minutes then stirred under a H₂ balloon. After 16 hours, the reaction mixture was filtered through a diatomaceous earth plug and concentrated under reduced pressure. The crude residue was purified by column chromatography (0% to 80% ethyl acetate in heptanes) to afford the title compound (0.255 g, 0.493 mmol, 79%) as a mixture of diastereomers. MS (ESI) m/z: 518.1 [M+H]⁺.

### Step S.3

### 2-(4-phenoxyphenyl)-7-(piperidin-3-yl)-4,5,6,7-tetrahydro-2H-pyrazolo[4,3-b]pyridine-3-carboxamide

To a solution of tert-butyl 3-[3-carbamoyl-2-(4-phenoxyphenyl)-4,5,6,7-tetrahydro-2H-pyrazolo[4,3-b]pyridin-7-yl]piperidine-1-carboxylate (0.224 g, 0.433 mmol, Step S.2) in dichloromethane (8.7 mL) was added HCl (4 M in 1,4-dioxane, 1.08 mL, 4.33 mmol) and the mixture was stirred at ambient temperature. After 30 minutes, the reaction mixture was concentrated under reduced pressure to afford the title compound. MS (ESI) m/z: 418.2 [M+H]⁺.

### Intermediate T

### tert-butyl 4-[2-(4-bromophenyl)-3-carbamoyl-2H-pyrazolo[4,3-b]pyridin-7-yl]piperidine-1-carboxylate

### Step T.1

### tert-butyl 4-{(2E)-2-[2-(4-bromophenyl)hydrazinylidene]-2-cyanoacetyl}piperidine-1-carboxylate

Step T.1 was prepared according to the procedure for step C.2, substituting 4-bromoaniline for 4-phenoxyaniline. MS (DCI) m/z: 452.3 [M+NH₄].

### Step T.2

### tert-butyl 4-[4-amino-1-(4-bromophenyl)-5-(ethoxycarbonyl)-1H-pyrazole-3-carbonyl]piperidine-1-carboxylate

Step T.2 was prepared according to the procedure for step C.3, substituting tert-butyl 4-{(2E)-2-[2-(4-bromophenyl)hydrazinylidene]-2-cyanoacetyl}piperidine-1-carboxylate (Step T.1) for tert-Butyl 4-{(2E)-2-cyano-2-[2-(4-phenoxyphenyl)hydrazinylidene]acetyl}piperidine-1-carboxylate. MS (ESI) m/z: 521.3 [M+H]⁺.

### Step T.3

### ethyl 2-(4-bromophenyl)-7-[1-(tert-butoxycarbonyl)piperidin-4-yl]-2H-pyrazolo[4,3-b]pyridine-3-carboxylate

Step T.3 was prepared according to the procedure for step C.5, substituting tert-butyl 4-[4-amino-1-(4-bromophenyl)-5-(ethoxycarbonyl)-1H-pyrazole-3-carbonyl]piperidine-1-carboxylate (Step T.2) for tert-butyl 4-[4-amino-5-(ethoxycarbonyl)-1-(4-phenoxyphenyl)-1H-pyrazole-3-carbonyl]piperidine-1-carboxylate. MS (ESI) m/z: 531.2 [M+H]⁺.

### Step T.4

### 2-(4-bromophenyl)-7-[1-(tert-butoxycarbonyl)piperidin-4-yl]-2H-pyrazolo[4,3-b]pyridine-3-carboxylic acid

Step T.4 was prepared according to the procedure for step C.6, substituting ethyl 2-(4-bromophenyl)-7-[1-(tert-butoxycarbonyl)piperidin-4-yl]-2H-pyrazolo[4,3-b]pyridine-3-carboxylate (Step T.3) for ethyl 7-[1-(tert-butoxycarbonyl)piperidin-4-yl]-2-(4-phenoxyphenyl)-2H-pyrazolo[4,3-b]pyridine-3-carboxylate. ¹H NMR (400 MHz, dimethyl sulfoxide-d₆) δ ppm 8.73 (d, J=4.4 Hz, 1H), 7.88-7.73 (m, 2H), 7.67-7.56 (m, 2H), 7.37 (d, J=4.4 Hz, 1H), 4.18-4.01 (m, 2H), 3.50-3.32 (m, 1H), 2.90 (br s, 2H), 2.00-1.92 (m, 2H), 1.78 (dq, J=3.9, 12.5 Hz, 2H), 1.41 (s, 9H).

### Step T.5

### tert-butyl 4-[2-(4-bromophenyl)-3-carbamoyl-2H-pyrazolo[4,3-b]pyridin-7-yl]piperidine-1-carboxylate

Step T.5 was prepared according to the procedure for step C.7, substituting 2-(4-bromophenyl)-7-[1-(tert-butoxycarbonyl)piperidin-4-yl]-2H-pyrazolo[4,3-b]pyridine-3-carboxylic acid (Step T.4) for 7-[1-(tert-butoxycarbonyl)piperidin-4-yl]-2-(4-phenoxyphenyl)-2H-pyrazolo[4,3-b]pyridine-3-carboxylic acid. ¹H NMR (400 MHz, dimethyl sulfoxide-d₆) 6 ppm 8.70 (d, J=4.4 Hz, 1H), 8.44 (br s, 1H), 8.06 (br s, 1H), 7.82-7.70 (m, 2H), 7.66-7.51 (m, 2H), 7.36 (d, J=4.4 Hz, 1H), 4.19-3.99 (m, 2H), 3.36-3.40 (m, 1H), 2.89 (br s, 2H), 1.96 (br d, J=12.0 Hz, 2H), 1.78 (dq, J=3.9, 12.5 Hz, 2H), 1.41 (s, 9H).

### Intermediate U

### [4-(3-cyclopropylphenoxy)phenyl]boronic acid

### Step U.1

### 1-bromo-3-(4-nitrophenoxy)benzene

Step U.1 was prepared according to the procedure for Step Q.1, substituting 3-bromophenol for 2-bromophenol. ¹H NMR (400 MHz, dimethyl sulfoxide-d₆) δ ppm 8.32-8.22 (m, 2H), 7.55-7.41 (m, 3H), 7.26-7.14 (m, 3H).

### Step U.2

### 1-cyclopropyl-3-(4-nitrophenoxy)benzene

Step U.2 was prepared according to the procedure for Step Q.2, substituting 1-bromo-3-(4-nitrophenoxy)benzene (Step U.1) for 1-bromo-2-(4-nitrophenoxy)benzene. MS (ESI) m/z: 256.0 [M+H]⁺.

### Step U.3

### 4-(3-cyclopropylphenoxy)aniline

Step U.3 was prepared according to the procedure for Step Q.3, substituting 1-cyclopropyl-3-(4-nitrophenoxy)benzene (Step U.2) for 1-cyclopropyl-2-(4-nitrophenoxy)benzene. MS (ESI) m/z: 226.5 [M+H]⁺.

### Step U.4

### [4-(3-cyclopropylphenoxy)phenyl]boronic acid

Step U.4 was prepared according to the procedure for Step Q.4, substituting 4-(3-cyclopropylphenoxy)aniline (Step U.3) for 4-(2-cyclopropylphenoxy)aniline. ¹H NMR (400 MHz, dimethyl sulfoxide-d₆) δ ppm 7.96 (s, 2H), 7.84-7.75 (m, 2H), 7.30-7.21 (m, 1H), 6.91 (d, J=8.5 Hz, 2H), 6.86 (dt, J=7.7, 1.3 Hz, 1H), 6.80-6.73 (m, 2H), 1.91 (tt, J=8.4, 5.0 Hz, 1H), 0.98-0.91 (m, 2H), 0.69-0.63 (m, 2H).

### Intermediate V

### ethyl 7-[4-(tert-butoxycarbonyl)piperazin-1-yl]-4,5,6,7-tetrahydro-2H-pyrazolo[4,3-b]pyridine-3-carboxylate

To a reactor (Thermo Barnstead Stem RS10) were added ethyl 7-[4-(tert-butoxycarbonyl)piperazin-1-yl]-2H-pyrazolo[4,3-b]pyridine-3-carboxylate (2.79 g, 7.43 mmol, Intermediate A), 5% Pd/C (2.79 g, 0.610 mmol) and tetrahydrofuran (32 mL). The vessel was pressurized with H₂ (60 psi), and the reaction mixture was stirred for 22 hours at ambient temperature. The resulting solution was filtered over diatomaceous earth, and the filtrate was concentrated under reduced pressure. The crude product was purified by silica gel chromatography (100% ethyl acetate) to provide the title compound (0.550 g, 1.45 mmol, 18%). MS (APCI) m/z: 380.2 [M+H]⁺.

### Intermediate W

### tert-butyl 4-[2-(4-bromo-2-fluorophenyl)-3-carbamoyl-2H-pyrazolo[4,3-b]pyridin-7-yl]piperidine-1-carboxylate

### Step W.1

### tert-butyl 4-[4-amino-1-(4-bromo-2-fluorophenyl)-5-(ethoxycarbonyl)-1H-pyrazole-3-carbonyl]piperidine-1-carboxylate

To a solution of 4-bromo-2-fluoroaniline (2.259 g, 11.89 mmol) in water (38 mL) was added HCl (37 wt. % in H₂O, 3.61 mL) at 0° C. The solution was stirred briefly before sodium nitrite (0.846 g, 11.9 mmol) in water (15 mL) was added. The reaction mixture was stirred for 30 minutes at 0° C. The resulting solution was added dropwise over 10 min via an addition funnel to a solution of sodium acetate (29.3 g, 357 mmol) and tert-butyl 4-(cyanoacetyl)piperidine-1-carboxylate (3.9 g, 15 mmol, Step C.1) in 4:3 water/ethanol (132 mL) at 0° C. Upon completion of the addition, the reaction vessel was removed from the cooling bath and the resulting suspension was stirred further at ambient temperature for 5 minutes. The precipitate was collected by filtration and dried in vacuo at 60° C. to provide a mixture of tert-butyl 4-{(2E)-2-[2-(4-bromo-2-fluorophenyl)hydrazinylidene]-2-cyanoacetyl}piperidine-1-carboxylate and tert-butyl 4-{(2Z)-2-[2-(4-bromo-2-fluorophenyl)hydrazinylidene]-2-cyanoacetyl}piperidine-1-carboxylate (5.39 g, approximately 3:1 by NMR analysis), which was used without further purification. To a solution of tert-butyl 4-{(2E)-2-[2-(4-bromo-2-fluorophenyl)hydrazinylidene]-2-cyanoacetyl}piperidine-1-carboxylate and tert-butyl 4-{(2Z)-2-[2-(4-bromo-2-fluorophenyl)hydrazinylidene]-2-cyanoacetyl}piperidine-1-carboxylate (5.39 g) and N,N-diisopropylethylamine (20.77 mL, 119 mmol) in dioxane (33 mL) was added ethyl 2-bromoacetate (4.04 mL, 35.7 mmol). The resulting solution was heated to 100° C. and stirred for 2 hours. The reaction mixture was cooled to ambient temperature, diluted with ethyl acetate (150 mL), and washed water (100 mL). The aqueous solution was extracted with ethyl acetate (2×50 mL). The combined organic layers were washed with brine (100 mL), dried over sodium sulfate, and concentrated under reduced pressure. The crude product was purified by silica gel chromatography (0-40% ethyl acetate in heptanes) to provide the title compound (5.51 g, 10.2 mmol, 86%). MS (DCI) m/z: 539.2 [M+H]⁺.

### Step W.2

### ethyl 2-(4-bromo-2-fluorophenyl)-7-(1-(tert-butoxycarbonyl)piperidin-4-yl)-2H-pyrazolo[4,3-b]pyridine-3-carboxylate

To a solution of N,N-diisopropylamine (4.58 ml, 32.7 mmol) in tetrahydrofuran (47 mL) was added n-butyllithium (12.7 mL, 31.7 mmol, 2.5 M in hexane) dropwise via a syringe at −78° C. (1E)-N-tert-Butylethanimine (4.44 ml, 32.7 mmol, Step C.4) was added dropwise at the same temperature. The solution was stirred for 20 minutes. A solution of tert-butyl 4-[4-amino-1-(4-bromo-2-fluorophenyl)-5-(ethoxycarbonyl)-1H-pyrazole-3-carbonyl]piperidine-1-carboxylate (5.51 g, 10.2 mmol, Step W.1) in tetrahydrofuran (28 mL) was added dropwise at −78° C. The reaction mixture was stirred further for 15 minutes and then quenched by the addition of 1 M aqueous HCl (50 mL) at −78° C. The resulting solution was warmed to ambient temperature and diluted with ethyl acetate. After stirring for 5 minutes, the layers were separated, and the organic layer was washed with 1 M aqueous HCl (2×), saturated NaHCO₃ (aq.), and brine. The organic layer was dried over sodium sulfate and concentrated under reduced pressure. The crude product was purified by silica gel chromatography (0-50% ethyl acetate in heptanes) to provide the title compound (2.93 g, 5.35 mmol, 52%). MS (ESI) m/z: 547.1 [M+H]⁺.

### Step W.3

### 2-(4-bromo-2-fluorophenyl)-7-[1-(tert-butoxycarbonyl)piperidin-4-yl]-2H-pyrazolo[4,3-b]pyridine-3-carboxylic acid

Step W.3 was prepared according to the procedure for Step C.6, substituting ethyl 2-(4-bromo-2-fluorophenyl)-7-(1-(tert-butoxycarbonyl)piperidin-4-yl)-2H-pyrazolo[4,3-b]pyridine-3-carboxylate (Step W.2) for ethyl 7-[1-(tert-butoxycarbonyl)piperidin-4-yl]-2-(4-phenoxyphenyl)-2H-pyrazolo[4,3-b]pyridine-3-carboxylate to provide the title compound (2.8 g). MS (ESI) m/z: 519.3 [M+H]⁺.

### Step W.4

### tert-butyl 4-[2-(4-bromo-2-fluorophenyl)-3-carbamoyl-2H-pyrazolo[4,3-b]pyridin-7-yl]piperidine-1-carboxylate

Step W.4 was prepared according to the procedure for Step C.7, substituting 2-(4-bromo-2-fluorophenyl)-7-[1-(tert-butoxycarbonyl)piperidin-4-yl]-2H-pyrazolo[4,3-b]pyridine-3-carboxylic acid (Step W.3) for 7-[1-(tert-butoxycarbonyl)piperidin-4-yl]-2-(4-phenoxyphenyl)-2H-pyrazolo[4,3-b]pyridine-3-carboxylic acid to provide the crude product. The crude product was purified by silica gel chromatography (0-50% ethyl acetate in heptanes) to provide the title compound (2.51 g, 4.84 mmol, 90%). MS (ESI) m/z: 518.2 [M+H]⁺.

### Intermediate X

### [4-(3,5-difluorophenoxy)phenyl]boronic acid

### Step X.1

### 2-[4-(3,5-difluorophenoxy)phenyl]-4,4,5,5-tetramethyl-1,3,2-dioxaborolane

Step X.1 was prepared according to the procedure for Step M.1, substituting (3,5-difluorophenyl)boronic acid for (3-fluorophenyl) boronic acid.

### Step X.2

### [4-(3,5-difluorophenoxy)phenyl]boronic acid

To a solution of 2-[4-(3,5-difluorophenoxy)phenyl]-4,4,5,5-tetramethyl-1,3,2-dioxaborolane (24.6 g, 74.1 mmol) in tetrahydrofuran (80 mL) and water (20 mL) was added sodium periodate (63.4 g, 296 mmol). The cloudy solution was stirred for 16 h at room temperature. The reaction mixture was filtered through a plug of diatomaceous earth and sodium sulfate. The plug was washed with ethyl acetate, and the combined filtrates were concentrated under reduced pressure. The crude product was purified by a silica gel column and eluted with 25% ethyl acetate in hexanes, then 50% ethyl acetate to 1% methanol in dichloromethane to afford the title compound (17.8 g, 96%). ¹H NMR (400 MHz, dimethyl sulfoxide-d₆) δ ppm 6.83-6.65 (m, 2H). 7.04-6.95 (m, 1H), 7.07 (d, J=8.3 Hz, 2H), 7.86 (d, J=8.3 Hz, 2H).

### Example 1

### Rac-2-(4-phenoxyphenyl)-7-[1-(prop-2-enoyl)piperidin-4-yl]-4,5,6,7-tetrahydro-2H-pyrazolo[4,3-b]pyridine-3-carboxamide

2-(4-phenoxyphenyl)-7-(piperidin-4-yl)-4,5,6,7-tetrahydro-2H-pyrazolo[4,3-b]pyridine-3-carboxamide-hydrogen chloride (1/3) (3.40 g, 6.45 mmol, Intermediate C) was suspended in dichloromethane (65 mL) and N,N-diisopropylethylamine (6.76 mL, 38.7 mmol) was added. The vial was cooled to −5° C. in an ice-water bath. Once a solution formed, acrylic acid (0.443 mL, 6.45 mmol) and 2,4,6-tripropyl-1,3,5,2,4,6-trioxatriphosphinane 2,4,6-trioxide (50% w/w in ethyl acetate, 5.76 mL, 9.68 mmol) were added dropwise, maintaining an internal temperature below 0° C. The reaction was stirred for 5 minutes at the same temperature, then diluted with water (50 mL), stirred 5 minutes at ambient temperature, and poured into a separatory funnel. The layers were separated, and the organic layer was washed with 50% saturated sodium bicarbonate, brine, dried over sodium sulfate, and concentrated under reduced pressure to afford a residue. The residue was purified by a silica gel column and eluted with methanol/dichloromethane (0/100 to 10/90 over 20 minutes) to afford the title compound (2.50 g, 82%). ¹H NMR (400 MHz, CDCl₃) δ ppm 7.38 (dt, J=8.3, 3.5 Hz, 4H), 7.17 (t, J=7.4 Hz, 1H), 7.13-6.99 (m, 4H), 6.57 (dd, J=16.8, 10.6 Hz, 1H), 6.24 (dd, J=16.9, 2.0 Hz, 1H), 5.65 (dd, J=10.5, 2.0 Hz, 1H), 5.42 (s, 2H), 5.07 (s, 1H), 4.71 (d, J=11.6 Hz, 1H), 4.04 (d, J=12.2 Hz, 1H), 3.45-3.19 (m, 2H), 3.04 (t, J=12.7 Hz, 1H), 2.86 (dd, J=24.5, 6.7 Hz, 1H), 2.68-2.51 (m, 1H), 2.22-2.01 (m, 1H), 2.01-1.66 (m, 4H), 1.42 (dtd, J=35.1, 14.1, 13.3, 7.3 Hz, 2H). MS (APCI) m/z: 472.3 [M+H]⁺.

### Example 2

### 7-[1-(but-2-ynoyl)piperidin-4-yl]-2-(4-phenoxyphenyl)-4,5,6,7-tetrahydro-2H-pyrazolo[4,3-b]pyridine-3-carboxamide

2-(4-phenoxyphenyl)-7-(piperidin-4-yl)-4,5,6,7-tetrahydro-2H-pyrazolo[4,3-b]pyridine-3-carboxamide-hydrogen chloride (1/3) (0.100 g, 0.204 mmol, Intermediate C) was suspended in dichloromethane (4.00 mL) and N-ethyl-N-isopropylpropan-2-amine (0.218 mL, 1.223 mmol) was added. The solution was cooled to <5° C. in an ice-water bath and but-2-ynoic acid (0.018 mL, 0.224 mmol) was added as a solution in dichloromethane (1 mL), followed by dropwise addition of 1-propanephosphonic anhydride (50% w/w in ethyl acetate, 0.243 mL, 0.408 mmol). The reaction was stirred for 5 minutes at the same temperature, then diluted with ethyl acetate, washed with water (3×), brine, dried over sodium sulfate, and concentrated under reduced pressure to afford a residue. The residue was slurried in tert-butyl methyl ether, isolated via filtration through a fritted funnel, and dried to constant weight in a vacuum oven at 40° C. to afford the title compound (68 mg, 69%). ¹H NMR (400 MHz, CDCl₃) δ ppm 7.38 (td, J=6.6, 3.1 Hz, 4H), 7.23-7.14 (m, 1H), 7.14-7.04 (m, 4H), 5.25 (s, 2H), 5.08 (s, 1H), 4.62 (dtd, J=12.9, 4.3, 2.2 Hz, 1H), 4.51-4.30 (m, 1H), 3.43-3.24 (m, 2H), 3.09-2.94 (m, 1H), 2.94-2.81 (m, 1H), 2.61 (ddt, J=16.6, 13.0, 3.3 Hz, 1H), 2.22-2.04 (m, 1H), 1.99 (s, 3H), 1.96-1.81 (m, 3H), 1.76 (dt, J=13.2, 2.8 Hz, 1H), 1.52-1.27 (m, 2H). MS (APCI) m/z: 484.4 [M+H]⁺.

### Example 3

2-(4-phenoxyphenyl)-7-[4-(prop-2-enoyl)piperazin-1-yl]-4,5,6,7-tetrahydro-2H-pyrazolo[4,3-b]pyridine-3-carboxamide 2-(4-phenoxyphenyl)-7-(piperazin-1-yl)-4,5,6,7-tetrahydro-2H-pyrazolo[4,3-b]pyridine-3-carboxamide (12.4 g, 29.6 mmol, Intermediate B) was suspended in dichloromethane (300 mL), and the suspension was cooled in an ice-water bath to <10° C. before addition of N-ethyl-N-isopropylpropan-2-amine (31.6 mL, 178 mmol) via syringe. The suspension was stirred for 5 minutes or until complete dissolution of the starting material had occurred. Once the internal temperature returned to <5° C., the solution was treated with acrylic acid (1.63 mL, 23.7 mmol), and 2,4,6-tripropyl-1,3,5,2,4,6-trioxatriphosphinane 2,4,6-trioxide (50 weight % in ethyl acetate, 17.6 mL, 59.3 mmol) was added dropwise over 2 minutes, maintaining an internal temperature below 12° C. Once the addition was complete, the reaction was stirred for 5 minutes. Water (200 mL) was added, and the reaction was stirred for 5 minutes at ambient temperature then poured into a separatory funnel. The organic layer was washed with water (2×), concentrated under reduced pressure to approximately 30 mL, loaded onto a silica gel column and eluted with methanol/dichloromethane (0/100 to 5/95 over 20 minutes then isocratic 5/95) to afford the title compound (5.00 g, 45% over 3 steps). ¹H NMR (500 MHz, CDCl₃) δ ppm 7.43-7.35 (m, 4H), 7.21-7.15 (m, 1H), 7.10-7.01 (m, 4H), 6.55 (dd, J=16.8, 10.6 Hz, 1H), 6.27 (dd, J=16.8, 2.0 Hz, 1H), 5.67 (dd, J=10.5, 2.0 Hz, 1H), 5.25 (br s, 2H), 5.14 (br s, 1H), 3.85 (dd, J=6.7, 5.0 Hz, 1H), 3.77 (d, J=9.1 Hz, 1H), 3.70-3.63 (m, 1H), 3.57 (d, J=4.7 Hz, 2H), 3.44 (dddd, J=10.0, 8.4, 3.9, 2.2 Hz, 1H), 3.31 (ddt, J=13.3, 7.5, 2.9 Hz, 1H), 2.83-2.61 (m, 4H), 2.18 (dtd, J=14.1, 7.2, 3.1 Hz, 1H), 1.96 (dtd, J=13.5, 5.0, 2.5 Hz, 1H). MS (APCI) m/z: 473.3 [M+H]⁺.

### Example 4

### (7R)-2-(4-phenoxyphenyl)-7-[1-(prop-2-enoyl)piperidin-4-yl]-4,5,6,7-tetrahydro-2H-pyrazolo[4,3-b]pyridine-3-carboxamide; and

### Example 5

### (7S)-2-(4-phenoxyphenyl)-7-[1-(prop-2-enoyl)piperidin-4-yl]-4,5,6,7-tetrahydro-2H-pyrazolo[4,3-b]pyridine-3-carboxamide

Rac-2-(4-phenoxyphenyl)-7-[1-(prop-2-enoyl)piperidin-4-yl]-4,5,6,7-tetrahydro-2H-pyrazolo[4,3-b]pyridine-3-carboxamide (1.13 g, 2.396 mmol, Example 1) was separated by chiral supercritical fluid chromatography using ChiralPak® AD-H, 30×250 mm, 5 micron, injecting a solution of crude material in methanol (125 mg/mL) and eluting with an isocratic 45% methanol/CO₂ mobile phase over 20 minutes to afford: Example 4 (0.479 g, 37%), ¹H NMR (400 MHz, CDCl₃) δ ppm 7.38 (dt, J=8.3, 3.5 Hz, 4H), 7.22-7.13 (m, 1H), 7.11-7.02 (m, 4H), 6.57 (dd, J=16.8, 10.6 Hz, 1H), 6.24 (dd, J=16.8, 2.0 Hz, 1H), 5.65 (dd, J=10.5, 2.0 Hz, 1H), 5.48 (s, 2H), 5.07 (s, 1H), 4.72 (d, J=12.1 Hz, 1H), 4.03 (t, J=11.7 Hz, 1H), 3.46-3.21 (m, 2H), 3.11-2.97 (m, 1H), 2.86 (dd, J=24.8, 6.7 Hz, 1H), 2.60 (t, J=12.1 Hz, 1H), 2.12 (d, J=36.3 Hz, 1H), 1.94 (t, J=5.8 Hz, 3H), 1.77 (t, J=14.8 Hz, 1H), 1.43 (dtt, J=32.8, 12.9, 5.8 Hz, 2H). MS (APCI) m/z: 472.4 [M+H]⁺; and Example 5 (0.471 g, 41.7%), ¹H NMR (400 MHz, CDCl₃) δ ppm 7.38 (dt, J=8.4, 3.5 Hz, 4H), 7.17 (td, J=7.4, 1.1 Hz, 1H), 7.12-6.99 (m, 4H), 6.57 (dd, J=16.8, 10.6 Hz, 1H), 6.24 (dd, J=16.9, 2.0 Hz, 1H), 5.65 (dd, J=10.6, 2.0 Hz, 1H), 5.51 (s, 2H), 5.07 (s, 1H), 4.71 (d, J=12.8 Hz, 1H), 4.04 (d, J=13.4 Hz, 1H), 3.48-3.22 (m, 2H), 3.12-2.96 (m, 1H), 2.86 (dd, J=24.8, 7.4 Hz, 1H), 2.60 (t, J=12.6 Hz, 1H), 2.12 (d, J=36.4 Hz, 1H), 1.92 (dt, J=22.2, 10.5 Hz, 3H), 1.76 (t, J=14.8 Hz, 1H), 1.52-1.31 (m, 2H). MS (APCI) m/z: 472.5 [M+H

### Example 6

### (7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide

### Step 6.1

### (7R)-2-(4-phenoxyphenyl)-7-(piperazin-1-yl)-4,5,6,7-tetrahydro-2H-pyrazolo[4,3-b]pyridine-3-carboxamide

2-(4-phenoxyphenyl)-7-(piperazin-1-yl)-4,5,6,7-tetrahydro-2H-pyrazolo[4,3-b]pyridine-3-carboxamide (1.00 g, 2.39 mmol, Intermediate B) was dissolved in ethanol (10 mL) in a 20-mL scintillation vial and the solution was heated to 60° C. before dropwise addition of a solution of (2R,3R)-2,3-bis((4-methylbenzoyl)oxy)succinic acid (0.231 g, 0.597 mmol) in ethanol (3 mL). The solution was heated at the same temperature while stirring for 15 minutes, during which a precipitate formed. The resulting suspension was heated for 5 hours at the same temperature, then cooled to ambient temperature. The resulting solid was isolated via filtration through a fritted funnel, washed with ice-cold ethanol (5 mL) and dried in the funnel. The solid was transferred to a round-bottomed flask and 1 M NaOH (10 mL) in dichloromethane (10 mL) was added. The product was extracted with additional dichloromethane (2×), dried over sodium sulfate, and concentrated under reduced pressure to afford the crude product (0.538 g), which was determined by ¹H NMR to be a 2:1 ratio of amine:tartrate. Enantiopurity was determined after reprotection of an aliquot as the Boc protected amine by heating in tetrahydrofuran with di-tert-butyl dicarbonate:Chiral supercritical fluid chromatography analysis showed a 75:25 ratio of enantiomers (ChiralCel® OD-H column, 5-50% methanol over 10 minutes with diethylamine modifier, Peak A=7.79 minutes, Peak B=8.23 minutes). Further enrichment of the enantiopurity was achieved through modification of the classical resolution: 2-(4-phenoxyphenyl)-7-(piperazin-1-yl)-4,5,6,7-tetrahydro-2H-pyrazolo[4,3-b]pyridine-3-carboxamide (Intermediate B enriched as a 75:25 mixture of enantiomers, 525 mg, 1.26 mmol) was dissolved in ethanol (10 mL), and the solution was heated to 60° C. before dropwise addition of a solution of (2R,3R)-2,3-bis((4-methylbenzoyl)oxy)succinic acid (170 mg, 0.439 mmol) in ethanol (5 mL). The reaction was stirred at the same temperature until a solid began to form, and heating was continued for 1 hour before cooling to ambient temperature. The resulting solid material was isolated via filtration, washed with cold ethanol, and dried in the funnel. The solid was transferred to a round-bottomed flask and treated with 1 M NaOH/dichloromethane to afford a solid after extraction into additional dichloromethane. The title compound was obtained (396 mg, 38%), of which chiral supercritical fluid chromatography showed an enantiomer ratio of at least 94:6 (ChiralCel® OD-H column, 5-50% methanol over 10 minutes with diethylamine modifier, Peak A=7.79 minutes, Peak B=8.23 minutes). The material was used without additional purification.

### Step 6.2

### (7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide

(7R)-2-(4-phenoxyphenyl)-7-(piperazin-1-yl)-4,5,6,7-tetrahydro-2H-pyrazolo[4,3-b]pyridine-3-carboxamide (1.60 g, 3.82 mmol, Step 6.1) was dissolved in dichloromethane (38.0 mL), and the solution was cooled in an acetone-ice bath to <−10° C. N-Ethyl-N-isopropylpropan-2-amine (2.04 mL, 11.5 mmol) was added, followed by acrylic acid (0.249 mL, 3.63 mmol) and dropwise addition of 2,4,6-tripropyl-1,3,5,2,4,6-trioxatriphosphinane 2,4,6-trioxide (50% w/w in ethyl acetate, 2.50 mL, 4.21 mmol). The reaction was stirred for 5 minutes at ambient temperature. 50% Saturated aqueous sodium chloride (20 mL) was added and the reaction mixture was stirred for another 10 minutes. The layers were separated, and the aqueous layer was extracted with additional dichloromethane (2×). The combined organic extracts were concentrated under reduced pressure to afford a crude residue, which was purified via flash chromatography, eluting with methanol/dichloromethane (0/100 to 10/90, with 2% triethylamine) to afford 1.67 g of crude product. The crude product was determined to have a 93:7 enantiomer ratio. The crude product (1.10 g) was dissolved in acetonitrile (5 mL), and tert-butyl methyl ether (10 mL) was added while stirring. Stirring at ambient temperature was continued overnight, forming a thin slurry. The slurry was filtered through a fritted funnel, and the filtrate was concentrated under reduced pressure to afford the title compound as a single enantiomer (1.19 g, 66%). ¹H NMR (500 MHz, CDCl₃) δ ppm 7.42-7.33 (m, 4H), 7.21-7.14 (m, 1H), 7.11-7.03 (m, 4H), 6.55 (dd, J=16.8, 10.6 Hz, 1H), 6.27 (dd, J=16.8, 2.0 Hz, 1H), 5.67 (dd, J=10.5, 2.0 Hz, 1H), 5.25 (s, 2H), 5.14 (s, 1H), 3.85 (dd, J=6.7, 5.0 Hz, 1H), 3.77 (d, J=9.1 Hz, 1H), 3.71-3.62 (m, 1H), 3.57 (d, J=4.7 Hz, 2H), 3.44 (dddd, J=10.0, 8.4, 3.9, 2.2 Hz, 1H), 3.36-3.24 (m, 1H), 2.86-2.53 (m, 4H), 2.18 (dtd, J=14.1, 7.2, 3.1 Hz, 1H), 1.96 (dddd, J=13.5, 8.3, 5.1, 3.3 Hz, 1H). MS (APCI) m/z: 473.4 [M+H]⁺.

### Example 7

### (7S)-2-(4-phenoxyphenyl)-7-[4-(prop-2-enoyl)piperazin-1-yl]-4,5,6,7-tetrahydro-2H-pyrazolo[4,3-b]pyridine-3-carboxamide

### Step 7.1

### 3-oxo-2-[2-(4-phenoxyphenyl)hydrazinylidene]butanenitrile

A 3000 mL 3-necked flask was charged with 4-phenoxyaniline (25 g, 135 mmol) and deionized water (675 mL). The stirred suspension was cooled to 10° C. and 37% aqueous HCl (122 mL, 1485 mmol) was added over 2 minutes. The resultant suspension was cooled to 4° C., and a solution of sodium nitrite (9.31 g, 135 mmol) in water (135 mL) (1 M) was added dropwise via addition funnel over 18 minutes. The temperature was slowly increased stepwise to 20° C. over 45 minutes, and the reaction was stirred at this temperature for an additional 15 minutes before cooling to 0° C. A solution of sodium acetate (332 g, 4049 mmol) and 3-oxobutanenitrile (17.24 mL, 202 mmol) in water (135 mL) was added to the reaction mixture via an addition funnel over 30 minutes. A precipitate formed during the addition, and the suspension was warmed to 16° C. and stirred overnight. The precipitate was collected by vacuum filtration and washed with water (2×). The solids were then suspended in 4:1 tert-butyl methyl ether/heptane mixture (600 mL) and stirred for 30 minutes at ambient temperature before collection by vacuum filtration. The solid was then dried in a vacuum oven at 50° C. for 2 hours to afford the title compound (27.67 g, 73%). ¹H NMR (500 MHz, CDCl₃) δ ppm 14.89 (s, 1H), 7.41-7.37 (m, 3H), 7.37-7.29 (m, 3H), 7.15 (tq, J=7.3, 1.2 Hz, 1H), 7.11-7.03 (m, 3H), 7.03-6.99 (m, 2H), 2.50 (s, 3H), 2.49 (s, 1H). MS (ESI) m/z: 280.1 [M+H]⁺.

### Step 7.2

### ethyl 3-acetyl-4-amino-1-(4-phenoxyphenyl)-1H-pyrazole-5-carboxylate

To a 12 L three-neck flask equipped with a reflux condenser, overhead stirring, thermocouple probe and nitrogen inlet, was added 3-oxo-2-[2-(4-phenoxyphenyl)hydrazinylidene]butanenitrile (404 g, 1447 mmol, Step 7.1) followed by anhydrous toluene (4000 mL). N-Ethyl-N-isopropylpropan-2-amine (1011 mL, 5786 mmol) was added, followed by ethyl 2-bromoacetate (401 mL, 3616 mmol). The solution was heated to 100° C. for 18 hours, then allowed to cool to ambient temperature. The reaction solution was decanted from a precipitate, diluted with ethyl acetate, washed with water (3×), and washed with saturated aqueous sodium chloride solution. The precipitate left from the decantation was taken up in ethyl acetate and extracted with water (2×) and saturated aqueous sodium chloride solution. The combined organic layers were dried over anhydrous magnesium sulfate, filtered, and concentrated under reduced pressure. The resultant crude material was slurried in anhydrous ethanol for 1 hour. The solids were collected by vacuum filtration, was washed with ethanol, dried by vacuum filtration, and dried in a vacuum oven at 40° C. for 4 days to afford the title compound (300 g, 57%). ¹H NMR (400 MHz, dimethyl sulfoxide-d₆) δ ppm 7.53-7.38 (m, 4H), 7.20 (tt, J=7.3, 1.1 Hz, 1H), 7.13-7.04 (m, 4H), 5.94 (s, 2H), 4.16 (q, J=7.1 Hz, 2H), 2.47 (s, 3H), 1.12 (t, J=7.1 Hz, 3H). MS (APCI) m/z: 366.1 [M+H]⁺.

### Step 7.3

### ethyl 7-hydroxy-2-(4-phenoxyphenyl)-2H-pyrazolo[4,3-b]pyridine-3-carboxylate-hydrogen chloride (1/1)

A 5 L jacketed three-necked flask was charged with ethyl 3-acetyl-4-amino-1-(4-phenoxyphenyl)-1H-pyrazole-5-carboxylate (210 g, 575 mmol, Step 7.2). Anhydrous tetrahydrofuran (3 L) was added and to the resultant solution at ambient temperature was added ethyl formate (0.187 L, 2299 mmol). The solution was then cooled to −10° C., and a solution of sodium tert-butoxide in tetrahydrofuran (0.603 L, 1207 mmol) was added over one hour and stirred at this temperature for an additional 90 minutes. Aqueous 3 N HCl solution (0.958 L, 2874 mmol) was added dropwise over one hour and the resultant suspension was warmed to ambient temperature and stirred overnight. The solids were collected by vacuum filtration, washed with water, air-dried on the filter pad overnight, and then treated with tert-butyl methyl ether. The suspension was stirred at ambient temperature overnight. The solids were collected by vacuum filtration to afford the title compound (160.5 g, 68%). ¹H NMR (400 MHz, dimethyl sulfoxide-d₆) δ ppm 12.03 (s, br, 2H), 8.02 (d, J=7.3 Hz, 1H), 7.63 (dt, J=8.9, 3.2, 2.3 Hz, 2H), 7.45 (ddt, J=9.0, 7.7, 2.3, 1.9 Hz, 2H), 7.22 (ddt, J=8.6, 7.3, 1.1 Hz, 1H), 7.17-7.08 (m, 4H), 6.33 (d, J=7.4 Hz, 1H), 4.33 (q, J=7.1 Hz, 2H), 1.23 (t, J=7.1 Hz, 3H); ¹³C NMR (101 MHz, dimethyl sulfoxide-d₆) δ ppm 171.37, 157.83, 157.31, 155.87, 141.96, 139.31, 134.86, 131.16, 130.30, 128.19, 124.29, 120.05, 119.35, 117.84, 109.03, 61.38, 14.02. MS (APCI) m/z: 376.1 [M+H]⁺.

### Step 7.4

### ethyl 7-chloro-2-(4-phenoxyphenyl)-2H-pyrazolo[4,3-b]pyridine-3-carboxylate

To a 5 L jacketed three-neck flask was added ethyl 7-hydroxy-2-(4-phenoxyphenyl)-2H-pyrazolo[4,3-b]pyridine-3-carboxylate-hydrogen chloride (1/1) (59 g, 143 mmol, step 7.3) followed by anhydrous acetonitrile (1.2 L). The slurry was cooled to 0° C. and phosphoryl trichloride (66.8 mL, 716 mmol) was added dropwise over 15 minutes, after which the reaction was warmed to ambient temperature and stirred for 48 hours. The reaction was then cooled to −2° C. and aqueous 1 M sodium hydroxide (2 L) was added until the pH of the reaction mixture reached 7. The resultant solid material was collected by vacuum filtration, washed with water, and dried in a vacuum oven at 50° C. overnight to afford the title compound (58 g, 100%). ¹H NMR (400 MHz, dimethyl sulfoxide-d₆) δ ppm 8.73 (d, J=4.6 Hz, 1H), 7.70 (d, J=4.6 Hz, 1H), 7.69-7.65 (m, 2H), 7.52-7.42 (m, 2H), 7.23 (tt, J=7.4, 0.9 Hz, 1H), 7.21-7.10 (m, 4H), 4.31 (q, J=7.1 Hz, 2H), 1.22 (t, J=7.1 Hz, 3H). MS (APCI) m/z: 394.1 [M+H]⁺.

### Step 7.5

### ethyl 7-[4-(tert-butoxycarbonyl)piperazin-1-yl]-2-(4-phenoxyphenyl)-2H-pyrazolo[4,3-b]pyridine-3-carboxylate

A 500 mL round bottom flask was charged with ethyl 7-chloro-2-(4-phenoxyphenyl)-2H-pyrazolo[4,3-b]pyridine-3-carboxylate (22.2 g, 56.4 mmol, Step 7.4) and tert-butyl piperazine-1-carboxylate (12.60 g, 67.6 mmol). Anhydrous dimethyl acetamide (188 mL) was added followed by N,N-diisopropylethylamine (19.69 mL, 113 mmol). The reaction was stirred at 100° C. overnight, allowed to cool to ambient temperature, and poured into water (500 mL). Ethyl acetate (500 mL) was added, and the biphasic mixture was stirred for 2 hours. The layers were separated, and the aqueous layer was extracted with ethyl acetate. The combined organic layers were washed with water (3×), washed with saturated aqueous sodium chloride solution, dried over anhydrous sodium sulfate, filtered, and concentrated. The resulting residue was dissolved in dichloromethane and purified via column chromatography on silica gel eluting with a 0-10% methanol dichloromethane gradient. The clean fractions were combined and concentrated to afford the title compound (28.1 g, 92%). ¹H NMR (400 MHz, dimethyl sulfoxide-d₆) δ ppm 8.35 (d, J=5.3 Hz, 1H), 7.64-7.56 (m, 2H), 7.51-7.41 (m, 2H), 7.27-7.17 (m, 1H), 7.17-7.09 (m, 4H), 6.54 (d, J=5.4 Hz, 1H), 4.26 (q, J=7.1 Hz, 2H), 3.85 (dd, J=6.7, 3.8 Hz, 4H), 3.54-3.47 (m, 4H), 1.41 (s, 9H), 1.19 (t, J=7.1 Hz, 3H). MS (APCI) m/z: 544.3 [M+H]⁺.

### Step 7.6

### tert-butyl 4-[3-carbamoyl-2-(4-phenoxyphenyl)-2H-pyrazolo[4,3-b]pyridin-7-yl]piperazine-1-carboxylate

Ethyl 7-[4-(tert-butoxycarbonyl)piperazin-1-yl]-2-(4-phenoxyphenyl)-2H-pyrazolo[4,3-b]pyridine-3-carboxylate (235 g, 432 mmol, Step 7.5) and anhydrous ammonia (7 M in methanol, 1500 mL) were added to a stainless steel reactor, and the reactor was closed. The reactor was pressurized with nitrogen (116 psi), and the reaction was heated to 70° C. with 1000 rpm stirring. After cooling the reactor, the reaction was concentrated under reduced pressure. Methanol was added, and the solids were stirred and then filtered to provide the title compound (145.3 g, 65%). ¹H NMR (400 MHz, CDCl₃) δ ppm 7.43-7.33 (m, 4H), 7.16 (tt, J=7.5, 1.0 Hz, 1H), 7.10-7.01 (m, 4H), 5.42 (s, 2H), 5.10 (s, 1H), 3.80 (dd, J=6.5, 4.9 Hz, 1H), 3.43 (dq, J=12.0, 4.0, 3.3 Hz, 5H), 3.28 (ddd, J=11.3, 7.6, 3.2 Hz, 1H), 2.72-2.57 (m, 4H), 2.17 (dtd, J=13.8, 7.0, 3.1 Hz, 1H), 1.93 (dddd, J=13.5, 8.3, 5.0, 3.3 Hz, 1H), 1.44 (s, 9H). MS (APCI) m/z: 515.2 [M+H]⁺.

### Step 7.7

### tert-butyl 4-[(7S)-3-carbamoyl-2-(4-phenoxyphenyl)-4,5,6,7-tetrahydro-2H-pyrazolo[4,3-b]pyridin-7-yl]piperazine-1-carboxylate

tert-Butyl 4-[3-carbamoyl-2-(4-phenoxyphenyl)-2H-pyrazolo[4,3-b]pyridin-7-yl]piperazine-1-carboxylate (20.27 g, 39.4 mmol, Step 7.6) was dissolved in a mixture of tetrahydrofuran (284 mL) and methanol (95 mL), and added to a 500 mL stainless steel reactor containing 10% Pd(OH)₂/C wet (20 g, 71.2 mmol). The reactor was pressurized with hydrogen (60 psi) and stirred at 50° C. After 24 hours, the reactor was cooled, vented, and filtered. The solvents were concentrated. The residue was dissolved in dichloromethane (25 mL) and purified via column chromatography on silica gel column eluting with a 0-5% methanol/dichloromethane gradient. The clean fractions were pooled and concentrated to produce a residue (18 g, 88%). The residue (0.799 g) was subjected to supercritical fluid chromatography using a ChiralPak® IB column (21×250 mm, 5 micron); concentration: 40 mg/mL in methanol; eluted at 30% ethanol/CO₂; injection volume: 0.5 mL to afford recovered peak A (R-enantiomer) (0.330 g, 83%) and Peak B (S-enantiomer) (0.370 g, 93%). ¹H NMR (400 MHz, dimethyl sulfoxide-d₆) δ ppm 8.80 (d, J=2.7 Hz, 1H), 8.32 (d, J=5.4 Hz, 1H), 7.88-7.83 (m, 1H), 7.57 (dt, J=8.6, 3.3 Hz, 2H), 7.47 (d, J=7.8 Hz, 2H), 7.23 (tt, J=7.5, 0.9 Hz, 1H), 7.19-7.05 (m, 4H), 6.58 (d, J=5.5 Hz, 1H), 3.92 (d, J=5.3 Hz, 4H), 3.52 (d, J=5.3 Hz, 4H), 1.42 (s, 9H). MS (APCI) m/z: 519.3 [M+H]⁺.

### Step 7.8

### (7S)-2-(4-phenoxyphenyl)-7-(piperazin-1-yl)-4,5,6,7-tetrahydro-2H-pyrazolo[4,3-b]pyridine-3-carboxamide

A 250 mL round bottom flask equipped with nitrogen inlet and needle thermocouple was charged with tert-butyl 4-[(7S)-3-carbamoyl-2-(4-phenoxyphenyl)-4,5,6,7-tetrahydro-2H-pyrazolo[4,3-b]pyridin-7-yl]piperazine-1-carboxylate (5 g, 9.64 mmol, Step 7.7) followed by anhydrous methanol (30 mL). In a separate 50 mL flask, was added methanol (11.70 mL, 289 mmol). The 50 mL flask was cooled in an ice bath and acetyl chloride (3.43 mL, 48.2 mmol) was added dropwise over 2 minutes. The solution was stirred for 10 minutes, and then cannulated material into the reaction flask. The reaction was warmed to 40° C. and stirred at this temperature for 1.5 hours. The reaction was then cooled and diethyl ether (200 mL) was added. The resultant precipitate was collected by vacuum filtration, washed with diethyl ether, and dried in a 55° C. vacuum oven overnight to afford 5.7 g of the HCl salt. The HCl salt was placed into a 1 L flask, and 10% methanol/dichloromethane (400 mL) was added followed by saturated sodium carbonate (100 mL). The biphasic reaction was stirred vigorously for 30 minutes and the layers separated. The aqueous layer was extracted with 10% methanol/dichloromethane (200 mL), and the combined organic layers were dried over anhydrous magnesium sulfate, filtered, and concentrated to afford the title compound (4.03 g). ¹H NMR (400 MHz, dimethyl sulfoxide-d₆) δ ppm 7.46-7.38 (m, 2H), 7.38-7.29 (m, 2H), 7.21-7.11 (m, 1H), 7.11-6.99 (m, 4H), 5.11 (t, J=3.0 Hz, 1H), 3.62-3.54 (m, 2H), 3.29-3.19 (m, 1H), 3.14-3.04 (m, 1H), 2.78 (t, J=5.0 Hz, 3H), 2.70-2.64 (m, 1H), 2.60 (td, J=12.5, 10.0, 4.8 Hz, 3H), 2.37 (s, 1H), 2.06 (dtt, J=13.5, 6.7, 2.9 Hz, 1H), 1.72 (ddt, J=13.5, 9.9, 3.9 Hz, 1H). MS (APCI) m/z: 419.2, [M+H]⁺.

### Step 7.9

### (7S)-2-(4-phenoxyphenyl)-7-[4-(prop-2-enoyl)piperazin-1-yl]-4,5,6,7-tetrahydro-2H-pyrazolo[4,3-b]pyridine-3-carboxamide

A 50 mL flask was charged with (7S)-2-(4-phenoxyphenyl)-7-(piperazin-1-yl)-4,5,6,7-tetrahydro-2H-pyrazolo[4,3-b]pyridine-3-carboxamide (0.500 g, 1.195 mmol, Step 7.8). Dichloromethane (12 mL), was added followed by N,N-diisopropylethylamine (0.313 mL, 1.792 mmol). The stirring reaction mixture was cooled to −78° C. and acryloyl chloride (1.0 M dichloromethane solution, 1.135 mL, 1.135 mmol) was added over 2 minutes. The reaction was stirred for 25 minutes and saturated aqueous sodium bicarbonate was added (15 mL). The reaction was warmed to ambient temperature and stirred for 10 minutes. The reaction was diluted with dichloromethane, and layers were separated. The organic layer was washed with water, dried over anhydrous magnesium sulfate, filtered, and concentrated. The residue was purified by flash column chromatography on silica (1-4.5% methanol/dichloromethane) to afford the title compound (0.329 g, 58%). ¹H NMR (400 MHz, CDCl₃) δ ppm 7.38 (dd, J=9.0, 7.2 Hz, 4H), 7.21-7.13 (m, 1H), 7.11-7.02 (m, 4H), 6.54 (dd, J=16.8, 10.5 Hz, 1H), 6.26 (dd, J=16.8, 2.0 Hz, 1H), 5.66 (dd, J=10.6, 2.0 Hz, 1H), 5.32 (s, 2H), 5.12 (d, J=2.3 Hz, 1H), 3.85 (dd, J=6.7, 5.0 Hz, 1H), 3.81-3.73 (m, 1H), 3.65 (dt, J=14.7, 6.8 Hz, 1H), 3.57 (d, J=5.2 Hz, 2H), 3.43 (ddt, J=11.2, 8.4, 2.7 Hz, 1H), 3.30 (tt, J=8.5, 7.1, 2.8 Hz, 1H), 2.78 (dt, J=11.2, 5.4 Hz, 1H), 2.74-2.61 (m, 3H), 2.17 (dtd, J=14.1, 7.2, 3.1 Hz, 1H), 1.95 (dddd, J=13.5, 8.3, 5.1, 3.3 Hz, 1H). MS (APCI) m/z: 473.2 [M+H]⁺.

### Example 8

### (7SR)-2-(4-phenoxyphenyl)-7-[(3RS)-1-(prop-2-enoyl)piperidin-3-yl]-4,5,6,7-tetrahydro-2H-pyrazolo[4,3-b]pyridine-3-carboxamide; and

### Example 9

### (7RS)-2-(4-phenoxyphenyl)-7-[(3RS)-1-(prop-2-enoyl)piperidin-3-yl]-4,5,6,7-tetrahydro-2H-pyrazolo[4,3-b]pyridine-3-carboxamide

Examples 8 and 9 were prepared according to the procedure for Step 78.5, substituting 2-(4-phenoxyphenyl)-7-(piperidin-3-yl)-4,5,6,7-tetrahydro-2H-pyrazolo[4,3-b]pyridine-3-carboxamide (Intermediate S) for (7R)-2-[4-(3-cyclopropylphenoxy)phenyl]-7-(piperazin-1-yl)-4,5,6,7-tetrahydro-2H-pyrazolo[4,3-b]pyridine-3-carboxamide to afford a mixture of the title compounds. The residue was purified by column chromatography on silica gel (0-100% ethyl acetate in heptanes, then 0-10% methanol in ethyl acetate to afford Example 9 (0.064 g, 0.136 mmol, 31%), ¹H NMR (400 MHz, dimethyl sulfoxide-d₆, 90° C.) δ ppm 7.45-7.31 (m, 4H), 7.14 (t, J=7.4 Hz, 1H), 7.09-6.98 (m, 4H), 6.76-6.57 (m, 3H), 5.98 (dd, J=16.8, 2.4 Hz, 1H), 5.50 (d, J=10.5 Hz, 1H), 4.89 (s, 1H), 4.20-4.01 (m, 3H), 3.30-3.21 (m, 1H), 3.13 (ddt, J=12.0, 8.8, 3.1 Hz, 1H), 2.82 (dt, J=8.4, 5.7 Hz, 2H), 1.95-1.82 (m, 3H), 1.82-1.68 (m, 2H), 1.57-1.45 (m, 1H), 1.38 (tdt, J=12.1, 8.1, 3.8 Hz, 1H). MS (ESI) m/z: 472.1 [M−H]⁺; and Example 8 (0.032 g, 0.068 mmol, 16%), ¹H NMR (400 MHz, dimethyl sulfoxide-d₆) δ ppm 7.43-7.32 (m, 4H), 7.14 (t, J=7.4 Hz, 1H), 7.07-7.01 (m, 4H), 6.74-6.65 (m, 3H), 5.98 (dd, J=16.8, 2.4 Hz, 1H), 5.49 (d, J=10.6 Hz, 1H), 4.90 (s, 1H), 4.62 (d, J=13.3 Hz, 1H), 4.19-4.05 (m, 1H), 3.30-3.20 (m, 1H), 3.16-3.07 (m, 1H), 2.84 (s, 2H), 2.71 (q, J=7.2 Hz, 1H), 1.87 (d, J=12.7 Hz, 2H), 1.82-1.66 (m, 3H), 1.53-1.27 (m, 2H). MS (ESI) m/z: 472.1 [M−H]⁺.

### Example 10

### 2-(4-phenoxyphenyl)-7-[1-(prop-2-enoyl)pyrrolidin-3-yl]-4,5,6,7-tetrahydro-2H-pyrazolo[4,3-b]pyridine-3-carboxamide

### Step 10.1

### tert-butyl 3-[3-carbamoyl-2-(4-phenoxyphenyl)-2H-pyrazolo[4,3-b]pyridin-7-yl]-2,5-dihydro-1H-pyrrole-1-carboxylate

Step 10.1 was prepared according to the procedure for Step S.1, substituting tert-butyl 3-(4,4,5,5-tetramethyl-1,3,2-dioxaborolan-2-yl)-2,5-dihydro-1H-pyrrole-1-carboxylate for tert-butyl 4-(4,4,5,5-tetramethyl-1,3,2-dioxaborolan-2-yl)-3,6-dihydropyridine-1(2H)-carboxylate. MS (ESI) m/z: 498.0 [M−H]⁺.

### Step 10.2

### tert-butyl 3-[3-carbamoyl-2-(4-phenoxyphenyl)-4,5,6,7-tetrahydro-2H-pyrazolo[4,3-b]pyridin-7-yl]pyrrolidine-1-carboxylate

Step 10.2 was prepared according to the procedure for Step S.2, substituting tert-butyl 3-[3-carbamoyl-2-(4-phenoxyphenyl)-2H-pyrazolo[4,3-b]pyridin-7-yl]-2,5-dihydro-1H-pyrrole-1-carboxylate (Step 10.1) for tert-butyl 5-[3-carbamoyl-2-(4-phenoxyphenyl)-2H-pyrazolo[4,3-b]pyridin-7-yl]-3,6-dihydropyridine-1(2H)-carboxylate. MS (ESI) m/z: 504.0 [M−H]⁺.

### Step 10.3

### 2-(4-phenoxyphenyl)-7-(pyrrolidin-3-yl)-4,5,6,7-tetrahydro-2H-pyrazolo[4,3-b]pyridine-3-carboxamide

Step 10.3 was prepared according to the procedure for Step S.3, substituting tert-butyl 3-[3-carbamoyl-2-(4-phenoxyphenyl)-4,5,6,7-tetrahydro-2H-pyrazolo[4,3-b]pyridin-7-yl]pyrrolidine-1-carboxylate (Step 10.2) for tert-butyl 3-[3-carbamoyl-2-(4-phenoxyphenyl)-4,5,6,7-tetrahydro-2H-pyrazolo[4,3-b]pyridin-7-yl]piperidine-1-carboxylate. MS (ESI) m/z: 404.2 [M−H]⁺.

### Step 10.4

### 2-(4-phenoxyphenyl)-7-[1-(prop-2-enoyl)pyrrolidin-3-yl]-4,5,6,7-tetrahydro-2H-pyrazolo[4,3-b]pyridine-3-carboxamide

Step 10.4 was prepared according to the procedure for Step 78.5 substituting 2-(4-phenoxyphenyl)-7-(pyrrolidin-3-yl)-4,5,6,7-tetrahydro-2H-pyrazolo[4,3-b]pyridine-3-carboxamide (Step 10.3) for (7R)-2-[4-(3-cyclopropylphenoxy)phenyl]-7-(piperazin-1-yl)-4,5,6,7-tetrahydro-2H-pyrazolo[4,3-b]pyridine-3-carboxamide to afford the title compound as a mixture of diastereomers. ¹H NMR (500 MHz, dimethyl sulfoxide-d₆) δ ppm 7.45-7.39 (m, 2H), 7.37-7.31 (m, 2H), 7.16 (tdd, J=7.5, 3.2, 1.9 Hz, 1H), 7.10-7.01 (m, 4H), 6.66-6.48 (m, 1H), 6.16-6.07 (m, 1H), 5.68-5.61 (m, 1H), 5.07 (s, 1H), 4.03-3.62 (m, 2H), 3.50-3.39 (m, 2H), 3.27-3.18 (m, 2H), 3.16-3.06 (m, 1H), 2.87-2.74 (m, 1H), 2.47-2.01 (m, 2H), 2.01-1.86 (m, 1H), 1.72-1.61 (m, 1H). MS (ESI) m/z: 458.1 [M−H]+.

### Example 11

### 2-[4-(4-fluorophenoxy)phenyl]-7-[1-(prop-2-enoyl)piperidin-4-yl]-4,5,6,7-tetrahydro-2H-pyrazolo[4,3-b]pyridine-3-carboxamide

### Step 11.1

### tert-butyl 4-[2-(4-bromophenyl)-3-cyano-2H-pyrazolo[4,3-b]pyridin-7-yl]piperidine-1-carboxylate

To a solution of pyridine (0.485 mL, 6.00 mmol) and tert-butyl 4-[2-(4-bromophenyl)-3-carbamoyl-2H-pyrazolo[4,3-b]pyridin-7-yl]piperidine-1-carboxylate (1.00 g, 2.00 mmol, Intermediate T) in dichloromethane (9.99 mL) was added trifluoroacetic acid (0.565 mL, 4.00 mmol) at ambient temperature. The reaction mixture was stirred at ambient temperature for 4 hours. Saturated aqueous NaHCO₃ was added, and the resulting solution was stirred for 30 minutes. The aqueous solution was extracted with dichloromethane (3×), and the combined organic fractions concentrated under reduced pressure. The crude product was purified by silica gel chromatography (0-50% ethyl acetate in heptanes) to provide the title compound (0.747 g, 1.55 mmol, 77%). MS (ESI) m/z: 483.9 [M+H]⁺.

### Step 11.2

### tert-butyl 4-{3-cyano-2-[4-(4-fluorophenoxy)phenyl]-2H-pyrazolo[4,3-b]pyridin-7-yl}piperidine-1-carboxylate

To a vial equipped with a stir bar were added 2,2,6,6-tetramethyl-3,5-heptanedione (0.014 g, 0.078 mmol), cesium carbonate (0.122 g, 0.373 mmol), copper(I) iodide (0.015 g, 0.078 mmol), 4-fluorophenol (0.042 mg, 0.373 mmol), and tert-butyl 4-[2-(4-bromophenyl)-3-cyano-2H-pyrazolo[4,3-b]pyridin-7-yl]piperidine-1-carboxylate (0.150 g, 0.311 mmol, Step 11.1). The vial was sealed and purged with N₂ (3×). N-Methyl-2-pyrrolidinone (3 mL) was added. The reaction mixture was warmed to 120° C. and stirred for 4 hours. The reaction mixture was cooled to ambient temperature and quenched with saturated aqueous NH₄Cl. The aqueous solution was extracted with ethyl acetate (3×). The combined organic layers were washed with brine (3×) and concentrated under reduced pressure. The crude product was purified by silica gel chromatography (0-100% ethyl acetate in heptanes) to provide the title compound (0.058 g, 0.11 mmol, 37%). MS (ESI) m/z: 514.0 [M+H]⁺.

### Step 11.3

### tert-butyl 4-{3-carbamoyl-2-[4-(4-fluorophenoxy)phenyl]-2H-pyrazolo[4,3-b]pyridin-7-yl}piperidine-1-carboxylate

To a solution of potassium carbonate (0.078 g, 0.57 mmol) and tert-butyl 4-{3-cyano-2-[4-(4-fluorophenoxy)phenyl]-2H-pyrazolo[4,3-b]pyridin-7-yl}piperidine-1-carboxylate (0.058 g, 0.11 mmol, Step 11.2) in 9:1 methanol/dimethyl sulfoxide (1.2 mL) was added 30% aqueous hydrogen peroxide (0.116 mL, 1.14 mmol), and the reaction mixture was stirred for 30 minutes at ambient temperature. The reaction was quenched with 1 M aqueous NaHSO₃, and the resulting solution was stirred for 15 minutes. The aqueous solution was extracted with ethyl acetate (3×), and the combined organic layers were concentrated under reduced pressure. The crude product was purified by silica gel chromatography (0-100% ethyl acetate in heptanes) to provide the title compound (0.023 g, 0.043 mmol, 38%). MS (ESI) m/z: 532.0 [M+H]⁺.

### Step 11.4

### tert-butyl 4-{3-carbamoyl-2-[4-(4-fluorophenoxy)phenyl]-4,5,6,7-tetrahydro-2H-pyrazolo[4,3-b]pyridin-7-yl}piperidine-1-carboxylate

To a reactor (Thermo Barnstead Stem RS10) were added tert-butyl 4-{3-carbamoyl-2-[4-(4-fluorophenoxy)phenyl]-2H-pyrazolo[4,3-b]pyridin-7-yl}piperidine-1-carboxylate (0.067 g, 0.13 mmol, Step 11.3), 5% Pd/C (0.068 g, 0.30 mmol) and tetrahydrofuran (2 mL). The vessel was pressurized with H₂ (50 psi), and the reaction mixture was stirred for 51 hours at ambient temperature. The resulting solution was filtered over diatomaceous earth, and the filtrate was concentrated under reduced pressure. The crude product was purified by silica gel chromatography (0-100% ethyl acetate in heptanes) to provide the title compound (0.022 g, 0.040 mmol, 32%). MS (ESI) m/z: 536.1 [M+H]⁺.

### Step 11.5

### 2-[4-(4-fluorophenoxy)phenyl]-7-(piperidin-4-yl)-4,5,6,7-tetrahydro-2H-pyrazolo[4,3-b]pyridine-3-carboxamide

To a solution of tert-butyl 4-{3-carbamoyl-2-[4-(4-fluorophenoxy)phenyl]-4,5,6,7-tetrahydro-2H-pyrazolo[4,3-b]pyridin-7-yl}piperidine-1-carboxylate (0.022 g, 0.040 mmol, Step 11.4) in 1,4-dioxane (0.4 mL) was added 4 M HCl in 1,4-dioxane (0.4 mL). The reaction mixture was stirred at ambient temperature for 30 minutes. The reaction mixture was concentrated under reduced pressure to provide the crude title compound, which was used without further purification. MS (APCI) m/z: 436.5 [M+H]⁺.

### Step 11.6

### 2-[4-(4-fluorophenoxy)phenyl]-7-[1-(prop-2-enoyl)piperidin-4-yl]-4,5,6,7-tetrahydro-2H-pyrazolo[4,3-b]pyridine-3-carboxamide

To a solution of acrylic acid (0.003 mL, 0.044 mmol), N,N-diisopropylethylamine (0.064 mL, 0.40 mmol), and 2-[4-(4-fluorophenoxy)phenyl]-7-(piperidin-4-yl)-4,5,6,7-tetrahydro-2H-pyrazolo[4,3-b]pyridine-3-carboxamide (0.017 g, 0.12 mmol, Step 11.5) in N,N-dimethylformamide (0.8 mL) was added 1-propanephosphonic anhydride (50% in N,N-dimethylformamide, 0.028 mL, 0.048 mmol) at ambient temperature. The reaction mixture was stirred for 15 minutes, and then quenched with saturated aqueous NaHCO₃. Dichloromethane (4 mL) was added, and the resulting heterogeneous solution was filtered through a phase separator. The organic layer was concentrated under reduced pressure. The crude product was diluted with N,N-dimethylformamide (2 mL) and purified by preparative HPLC (5-20% acetonitrile in 0.1% aqueous trifluoroacetic acid over 4 minutes, 20-45% acetonitrile in 0.1% aqueous trifluoroacetic acid over 10 minutes; Column: XBridge™ BEH C18 OBD Prep Column, 30 mm×100 mm, 5 μm particle size; Flow Rate: 40 mL/min; Detection wavelength: 220 nm and 254 nm). The fractions containing product were concentrated under reduced pressure and purified further by silica gel chromatography (0-50% methanol in dichloromethane) to provide the title compound (0.0032 g, 16%). ¹H NMR (400 MHz, dimethyl sulfoxide-d₆) δ ppm 7.35-7.20 (m, 4H), 7.16-7.06 (m, 2H), 7.04-6.97 (m, 2H), 6.79 (dd, J=16.5, 10.5 Hz, 1H), 6.06 (dd, J=16.7, 2.5 Hz, 1H), 5.63 (dd, J=10.4, 2.5 Hz, 1H), 5.05-4.99 (m, 1H), 4.47 (d, J=12.8 Hz, 1H), 4.15-4.01 (m, 1H), 3.42-3.28 (m, 1H), 3.27-3.14 (m, 1H), 3.13-2.92 (m, 2H), 2.73 (s, 1H), 2.56 (q, J=11.7, 10.2 Hz, 1H), 2.05-1.60 (m, 5H), 1.24 (s, 2H). MS (ESI) m/z: 489.9 [M+H]⁺.

### Example 12

### 2-[4-(3-fluorophenoxy)phenyl]-7-[1-(prop-2-enoyl)piperidin-4-yl]-4,5,6,7-tetrahydro-2H-pyrazolo[4,3-b]pyridine-3-carboxamide

### Step 12.1

### tert-butyl 4-{3-carbamoyl-2-[4-(3-fluorophenoxy)phenyl]-2H-pyrazolo[4,3-b]pyridin-7-yl}piperidine-1-carboxylate

To a vial equipped with a stir bar were added 2,2,6,6-tetramethyl-3,5-heptanedione (0.184 g, 0.999 mmol), cesium carbonate (0.391 g, 1.199 mmol), copper(I) iodide (0.190 g, 0.999 mmol), 3-fluorophenol (0.109 mL, 1.199 mmol), and tert-butyl 4-[2-(4-bromophenyl)-3-carbamoyl-2H-pyrazolo[4,3-b]pyridin-7-yl]piperidine-1-carboxylate (0.500 g, 0.999 mmol, Intermediate T). The vial was sealed and purged with N₂ (3×). N-Methyl-2-pyrrolidinone (10 mL) was added. The reaction mixture was warmed to 120° C. and stirred for 4 hours. The reaction mixture was cooled to ambient temperature and quenched with saturated aqueous NH₄Cl. The aqueous solution was extracted with ethyl acetate (3×). The combined organic layers were washed with brine (3×) and concentrated under reduced pressure. The crude product was purified by silica gel chromatography (0-100% ethyl acetate in heptanes) to provide the title compound (0.125 g, 0.235 mmol, 24%). MS (ESI) m/z: 532.0 [M+H]⁺.

### Step 12.2

### tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate

A solution of tert-butyl 4-{3-carbamoyl-2-[4-(3-fluorophenoxy)phenyl]-2H-pyrazolo[4,3-b]pyridin-7-yl}piperidine-1-carboxylate (0.125 g, 0.235 mmol, Step 12.1) in 1:1 tetrahydrofuran/methanol (4 mL) was injected into a flow hydrogenation system (ThalesNano H-Cube® Pro equipped with a 70 mm 10% Pd/C CatCart®, flow rate: 1.0 mL/min, eluting with 100% methanol, temperature: ambient temperature, H₂ pressure: 20 bar). The reaction mixture was concentrated under reduced pressure, and the crude residue was purified by silica gel chromatography (0-100% ethyl acetate in heptanes) to provide the title compound (0.0658 g, 52%). MS (ESI) m/z: 536.1 [M+H]⁺.

### Step 12.3

### 2-[4-(3-fluorophenoxy)phenyl]-7-(piperidin-4-yl)-4,5,6,7-tetrahydro-2H-pyrazolo[4,3-b]pyridine-3-carboxamide

To a solution of tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate (0.0658 g, 0.123 mmol, Step 12.2) in 1,4-dioxane (1.2 mL) was added 4 M HCl in 1,4-dioxane (1.2 mL). The reaction mixture was stirred at ambient temperature for 30 minutes. The reaction mixture was concentrated under reduced pressure to provide the crude title compound, which was used without further purification. MS (ESI) m/z: 436.2 [M+H]⁺.

### Step 12.4

### 2-[4-(3-fluorophenoxy)phenyl]-7-[1-(prop-2-enoyl)piperidin-4-yl]-4,5,6,7-tetrahydro-2H-pyrazolo[4,3-b]pyridine-3-carboxamide

To a solution of acrylic acid (0.00929 mL, 0.135 mmol), N,N-diisopropylethylamine (0.215 mL, 1.230 mmol), and 2-[4-(3-fluorophenoxy)phenyl]-7-(piperidin-4-yl)-4,5,6,7-tetrahydro-2H-pyrazolo[4,3-b]pyridine-3-carboxamide (0.0536 g, 0.123 mmol, Step 12.3) in N,N-dimethylformamide (2.5 mL) was added 1-propanephosphonic anhydride (0.094 g, 0.15 mmol) at ambient temperature. The reaction mixture was stirred for 15 minutes. The reaction mixture was quenched with saturated aqueous NaHCO₃. Dichloromethane (4 mL) was added, and the resulting heterogeneous solution was filtered through a phase separator. The organic layer was concentrated under reduced pressure. The crude product was diluted with N,N-dimethylformamide (2 mL) and purified by preparative HPLC (5-20% acetonitrile in 0.1% aqueous trifluoroacetic acid over 4 minutes, 20-45% acetonitrile in 0.1% aqueous trifluoroacetic acid over 10 minutes; Column: XBridge™ BEH C18 OBD Prep Column, 30 mm×100 mm, 5 μm particle size; Flow Rate: 40 mL/min; detection wavelength: 220 nm and 254 nm). The fractions containing product were concentrated under reduced pressure and purified further by silica gel chromatography (0-15% methanol in dichloromethane) to provide the title compound (0.0236 g, 0.048 mmol, 39%). ¹H NMR (400 MHz, dimethyl sulfoxide-d₆) δ ppm 7.49-7.39 (m, 1H), 7.38-7.31 (m, 2H), 7.14-7.06 (m, 2H), 6.99 (tdd, J=8.4, 2.4, 0.9 Hz, 1H), 6.94-6.85 (m, 2H), 6.85-6.72 (m, 1H), 6.06 (dd, J=16.7, 2.5 Hz, 1H), 5.63 (dd, J=10.5, 2.5 Hz, 1H), 4.47 (d, J=12.8 Hz, 1H), 4.13-4.02 (m, 1H), 3.47-3.16 (m, 2H), 3.14-2.90 (m, 2H), 2.80-2.69 (m, 1H), 2.64-2.53 (m, 1H), 2.05-1.57 (m, 5H), 1.36-1.15 (m, 2H). MS (ESI) m/z: 489.9 [M+H]⁺.

### Example 13

### 2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide

### Step 13.1

### tert-butyl 4-{3-carbamoyl-2-[4-(2,4-difluorophenoxy)phenyl]-2H-pyrazolo[4,3-b]pyridin-7-yl}piperidine-1-carboxylate

Step 13.1 was prepared according to the procedure for Step 12.1, substituting 2,4-difluorophenol for 3-fluorophenol. MS (ESI) m/z: 550.0 [M+H]⁺.

### Step 13.2

### tert-butyl 4-{3-carbamoyl-2-[4-(2,4-difluorophenoxy)phenyl]-4,5,6,7-tetrahydro-2H-pyrazolo[4,3-b]pyridin-7-yl}piperidine-1-carboxylate

Step 13.2 was prepared according to the procedure for Step 12.2, substituting tert-butyl 4-{3-carbamoyl-2-[4-(2,4-difluorophenoxy)phenyl]-2H-pyrazolo[4,3-b]pyridin-7-yl}piperidine-1-carboxylate (Step 13.1) for tert-butyl 4-{3-carbamoyl-2-[4-(3-fluorophenoxy)phenyl]-2H-pyrazolo[4,3-b]pyridin-7-yl}piperidine-1-carboxylate. MS (ESI) m/z: 554.0 [M+H]⁺.

### Step 13.3

### 2-[4-(2,4-difluorophenoxy)phenyl]-7-(piperidin-4-yl)-4,5,6,7-tetrahydro-2H-pyrazolo[4,3-b]pyridine-3-carboxamide

Step 13.3 was prepared according to the procedure for Step 12.3, substituting tert-butyl 4-{3-carbamoyl-2-[4-(2,4-difluorophenoxy)phenyl]-4,5,6,7-tetrahydro-2H-pyrazolo[4,3-b]pyridin-7-yl}piperidine-1-carboxylate (Step 13.2) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. MS (ESI) m/z: 454.1 [M+H]⁺.

### Step 13.4

### 2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide

Step 13.4 was prepared according to the procedure for Step 12.4, substituting 2-[4-(2,4-difluorophenoxy)phenyl]-7-(piperidin-4-yl)-4,5,6,7-tetrahydro-2H-pyrazolo[4,3-b]pyridine-3-carboxamide (Step 13.3) for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, dimethyl sulfoxide-d₆) δ ppm 7.52-7.42 (m, 1H), 7.38-7.25 (m, 3H), 7.17-7.08 (m, 1H), 7.01-6.92 (m, 2H), 6.80-6.69 (m, 1H), 6.03 (dd, J=16.7, 2.4 Hz, 1H), 5.60 (dd, J=10.5, 2.4 Hz, 1H), 4.43 (d, J=12.8 Hz, 1H), 4.13-3.97 (m, 1H), 3.52-3.12 (m, 2H), 3.11-2.86 (m, 2H), 2.76-2.65 (m, 1H), 2.57-2.49 (m, 1H), 2.03-1.57 (m, 5H), 1.38-1.09 (m, 2H). MS (ESI) m/z: 508.0 [M+H]⁺.

### Example 14

### 2-(2-methoxy-4-phenoxyphenyl)-7-[1-(prop-2-enoyl)piperidin-4-yl]-4,5,6,7-tetrahydro-2H-pyrazolo[4,3-b]pyridine-3-carboxamide

### Step 14.1

### tert-butyl 4-[3-carbamoyl-2-(2-methoxy-4-phenoxyphenyl)-2H-pyrazolo[4,3-b]pyridin-7-yl]piperidine-1-carboxylate

Step 14.1 was prepared according to the procedure for Step 12.1, substituting tert-butyl 4-[2-(4-bromo-2-methoxyphenyl)-3-carbamoyl-2H-pyrazolo[4,3-b]pyridin-7-yl]piperidine-1-carboxylate (Intermediate F) for tert-butyl 4-[2-(4-bromophenyl)-3-carbamoyl-2H-pyrazolo[4,3-b]pyridin-7-yl]piperidine-1-carboxylate and phenol for 3-fluorophenol. MS (ESI) m/z: 544.1 [M+H]⁺.

### Step 14.2

### tert-butyl 4-[3-carbamoyl-2-(2-methoxy-4-phenoxyphenyl)-4,5,6,7-tetrahydro-2H-pyrazolo[4,3-b]pyridin-7-yl]piperidine-1-carboxylate

A solution of tert-butyl 4-[3-carbamoyl-2-(2-methoxy-4-phenoxyphenyl)-2H-pyrazolo[4,3-b]pyridin-7-yl]piperidine-1-carboxylate (0.126 g, 0.232 mmol, Step 14.1) in 1:1 tetrahydrofuran/methanol (4 mL) was injected into a flow hydrogenation system (ThalesNano H-Cube® Pro equipped with a 70 mm 10% Pd/C CatCart©, flow rate: 1.0 mL/min, eluting with 100% methanol, temperature: ambient temperature, H₂ pressure: 20 bar). The reaction mixture was concentrated under reduced pressure, and the crude residue was purified by silica gel chromatography (0-100% ethyl acetate in heptanes) to provide the title compound (0.0586 g, 0.107 mmol, 46%). MS (ESI) m/z: 548.0 [M+H]⁺.

### Step 14.3

### 2-(2-methoxy-4-phenoxyphenyl)-7-(piperidin-4-yl)-4,5,6,7-tetrahydro-2H-pyrazolo[4,3-b]pyridine-3-carboxamide

Step 14.3 was prepared according to the procedure for Step 12.3, substituting tert-butyl 4-[3-carbamoyl-2-(2-methoxy-4-phenoxyphenyl)-4,5,6,7-tetrahydro-2H-pyrazolo[4,3-b]pyridin-7-yl]piperidine-1-carboxylate (Step 14.2) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. MS (ESI) m/z: 448.2 [M+H]⁺.

### Step 14.4

### 2-(2-methoxy-4-phenoxyphenyl)-7-[1-(prop-2-enoyl)piperidin-4-yl]-4,5,6,7-tetrahydro-2H-pyrazolo[4,3-b]pyridine-3-carboxamide

Step 14.4 was prepared according to the procedure for Step 12.4, substituting 2-(2-methoxy-4-phenoxyphenyl)-7-(piperidin-4-yl)-4,5,6,7-tetrahydro-2H-pyrazolo[4,3-b]pyridine-3-carboxamide (Step 14.3) for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (500 MHz, dimethyl sulfoxide-d₆) δ ppm 7.50-7.42 (m, 2H), 7.33 (d, J=8.6 Hz, 1H), 7.25-7.18 (m, 1H), 7.16-7.10 (m, 2H), 6.87 (d, J=2.6 Hz, 1H), 6.85-6.76 (m, 1H), 6.57 (dd, J=8.5, 2.6 Hz, 1H), 6.08 (dd, J=16.7, 2.5 Hz, 1H), 5.65 (dd, J=10.5, 2.5 Hz, 1H), 4.52-4.46 (m, 1H), 4.14-4.04 (m, 1H), 3.69 (s, 3H), 3.40-3.30 (m, 1H), 3.20-3.12 (m, 2H), 3.06-2.94 (m, 1H), 2.87-2.76 (m, 1H), 2.63-2.54 (m, 1H), 2.10-1.96 (m, 1H), 1.95-1.84 (m, 2H), 1.82-1.70 (m, 1H), 1.64 (d, J=12.9 Hz, 1H), 1.36-1.16 (m, 2H). MS (ESI) m/z: 502.1 [M+H]⁺.

### Example 15

### 2-[4-(3-methoxyphenoxy)phenyl]-7-[1-(prop-2-enoyl)piperidin-4-yl]-4,5,6,7-tetrahydro-2H-pyrazolo[4,3-b]pyridine-3-carboxamide

### Step 15.1

### tert-butyl 4-{3-carbamoyl-2-[4-(3-methoxyphenoxy)phenyl]-2H-pyrazolo[4,3-b]pyridin-7-yl}piperidine-1-carboxylate

Step 15.1 was prepared according to the procedure for Step 12.1, substituting 3-methoxyphenol for 3-fluorophenol. MS (APCI) m/z: 544.6 [M+H]⁺.

### Step 15.2

### tert-butyl 4-{3-carbamoyl-2-[4-(3-methoxyphenoxy)phenyl]-4,5,6,7-tetrahydro-2H-pyrazolo[4,3-b]pyridin-7-yl}piperidine-1-carboxylate

A solution of tert-butyl 4-{3-carbamoyl-2-[4-(3-methoxyphenoxy)phenyl]-2H-pyrazolo[4,3-b]pyridin-7-yl}piperidine-1-carboxylate (0.126 g, 0.232 mmol, Step 15.1) in tetrahydrofuran (4 mL) was injected into a flow hydrogenation system (ThalesNano H-Cube© Pro equipped with a 70 mm 10% Pd/C CatCart®, flow rate: 1.0 mL/min, eluting with 100% methanol, temperature: 30° C., H₂ pressure: 20 bar). The reaction mixture was concentrated under reduced pressure, and the crude residue was purified by silica gel chromatography (0-100% ethyl acetate in heptanes) to provide the title compound (0.0698 g, 55%). MS (ESI) m/z: 548.1 [M+H]⁺.

### Step 15.3

### 2-[4-(3-methoxyphenoxy)phenyl]-7-(piperidin-4-yl)-4,5,6,7-tetrahydro-2H-pyrazolo[4,3-b]pyridine-3-carboxamide

Step 15.3 was prepared according to the procedure for Step 12.3, substituting tert-butyl 4-{3-carbamoyl-2-[4-(3-methoxyphenoxy)phenyl]-4,5,6,7-tetrahydro-2H-pyrazolo[4,3-b]pyridin-7-yl}piperidine-1-carboxylate (Step 15.2) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. MS (ESI) m/z: 448.2 [M+H]⁺.

### Step 15.4

### 2-[4-(3-methoxyphenoxy)phenyl]-7-[1-(prop-2-enoyl)piperidin-4-yl]-4,5,6,7-tetrahydro-2H-pyrazolo[4,3-b]pyridine-3-carboxamide

Step 15.4 was prepared according to the procedure for Step 12.4, substituting 2-[4-(3-methoxyphenoxy)phenyl]-7-(piperidin-4-yl)-4,5,6,7-tetrahydro-2H-pyrazolo[4,3-b]pyridine-3-carboxamide (Step 15.3) for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, dimethyl sulfoxide-d₆) δ ppm 7.36-7.26 (m, 3H), 7.08-7.00 (m, 2H), 6.85-6.70 (m, 2H), 6.66-6.56 (m, 2H), 6.06 (dd, J=16.7, 2.5 Hz, 1H), 5.63 (dd, J=10.5, 2.5 Hz, 1H), 4.47 (d, J=12.8 Hz, 1H), 4.12-4.04 (m, 1H), 3.75 (s, 3H), 3.51-3.14 (m, 2H), 3.14-2.92 (m, 2H), 2.73 (s, 1H), 2.64-2.53 (m, 1H), 2.06-1.60 (m, 5H), 1.35-1.17 (m, 2H). MS (ESI) m/z: 502.1 [M+H]⁺.

### Example 16

### 2-(4-phenoxyphenyl)-7-[1-(prop-2-enoyl)piperidin-4-yl]-4,5,6,7-tetrahydro-2H-pyrazolo[3,4-b]pyrazine-3-carboxamide

### Step 16.1

### 4-nitro-3-[(piperidin-4-yl)(prop-2-en-1-yl)amino]-1H-pyrazole-5-carboxamide

tert-Butyl 4-[{5-carbamoyl-1-[(4-methoxyphenyl)methyl]-4-nitro-1H-pyrazol-3-yl}(prop-2-en-1-yl)amino]piperidine-1-carboxylate (28.7 g, 55.7 mmol, prepared according to the procedure for Step J.2, substituting tert-butyl 4-aminopiperidine-1-carboxylate for tert-butyl 3-aminoazetidine-1-carboxylate) was weighed into a 500-mL flask, and 2,2,2-trifluoroacetic acid (127 g, 1.12 mol) and anisole (18.1 g, 167 mmol) were added. The resulting solution was heated to 65° C. for 18 hours. The reaction flask was cooled to ambient temperature and concentrated in vacuo. The resulting crude oil was concentrated from toluene and then sonicated with heptanes (4×), decanting the solvent layer in between. The resulting crude oil was used without additional purification (16.4 g). MS (APCI) m/z: 295.0 [M+H]⁺.

### Step 16.2

### tert-butyl 4-[(5-carbamoyl-4-nitro-1H-pyrazol-3-yl)(prop-2-en-1-yl)amino]piperidine-1-carboxylate

4-nitro-3-[(piperidin-4-yl)(prop-2-en-1-yl)amino]-1H-pyrazole-5-carboxamide (16.4 g, 55.7 mmol, Step 16.1) was dissolved in dichloromethane (279 mL) and N,N-diisopropylethylamine (48.7 mL, 279 mmol) was added. The resulting dark solution was treated with di-tert-butyl decarbonate (15.5 mL, 66.9 mmol), and the reaction was stirred for 60 minutes at ambient temperature, at which point complete consumption of the starting material was observed. 1H-Imidazole was added (3.79 g, 55.7 mmol), and the reaction was stirred for 30 minutes at ambient temperature. The reaction mixture was diluted with dichloromethane and washed with saturated NaHCO₃ (2×), then brine, dried over sodium sulfate, and concentrated under reduced pressure. The crude residue was purified via flash chromatography, eluting with methanol/ethyl acetate (0/100 to 5/95) over 20 minutes on a 330 g silica gel column to afford the title compound (17.0 g, 77% over two steps). ¹H NMR (500 MHz, CDCl₃) δ ppm 8.79 (s, 1H), 6.74-6.63 (m, 1H), 5.72 (ddt, J=17.2, 10.2, 5.6 Hz, 1H), 5.17-5.13 (m, 1H), 5.04 (dd, J=10.2, 1.6 Hz, 1H), 4.19 (s, 2H), 3.91 (d, J=5.7 Hz, 2H), 3.53 (tt, J=11.7, 3.7 Hz, 1H), 2.73 (s, 2H), 1.93-1.85 (m, 2H), 1.71 (tt, J=12.3, 6.2 Hz, 2H), 1.47 (s, 9H). MS (APCI) m/z: 339.1 [M+H]⁺.

### Step 16.3

### tert-butyl 4-{[5-carbamoyl-4-nitro-1-(4-phenoxyphenyl)-1H-pyrazol-3-yl](prop-2-en-1-yl)amino}piperidine-1-carboxylate

tert-Butyl 4-[(5-carbamoyl-4-nitro-1H-pyrazol-3-yl)(prop-2-en-1-yl)amino]piperidine-1-carboxylate (15.2 g, 71.0 mmol, Step 16.2), copper (II) acetate (9.67 g, 53.2 mmol), and pyridine (14.4 mL, 177 mmol) were dissolved in dichloromethane (177 mL), and the reaction mixture was stirred at ambient temperature for 72 hours. The reaction mixture was poured over a pad of silica (200 g) and eluted with dichloromethane and ethyl acetate. The filtrate was concentrated under reduced pressure and loaded onto a silica gel column and eluted ethyl acetate/heptanes (0/100 to 50/50) over 20 minutes to afford the title compound (12.0 g, 60%). ¹H NMR (500 MHz, CDCl₃) δ ppm 7.54-7.43 (m, 2H), 7.43-7.32 (m, 2H), 7.21-7.14 (m, 1H), 7.12-6.98 (m, 4H), 6.37 (s, 1H), 6.13 (s, 1H), 5.81 (ddt, J=17.2, 10.2, 5.7 Hz, 1H), 5.21 (dd, J=17.1, 1.7 Hz, 1H), 5.09 (dd, J=10.2, 1.6 Hz, 1H), 4.18 (s, 2H), 3.89 (d, J=5.7 Hz, 2H), 3.58 (tt, J=11.7, 3.7 Hz, 1H), 2.71 (s, 2H), 1.96-1.80 (m, 2H), 1.72 (qd, J=12.2, 4.4 Hz, 2H), 1.45 (s, 9H). MS (APCI) m/z: 563.5 [M+H]⁺.

### Step 16.4

### tert-butyl 4-{[5-carbamoyl-4-nitro-1-(4-phenoxyphenyl)-1H-pyrazol-3-yl](2,3-dihydroxypropyl)amino}piperidine-1-carboxylate

tert-butyl 4-{[5-carbamoyl-4-nitro-1-(4-phenoxyphenyl)-1H-pyrazol-3-yl](prop-2-en-1-yl)amino}piperidine-1-carboxylate (12.0 g, 21.3 mmol, Step 16.3) was dissolved in a mixture of tetrahydrofuran (203 mL) and water (10.2 mL). The solution was treated with osmium tetroxide (4% w/w in water, 6.52 mL, 1.07 mmol) and 4-methylmorpholine N-oxide (5.00 g, 42.7 mmol), and the reaction mixture was stirred at ambient temperature for 16 hours. An aqueous saturated solution of sodium bisulfite (213 mL, 213 mmol) was added, and the reaction mixture was stirred at ambient temperature for 10 minutes. The mixture was diluted with brine and ethyl acetate. The layers were separated, and the aqueous layer was extracted with ethyl acetate. The combined organic extracts were washed with 50% saturated aqueous sodium chloride (2×), then brine, dried over sodium sulfate, concentrated in vacuo, to afford the title compound (12.7 g, 100%). MS (APCI) m/z: 597.5 [M+H]⁺.

### Step 16.5

### tert-butyl 4-{[5-carbamoyl-4-nitro-1-(4-phenoxyphenyl)-1H-pyrazol-3-yl](2-oxoethyl)amino}piperidine-1-carboxylate

tert-Butyl 4-{[5-carbamoyl-4-nitro-1-(4-phenoxyphenyl)-1H-pyrazol-3-yl](2,3-dihydroxypropyl)amino}piperidine-1-carboxylate (12.7 g, 21.3 mmol, Step 16.4) was dissolved in acetone (200 mL), and the solution was treated with water (80 mL). The resulting suspension was stirred at ambient temperature for 6 hours, the mixture was diluted with brine and ethyl acetate and the layers were separated. The organic layer was washed with brine, dried over sodium sulfate, and concentrated under reduced pressure to afford the title compound (12.0 g, 100%). ¹H NMR (500 MHz, CDCl₃) δ ppm 9.62 (s, 1H), 7.54-7.43 (m, 2H), 7.41-7.34 (m, 2H), 7.18 (tt, J=7.5, 1.1 Hz, 1H), 7.10-6.94 (m, 4H), 6.57 (s, 1H), 6.22 (s, 1H), 4.20 (s, 2H), 3.95 (s, 2H), 3.77 (tt, J=11.9, 3.7 Hz, 1H), 2.73 (s, 2H), 1.96-1.88 (m, 2H), 1.57 (tt, J=12.3, 6.1 Hz, 2H), 1.45 (s, 9H). MS (APCI) m/z: 565.5 [M+H]⁺.

### Step 16.6

### tert-butyl 4-[3-carbamoyl-2-(4-phenoxyphenyl)-2,4,5,6-tetrahydro-7H-pyrazolo[3,4-b]pyrazin-7-yl]piperidine-1-carboxylate

tert-Butyl 4-{[5-carbamoyl-4-nitro-1-(4-phenoxyphenyl)-1H-pyrazol-3-yl](2-oxoethyl)amino}piperidine-1-carboxylate (12.8 g, 22.7 mmol, Step 16.5) in tetrahydrofuran (162 mL) was added to Raney-Ni 2800, water slurry (7.63 g, 58.5 mmol) in a 300 mL stainless steel reactor. The reactor was purged with nitrogen. The mixture was stirred at 1000 RPM under 80 psi of H₂ at 50° C. for 18 hours. The reactor was cooled to ambient temperature and purged with N₂, and the resulting slurry was filtered through a pad of diatomaceous earth using a fritted funnel. The filtrate was concentrated under reduced pressure to afford a residue which was slurried in tert-butyl methyl ether. The resulting solid was isolated via filtration through a fritted funnel and dried to constant weight to afford the title compound (7.50 g, 64%). ¹H NMR (400 MHz, CDCl₃) δ ppm 7.35 (dddd, J=9.4, 7.1, 5.1, 2.7 Hz, 4H), 7.18-7.09 (m, 1H), 7.09-6.97 (m, 4H), 5.26 (s, 2H), 4.20 (s, 2H), 3.95 (ddt, J=12.0, 8.0, 4.1 Hz, 1H), 3.52-3.36 (m, 2H), 3.26 (t, J=4.7 Hz, 2H), 2.79 (t, J=12.4 Hz, 2H), 1.84-1.53 (m, 4H), 1.46 (s, 9H). MS (APCI) m/z: 519.6 [M+H]⁺.

### Step 16.7

### 2-(4-phenoxyphenyl)-7-(piperidin-4-yl)-4,5,6,7-tetrahydro-2H-pyrazolo[3,4-b]pyrazine-3-carboxamide-hydrogen chloride (1/2)

Acetyl chloride (2.74 mL, 38.6 mmol) was added slowly to a round-bottomed flask containing methanol (40.0 mL, 989 mmol), and the resulting solution was treated with tert-butyl 4-[3-carbamoyl-2-(4-phenoxyphenyl)-2,4,5,6-tetrahydro-7H-pyrazolo[3,4-b]pyrazin-7-yl]piperidine-1-carboxylate (4.00 g, 7.71 mmol, Step 16.6) in one portion. The reaction was heated to 50° C. for 1 hour, then cooled to ambient temperature and concentrated under reduced pressure to afford the title compound (3.79 g). MS (APCI) m/z: 419.3 [M+H]⁺.

### Step 16.8

### 2-(4-phenoxyphenyl)-7-[1-(prop-2-enoyl)piperidin-4-yl]-4,5,6,7-tetrahydro-2H-pyrazolo[3,4-b]pyrazine-3-carboxamide

2-(4-Phenoxyphenyl)-7-(piperidin-4-yl)-4,5,6,7-tetrahydro-2H-pyrazolo[3,4-b]pyrazine-3-carboxamide-hydrogen chloride (1/2) (3.23 g, 7.72 mmol, Step 16.7) was dissolved in dichloromethane (77 mL) in a 500-mL round-bottomed flask, and the resulting solution was cooled to −20° C. before addition of N,N-diisopropylethylamine (8.09 mL, 46.3 mmol) and acrylic acid (0.530 mL, 7.72 mmol). 2,4,6-Tripropyl-1,3,5,2,4,6-trioxatriphosphinane 2,4,6-trioxide (50% w/w in ethyl acetate, 4.82 mL, 8.10 mmol) was added dropwise via syringe, and the solution was stirred for 15 minutes before warming to 0° C. and quenching with water. The layers were separated, and the organic layer was washed with water (2×), then brine, dried over sodium sulfate and concentrated under reduced pressure to afford a crude residue. The crude residue was purified via flash chromatography, eluting with methanol/dichloromethane (0/100 to 5/95) over 20 minutes on an 80 g silica gel column to afford the title compound (2.10 g, 57.6% over 2 steps). ¹H NMR (500 MHz, CDCl₃) δ ppm 7.45-7.29 (m, 4H), 7.20-7.11 (m, 1H), 7.10-6.92 (m, 4H), 6.60 (dd, J=16.8, 10.6 Hz, 1H), 6.28 (dd, J=16.8, 1.9 Hz, 1H), 5.69 (dd, J=10.6, 1.9 Hz, 1H), 5.31 (br s, 2H), 4.99 (br s, 1H), 4.80 (br d, J=13.3 Hz, 1H), 4.07 (td, J=11.9, 10.4, 4.4 Hz, 2H), 3.43 (t, J=4.8 Hz, 2H), 3.28-3.22 (m, 2H), 3.15 (t, J=12.9 Hz, 1H), 2.76-2.63 (m, 1H), 1.90 (t, J=14.4 Hz, 2H), 1.71 (dtd, J=25.9, 13.6, 12.7, 6.8 Hz, 2H). MS (APCI) m/z: 473.4 [M+H]⁺.

### Example 17

### 2-[4-(4-methoxyphenoxy)phenyl]-7-[1-(prop-2-enoyl)piperidin-4-yl]-4,5,6,7-tetrahydro-2H-pyrazolo[4,3-b]pyridine-3-carboxamide

### Step 17.1

### tert-butyl 4-{3-carbamoyl-2-[4-(4-methoxyphenoxy)phenyl]-2H-pyrazolo[4,3-b]pyridin-7-yl}piperidine-1-carboxylate

Step 17.1 was prepared according to the procedure for Step 12.1, substituting 4-methoxyphenol for 3-fluorophenol MS (ESI) m/z: 544.1 [M+H]⁺.

### Step 17.2

### tert-butyl 4-{3-carbamoyl-2-[4-(4-methoxyphenoxy)phenyl]-4,5,6,7-tetrahydro-2H-pyrazolo[4,3-b]pyridin-7-yl}piperidine-1-carboxylate

A solution of tert-butyl 4-{3-carbamoyl-2-[4-(4-methoxyphenoxy)phenyl]-2H-pyrazolo[4,3-b]pyridin-7-yl}piperidine-1-carboxylate (0.153 g, 0.281 mmol, Step 17.1) in tetrahydrofuran (5.6 mL) was injected into a flow hydrogenation system (ThalesNano H-Cube® Pro equipped with a 70 mm 10% Pd/C CatCart®, flow rate: 1.0 mL/min, eluting with 100% methanol, temperature: 30° C., H₂ pressure: 20 bar). The reaction mixture was concentrated under reduced pressure, and the crude residue was purified by silica gel chromatography (0-100% ethyl acetate in heptanes) to provide the title compound (0.065 g, 42%). MS (ESI) m/z: 548.1 [M+H]⁺.

### Step 17.3

### 2-[4-(4-methoxyphenoxy)phenyl]-7-[1-(prop-2-enoyl)piperidin-4-yl]-4,5,6,7-tetrahydro-2H-pyrazolo[4,3-b]pyridine-3-carboxamide

Step 17.3 was prepared according to the procedure for, Step 12.3, substituting tert-butyl 4-{3-carbamoyl-2-[4-(4-methoxyphenoxy)phenyl]-4,5,6,7-tetrahydro-2H-pyrazolo[4,3-b]pyridin-7-yl}piperidine-1-carboxylate (Step 17.2) for 4-{3-carbamoyl-2-[4-(3-fluorophenoxy)phenyl]-4,5,6,7-tetrahydro-2H-pyrazolo[4,3-b]pyridin-7-yl}piperidine-1-carboxylate to provide 2-[4-(4-methoxyphenoxy)phenyl]-7-(piperidin-4-yl)-4,5,6,7-tetrahydro-2H-pyrazolo[4,3-b]pyridine-3-carboxamide. The title compound was prepared according to the procedure for Step 12.4, substituting -[4-(4-methoxyphenoxy)phenyl]-7-(piperidin-4-yl)-4,5,6,7-tetrahydro-2H-pyrazolo[4,3-b]pyridine-3-carboxamide for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, dimethyl sulfoxide-d₆) δ ppm 7.32-7.23 (m, 2H), 7.09-6.89 (m, 6H), 6.84-6.73 (m, 1H), 6.06 (dd, J=16.7, 2.5 Hz, 1H), 5.63 (dd, J=10.4, 2.5 Hz, 1H), 4.47 (d, J=13.0 Hz, 1H), 4.08 (d, J=13.2 Hz, 1H), 3.76 (s, 3H), 3.34 (s, 2H), 3.13-2.91 (m, 2H), 2.73 (s, 1H), 2.57 (t, J=13.3 Hz, 1H), 2.07-1.63 (m, 5H), 1.36-1.15 (m, 2H). MS (APCI) m/z: 502.4 [M+H]⁺.

### Example 18

### 7-[(2S,5R)-2,5-dimethyl-4-(prop-2-enoyl)piperazin-1-yl]-2-(4-phenoxyphenyl)-4,5,6,7-tetrahydro-2H-pyrazolo[4,3-b]pyridine-3-carboxamide

### Step 18.1

### tert-butyl (2R,5S)-4-[3-carbamoyl-2-(4-phenoxyphenyl)-2H-pyrazolo[4,3-b]pyridin-7-yl]-2,5-dimethylpiperazine-1-carboxylate

Step 18.1 was prepared according to the procedure for Step B.7, substituting (2R,5S)-tert-butyl 2,5-dimethylpiperazine-1-carboxylate for tert-butyl piperazine-1-carboxylate to afford the title compound. MS (ESI) m/z: 543.2 [M+H]⁺.

### Step 18.2

### tert-butyl (2R,5S)-4-[3-carbamoyl-2-(4-phenoxyphenyl)-4,5,6,7-tetrahydro-2H-pyrazolo[4,3-b]pyridin-7-yl]-2,5-dimethylpiperazine-1-carboxylate

Example 18.2 was prepared according to the procedure for Step B.8, substituting tert-butyl (2R,5S)-4-[3-carbamoyl-2-(4-phenoxyphenyl)-2H-pyrazolo[4,3-b]pyridin-7-yl]-2,5-dimethylpiperazine-1-carboxylate (Step 18.1) for tert-butyl 4-[3-carbamoyl-2-(4-phenoxyphenyl)-2H-pyrazolo[4,3-b]pyridin-7-yl]piperazine-1-carboxylate to afford the title compound. MS (ESI) m/z: 545.0 [M−H]⁻.

### Step 18.3

### 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide

Acetyl chloride (0.151 mL, 2.122 mmol) was added to a chilled (0° C.) vial containing methanol (5.30 mL). This was then added to a vial containing tert-butyl (2R,5S)-4-[3-carbamoyl-2-(4-phenoxyphenyl)-4,5,6,7-tetrahydro-2H-pyrazolo[4,3-b]pyridin-7-yl]-2,5-dimethylpiperazine-1-carboxylate (0.29 g, 0.530 mmol, Step 18.2). The resulting solution was heated at 50° C. for 90 minutes, and then concentrated under reduced pressure. The residue was treated with NaOH in dichloromethane to afford the title compound (0.19 g, 80%). MS (APCI) m/z: 447.4 [M+H]⁺.

### Step 18.4

### 7-[(2S,5R)-2,5-dimethyl-4-(prop-2-enoyl)piperazin-1-yl]-2-(4-phenoxyphenyl)-4,5,6,7-tetrahydro-2H-pyrazolo[4,3-b]pyridine-3-carboxamide

A flask containing 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide (0.19 g, 0.425 mmol, Step 18.3) in dichloromethane (5.0 mL) was chilled to 0° C. and then N-ethyl-N-isopropylpropan-2-amine (0.454 mL, 2.55 mmol) added. The solution was stirred in an ice-water bath, followed by the addition of acrylic acid (0.026 mL, 0.383 mmol), dropwise addition of 2,4,6-tripropyl-1,3,5,2,4,6-trioxatriphosphinane 2,4,6-trioxide (0.507 mL, 0.851 mmol), and followed immediately by addition added water (1 mL). The reaction was stirred at ambient temperature for 10 minutes, and the layers separated. The dichloromethane layer was loaded onto a silica gel column (eluted with 0-10% methanol in dichloromethane over 10 minutes, then isocratic 10% methanol in dichloromethane) to afford the title compound (0.0576 g, 27.0%). ¹H NMR (400 MHz, dimethyl sulfoxide-d₆, 120° C.) δ ppm 7.42-7.32 (m, 4H), 7.14 (tt, J=7.4, 1.2 Hz, 1H), 7.08-6.98 (m, 4H), 6.62 (dd, J=16.8, 10.6 Hz, 1H), 6.49 (s, 2H), 6.01 (dt, J=16.8, 2.3 Hz, 1H), 5.57 (dt, J=10.6, 2.0 Hz, 1H), 4.92 (s, 1H), 4.34 (d, J=34.3 Hz, 1H), 3.75 (q, J=4.6 Hz, 2H), 3.40-3.10 (m, 4H), 2.78 (t, J=3.9 Hz, 1H), 2.60 (ddd, J=12.3, 5.3, 3.1 Hz, 1H), 2.05 (ddt, J=11.1, 5.6, 2.9 Hz, 1H), 1.88-1.63 (m, 1H), 1.22-0.97 (m, 6H). MS (ESI) m/z: 501.4 [M+H]⁺.

### Example 19

### 7-[1-(but-2-ynoyl)piperidin-4-yl]-2-(4-phenoxyphenyl)-4,5,6,7-tetrahydro-2H-pyrazolo[3,4-b]pyrazine-3-carboxamide

### Step 19.1

### 2-(4-phenoxyphenyl)-7-(piperidin-4-yl)-4,5,6,7-tetrahydro-2H-pyrazolo[3,4-b]pyrazine-3-carboxamide-hydrogen chloride (1/3)

To a solution of tert-butyl 4-[3-carbamoyl-2-(4-phenoxyphenyl)-2,4,5,6-tetrahydro-7H-pyrazolo[3,4-b]pyrazin-7-yl]piperidine-1-carboxylate (0.664 g, 1.280 mmol, Step 16.6) in 1,4-dioxane (5 mL) was added 4 M HCl in 1,4-dioxane (3.20 mL, 12.80 mmol). The solution was stirred at ambient temperature for 1 hour. The mixture was concentrated under reduced pressure to afford the title compound (0.69 g, 102%). MS (APCI) m/z: 419.31 [M+H]⁺.

### Step 19.2

### 7-[1-(but-2-ynoyl)piperidin-4-yl]-2-(4-phenoxyphenyl)-4,5,6,7-tetrahydro-2H-pyrazolo[3,4-b]pyrazine-3-carboxamide

Step 19.2 was prepared according to the procedure for Step 16.8, substituting 2-(4-phenoxyphenyl)-7-(piperidin-4-yl)-4,5,6,7-tetrahydro-2H-pyrazolo[3,4-b]pyrazine-3-carboxamide-hydrogen chloride (1/3) (Step 19.1) for 2-(4-phenoxyphenyl)-7-(piperidin-4-yl)-4,5,6,7-tetrahydro-2H-pyrazolo[3,4-b]pyrazine-3-carboxamide-hydrogen chloride (1/2) and but-2-ynoic acid for acrylic acid. ¹H NMR (500 MHz, CDCl₃) δ ppm 7.41-7.31 (m, 4H), 7.15 (tt, J=7.3, 1.1 Hz, 1H), 7.08-7.00 (m, 4H), 5.29 (s, 1H), 4.84-4.58 (m, 1H), 4.48 (dq, J=11.3, 2.4 Hz, 1H), 4.05 (tt, J=12.0, 3.9 Hz, 1H), 3.47-3.41 (m, 2H), 3.30-3.22 (m, 2H), 3.14 (td, J=13.1, 2.8 Hz, 1H), 2.69 (td, J=13.0, 3.0 Hz, 1H), 2.01 (s, 3H), 1.89 (dd, J=29.3, 12.5 Hz, 2H), 1.70 (dqd, J=24.8, 12.4, 4.6 Hz, 2H). MS (APCI) m/z: 485.5 [M+H]⁺.

### Example 20

### 2-(4-phenoxyphenyl)-7-[(3R)-3-(propan-2-yl)-4-(prop-2-enoyl)piperazin-1-yl]-4,5,6,7-tetrahydro-2H-pyrazolo[4,3-b]pyridine-3-carboxamide

### Step 20.1

### 2-(4-phenoxyphenyl)-7-[(3R)-3-(propan-2-yl)piperazin-1-yl]-2H-pyrazolo[4,3-b]pyridine-3-carboxamide

A solution of 7-bromo-2-(4-phenoxyphenyl)-2H-pyrazolo[4,3-b]pyridine-3-carboxamide (0.15 g, 0.367 mmol, Step B.6) and (S)-tert-butyl 3-isopropylpiperazine-1-carboxylate (0.502 g, 2.199 mmol) were suspended in N,N-dimethylacetamide (15 mL) and heated to 180° C. for 2 hours. Upon cooling to 25° C., the crude reaction was diluted with water and extracted with dichloromethane (4×20 mL). The combined organic layers were washed with brine, dried over Na₂SO₄, filtered, and concentrated under reduced pressure. The resulting residue was purified by column chromatography on silica gel (eluted with ethyl acetate:ethanol 0-100%) to afford the title compound (0.105 g, 62.7%). MS (APCI) m/z: 457.2 [M+H]⁺.

### Step 20.2

### 2-(4-phenoxyphenyl)-7-[(3R)-3-(propan-2-yl)piperazin-1-yl]-4,5,6,7-tetrahydro-2H-pyrazolo[4,3-b]pyridine-3-carboxamide

Step 20.2 was prepared according to the procedure for, Step 35.2, substituting 2-(4-phenoxyphenyl)-7-[(3R)-3-(propan-2-yl)piperazin-1-yl]-2H-pyrazolo[4,3-b]pyridine-3-carboxamide (Step 20.1) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate. MS (APCI) m/z: 461.6 [M+H]⁺.

### Step 20.3

### 2-(4-phenoxyphenyl)-7-[(3R)-3-(propan-2-yl)-4-(prop-2-enoyl)piperazin-1-yl]-4,5,6,7-tetrahydro-2H-pyrazolo[4,3-b]pyridine-3-carboxamide

Step 20.3 was prepared according to the procedure for Step 35.4, substituting 2-(4-phenoxyphenyl)-7-[(3R)-3-(propan-2-yl)piperazin-1-yl]-4,5,6,7-tetrahydro-2H-pyrazolo[4,3-b]pyridine-3-carboxamide (Step 20.2) for 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide. ¹H NMR (400 MHz, dimethyl sulfoxide-d₆) δ ppm 7.42-7.30 (m, 3H), 7.14 (t, J=7.4 Hz, 1H), 7.06-7.00 (m, 3H), 6.74-6.64 (m, 2H), 6.03 (dd, J=16.8, 2.4 Hz, 1H), 5.61 (dd, J=10.6, 2.3 Hz, 1H), 4.37-3.72 (m, 3H), 3.65-3.37 (m, 1H), 3.39-3.23 (m, 1H), 2.83-2.52 (m, 1H), 2.43-2.25 (m, 1H), 2.16-2.03 (m, 1H), 1.89 (s, 1H), 1.73 (d, J=14.7 Hz, 1H), 1.61-1.20 (m, 1H), 0.88 (d, J=6.5 Hz, 2H), 0.82 (d, J=6.6 Hz, 2H), 0.72 (dd, J=6.8, 2.7 Hz, 3H). MS (APCI) m/z: 515.6 [M+H]⁺.

### Example 21

### 7-[(2R,5S)-2,5-dimethyl-4-(prop-2-enoyl)piperazin-1-yl]-2-(4-phenoxyphenyl)-4,5,6,7-tetrahydro-2H-pyrazolo[4,3-b]pyridine-3-carboxamide

### Step 21.1

### tert-butyl (2S,5R)-4-[3-carbamoyl-2-(4-phenoxyphenyl)-2H-pyrazolo[4,3-b]pyridin-7-yl]-2,5-dimethylpiperazine-1-carboxylate

Step 21.1 was prepared according to the procedure for Step B.7, substituting (2S,5R)-tert-butyl 2,5-dimethylpiperazine-1-carboxylate for tert-butyl piperazine-1-carboxylate to afford the title compound. MS (ESI) m/z: 543.2 [M+H]⁺.

### Step 21.2

### tert-butyl (2S,5R)-4-[3-carbamoyl-2-(4-phenoxyphenyl)-4,5,6,7-tetrahydro-2H-pyrazolo[4,3-b]pyridin-7-yl]-2,5-dimethylpiperazine-1-carboxylate

Step 21.2 was prepared according to the procedure for Step 57.2, substituting tert-butyl (2S,5R)-4-[3-carbamoyl-2-(4-phenoxyphenyl)-2H-pyrazolo[4,3-b]pyridin-7-yl]-2,5-dimethylpiperazine-1-carboxylate (Step 21.1) for ethyl 7-[4-(tert-butoxycarbonyl)piperazin-1-yl]-2-(4-hydroxyphenyl)-2H-pyrazolo[4,3-b]pyridine-3-carboxylate and heating 60° C. to afford the title compound. MS (APCI) m/z: 547.5 [M+H]⁺.

### Step 21.3

### 7-[(2R,5S)-2,5-dimethylpiperazin-1-yl]-2-(4-phenoxyphenyl)-4,5,6,7-tetrahydro-2H-pyrazolo[4,3-b]pyridine-3-carboxamide

Step 21.3 was prepared according to the procedure for Step 35.3, substituting tert-butyl (2S,5R)-4-[3-carbamoyl-2-(4-phenoxyphenyl)-4,5,6,7-tetrahydro-2H-pyrazolo[4,3-b]pyridin-7-yl]-2,5-dimethylpiperazine-1-carboxylate (Step 21.2) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate to afford the title compound. MS (APCI) m/z: 447.5 [M+H]⁺.

### Step 21.4

### 7-[(2R,5S)-2,5-dimethyl-4-(prop-2-enoyl)piperazin-1-yl]-2-(4-phenoxyphenyl)-4,5,6,7-tetrahydro-2H-pyrazolo[4,3-b]pyridine-3-carboxamide

Step 21.4 was prepared according to the procedure for Step 18.4, substituting 7-[(2R,5S)-2,5-dimethylpiperazin-1-yl]-2-(4-phenoxyphenyl)-4,5,6,7-tetrahydro-2H-pyrazolo[4,3-b]pyridine-3-carboxamide (Step 21.3) for 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide to afford the title compound. ¹H NMR (400 MHz, dimethyl sulfoxide-d₆) δ ppm 7.53-7.32 (m, 4H), 7.27-7.08 (m, 1H), 7.07 (s, 4H), 6.91-6.61 (m, 3H), 6.27-5.94 (m, 1H), 5.92-5.48 (m, 1H), 4.39 (d, J=103.9 Hz, 1H), 3.54-2.88 (m, 4H), 2.17 (s, 1H), 1.98 (d, J=30.4 Hz, 1H), 1.27-1.15 (m, 6H). MS (APCI) m/z: 501.4 [M+H]⁺.

### Example 22

### 2-(4-phenoxyphenyl)-7-[1-(prop-2-enoyl)azetidin-3-yl]-4,5,6,7-tetrahydro-2H-pyrazolo[3,4-b]pyrazine-3-carboxamide

To a solution of tert-butyl 3-[3-carbamoyl-2-(4-phenoxyphenyl)-2,4,5,6-tetrahydro-7H-pyrazolo[3,4-b]pyrazin-7-yl]azetidine-1-carboxylate (0.0664 g, 0.087 mmol, Intermediate J) in 1,4-dioxane (0.8 mL) was added 4 M HCl in 1,4-dioxane (0.217 mL, 0.868 mmol). The solution was stirred at ambient temperature for 2 hours. The mixture was filtered. The filter cake was washed with 1,4-dioxane, diethyl ether, and dried overnight under vacuum. The crude material was suspended in dichloromethane (1 mL), cooled in an ice-water bath to <10° C. before addition of N-ethyl-N-isopropylpropan-2-amine (60.8 μL, 0.348 mmol) via syringe. The suspension was stirred for 5 minutes. Once the internal temperature returned to <5° C., the solution was treated with acrylic acid (4.5 μL, 0.066 mmol) and dropwise addition of 1-propanephosphonic anhydride (0.10 mL, 0.174 mmol) over 2 minutes. The reaction was stirred for 5 minutes. Water was added, and the mixture was stirred for 5 minutes, and then poured into a separatory funnel. The organic layer was washed with water, concentrated, and loaded onto a silica gel column (eluted with 0-10% methanol in dichloromethane) to afford the title compound (0.01 g, 23.3%). ¹H NMR (500 MHz, CDCl₃) δ ppm 7.40-7.35 (m, 3H), 7.35-7.32 (m, 2H), 7.16 (ddd, J=7.4, 6.8, 1.1 Hz, 1H), 7.08-7.01 (m, 4H), 6.37-6.29 (m, 1H), 6.26-6.16 (m, 1H), 5.67 (ddd, J=10.3, 5.0, 1.9 Hz, 1H), 5.24 (s, 2H), 4.45 (dt, J=19.0, 6.2 Hz, 3H), 4.33-4.24 (m, 2H), 3.53 (t, J=4.7 Hz, 2H), 3.26 (dt, J=21.1, 5.1 Hz, 2H). MS (APCI) m/z: 445.4 [M+H]⁺.

### Example 23

### 7-[1-(prop-2-enoyl)piperidin-4-yl]-2-{4-[3-(trifluoromethyl)phenoxy]phenyl}-4,5,6,7-tetrahydro-2H-pyrazolo[4,3-b]pyridine-3-carboxamide

### Step 23.1

### tert-butyl 4-(3-carbamoyl-2-{4-[3-(trifluoromethyl)phenoxy]phenyl}-2H-pyrazolo[4,3-b]pyridin-7-yl)piperidine-1-carboxylate

Step 23.1 was prepared according to the procedure for Step 12.1, substituting 3-(trifluoromethyl)phenol for 3-fluorophenol and 2-(dimethylamino)acetic acid for 2,2,6,6-tetramethyl-3,5-heptanedione. MS (APCI) m/z: 582.0 [M+H]⁺.

### Step 23.2

### tert-butyl 4-(3-carbamoyl-2-{4-[3-(trifluoromethyl)phenoxy]phenyl}-4,5,6,7-tetrahydro-2H-pyrazolo[4,3-b]pyridin-7-yl)piperidine-1-carboxylate

A solution of tert-butyl 4-(3-carbamoyl-2-{4-[3-(trifluoromethyl)phenoxy]phenyl}-2H-pyrazolo[4,3-b]pyridin-7-yl)piperidine-1-carboxylate (0.101 g, 0.174 mmol, Step 23.1) in tetrahydrofuran (3.5 mL) was injected into a flow hydrogenation system (ThalesNano H-Cube© Pro equipped with a 70 mm 10% Pd/C CatCart®, flow rate: 1.0 mL/min, eluting with 100% methanol, temperature: 40° C., H₂ pressure: 30 bar). The reaction mixture was concentrated under reduced pressure, and the crude residue was purified by silica gel chromatography (0-100% ethyl acetate in heptanes) to provide the title compound (0.055 g, 54%). MS (ESI) m/z: 586.5 [M+H]⁺.

### Step 23.3

### 7-[1-(prop-2-enoyl)piperidin-4-yl]-2-{4-[3-(trifluoromethyl)phenoxy]phenyl}-4,5,6,7-tetrahydro-2H-pyrazolo[4,3-b]pyridine-3-carboxamide

Step 23.3 was prepared according to the procedure for Step 12.3, substituting tert-butyl 4-(3-carbamoyl-2-{4-[3-(trifluoromethyl)phenoxy]phenyl}-4,5,6,7-tetrahydro-2H-pyrazolo[4,3-b]pyridin-7-yl)piperidine-1-carboxylate (Step 23.2) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate to provide 7-(piperidin-4-yl)-2-{4-[3-(trifluoromethyl)phenoxy]phenyl}-4,5,6,7-tetrahydro-2H-pyrazolo[4,3-b]pyridine-3-carboxamide. The title compound was prepared according to the procedure for Step 12.4, substituting 7-(piperidin-4-yl)-2-{4-[3-(trifluoromethyl)phenoxy]phenyl}-4,5,6,7-tetrahydro-2H-pyrazolo[4,3-b]pyridine-3-carboxamide for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, CDCl₃) δ ppm 7.54-7.38 (m, 4H), 7.34-7.29 (m, 1H), 7.25-7.19 (m, 1H), 7.15-7.07 (m, 2H), 6.58 (dd, J=16.8, 10.6 Hz, 1H), 6.25 (dd, J=16.8, 2.0 Hz, 1H), 5.65 (dd, J=10.6, 2.0 Hz, 1H), 5.34 (s, 2H), 4.77-4.69 (m, 1H), 4.10-3.97 (m, 1H), 3.42-3.23 (m, 2H), 3.10-2.99 (m, 1H), 2.95-2.79 (m, 1H), 2.62 (d, J=5.8 Hz, 1H), 2.34-1.69 (m, 6H), 1.53-1.34 (m, 2H). MS (ESI) m/z: 540.0 [M+H]⁺.

### Example 24

### 2-[4-(3-methylphenoxy)phenyl]-7-[1-(prop-2-enoyl)piperidin-4-yl]-4,5,6,7-tetrahydro-2H-pyrazolo[4,3-b]pyridine-3-carboxamide

### Step 24.1

### tert-butyl 4-{3-carbamoyl-2-[4-(3-methylphenoxy)phenyl]-2H-pyrazolo[4,3-b]pyridin-7-yl}piperidine-1-carboxylate

Step 24.1 was prepared according to the procedure for Step 12.1, substituting m-cresol for 3-fluorophenol. MS (ESI) m/z: 528.1 [M+H]⁺.

### Step 24.2

### tert-butyl 4-{3-carbamoyl-2-[4-(3-methylphenoxy)phenyl]-4,5,6,7-tetrahydro-2H-pyrazolo[4,3-b]pyridin-7-yl}piperidine-1-carboxylate

A solution of tert-butyl 4-{3-carbamoyl-2-[4-(3-methylphenoxy)phenyl]-2H-pyrazolo[4,3-b]pyridin-7-yl}piperidine-1-carboxylate (0.116 g, 0.220 mmol, Step 24.1) in tetrahydrofuran (4 mL) was injected into a flow hydrogenation system (ThalesNano H-Cube© Pro equipped with a 70 mm 10% Pd/C CatCart®, flow rate: 1.0 mL/min, eluting with 100% methanol, temperature: 40° C., H₂ pressure: 30 bar). The reaction mixture was concentrated under reduced pressure, and the crude residue was purified by silica gel chromatography (0-100% ethyl acetate in heptanes) to provide the title compound (0.027 g, 0.051 mmol, 23%). MS (ESI) m/z: 532.2 [M+H]⁺.

### Step 24.3

### 2-[4-(3-methylphenoxy)phenyl]-7-[1-(prop-2-enoyl)piperidin-4-yl]-4,5,6,7-tetrahydro-2H-pyrazolo[4,3-b]pyridine-3-carboxamide

Step 24.3 was prepared according to the procedure for Step 12.3, substituting tert-butyl 4-{3-carbamoyl-2-[4-(3-methylphenoxy)phenyl]-4,5,6,7-tetrahydro-2H-pyrazolo[4,3-b]pyridin-7-yl}piperidine-1-carboxylate (Step 24.2) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate to provide 2-[4-(3-methylphenoxy)phenyl]-7-(piperidin-4-yl)-4,5,6,7-tetrahydro-2H-pyrazolo[4,3-b]pyridine-3-carboxamide. The title compound was prepared according to the procedure for Step 12.4, substituting 2-[4-(3-methylphenoxy)phenyl]-7-(piperidin-4-yl)-4,5,6,7-tetrahydro-2H-pyrazolo[4,3-b]pyridine-3-carboxamide for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, CDCl₃) δ ppm 7.42-7.34 (m, 2H), 7.30-7.22 (m, 1H), 7.11-7.02 (m, 2H), 7.01-6.95 (m, 1H), 6.91-6.82 (m, 2H), 6.57 (dd, J=16.9, 10.6 Hz, 1H), 6.24 (dd, J=16.9, 2.0 Hz, 1H), 5.65 (dd, J=10.5, 2.0 Hz, 1H), 5.50-5.18 (m, 2H), 4.75-4.71 (m, 1H), 4.05-4.01 (m, 1H), 3.40-3.23 (m, 2H), 3.04 (t, J=12.7 Hz, 1H), 2.91-2.80 (m, 1H), 2.62 (d, J=8.6 Hz, 1H), 2.35 (s, 3H), 2.24-1.20 (m, 8H). MS (APCI) m/z: 486.5 [M+H]⁺.

### Example 25

### trifluoroacetic acid-2-(4-phenoxyphenyl)-7-[4-(prop-2-enoyl)-1,4-diazepan-1-yl]-4,5,6,7-tetrahydro-2H-pyrazolo[4,3-b]pyridine-3-carboxamide (1/1)

### Step 25.1

### tert-butyl 4-[3-carbamoyl-2-(4-phenoxyphenyl)-2H-pyrazolo[4,3-b]pyridin-7-yl]-1,4-diazepane-1-carboxylate

Step 25.1 was prepared according to the procedure for Step B.7, substituting tert-butyl 1,4-diazepane-1-carboxylate for tert-butyl piperazine-1-carboxylate to afford the title compound. MS (APCI) m/z: 529.5 [M+H]⁺.

### Step 25.2

### tert-butyl 4-[3-carbamoyl-2-(4-phenoxyphenyl)-4,5,6,7-tetrahydro-2H-pyrazolo[4,3-b]pyridin-7-yl]-1,4-diazepane-1-carboxylate

Step 25.2 was prepared according to the procedure for Step B.8, substituting tert-butyl 4-[3-carbamoyl-2-(4-phenoxyphenyl)-2H-pyrazolo[4,3-b]pyridin-7-yl]-1,4-diazepane-1-carboxylate (Step 25.1) for tert-butyl 4-[3-carbamoyl-2-(4-phenoxyphenyl)-2H-pyrazolo[4,3-b]pyridin-7-yl]piperazine-1-carboxylate to afford the title compound. MS (APCI) m/z: 533.5 [M+H]⁺.

### Step 25.3

### 7-(1,4-diazepan-1-yl)-2-(4-phenoxyphenyl)-4,5,6,7-tetrahydro-2H-pyrazolo[4,3-b]pyridine-3-carboxamide

Step 25.3 was prepared according to the procedure for Step 35.3, substituting tert-butyl 4-[3-carbamoyl-2-(4-phenoxyphenyl)-4,5,6,7-tetrahydro-2H-pyrazolo[4,3-b]pyridin-7-yl]-1,4-diazepane-1-carboxylate (Step 25.2) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate to afford the title compound. MS (APCI) m/z: 433.4 [M+H]⁺.

### Step 25.4

### trifluoroacetic acid-2-(4-phenoxyphenyl)-7-[4-(prop-2-enoyl)-1,4-diazepan-1-yl]-4,5,6,7-tetrahydro-2H-pyrazolo[4,3-b]pyridine-3-carboxamide (1/1)

Step 25.4 was prepared according to the procedure for Step 18.4, substituting 7-(1,4-diazepan-1-yl)-2-(4-phenoxyphenyl)-4,5,6,7-tetrahydro-2H-pyrazolo[4,3-b]pyridine-3-carboxamide (Step 25.3) for 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide to afford the title compound. MS (APCI) m/z: 487.4 [M+H]⁺.

### Example 26

### 2-(4-phenoxyphenyl)-7-[5-(prop-2-enoyl)-5,8-diazaspiro[3.5]nonan-8-yl]-4,5,6,7-tetrahydro-2H-pyrazolo[4,3-b]pyridine-3-carboxamide

### Step 26.1

### tert-butyl 8-(3-carbamoyl-2-(4-phenoxyphenyl)-2H-pyrazolo[4,3-b]pyridin-7-yl)-5,8-diazaspiro[3.5]nonane-5-carboxylate

Step 26.1 was prepared according to the procedure for Step 35.1, substituting tert-butyl 5,8-diazaspiro[3.5]nonane-5-carboxylate for (1R,5S)-tert-butyl 3,8-diazabicyclo[3.2.1]octane-8-carboxylate. MS (DCI) m/z: 555.3 [M+H]⁺.

### Step 26.2

### tert-butyl 8-[3-carbamoyl-2-(4-phenoxyphenyl)-4,5,6,7-tetrahydro-2H-pyrazolo[4,3-b]pyridin-7-yl]-5,8-diazaspiro[3.5]nonane-5-carboxylate

Step 26.2 was prepared according to the procedure for Step 35.2, substituting tert-butyl 8-(3-carbamoyl-2-(4-phenoxyphenyl)-2H-pyrazolo[4,3-b]pyridin-7-yl)-5,8-diazaspiro[3.5]nonane-5-carboxylate (Step 26.1) for tert-butyl (1R,5S)-3-[3-carbamoyl-2-(4-phenoxyphenyl)-2H-pyrazolo[4,3-b]pyridin-7-yl]-3,8-diazabicyclo[3.2.1]octane-8-carboxylate. MS (APCI) m/z: 559.5 [M+H]⁺.

### Step 26.3

### 7-(5,8-diazaspiro[3.5]nonan-8-yl)-2-(4-phenoxyphenyl)-4,5,6,7-tetrahydro-2H-pyrazolo[4,3-b]pyridine-3-carboxamide

Step 26.3 was prepared according to the procedure for Step 35.3, substituting tert-butyl 8-[3-carbamoyl-2-(4-phenoxyphenyl)-4,5,6,7-tetrahydro-2H-pyrazolo[4,3-b]pyridin-7-yl]-5,8-diazaspiro[3.5]nonane-5-carboxylate (Step 26.2) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate. MS (APCI) m/z: 459.5 [M+H]⁺.

### Step 26.4

### 2-(4-phenoxyphenyl)-7-[5-(prop-2-enoyl)-5,8-diazaspiro[3.5]nonan-8-yl]-4,5,6,7-tetrahydro-2H-pyrazolo[4,3-b]pyridine-3-carboxamide

Step 26.4 was prepared according to the procedure for Step 35.4, substituting 7-(5,8-diazaspiro[3.5]nonan-8-yl)-2-(4-phenoxyphenyl)-4,5,6,7-tetrahydro-2H-pyrazolo[4,3-b]pyridine-3-carboxamide (Step 26.3) for 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide. ¹H NMR (400 MHz, dimethyl sulfoxide-d₆) δ ppm 7.42-7.33 (m, 4H), 7.18-7.10 (m, 1H), 7.08-7.00 (m, 4H), 6.56 (d, J=11.5 Hz, 2H), 6.48 (dd, J=16.9, 10.5 Hz, 1H), 5.96 (dd, J=16.9, 2.2 Hz, 1H), 5.54 (dd, J=10.6, 2.2 Hz, 1H), 4.11 (d, J=6.4 Hz, 2H), 3.60-3.50 (m, 1H), 3.50-3.38 (m, 1H), 3.31 (ddd, J=11.6, 8.1, 3.5 Hz, 1H), 3.20 (ddd, J=12.0, 7.8, 3.4 Hz, 1H), 3.17-2.94 (m, 3H), 2.79-2.68 (m, 1H), 2.40 (t, J=10.6 Hz, 1H), 2.34-2.08 (m, 4H), 2.05-1.93 (m, 1H), 1.79-1.59 (m, 1H). MS (APCI) m/z: 513.4 [M+H]⁺.

### Example 27

### 7-[4-(but-2-ynoyl)piperazin-1-yl]-2-(4-phenoxyphenyl)-4,5,6,7-tetrahydro-2H-pyrazolo[4,3-b]pyridine-3-carboxamide

But-2-ynoic acid (0.045 mL, 0.568 mmol) was added into a vial containing 2-(4-phenoxyphenyl)-7-(piperazin-1-yl)-4,5,6,7-tetrahydro-2H-pyrazolo[4,3-b]pyridine-3-carboxamide (0.250 g, 0.597 mmol, Intermediate B). Dichloromethane (6.00 mL) was added, and the solution was cooled in an ice-water bath to <5° C., followed by addition of N-ethyl-N-isopropylpropan-2-amine (0.425 mL, 2.39 mmol) and dropwise addition of 2,4,6-tripropyl-1,3,5,2,4,6-trioxatriphosphinane 2,4,6-trioxide (50% solution in ethyl acetate, 0.213 mL, 0.717 mmol). The reaction mixture was stirred for 5 minutes at ambient temperature, and then diluted with water (5 mL) and dichloromethane (10 mL), and stirred 5 minutes at ambient temperature. The layers were separated and the organic layer was dried over sodium sulfate and concentrated under reduced pressure. The crude residue was purified by column chromatography on silica gel (0-5% methanol in dichloromethane then isocratic 5% methanol in dichloromethane) to afford the title compound (240 mg, 83%). ¹H NMR (500 MHz, CDCl₃) δ ppm 7.44-7.34 (m, 4H), 7.22-7.13 (m, 1H), 7.12-7.02 (m, 4H), 5.25 (br s, 2H), 5.13 (br s, 1H), 3.87 (dd, J=6.9, 5.1 Hz, 1H), 3.80-3.66 (m, 3H), 3.59 (dq, J=10.6, 3.3 Hz, 1H), 3.44 (ddd, J=11.3, 8.0, 3.1 Hz, 1H), 3.38-3.26 (m, 1H), 2.81 (ddd, J=10.5, 6.3, 3.6 Hz, 1H), 2.69 (ddq, J=10.8, 7.2, 3.7 Hz, 3H), 2.17 (dtd, J=14.3, 7.4, 3.2 Hz, 1H), 1.99 (s, 3H), 1.98-1.89 (m, 1H). MS (APCI) m/z: 485.4 [M+H]⁺.

### Example 28

### 2-[4-(4-chlorophenoxy)phenyl]-7-[1-(prop-2-enoyl)piperidin-4-yl]-4,5,6,7-tetrahydro-2H-pyrazolo[4,3-b]pyridine-3-carboxamide

### Step 28.1

### tert-butyl 4-{3-carbamoyl-2-[4-(4-chlorophenoxy)phenyl]-2H-pyrazolo[4,3-b]pyridin-7-yl}piperidine-1-carboxylate

Step 28.1 was prepared according to the procedure for Step 12.1, substituting 4-chlorophenol for 3-fluorophenol and 2-(dimethylamino)acetic acid for 2,2,6,6-tetramethyl-3,5-heptanedione. ¹H NMR (500 MHz, dimethyl sulfoxide-d₆) δ ppm 8.69 (d, J=4.4 Hz, 1H), 8.47 (d, J=2.4 Hz, 1H), 8.01 (d, J=2.6 Hz, 1H), 7.66-7.58 (m, 2H), 7.54-7.48 (m, 2H), 7.36 (d, J=4.4 Hz, 1H), 7.22-7.13 (m, 4H), 4.20-4.02 (m, 2H), 3.39 (ddt, J=12.0, 8.5, 3.5 Hz, 1H), 3.05-2.76 (m, 2H), 2.00-1.94 (m, 2H), 1.85-1.72 (m, 2H), 1.42 (s, 9H).

### Step 28.2

### tert-butyl 4-{3-carbamoyl-2-[4-(4-chlorophenoxy)phenyl]-4,5,6,7-tetrahydro-2H-pyrazolo[4,3-b]pyridin-7-yl}piperidine-1-carboxylate

To a reactor (Thermo Barnstead Stem RS10) was added tert-butyl 4-{3-carbamoyl-2-[4-(4-chlorophenoxy)phenyl]-2H-pyrazolo[4,3-b]pyridin-7-yl}piperidine-1-carboxylate (0.305 g, 0.556 mmol, Step 28.1), 5% Pt/C (0.030 g, 0.063 mmol) and 1:1 ethyl acetate/tetrahydrofuran (6 mL). The vessel was pressurized with H₂ (50 psi). The reaction mixture was stirred for 6 hours at ambient temperature. The resulting solution was filtered over diatomaceous earth, and the filtrate was concentrated under reduced pressure. The crude product was purified by silica gel chromatography (0-100% ethyl acetate in heptanes) to provide the title compound (0.202 mg, 0.365 mmol, 66%). MS (ESI) m/z: 551.6 [M+H]⁺.

### Step 28.3

### 2-[4-(4-chlorophenoxy)phenyl]-7-(piperidin-4-yl)-4,5,6,7-tetrahydro-2H-pyrazolo[4,3-b]pyridine-3-carboxamide

Step 28.3 was prepared according to the procedure for Step 12.3, substituting tert-butyl 4-{3-carbamoyl-2-[4-(4-chlorophenoxy)phenyl]-4,5,6,7-tetrahydro-2H-pyrazolo[4,3-b]pyridin-7-yl}piperidine-1-carboxylate (Step 28.2) for 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. MS (ESI) m/z: 452.2 [M+H]⁺.

### Step 28.4

### 2-[4-(4-chlorophenoxy)phenyl]-7-[1-(prop-2-enoyl)piperidin-4-yl]-4,5,6,7-tetrahydro-2H-pyrazolo[4,3-b]pyridine-3-carboxamide

Step 28.4 was prepared according to the procedure for Step 12.4, substituting 2-[4-(4-chlorophenoxy)phenyl]-7-(piperidin-4-yl)-4,5,6,7-tetrahydro-2H-pyrazolo[4,3-b]pyridine-3-carboxamide (Step 28.3) for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, dimethyl sulfoxide-d₆) δ ppm 7.47-7.39 (m, 2H), 7.37-7.29 (m, 2H), 7.06 (d, J=8.7 Hz, 4H), 6.81-6.69 (m, 1H), 6.03 (dd, J=16.7, 2.5 Hz, 1H), 5.60 (dd, J=10.4, 2.5 Hz, 1H), 4.44 (d, J=12.9 Hz, 1H), 4.11-3.96 (m, 1H), 3.88-3.20 (m, 2H), 3.12 (d, J=13.1 Hz, 1H), 2.96 (d, J=13.3 Hz, 1H), 2.74 (d, J=10.2 Hz, 1H), 2.61-2.50 (m, 1H), 2.05-1.56 (m, 5H), 1.30-1.15 (m, 2H). MS (ESI) m/z: 506.4 [M+H]⁺.

### Example 29

### (7S)-2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide

The title compound was obtained by separating the enantiomers of 2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide (Example 13) with preparative supercritical fluid chromatography purification on a THAR/Waters SFC 80 system running under SuperChrom software control. The preparative supercritical fluid chromatography system was equipped with an 8-way preparative column switcher, CO₂ pump, modifier pump, automated back pressure regulator (ABPR), UV detector, and 6-position fraction collector. The mobile phase was comprised of supercritical CO₂ supplied by a dewar of bone-dry non-certified CO₂ pressurized to 350 psi with a modifier of methanol at a flow rate of 70 g/minutes. The column was at ambient temperature and the backpressure regulator was set to maintain 120 bar. The sample was dissolved in methanol at a concentration of 21.5 mg/mL. The sample was loaded into the modifier stream in 1 mL (21.5 mg) injections. The mobile phase was held isocratically at 25% methanol:CO₂. Fraction collection was threshold triggered. The instrument was fitted with a ChiralCel® OJ-J column with dimensions 21 mm i.d.×250 mm length with 5 μm particles. The peak that eluted second was concentrated under reduced pressure to provide the title compound. ¹H NMR (400 MHz, CDCl₃) δ ppm 7.43-7.35 (m, 2H), 7.16 (td, J=9.0, 5.5 Hz, 1H), 7.07-6.88 (m, 4H), 6.58 (dd, J=16.9, 10.6 Hz, 1H), 6.25 (dd, J=16.9, 1.8 Hz, 1H), 5.69 (dd, J=10.6, 1.8 Hz, 1H), 5.31 (s, 2H), 4.74 (s, 1H), 4.04 (s, 1H), 3.47-3.23 (m, 2H), 3.06 (t, J=12.5 Hz, 1H), 2.96-2.79 (m, 1H), 2.74-2.53 (m, 1H), 2.33-1.21 (m, 8H). MS (APCI) m/z: 508.3 [M+H]⁺.

### Example 30

### (7R)-2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide

The title compound was obtained by separating the enantiomers of 2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide (Example 13) with preparative supercritical fluid chromatography purification. Preparative supercritical fluid chromatography was performed on a THAR/Waters SFC 80 system running under SuperChrom software control. The preparative supercritical fluid chromatography system was equipped with an 8-way preparative column switcher, CO₂ pump, modifier pump, automated back pressure regulator (ABPR), UV detector, and 6-position fraction collector. The mobile phase was comprised of supercritical CO₂ supplied by a dewar of bone-dry non-certified CO₂ pressurized to 350 psi with a modifier of methanol at a flow rate of 70 g/minutes. The column was at ambient temperature and the backpressure regulator was set to maintain 120 bar. The sample was dissolved in methanol at a concentration of 21.5 mg/mL. The sample was loaded into the modifier stream in 1 mL (21.5 mg) injections. The mobile phase was held isocratically at 25% methanol:CO₂. Fraction collection was threshold triggered. The instrument was fitted with a ChiralCel® OJ-J column with dimensions 21 mm i.d.×250 mm length with 5 μm particles. The peak that eluted first was concentrated under reduced pressure to provide the title compound. ¹H NMR (400 MHz, CDCl₃) δ ppm 7.39 (d, J=8.9 Hz, 2H), 7.19-7.11 (m, 1H), 7.05-6.96 (m, 3H), 6.95-6.87 (m, 1H), 6.58 (dd, J=16.8, 10.6 Hz, 1H), 6.25 (dd, J=16.9, 2.0 Hz, 1H), 5.66 (dd, J=10.6, 2.0 Hz, 1H), 5.26 (s, 2H), 4.74 (s, 1H), 4.11-3.97 (m, 1H), 3.41-3.33 (m, 1H), 3.33-3.23 (m, 1H), 3.04 (t, J=12.9 Hz, 1H), 2.94-2.77 (m, 1H), 2.69-2.54 (m, 1H), 2.28-1.24 (m, 8H). MS (APCI) m/z: 508.0 [M+H]⁺.

### Example 31

### (7S)-7-[(2S)-2-methyl-4-(prop-2-enoyl)piperazin-1-yl]-2-(4-phenoxyphenyl)-4,5,6,7-tetrahydro-2H-pyrazolo[4,3-b]pyridine-3-carboxamide

### Step 31.1

### tert-butyl (3S)-4-[3-carbamoyl-2-(4-phenoxyphenyl)-2H-pyrazolo[4,3-b]pyridin-7-yl]-3-methylpiperazine-1-carboxylate

Step 31.1 was prepared according to the procedure for Step 35.1, substituting (S)-tert-butyl 3-methylpiperazine-1-carboxylate for (1R,5S)-tert-butyl 3,8-diazabicyclo[3.2.1]octane-8-carboxylate. MS (APCI) m/z: 529.5 [M+H]⁺.

### Step 31.2

### tert-butyl (3S)-4-[(7S)-3-carbamoyl-2-(4-phenoxyphenyl)-4,5,6,7-tetrahydro-2H-pyrazolo[4,3-b]pyridin-7-yl]-3-methylpiperazine-1-carboxylate

Step 31.2 was prepared according to the procedure for Step B.8, substituting (S)-tert-butyl 4-(3-carbamoyl-2-(4-phenoxyphenyl)-2H-pyrazolo[4,3-b]pyridin-7-yl)-3-methylpiperazine-1-carboxylate (Step 31.1) for tert-butyl 4-[3-carbamoyl-2-(4-phenoxyphenyl)-2H-pyrazolo[4,3-b]pyridin-7-yl]piperazine-1-carboxylate and the reactor pressurized to 100 psi with hydrogen. The two diastereomers were separated via column chromatography on silica gel (eluted with 70-100% ethyl acetate/heptane, then 0-30% (3:1 ethyl acetate/ethanol)/ethyl acetate) to afford the title compound (0.24 g, 42.5%, stereochemistry arbitrarily assigned), MS m/z: 533.5 [M+H]⁺; and tert-butyl (3S)-4-[(7R)-3-carbamoyl-2-(4-phenoxyphenyl)-4,5,6,7-tetrahydro-2H-pyrazolo[4,3-b]pyridin-7-yl]-3-methylpiperazine-1-carboxylate (0.020 g, 35.4%, stereochemistry arbitrarily assigned) MS (APCI) m/z: 533.5 [M+H]⁺.

### Step 31.3

### (7S)-7-[(2S)-2-methylpiperazin-1-yl]-2-(4-phenoxyphenyl)-4,5,6,7-tetrahydro-2H-pyrazolo[4,3-b]pyridine-3-carboxamide

Step 31.3 was prepared according to the procedure for Step 35.3, substituting tert-butyl (3S)-4-[(7S)-3-carbamoyl-2-(4-phenoxyphenyl)-4,5,6,7-tetrahydro-2H-pyrazolo[4,3-b]pyridin-7-yl]-3-methylpiperazine-1-carboxylate (Step 31.2) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate. MS (APCI) m/z: 433.5 [M+H]⁺.

### Step 31.4

### (7S)-7-[(2S)-2-methyl-4-(prop-2-enoyl)piperazin-1-yl]-2-(4-phenoxyphenyl)-4,5,6,7-tetrahydro-2H-pyrazolo[4,3-b]pyridine-3-carboxamide

Step 31.4 was prepared according to the procedure for Step 18.4, substituting (7S)-7-[(2S)-2-methylpiperazin-1-yl]-2-(4-phenoxyphenyl)-4,5,6,7-tetrahydro-2H-pyrazolo[4,3-b]pyridine-3-carboxamide (Step 31.3) for 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide to afford the title compound. ¹H NMR (400 MHz, dimethyl sulfoxide-d₆, 120° C.) δ ppm 7.47-7.31 (m, 4H), 7.19-7.10 (m, 1H), 7.10-6.97 (m, 4H), 6.64 (dd, J=16.8, 10.6 Hz, 1H), 6.50 (s, 2H), 6.02 (dd, J=16.8, 2.4 Hz, 1H), 5.69-5.50 (m, 1H), 4.87 (d, J=3.5 Hz, 1H), 4.08 (dd, J=6.8, 5.0 Hz, 1H), 3.63 (ddd, J=12.8, 3.4, 0.9 Hz, 1H), 3.57-3.36 (m, 2H), 3.36-3.22 (m, 2H), 3.16 (ddt, J=11.4, 7.7, 3.3 Hz, 1H), 3.07-2.91 (m, 1H), 2.71 (ddd, J=11.8, 6.6, 3.5 Hz, 1H), 2.56 (ddd, J=11.9, 7.1, 3.5 Hz, 1H), 2.09-1.92 (m, 1H), 1.77 (dddd, J=13.4, 8.2, 5.1, 3.3 Hz, 1H), 1.06 (d, J=6.4 Hz, 3H). MS (APCI) m/z: 487.2 [M+H]⁺.

### Example 32

### (7R)-7-[(2S)-2-methyl-4-(prop-2-enoyl)piperazin-1-yl]-2-(4-phenoxyphenyl)-4,5,6,7-tetrahydro-2H-pyrazolo[4,3-b]pyridine-3-carboxamide

### Step 32.1

### (7R)-7-[(2S)-2-methylpiperazin-1-yl]-2-(4-phenoxyphenyl)-4,5,6,7-tetrahydro-2H-pyrazolo[4,3-b]pyridine-3-carboxamide

Step 32.1 was prepared according to the procedure for Step 35.3, substituting tert-butyl (3S)-4-[(7R)-3-carbamoyl-2-(4-phenoxyphenyl)-4,5,6,7-tetrahydro-2H-pyrazolo[4,3-b]pyridin-7-yl]-3-methylpiperazine-1-carboxylate (Step 31.2) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate. MS (APCI) m/z: 433.5 [M+H]⁺.

### Step 32.2

### (7R)-7-[(2S)-2-methyl-4-(prop-2-enoyl)piperazin-1-yl]-2-(4-phenoxyphenyl)-4,5,6,7-tetrahydro-2H-pyrazolo[4,3-b]pyridine-3-carboxamide

Step 32.2 was prepared according to the procedure for Step 18.4, substituting (7R)-7-[(2S)-2-methylpiperazin-1-yl]-2-(4-phenoxyphenyl)-4,5,6,7-tetrahydro-2H-pyrazolo[4,3-b]pyridine-3-carboxamide (Step 32.1) for 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide to afford the title compound. ¹H NMR (400 MHz, dimethyl sulfoxide-d₆, 120° C.) δ ppm 7.44-7.28 (m, 4H), 7.20-7.09 (m, 1H), 7.09-6.95 (m, 4H), 6.64 (dd, J=16.8, 10.6 Hz, 1H), 6.50 (s, 2H), 6.00 (dd, J=16.8, 2.4 Hz, 1H), 5.64-5.54 (m, 1H), 4.83 (s, 1H), 4.14 (dd, J=7.2, 5.5 Hz, 1H), 3.77-3.63 (m, 2H), 3.42-3.25 (m, 2H), 3.25-3.12 (m, 2H), 3.04 (dd, J=12.8, 7.6 Hz, 1H), 2.72 (ddd, J=11.9, 5.6, 3.4 Hz, 1H), 2.51-2.43 (m, 1H), 2.14-1.96 (m, 1H), 1.85 (dddd, J=13.4, 7.5, 5.6, 3.3 Hz, 1H), 1.10 (d, J=6.2 Hz, 3H). MS (APCI) m/z: 487.4 [M+H]⁺.

### Example 33

### 2-(4-phenoxyphenyl)-7-[(1R,4R)-5-(prop-2-enoyl)-2,5-diazabicyclo[2.2.1]heptan-2-yl]-4,5,6,7-tetrahydro-2H-pyrazolo[4,3-b]pyridine-3-carboxamide

### Step 33.1

### tert-butyl (1R,4R)-5-[3-carbamoyl-2-(4-phenoxyphenyl)-2H-pyrazolo[4,3-b]pyridin-7-yl]-2,5-diazabicyclo[2.2.1]heptane-2-carboxylate

Step 33.1 was prepared according to the procedure for Step 35.1, substituting (1R,4R)-tert-butyl 2,5-diazabicyclo[2.2.1]heptane-2-carboxylate for (1R,5S)-tert-butyl 3,8-diazabicyclo[3.2.1]octane-8-carboxylate. MS (APCI) m/z: 527.4 [M+H]⁺.

### Step 33.2

### tert-butyl (1R,4R)-5-[3-carbamoyl-2-(4-phenoxyphenyl)-4,5,6,7-tetrahydro-2H-pyrazolo[4,3-b]pyridin-7-yl]-2,5-diazabicyclo[2.2.1]heptane-2-carboxylate

Step 33.2 was prepared according to the procedure for Step 36.2, substituting tert-butyl (1R,4R)-5-[3-carbamoyl-2-(4-phenoxyphenyl)-2H-pyrazolo[4,3-b]pyridin-7-yl]-2,5-diazabicyclo[2.2.1]heptane-2-carboxylate (Step 33.1) for tert-butyl 4-{3-carbamoyl-2-[4-(2,4-difluorophenoxy)phenyl]-2H-pyrazolo[4,3-b]pyridin-7-yl}piperazine-1-carboxylate and the reactor pressurized to 128 psi with hydrogen. MS (APCI) m/z: 531.5 [M+H]⁺.

### Step 33.3

### 7-[(1R,4R)-2,5-diazabicyclo[2.2.1]heptan-2-yl]-2-(4-phenoxyphenyl)-4,5,6,7-tetrahydro-2H-pyrazolo[4,3-b]pyridine-3-carboxamide

Step 33.3 was prepared according to the procedure for Step 35.3, substituting tert-butyl (1R,4R)-5-[3-carbamoyl-2-(4-phenoxyphenyl)-4,5,6,7-tetrahydro-2H-pyrazolo[4,3-b]pyridin-7-yl]-2,5-diazabicyclo[2.2.1]heptane-2-carboxylate (Step 33.2) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate. MS (APCI) m/z: 431.4 [M+H]⁺.

### Step 33.4

### 2-(4-phenoxyphenyl)-7-[(1R,4R)-5-(prop-2-enoyl)-2,5-diazabicyclo[2.2.1]heptan-2-yl]-4,5,6,7-tetrahydro-2H-pyrazolo[4,3-b]pyridine-3-carboxamide

Step 33.4 was prepared according to the procedure for the Step 18.4, substituting 7-[(1R,4R)-2,5-diazabicyclo[2.2.1]heptan-2-yl]-2-(4-phenoxyphenyl)-4,5,6,7-tetrahydro-2H-pyrazolo[4,3-b]pyridine-3-carboxamide (Step 33.3) for 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide to afford the title compound. ¹H NMR (400 MHz, dimethyl sulfoxide-d₆, 90° C.) δ ppm 7.43-7.30 (m, 4H), 7.21-7.10 (m, 1H), 7.10-6.96 (m, 4H), 6.67 (s, 3H), 6.09 (dd, J=16.8, 2.4 Hz, 1H), 5.69-5.52 (m, 1H), 4.99 (brs, 1H), 4.59 (s, 1H), 3.98 (d, J=15.7 Hz, 1H), 3.67 (s, 1H), 3.50-3.26 (m, 2H), 3.26-3.08 (m, 2H), 2.87 (s, 2H), 1.94 (ddt, J=11.9, 4.8, 2.8 Hz, 1H), 1.83 (s, 1H), 1.77-1.55 (m, 2H). MS (APCI) m/z: 485.4 [M+H]⁺.

### Example 34

### 2-(4-phenoxyphenyl)-7-[(1S,4S)-5-(prop-2-enoyl)-2,5-diazabicyclo[2.2.1]heptan-2-yl]-4,5,6,7-tetrahydro-2H-pyrazolo[4,3-b]pyridine-3-carboxamide

### Step 34.1

### tert-butyl (1S,4S)-5-[3-carbamoyl-2-(4-phenoxyphenyl)-2H-pyrazolo[4,3-b]pyridin-7-yl]-2,5-diazabicyclo[2.2.1]heptane-2-carboxylate

Step 34.1 was prepared according to the procedure for Step 35.1, substituting (1S,4S)-tert-butyl 2,5-diazabicyclo[2.2.1]heptane-2-carboxylate for (1R,5S)-tert-butyl 3,8-diazabicyclo[3.2.1]octane-8-carboxylate. MS (APCI) m/z: 527.5 [M+H]⁺.

### Step 34.2 tert-butyl (1S,4S)-5-[3-carbamoyl-2-(4-phenoxyphenyl)-4,5,6,7-tetrahydro-2H-pyrazolo[4,3-b]pyridin-7-yl]-2,5-diazabicyclo[2.2.1]heptane-2-carboxylate

Step 34.2 was prepared according to the procedure for Step 36.2, substituting tert-butyl (1S,4S)-5-[3-carbamoyl-2-(4-phenoxyphenyl)-2H-pyrazolo[4,3-b]pyridin-7-yl]-2,5-diazabicyclo[2.2.1]heptane-2-carboxylate (Step 34.1) for tert-butyl 4-{3-carbamoyl-2-[4-(2,4-difluorophenoxy)phenyl]-2H-pyrazolo[4,3-b]pyridin-7-yl}piperazine-1-carboxylate and the reactor pressurized to 125 psi with hydrogen. MS (APCI) m/z: 531.5 [M+H]⁺.

### Step 34.3

### 7-[(1S,4S)-2,5-diazabicyclo[2.2.1]heptan-2-yl]-2-(4-phenoxyphenyl)-4,5,6,7-tetrahydro-2H-pyrazolo[4,3-b]pyridine-3-carboxamide

Step 34.3 was prepared according to the procedure for Step 35.3, substituting tert-butyl (1S,4S)-5-[3-carbamoyl-2-(4-phenoxyphenyl)-4,5,6,7-tetrahydro-2H-pyrazolo[4,3-b]pyridin-7-yl]-2,5-diazabicyclo[2.2.1]heptane-2-carboxylate (Step 34.2) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate. MS (APCI) m/z: 431.5 [M+H]⁺.

### Step 34.4

### 2-(4-phenoxyphenyl)-7-[(1S,4S)-5-(prop-2-enoyl)-2,5-diazabicyclo[2.2.1]heptan-2-yl]-4,5,6,7-tetrahydro-2H-pyrazolo[4,3-b]pyridine-3-carboxamide

Step 34.4 was prepared according to the procedure for the Step 18.4, substituting 7-[(1S,4S)-2,5-diazabicyclo[2.2.1]heptan-2-yl]-2-(4-phenoxyphenyl)-4,5,6,7-tetrahydro-2H-pyrazolo[4,3-b]pyridine-3-carboxamide (Step 34.3) for 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide to afford the title compound. ¹H NMR (400 MHz, dimethyl sulfoxide-d₆, 90° C.) δ ppm 7.45-7.30 (m, 4H), 7.15 (td, J=7.3, 1.1 Hz, 1H), 7.07-6.96 (m, 4H), 6.67 (s, 3H), 6.09 (dd, J=16.8, 2.4 Hz, 1H), 5.58 (dd, J=10.4, 2.4 Hz, 1H), 4.99 (t, J=3.1 Hz, 1H), 4.59 (s, 1H), 4.04-3.90 (m, 1H), 3.78-3.55 (m, 1H), 3.49-3.28 (m, 2H), 3.13 (ddt, J=11.9, 5.0, 3.5 Hz, 2H), 2.93-2.76 (m, 2H), 1.97-1.91 (m, 1H), 1.82 (d, J=13.2 Hz, 1H), 1.78-1.58 (m, 2H). MS (APCI) m/z: 485.4 [M+H]⁺.

### Example 35

### 2-(4-phenoxyphenyl)-7-[(1R,5S)-8-(prop-2-enoyl)-3,8-diazabicyclo[3.2.1]octan-3-yl]-4,5,6,7-tetrahydro-2H-pyrazolo[4,3-b]pyridine-3-carboxamide

### Step 35.1

### tert-butyl (1R,5S)-3-[3-carbamoyl-2-(4-phenoxyphenyl)-2H-pyrazolo[4,3-b]pyridin-7-yl]-3,8-diazabicyclo[3.2.1]octane-8-carboxylate

7-bromo-2-(4-phenoxyphenyl)-2H-pyrazolo[4,3-b]pyridine-3-carboxamide (0.321 g, 0.785 mmol, Step B.6) and (1R,5S)-tert-butyl 3,8-diazabicyclo[3.2.1]octane-8-carboxylate (0.50 g, 2.355 mmol) were suspended in N,N-dimethylacetamide (10 mL) and N-ethyl-N-isopropylpropan-2-amine (0.55 mL, 3.14 mmol) added. The reaction was heated to 140° C. for 2 hours. After cooling to 25° C., the reaction mixture was diluted with water (25 mL) and extracted with ethyl acetate (4×20 mL). The combined organic layer was washed with brine, dried over Na₂SO₄, filtered and concentrated under reduced pressure to afford a residue, which was purified by column chromatography on silica gel (eluted with 40-70% heptane:ethyl acetate (+20% dichloromethane)) to afford the title compound (0.64 g, 151% yield). MS (APCI) m/z: 541.5 [M+H]⁺.

### Step 35.2

### tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate

To a reactor was added tert-butyl (1R,5S)-3-[3-carbamoyl-2-(4-phenoxyphenyl)-2H-pyrazolo[4,3-b]pyridin-7-yl]-3,8-diazabicyclo[3.2.1]octane-8-carboxylate (0.64 g, 1.18 mmol, Step 35.1), tetrahydrofuran (8 mL) and 10% palladium hydroxide on carbon (0.39 g, 1.18 mmol). The reaction was purged with nitrogen then hydrogen, sealed and heated at 50° C. for 18 hours. The mixture was cooled to 25° C., filtered through diatomaceous earth, and concentrated. The crude product was purified by column chromatography on silica gel (eluted with 0-100% ethyl acetate/ethyl acetate:ethanol (3:1)). MS (APCI) m/z: 545.5 [M+H]⁺.

### Step 35.3

### 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide

To a solution of tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate (0.59 g, 1.08 mmol, Step 35.2) in 1,4-dioxane (8 mL) under nitrogen was added dropwise 4 M HCl in 1,4-dioxane (4.1 mL). The solution was stirred for 18 hours and concentrated. The residue was taken up in dichloromethane (30 mL) and washed with saturated sodium bicarbonate and then brine. It was dried over Na₂SO₄, filtered and concentrated under reduced pressure to afford the title compound (0.47 g, 1.06 mmol), which was directly in next step without purification.

### Step 35.4

### 2-(4-phenoxyphenyl)-7-[(1R,5S)-8-(prop-2-enoyl)-3,8-diazabicyclo[3.2.1]octan-3-yl]-4,5,6,7-tetrahydro-2H-pyrazolo[4,3-b]pyridine-3-carboxamide

A solution of acrylic acid (0.058 mL, 0.850 mmol) and N,N-diisopropylethylamine (0.75 mL, 4.23 mmol), 2,4,6-tripropyl-1,3,5,2,4,6-trioxatriphosphinane 2,4,6-trioxide (1.26 mL, 0.85 mmol) in dichloromethane (21 mL) was stirred at 0° C. for 1 hour. To the mixture was added to 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide (0.47 g, 1.06 mmol, Step 35.3) in dichloromethane (3 mL). The mixture was stirred at 0° C. for 5 minutes, and then diluted with water, and extracted with ethyl acetate (3×). The combined organic phase was washed with brine, dried over Na₂SO₄, filtered, and concentrated under reduce pressure. The crude product was purified by column chromatography on silica gel (eluted with 0-100% ethyl acetate/ethyl acetate:ethanol (3:1)). ¹H NMR (400 MHz, dimethyl sulfoxide-d₆) d 7.42-7.35 (m, 2H), 7.35-7.29 (m, 2H), 7.17-7.10 (m, 1H), 7.07-6.97 (m, 4H), 6.74-6.64 (m, 2H), 6.59 (dd, J=16.8, 10.4 Hz, 1H), 6.08 (dd, J=16.8, 2.4 Hz, 1H), 5.59 (dd, J=10.4, 2.4 Hz, 1H), 4.91 (t, J=3.1 Hz, 1H), 4.42 (s, 2H), 3.65 (t, J=5.5 Hz, 1H), 3.25 (ddt, J=11.8, 8.7, 3.0 Hz, 1H), 3.19-3.09 (m, 1H), 2.97 (d, 2H), 2.87-2.78 (m, 1H), 2.68 (d, J=10.8 Hz, 1H), 2.59 (dd, J=10.5, 1.8 Hz, 1H), 2.43 (d, J=11.0 Hz, 1H), 2.06-1.94 (m, 1H), 1.94-1.62 (m, 1H), MS (APCI) m/z: 499.4 [M+H]⁺.

### Example 36

### 2-[4-(2,4-difluorophenoxy)phenyl]-7-[4-(prop-2-enoyl)piperazin-1-yl]-4,5,6,7-tetrahydro-2H-pyrazolo[4,3-b]pyridine-3-carboxamide

### Step 36.1

### tert-butyl 4-{3-carbamoyl-2-[4-(2,4-difluorophenoxy)phenyl]-2H-pyrazolo[4,3-b]pyridin-7-yl}piperazine-1-carboxylate

To a vial equipped with a stir bar was added 2-(dimethylamino)acetic acid (0.0473 g, 0.459 mmol), cesium carbonate (0.224 g, 0.688 mmol), copper(I) iodide (0.0437 g, 0.229 mmol), 2,4-difluorophenol (0.0537 mL, 0.550 mmol), and tert-butyl 4-[2-(4-bromophenyl)-3-carbamoyl-2H-pyrazolo[4,3-b]pyridin-7-yl]piperazine-1-carboxylate (0.230 g, 0.459 mmol, Intermediate E). The vial was sealed and purged with N₂ (3×). N-Methyl-2-pyrrolidinone (4.6 mL) was added. The reaction mixture was warmed to 120° C. and stirred for 4 hours. The reaction mixture was cooled to ambient temperature and quenched with saturated aqueous NH₄Cl. The aqueous solution was extracted with ethyl acetate (3×). The combined organic layers were washed with brine (3×) and concentrated under reduced pressure. The crude product was purified by silica gel chromatography (0-100% ethyl acetate in heptanes) to provide the title compound (0.0438 g, 17%). MS (ESI) m/z: 550.97 [M+H]⁺.

### Step 36.2

### tert-butyl 4-{3-carbamoyl-2-[4-(2,4-difluorophenoxy)phenyl]-4,5,6,7-tetrahydro-2H-pyrazolo[4,3-b]pyridin-7-yl}piperazine-1-carboxylate

To a reactor (Thermo Barnstead Stem RS10) were added tert-butyl 4-{3-carbamoyl-2-[4-(2,4-difluorophenoxy)phenyl]-2H-pyrazolo[4,3-b]pyridin-7-yl}piperazine-1-carboxylate (0.117 g, 0.212 mmol, Step 36.1), 10% Pd(OH)₂/C (0.113 g, 0.0338 mmol) and tetrahydrofuran (8 mL). The vessel was pressurized with H₂ (120 psi). The reaction mixture was stirred for 20 hours at 50° C. The resulting solution was filtered over diatomaceous earth, and the filtrate was concentrated under reduced pressure. The crude product was purified by silica gel chromatography (0-100% ethyl acetate in heptanes) to provide the title compound (0.0616 mg, 69%). MS (APCI) m/z: 555.38 [M+H]⁺.

### Step 36.3

### 2-[4-(2,4-difluorophenoxy)phenyl]-7-[4-(prop-2-enoyl)piperazin-1-yl]-4,5,6,7-tetrahydro-2H-pyrazolo[4,3-b]pyridine-3-carboxamide

2-[4-(2,4-difluorophenoxy)phenyl]-7-(piperazin-1-yl)-4,5,6,7-tetrahydro-2H-pyrazolo[4,3-b]pyridine-3-carboxamide was prepared according to the procedure for Step 12.3, substituting tert-butyl 4-{3-carbamoyl-2-[4-(2,4-difluorophenoxy)phenyl]-4,5,6,7-tetrahydro-2H-pyrazolo[4,3-b]pyridin-7-yl}piperazine-1-carboxylate (Step 36.2) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. The title compound was prepared according to the procedure for Step 12.4, substituting 2-[4-(2,4-difluorophenoxy)phenyl]-7-(piperazin-1-yl)-4,5,6,7-tetrahydro-2H-pyrazolo[4,3-b]pyridine-3-carboxamide for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, dimethyl sulfoxide-d₆) δ ppm 7.47 (ddd, J=11.6, 8.8, 3.0 Hz, 1H), 7.35-7.24 (m, 3H), 7.17-7.07 (m, 1H), 6.98-6.93 (m, 2H), 6.73 (dd, J=16.7, 10.4 Hz, 1H), 6.05 (dd, J=16.7, 2.4 Hz, 1H), 5.62 (dd, J=10.4, 2.4 Hz, 1H), 5.08-5.02 (m, 1H), 3.61 (dd, J=6.1, 4.8 Hz, 1H), 3.57-3.18 (m, 5H), 3.12-3.02 (m, 1H), 2.71-2.48 (m, 4H), 2.11-1.97 (m, 1H), 1.76-1.64 (m, 1H). MS (ESI) m/z: 509.2 [M+H]⁺.

### Example 37

### 2-(2-fluoro-4-phenoxyphenyl)-7-[1-(prop-2-enoyl)piperidin-4-yl]-4,5,6,7-tetrahydro-2H-pyrazolo[4,3-b]pyridine-3-carboxamide

### Step 37.1

### tert-butyl 4-[2-(4-bromo-2-fluorophenyl)-3-cyano-2H-pyrazolo[4,3-b]pyridin-7-yl]piperidine-1-carboxylate

To a solution of tert-butyl 4-[2-(4-bromo-2-fluorophenyl)-3-carbamoyl-2H-pyrazolo[4,3-b]pyridin-7-yl]piperidine-1-carboxylate (0.815 g, 1.630 mmol, Intermediate W) and pyridine (0.398 mL, 4.92 mmol) in dichloromethane (16 mL) was added trifluoroacetic anhydride (0.463 mL, 3.28 mmol) at room temperature. The resulting solution was stirred for 4 h. The reaction mixture was quenched by the addition of saturated aqueous NaHCO₃. The resulting solution was stirred for 30 minutes and then extracted with dichloromethane (3×). The combined organic fractions were concentrated under reduced pressure. The crude product was purified by silica gel chromatography (0-50% ethyl acetate in heptanes) to provide the title compound (0.816 g, 1.63 mmol). MS (APCI) m/z: 500.6 [M+H]⁺.

### Step 37.2

### tert-butyl 4-[3-cyano-2-(2-fluoro-4-phenoxyphenyl)-2H-pyrazolo[4,3-b]pyridin-7-yl]piperidine-1-carboxylate

Step 39.2 was prepared according to the procedure for Step 12.1, substituting phenol for 3-fluorophenol, tert-butyl 4-[2-(4-bromo-2-fluorophenyl)-3-cyano-2H-pyrazolo[4,3-b]pyridin-7-yl]piperidine-1-carboxylate (Step 37.1) for tert-butyl 4-[2-(4-bromophenyl)-3-carbamoyl-2H-pyrazolo[4,3-b]pyridin-7-yl]piperidine-1-carboxylate and 2-(dimethylamino)acetic acid for 2,2,6,6-tetramethyl-3,5-heptanedione. MS (ESI) m/z: 514.1[M+H]⁺.

### Step 37.3

### tert-butyl 4-[3-carbamoyl-2-(2-fluoro-4-phenoxyphenyl)-2H-pyrazolo[4,3-b]pyridin-7-yl]piperidine-1-carboxylate

Step 37.3 was prepared according to the procedure for Step 11.3, substituting tert-butyl 4-[3-cyano-2-(2-fluoro-4-phenoxyphenyl)-2H-pyrazolo[4,3-b]pyridin-7-yl]piperidine-1-carboxylate (Step 37.2) for tert-butyl 4-(3-cyano-2-(4-(4-fluorophenoxy)phenyl)-2H-pyrazolo[4,3-b]pyridin-7-yl)piperidine-1-carboxylate. MS (ESI) m/z: 532.1 [M+H]⁺.

### Step 37.4

### tert-butyl 4-[3-carbamoyl-2-(2-fluoro-4-phenoxyphenyl)-4,5,6,7-tetrahydro-2H-pyrazolo[4,3-b]pyridin-7-yl]piperidine-1-carboxylate

Step 13.4 was prepared according to the procedure for Step 23.2, substituting tert-butyl 4-[3-carbamoyl-2-(2-fluoro-4-phenoxyphenyl)-2H-pyrazolo[4,3-b]pyridin-7-yl]piperidine-1-carboxylate (Step 37.3) for tert-butyl 4-(3-carbamoyl-2-{4-[3-(trifluoromethyl)phenoxy]phenyl}-2H-pyrazolo[4,3-b]pyridin-7-yl)piperidine-1-carboxylate. MS (ESI) m/z: 536.2 [M+H]⁺.

### Step 37.5

### 2-(2-fluoro-4-phenoxyphenyl)-7-[1-(prop-2-enoyl)piperidin-4-yl]-4,5,6,7-tetrahydro-2H-pyrazolo[4,3-b]pyridine-3-carboxamide

2-(2-Fluoro-4-phenoxyphenyl)-7-(piperidin-4-yl)-4,5,6,7-tetrahydro-2H-pyrazolo[4,3-b]pyridine-3-carboxamide was prepared according to the procedure for Step 12.3, substituting tert-butyl 4-[3-carbamoyl-2-(2-fluoro-4-phenoxyphenyl)-4,5,6,7-tetrahydro-2H-pyrazolo[4,3-b]pyridin-7-yl]piperidine-1-carboxylate (Step 37.4) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. The title compound was prepared according to the procedure for Step 12.4, substituting 2-(2-fluoro-4-phenoxyphenyl)-7-(piperidin-4-yl)-4,5,6,7-tetrahydro-2H-pyrazolo[4,3-b]pyridine-3-carboxamide for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, dimethyl sulfoxide-d₆) δ ppm 7.52-7.41 (m, 3H), 7.24 (t, J=7.4 Hz, 1H), 7.18-7.05 (m, 2H), 6.99 (dd, J=11.5, 2.7 Hz, 1H), 6.86 (dd, J=8.8, 2.7 Hz, 1H), 6.78 (dd, J=16.7, 10.4 Hz, 1H), 6.06 (dd, J=16.7, 2.5 Hz, 1H), 5.63 (dd, J=10.4, 2.5 Hz, 1H), 4.47 (d, J=12.9 Hz, 1H), 4.30-3.56 (m, 2H), 3.38-3.28 (m, 1H), 3.19-3.09 (m, 1H), 3.06-2.92 (m, 1H), 2.84-2.75 (m, 1H), 2.64-2.52 (m, 1H), 2.07-1.56 (m, 5H), 1.36-1.15 (m, 2H). MS (ESI) m/z: 490.0 [M+H]⁺.

### Example 38

### 2-[4-(2-fluorophenoxy)phenyl]-7-[4-(prop-2-enoyl)piperazin-1-yl]-4,5,6,7-tetrahydro-2H-pyrazolo[4,3-b]pyridine-3-carboxamide

### Step 38.1

### tert-butyl 4-{3-carbamoyl-2-[4-(2-fluorophenoxy)phenyl]-2H-pyrazolo[4,3-b]pyridin-7-yl}piperazine-1-carboxylate

To a vial equipped with a stir bar was added Cs₂CO₃ (0.487 g, 1.50 mmol), 2-(dimethylamino)acetic acid (0.103 g, 0.997 mmol), copper(I) iodide (0.095 g, 0.499 mmol), 2-fluorophenol (0.101 mL, 1.20 mmol), and tert-butyl 4-[2-(4-bromophenyl)-3-carbamoyl-2H-pyrazolo[4,3-b]pyridin-7-yl]piperidine-1-carboxylate (0.500 g, 0.999 mmol, Intermediate T). The vial was sealed and purged with N₂ (3×). N-Methyl-2-pyrrolidinone (10 mL) was added. The reaction mixture was warmed to 120° C. and stirred for 4 hours. The reaction mixture was cooled to ambient temperature and quenched with saturated aqueous NH₄Cl. The aqueous solution was extracted with ethyl acetate (3×). The combined organic layers were washed with brine (3×) and concentrated under reduced pressure. The crude product was purified by silica gel chromatography (0-100% ethyl acetate in heptanes) to provide the title compound (0.080 g, 15%). MS (APCI) m/z: 533.1 [M+H]⁺.

### Step 38.2

### tert-butyl 4-{3-carbamoyl-2-[4-(2-fluorophenoxy)phenyl]-4,5,6,7-tetrahydro-2H-pyrazolo[4,3-b]pyridin-7-yl}piperazine-1-carboxylate

To a Parr reactor were added tert-butyl 4-{3-carbamoyl-2-[4-(2-fluorophenoxy)phenyl]-2H-pyrazolo[4,3-b]pyridin-7-yl}piperazine-1-carboxylate (0.089 g, 0.12 mmol, Step 38.1), 10% Pd(OH)₂/C (0.091 g, 0.27 mmol) and tetrahydrofuran (3 mL). The vessel was pressurized with H₂ (120 psi) and warmed to 50° C. The reaction mixture was stirred for 24 hours at ambient temperature. The vessel was cooled to ambient temperature and vented. The reaction mixture was filtered over diatomaceous earth, and the filtrate was concentrated under reduced pressure. The crude product was purified by silica gel chromatography (0-100% ethyl acetate in heptanes) to provide the title compound (0.062 g, 69%). MS (ESI) m/z: 537.6 [M+H]⁺.

### Step 38.3

### 2-[4-(2-fluorophenoxy)phenyl]-7-[4-(prop-2-enoyl)piperazin-1-yl]-4,5,6,7-tetrahydro-2H-pyrazolo[4,3-b]pyridine-3-carboxamide

2-[4-(2-fluorophenoxy)phenyl]-7-(piperazin-1-yl)-4,5,6,7-tetrahydro-2H-pyrazolo[4,3-b]pyridine-3-carboxamide was prepared according to the procedure for Step 12.3, substituting tert-butyl 4-{3-carbamoyl-2-[4-(2-fluorophenoxy)phenyl]-4,5,6,7-tetrahydro-2H-pyrazolo[4,3-b]pyridin-7-yl}piperazine-1-carboxylate (Step 38.2) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. The title compound was prepared according to the procedure for Step 12.4, substituting 2-[4-(2-fluorophenoxy)phenyl]-7-(piperazin-1-yl)-4,5,6,7-tetrahydro-2H-pyrazolo[4,3-b]pyridine-3-carboxamide for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (400 MHz, dimethyl sulfoxide-d₆) δ ppm 7.44-7.30 (m, 3H), 7.30-7.18 (m, 3H), 7.05-6.95 (m, 2H), 6.76 (dd, J=16.7, 10.5 Hz, 1H), 6.11 (dd, J=16.7, 2.3 Hz, 1H), 5.70 (d, J=10.6 Hz, 1H), 4.85-2.67 (m, 12H), 2.37-2.12 (m, 1H), 2.10-1.81 (m, 1H). MS (ESI) m/z: 491.1 [M+H]⁺.

### Example 39

### (7S)-7-[4-(but-2-ynoyl)piperazin-1-yl]-2-(4-phenoxyphenyl)-4,5,6,7-tetrahydro-2H-pyrazolo[4,3-b]pyridine-3-carboxamide; and

### Example 40

### (7R)-7-[4-(but-2-ynoyl)piperazin-1-yl]-2-(4-phenoxyphenyl)-4,5,6,7-tetrahydro-2H-pyrazolo[4,3-b]pyridine-3-carboxamide

7-[4-(But-2-ynoyl)piperazin-1-yl]-2-(4-phenoxyphenyl)-4,5,6,7-tetrahydro-2H-pyrazolo[4,3-b]pyridine-3-carboxamide (0.240 g, 0.495 mmol, Example 27) was separated via chiral supercritical fluid chromatography to afford the individual enantiomers on a YMC Amylose-SA column (21×250 mm, 5 micron) using 21 mg/mL loading of compound in methanol. Elution was accomplished using an isocratic method of 35% methanol/CO₂ with a total flow rate of 80 g/minutes. Product elution was visualized using UV-absorption at 254 nm. The first-eluting peak (Example 39, 42.5 mg, 5.1 minutes ambient temperature) and second-eluting peak (Example 40, 53.9 mg, 5.92 minutes ambient temperature) were separated and identified as the two enantiomers indicated. Example 39: ¹H NMR (500 MHz, CDCl₃) δ ppm 7.44-7.34 (m, 4H), 7.22-7.13 (m, 1H), 7.12-7.02 (m, 4H), 5.25 (br s, 2H), 5.13 (br s, 1H), 3.87 (dd, J=6.9, 5.1 Hz, 1H), 3.80-3.66 (m, 3H), 3.59 (dq, J=10.6, 3.3 Hz, 1H), 3.44 (ddd, J=11.3, 8.0, 3.1 Hz, 1H), 3.38-3.26 (m, 1H), 2.81 (ddd, J=10.5, 6.3, 3.6 Hz, 1H), 2.69 (ddq, J=10.8, 7.2, 3.7 Hz, 3H), 2.17 (dtd, J=14.3, 7.4, 3.2 Hz, 1H), 1.99 (s, 3H), 1.98-1.89 (m, 1H). MS (APCI) m/z: 485.4 [M+H]⁺. Example 40: ¹H NMR (500 MHz, CDCl₃) δ ppm 7.44-7.34 (m, 4H), 7.22-7.13 (m, 1H), 7.12-7.02 (m, 4H), 5.25 (br s, 2H), 5.13 (br s, 1H), 3.87 (dd, J=6.9, 5.1 Hz, 1H), 3.80-3.66 (m, 3H), 3.59 (dq, J=10.6, 3.3 Hz, 1H), 3.44 (ddd, J=11.3, 8.0, 3.1 Hz, 1H), 3.38-3.26 (m, 1H), 2.81 (ddd, J=10.5, 6.3, 3.6 Hz, 1H), 2.69 (ddq, J=10.8, 7.2, 3.7 Hz, 3H), 2.17 (dtd, J=14.3, 7.4, 3.2 Hz, 1H), 1.99 (s, 3H), 1.98-1.89 (m, 1H). MS (APCI) m/z: 485.4 [M+H]⁺.

### Example 41

### 2-(4-phenoxyphenyl)-7-[3-(prop-2-enoyl)-3,6-diazabicyclo[3.1.1]heptan-6-yl]-4,5,6,7-tetrahydro-2H-pyrazolo[4,3-b]pyridine-3-carboxamide

### Step 41.1

### tert-butyl 6-[3-carbamoyl-2-(4-phenoxyphenyl)-2H-pyrazolo[4,3-b]pyridin-7-yl]-3,6-diazabicyclo[3.1.1]heptane-3-carboxylate

Step 41.1 was prepared according to the procedure for Step 35.1, substituting tert-butyl 3,6-diazabicyclo[3.1.1]heptane-3-carboxylate for (1R,5S)-tert-butyl 3,8-diazabicyclo[3.2.1]octane-8-carboxylate. MS (APCI) m/z: 527.5 [M+H]⁺.

### Step 41.2

### tert-butyl 6-[3-carbamoyl-2-(4-phenoxyphenyl)-4,5,6,7-tetrahydro-2H-pyrazolo[4,3-b]pyridin-7-yl]-3,6-diazabicyclo[3.1.1]heptane-3-carboxylate

Step 41.2 was prepared according to the procedure for Step 36.2, substituting tert-butyl 6-[3-carbamoyl-2-(4-phenoxyphenyl)-2H-pyrazolo[4,3-b]pyridin-7-yl]-3,6-diazabicyclo[3.1.1]heptane-3-carboxylate (Step 41.1) for tert-butyl 4-{3-carbamoyl-2-[4-(2,4-difluorophenoxy)phenyl]-2H-pyrazolo[4,3-b]pyridin-7-yl}piperazine-1-carboxylate. MS (APCI) m/z: 531.5 [M+H]⁺.

### Step 41.3

### 7-(3,6-diazabicyclo[3.1.1]heptan-6-yl)-2-(4-phenoxyphenyl)-4,5,6,7-tetrahydro-2H-pyrazolo[4,3-b]pyridine-3-carboxamide

Step 41.3 was prepared according to the procedure for Step 35.3, substituting tert-butyl 6-[3-carbamoyl-2-(4-phenoxyphenyl)-4,5,6,7-tetrahydro-2H-pyrazolo[4,3-b]pyridin-7-yl]-3,6-diazabicyclo[3.1.1]heptane-3-carboxylate (Step 41.2) for tert-butyl (1R,5S)-3-[3-carbamoyl-2-(4-phenoxyphenyl)-4,5,6,7-tetrahydro-2H-pyrazolo[4,3-b]pyridin-7-yl]-3,8-diazabicyclo[3.2.1]octane-8-carboxylate. MS (APCI) m/z: 431.5 [M+H]⁺.

### Step 41.4

### 2-(4-phenoxyphenyl)-7-[3-(prop-2-enoyl)-3,6-diazabicyclo[3.1.1]heptan-6-yl]-4,5,6,7-tetrahydro-2H-pyrazolo[4,3-b]pyridine-3-carboxamide

Step 41.4 was prepared according to the procedure for the Step 18.4, substituting 7-(3,6-diazabicyclo[3.1.1]heptan-6-yl)-2-(4-phenoxyphenyl)-4,5,6,7-tetrahydro-2H-pyrazolo[4,3-b]pyridine-3-carboxamide (Step 41.3) for 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide to afford the title compound. ¹H NMR (400 MHz, dimethyl sulfoxide-d₆, 90° C.) δ ppm 7.41-7.35 (m, 2H), 7.35-7.28 (m, 2H), 7.13 (tt, J=7.4, 1.2 Hz, 1H), 7.09-6.96 (m, 4H), 6.77-6.56 (m, 3H), 6.14 (dd, J=16.8, 2.5 Hz, 1H), 5.64 (dd, J=10.4, 2.5 Hz, 1H), 4.92 (s, 1H), 4.23 (dd, J=75.5, 12.5 Hz, 1H), 3.98-3.55 (m, 4H), 3.46 (dd, J=13.7, 6.8 Hz, 1H), 3.31 (q, J=5.3 Hz, 1H), 3.08 (d, J=11.9 Hz, 2H), 2.34 (q, J=6.9 Hz, 1H), 1.75 (p, J=4.0, 3.6 Hz, 2H). MS (APCI) m/z: 485.5 [M+H]⁺.

### Example 42

### 7-[(3R)-3-methyl-4-(prop-2-enoyl)piperazin-1-yl]-2-(4-phenoxyphenyl)-4,5,6,7-tetrahydro-2H-pyrazolo[4,3-b]pyridine-3-carboxamide

Example 42 was prepared according to the procedure for Example 43, substituting (R)-tert-butyl 2-methylpiperazine-1-carboxylate for (S)-tert-butyl 2-methylpiperazine-1-carboxylate. ¹H NMR (400 MHz, CDCl₃) δ ppm 7.32 (t, J=7.5 Hz, 4H), 7.11 (t, J=7.3 Hz, 1H), 7.00 (t, J=7.2 Hz, 4H), 6.47 (dd, J=16.7, 10.4 Hz, 1H), 6.19 (d, J=16.6 Hz, 1H), 5.59 (d, J=10.5 Hz, 1H), 5.31-5.10 (m, 2H), 4.75 (s, 1H), 4.39-4.05 (m, 1H), 3.79-3.63 (m, 1H), 3.44-3.32 (m, 1H), 3.28-3.17 (m, 1H), 2.99-2.72 (m, 2H), 2.59-2.27 (m, 2H), 2.09-1.83 (m, 2H), 1.29-1.13 (m, 4H), 0.81 (s, 1H). MS (ESI) m/z: 487.1 [M+H]⁺.

### Example 43

### 7-[(3S)-3-methyl-4-(prop-2-enoyl)piperazin-1-yl]-2-(4-phenoxyphenyl)-4,5,6,7-tetrahydro-2H-pyrazolo[4,3-b]pyridine-3-carboxamide

### Step 43.1

### tert-butyl (2S)-4-{3-carbamoyl-4-[(4-methoxyphenyl)methyl]-2-(4-phenoxyphenyl)-4,5,6,7-tetrahydro-2H-pyrazolo[4,3-b]pyridin-7-yl}-2-methylpiperazine-1-carboxylate

A round-bottomed flask was charged with 4-[(4-methoxyphenyl)methyl]-7-oxo-2-(4-phenoxyphenyl)-4,5,6,7-tetrahydro-2H-pyrazolo[4,3-b]pyridine-3-carboxamide (140 mg, 0.299 mmol, Intermediate K), zinc chloride (81 mg, 0.598 mmol), (S)-tert-butyl 2-methylpiperazine-1-carboxylate (90 mg, 0.448 mmol) and ethanol (30 mL), and the resulting mixture was heated at reflux for 2 hours. Sodium cyanoborohydride (56.3 mg, 0.896 mmol) was then added, and the resulting mixture was heated to 88° C. for 5 hours. The mixture was cooled to ambient temperature and concentrated under reduced pressure. The residue was dissolved in dichloromethane. The organic layer was washed with water, brine, dried over anhydrous Na₂SO₄, and concentrated under reduced pressure. The material was purified via column chromatography (0-80% of ethyl acetate in petroleum ether) to afford the title compound (130 mg, 66.6%). MS (ESI) m/z: 653.34 [M+H]⁺.

### Step 43.2

### 7-[(3S)-3-methylpiperazin-1-yl]-2-(4-phenoxyphenyl)-4,5,6,7-tetrahydro-2H-pyrazolo[4,3-b]pyridine-3-carboxamide

To a round-bottomed flask charged with tert-butyl (2S)-4-{3-carbamoyl-4-[(4-methoxyphenyl)methyl]-2-(4-phenoxyphenyl)-4,5,6,7-tetrahydro-2H-pyrazolo[4,3-b]pyridin-7-yl}-2-methylpiperazine-1-carboxylate (130 mg, 0.199 mmol, Step 43.1) was added trifluoroacetic acid (7 mL). The reaction mixture was heated to 60° C. for 30 minutes with stirring. The reaction mixture was cooled down to ambient temperature, diluted with dichloromethane and concentrated under reduced pressure. The residue was dissolved in dichloromethane and the resulting solution was neutralized by basic washes with the saturated solution of NaHCO₃. The organic layer was separated, washed with water and brine, dried over anhydrous Na₂SO₄, and concentrated under reduced pressure to afford the title compound (86 mg, 100%). MS (ESI) m/z: 433.23 [M+H]⁺.

### Step 43.3

### 7-[(3S)-3-methyl-4-(prop-2-enoyl)piperazin-1-yl]-2-(4-phenoxyphenyl)-4,5,6,7-tetrahydro-2H-pyrazolo[4,3-b]pyridine-3-carboxamide

Step 43.3 was prepared according to the procedure for Step 100.4, substituting 7-[(3S)-3-methylpiperazin-1-yl]-2-(4-phenoxyphenyl)-4,5,6,7-tetrahydro-2H-pyrazolo[4,3-b]pyridine-3-carboxamide (Step 43.2) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide and triethylamine for N,N-diisopropylethylamine. ¹H NMR (400 MHz, dimethyl sulfoxide-d₆) δ ppm 8.27 (s, 1H), 7.45-7.38 (m, 2H), 7.33 (t, J=5.9 Hz, 2H), 7.16 (td, J=7.5, 0.9 Hz, 2H), 7.04 (ddd, J=9.8, 7.0, 2.1 Hz, 4H), 6.75 (dd, J=16.7, 10.5 Hz, 1H), 6.08 (dd, J=16.7, 2.2 Hz, 1H), 5.64 (dd, J=10.4, 2.4 Hz, 1H), 5.10 (d, J=17.0 Hz, 1H), 4.56 (s, 1H), 4.16 (s, 1H), 3.97-3.67 (m, 2H), 3.30-3.20 (m, 1H), 3.13 (d, J=7.9 Hz, 1H), 3.00-2.87 (m, 1H), 2.79-2.64 (m, 1H), 2.33 (s, 1H), 2.17-1.98 (m, 2H), 1.80-1.66 (m, 1H), 1.27-1.09 (m, 3H). MS (ESI) m/z: 487.24 [M+H]⁺.

### Example 44

### (7R)-2-[4-(4-hydroxyphenoxy)phenyl]-7-[4-(prop-2-enoyl)piperazin-1-yl]-4,5,6,7-tetrahydro-2H-pyrazolo[4,3-b]pyridine-3-carboxamide

Nicotinamide adenine dinucleotide phosphate hydrogen (NADPH, 2.5 mL, 10 mM in water) was added to each of three solutions containing (7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide (213 μL, 10 mM in dimethyl sulfoxide, Example 6), potassium phosphate buffer (12.5 mL, 100 mM, pH 7.4), water (7.75 mL), and a solution containing microsomes (1.25 mL, 20 mg/mL, male Cynomolgus monkey liver). The reactions were incubated at 37° C. for 1.5 hours, then quenched with methanol/acetonitrile (50:50 v/v). Solutions were concentrated to 100 μL each under N₂. The resulting product was purified by HPLC (10-98% acetonitrile in water with 0.1% formic acid over 10 minutes; Column: Phenomenex® Luna® C18(2) 100×4.6, 3 μm particle size; Flow rate 1 mL/min; Offline detection: Thermo QExactive HRMS) to provide the title compound (177 μg). ¹H NMR (600 MHz, pyridine-d5) δ ppm 7.69-7.65 (m, 2H), 7.20-7.16 (m, 2H), 7.08 (d, J=7.0 Hz, 2H), 7.07-7.04 (m, 2H), 6.73 (dd, J=16.7, 10.5 Hz, 1H), 6.46 (dd, J=16.7, 2.5 Hz, 1H), 5.61 (dd, J=10.4, 2.5 Hz, 1H), 3.86 (s, 1H), 3.83 (dd, J=6.1, 4.9 Hz, 1H), 3.77 (d, J=12.8 Hz, 1H), 3.54 (s, 2H), 3.46 (ddd, J=11.7, 8.8, 2.9 Hz, 1H), 3.25 (ddd, J=11.1, 7.1, 3.1 Hz, 1H), 2.85 (s, 1H), 2.76 (s, 2H), 2.70 (s, 1H), 2.14 (dtd, J=13.5, 6.7, 2.8 Hz, 1H), 1.85 (dddd, J=13.6, 8.4, 4.6, 3.1 Hz, 1H) MS (ESI) m/z: 489.2 [M+H]⁺.

### Example 45

### 2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide

### Step 45.1

### ethyl 7-[6-(tert-butoxycarbonyl)-3,6-diazabicyclo[3.1.1]heptan-3-yl]-2-(4-phenoxyphenyl)-2H-pyrazolo[4,3-b]pyridine-3-carboxylate

Ethyl 7-bromo-2-(4-phenoxyphenyl)-2H-pyrazolo[4,3-b]pyridine-3-carboxylate (1.98 g, 4.52 mmol, Step B.4), ethyl 7-chloro-2-(4-phenoxyphenyl)-2H-pyrazolo[4,3-b]pyridine-3-carboxylate (1.779 g, 4.52 mmol, Step 7.4) and tert-butyl 3,6-diazabicyclo[3.1.1]heptane-6-carboxylate (2.69 g, 13.55 mmol) were suspended in N,N-dimethylacetamide (45.2 mL) and N-ethyl-N-isopropylpropan-2-amine (3.16 mL, 18.07 mmol) added. The reaction was heated to 140° C. for 2 hours. After cooling to 25° C., the reaction mixture was diluted with water and extracted with ethyl acetate (4×). The combined organic layer was washed with brine, dried over Na₂SO₄, filtered and concentrated under reduced pressure to afford a residue, which was purified by column chromatography on silica gel (eluted with 40-70% heptane/ethyl acetate (+20% dichloromethane)) to afford the title compound (3.94 g, 157% yield). MS (APCI) m/z: 556.4 [M+H]⁺.

### Step 45.2

### tert-butyl 3-[3-carbamoyl-2-(4-phenoxyphenyl)-2H-pyrazolo[4,3-b]pyridin-7-yl]-3,6-diazabicyclo[3.1.1]heptane-6-carboxylate

Ethyl 7-[6-(tert-butoxycarbonyl)-3,6-diazabicyclo[3.1.1]heptan-3-yl]-2-(4-phenoxyphenyl)-2H-pyrazolo[4,3-b]pyridine-3-carboxylate (2.51 g, 4.52 mmol, Step 45.1) was added to a flask, purged with nitrogen for 1 minute, and then dissolved in 7 M ammonia in methanol (45 mL, 315 mmol). The reaction was heated at 65° C. for 16 hours and then concentrated under reduced pressure. The residue was purified by column chromatography on silica gel (0-100% heptane/ethyl acetate) to afford the title compound (1.78 g, 74.8%). MS (APCI) m/z: 527.4 [M+H]⁺.

### Step 45.3

### tert-butyl 3-[3-carbamoyl-2-(4-phenoxyphenyl)-4,5,6,7-tetrahydro-2H-pyrazolo[4,3-b]pyridin-7-yl]-3,6-diazabicyclo[3.1.1]heptane-6-carboxylate

Step 45.3 was prepared according to the procedure for Step 35.2, substituting tert-butyl 3-[3-carbamoyl-2-(4-phenoxyphenyl)-2H-pyrazolo[4,3-b]pyridin-7-yl]-3,6-diazabicyclo[3.1.1]heptane-6-carboxylate (Step 45.2) for tert-butyl (1R,5S)-3-[3-carbamoyl-2-(4-phenoxyphenyl)-2H-pyrazolo[4,3-b]pyridin-7-yl]-3,8-diazabicyclo[3.2.1]octane-8-carboxylate. MS (APCI) m/z: 531.4 [M+H]⁺.

### Step 45.4

### 7-(3,6-diazabicyclo[3.1.1]heptan-3-yl)-2-(4-phenoxyphenyl)-4,5,6,7-tetrahydro-2H-pyrazolo[4,3-b]pyridine-3-carboxamide

Step 45.4 was prepared according to the procedure for Step 56.4, substituting tert-butyl 3-[3-carbamoyl-2-(4-phenoxyphenyl)-4,5,6,7-tetrahydro-2H-pyrazolo[4,3-b]pyridin-7-yl]-3,6-diazabicyclo[3.1.1]heptane-6-carboxylate (Step 45.3) for tert-butyl 4-{3-carbamoyl-2-[4-(4-fluorophenoxy)phenyl]-4,5,6,7-tetrahydro-2H-pyrazolo[4,3-b]pyridin-7-yl}piperazine-1-carboxylate. MS (APCI) m/z: 431.4 [M+H]⁺.

### Step 45.5

### 2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide

Step 45.5 was prepared according to the procedure for Step 16.8, substituting 7-(3,6-diazabicyclo[3.1.1]heptan-3-yl)-2-(4-phenoxyphenyl)-4,5,6,7-tetrahydro-2H-pyrazolo[4,3-b]pyridine-3-carboxamide (Step 45.4) for 2-(4-Phenoxyphenyl)-7-(piperidin-4-yl)-4,5,6,7-tetrahydro-2H-pyrazolo[3,4-b]pyrazine-3-carboxamide-hydrogen chloride (1/2) and cooled to −50° C. ¹H NMR (400 MHz, dimethyl sulfoxide-d₆) δ ppm 7.46-7.38 (m, 2H), 7.36-7.27 (m, 2H), 7.16 (td, J=7.4, 1.1 Hz, 1H), 7.09-6.97 (m, 4H), 6.33 (dd, J=16.9, 10.2 Hz, 1H), 6.07 (ddd, J=16.9, 6.1, 2.3 Hz, 1H), 5.59 (dd, J=10.2, 2.3 Hz, 1H), 5.06 (dt, J=12.9, 3.0 Hz, 1H), 4.64-4.47 (m, 1H), 4.30-4.20 (m, 1H), 3.82 (ddd, J=26.3, 6.7, 4.9 Hz, 1H), 3.32-3.07 (m, 4H), 3.06-2.85 (m, 2H), 2.41-2.32 (m, 1H), 2.12-1.95 (m, 1H), 1.91-1.81 (m, 1H), 1.73 (qd, J=9.6, 9.1, 5.4 Hz, 1H). MS (ESI) m/z: 485.4 [M+H]⁺.

### Example 46

### (7R)-7-{4-[(2E)-4-(dimethylamino)but-2-enoyl]piperazin-1-yl}-2-(4-phenoxyphenyl)-4,5,6,7-tetrahydro-2H-pyrazolo[4,3-b]pyridine-3-carboxamide

Example 46 was prepared according to the procedure for Example 48, substituting ((E)-4-(dimethylamino)but-2-enoic acid (0.0075 g, 0.050 mmol) for (E)-4-aminobut-2-enoic acid. The residue was purified by reverse-phase preparative HPLC on a Phenomenex® Luna® C8(2) 5 μm 100 Å AXIA™ column (50 mm×30 mm). A gradient of acetonitrile (A) and 0.1% trifluoroacetic acid in water (B) was used, at a flow rate of 40 mL/minute (0-0.5 minutes 5% A, 0.5-8.0 minutes linear gradient 5-100% A, 8.0-9.0 minutes 100% A, 9.0-9.1 minutes linear gradient 100-5% A, 9.1-10.0 minutes 5% A) to provide the title compound (0.0016 g, 6.3%). ¹H NMR (400 MHz, pyridine-d₅) δ ppm 7.65-7.57 (m, 2H), 7.36-7.27 (m, 2H), 7.14-7.02 (m, 4H), 7.00-6.94 (m, 1H), 6.91 (s, 1H), 6.62-6.55 (m, 1H), 3.84 (t, J=5.5 Hz, 2H), 3.68 (s, 2H), 3.49-3.37 (m, 2H), 3.29-3.17 (m, 2H), 3.08 (dd, J=6.1, 1.6 Hz, 2H), 2.80 (ddd, J=34.3, 11.7, 6.0 Hz, 4H), 2.22 (s, 6H), 2.15 (dd, J=8.3, 5.3 Hz, 2H), 1.93-1.81 (m, 2H). MS m/z: 530.3 [M+H]⁺.

### Example 47

### (7R)-7-{4-[(2E)-but-2-enoyl]piperazin-1-yl}-2-(4-phenoxyphenyl)-4,5,6,7-tetrahydro-2H-pyrazolo[4,3-b]pyridine-3-carboxamide

Example 47 was prepared according to the procedure for Example 48, substituting (E)-but-2-enoic acid (0.0039 g, 0.05 mmol) for (E)-4-aminobut-2-enoic acid. The residue was purified by reverse-phase preparative HPLC on a Phenomenex® Luna® C8(2) 5 μm 100 Å AXIA™ column (50 mm×30 mm). A gradient of acetonitrile (A) and 0.1% trifluoroacetic acid in water (B) was used, at a flow rate of 40 mL/minute (0-0.5 minutes 5% A, 0.5-8.0 minutes linear gradient 5-100% A, 8.0-9.0 minutes 100% A, 9.0-9.1 minutes linear gradient 100-5% A, 9.1-10.0 minutes 5% A) to provide the title compound (0.0016 g, 6.9%). ¹H NMR (400 MHz, pyridine-d₅) δ ppm 7.66-7.58 (m, 2H), 7.36-7.27 (m, 2H), 7.14-7.01 (m, 4H), 6.96-6.84 (m, 3H), 6.35 (dd, J=15.2, 1.8 Hz, 2H), 3.84 (t, J=5.6 Hz, 2H), 3.65 (s, 2H), 3.44 (ddd, J=11.8, 8.8, 3.1 Hz, 2H), 3.23 (ddd, J=11.1, 7.1, 3.3 Hz, 2H), 2.88-2.71 (m, 4H), 2.21-2.09 (m, 2H), 1.88 (dt, J=8.7, 4.9 Hz, 2H), 1.72 (dd, J=6.8, 1.7 Hz, 3H). MS m/z: 487.1 [M+H]⁺.

### Example 48

### (7R)-7-{4-[(2E)-4-amino-4-oxobut-2-enoyl]piperazin-1-yl}-2-(4-phenoxyphenyl)-4,5,6,7-tetrahydro-2H-pyrazolo[4,3-b]pyridine-3-carboxamide

(7R)-2-(4-phenoxyphenyl)-7-(piperazin-1-yl)-4,5,6,7-tetrahydro-2H-pyrazolo[4,3-b]pyridine-3-carboxamide (0.021 g, 0.050 mmol, Step 6.1) was dissolved in dichloromethane (0.25 mL) and cooled to 0° C. N, N-Diisopropylethylamine (0.033 mL, 0.19 mmol) was added, followed by (E)-4-aminobut-2-enoic acid (0.0052 g, 0.050 mmol) in dichloromethane (0.25 mL). Propylphosphonic anhydride solution (50% by weight in ethyl acetate, 0.017 mL, 0.061 mmol) was added dropwise, and the reaction was stirred for 5 minutes. Brine (0.50 mL) was added, the layers were separated using a phase separator cartridge, and the organic layer was concentrated. The residue was purified by reverse-phase preparative HPLC on a Phenomenex® Luna® C8(2) 5 μm 100 Å AXIA™ column (50 mm×30 mm). A gradient of acetonitrile (A) and 0.1% trifluoroacetic acid in water (B) was used, at a flow rate of 40 mL/minute (0-0.5 minutes 5% A, 0.5-8.0 minutes linear gradient 5-100% A, 8.0-9.0 minutes 100% A, 9.0-9.1 minutes linear gradient 100-5% A, 9.1-10.0 minutes 5% A) to provide the title compound (0.0014 g, 6.0%). ¹H NMR (400 MHz, pyridine-d₅) δ ppm 7.63 (d, J=8.9 Hz, 2H), 7.40-7.27 (m, 2H), 7.12-7.01 (m, 4H), 6.96 (s, 1H), 6.44 (d, J=12.4 Hz, 1H), 6.26 (d, J=12.5 Hz, 1H), 3.41 (d, J=8.8 Hz, 2H), 3.21 (s, 2H), 2.81 (d, J=34.2 Hz, 4H), 2.16-2.04 (m, 2H), 1.95-1.73 (m, 2H), 1.40-1.21 (m, 2H). MS m/z: 516.3 [M+H]⁺.

### Example 49

### 7-[4-(fluoroacetyl)piperazin-1-yl]-2-(4-phenoxyphenyl)-4,5,6,7-tetrahydro-2H-pyrazolo[4,3-b]pyridine-3-carboxamide

Example 49 was prepared according to the procedure for Example 48, substituting 2-fluoroacetic acid (0.0035 g, 0.050 mmol) for (E)-4-aminobut-2-enoic acid. The residue was purified by reverse-phase preparative HPLC on a Phenomenex® Luna® C8(2) 5 μm 100 Å AXIA™ column (50 mm×30 mm). A gradient of acetonitrile (A) and 0.1% trifluoroacetic acid in water (B) was used, at a flow rate of 40 mL/minute (0-0.5 minutes 5% A, 0.5-8.0 minutes linear gradient 5-100% A, 8.0-9.0 minutes 100% A, 9.0-9.1 minutes linear gradient 100-5% A, 9.1-10.0 minutes 5% A) to provide the title compound (0.0012 g, 5.3%). ¹H NMR (400 MHz, pyridine-d₅) δ ppm 7.65-7.57 (m, 2H), 7.36-7.27 (m, 2H), 7.15-7.02 (m, 4H), 6.91 (s, 1H), 5.10 (s, 1H), 4.98 (s, 1H), 3.84 (dd, J=6.2, 5.0 Hz, 2H), 3.53 (s, 2H), 3.42 (ddd, J=11.8, 8.8, 3.1 Hz, 2H), 3.27-3.17 (m, 2H), 2.78 (ddd, J=34.7, 11.8, 6.1 Hz, 4H), 2.12 (td, J=6.8, 2.9 Hz, 2H), 1.87 (dd, J=8.6, 5.2 Hz, 2H). MS m/z: 479.3 [M+H]⁺.

### Example 50

### (7R)-7-{4-[(2E)-3-ethoxyprop-2-enoyl]piperazin-1-yl}-2-(4-phenoxyphenyl)-4,5,6,7-tetrahydro-2H-pyrazolo[4,3-b]pyridine-3-carboxamide

Example 50 was prepared according to the procedure for Example 48, substituting (E)-3-ethoxyacrylic acid (0.0053 g, 0.050 mmol) for (E)-4-aminobut-2-enoic acid. The residue was purified by reverse-phase preparative HPLC on a Phenomenex® Luna® C8(2) 5 μm 100 Å AXIA™ column (50 mm×30 mm). A gradient of acetonitrile (A) and 0.1% trifluoroacetic acid in water (B) was used, at a flow rate of 40 mL/minute (0-0.5 minutes 5% A, 0.5-8.0 minutes linear gradient 5-100% A, 8.0-9.0 minutes 100% A, 9.0-9.1 minutes linear gradient 100-5% A, 9.1-10.0 minutes 5% A) to provide the title compound (0.0042 g, 17%). ¹H NMR (400 MHz, pyridine-d₅) δ ppm 7.82-7.72 (m, 1H), 7.65-7.57 (m, 2H), 7.34-7.26 (m, 2H), 7.13-7.02 (m, 4H), 6.91 (s, 1H), 5.86 (dd, J=11.8, 6.2 Hz, 1H), 3.87-3.72 (m, 3H), 3.66 (dt, J=6.4, 3.9 Hz, 2H), 3.49-3.35 (m, 2H), 3.22 (ddd, J=11.4, 7.1, 3.5 Hz, 1H), 2.90-2.67 (m, 4H), 2.22-2.04 (m, 2H), 1.88 (dq, J=14.6, 5.7, 5.0 Hz, 2H), 1.16 (t, J=7.0 Hz, 3H). MS m/z: 517.1 [M+H]⁺.

### Example 51

### methyl (2E)-4-{4-[3-carbamoyl-2-(4-phenoxyphenyl)-4,5,6,7-tetrahydro-2H-pyrazolo[4,3-b]pyridin-7-yl]piperazin-1-yl}-4-oxobut-2-enoate

Example 51 was prepared according to the procedure for Example 48, substituting (E)-4-methoxy-4-oxobut-2-enoic acid (0.0059 g, 0.005 mmol) for (E)-4-aminobut-2-enoic acid. The residue was purified by reverse-phase preparative HPLC on a Phenomenex® Luna® C8(2) 5 μm 100 Å AXIA™ column (50 mm×30 mm). A gradient of acetonitrile (A) and 0.1% trifluoroacetic acid in water (B) was used, at a flow rate of 40 mL/minute (0-0.5 minutes 5% A, 0.5-8.0 minutes linear gradient 5-100% A, 8.0-9.0 minutes 100% A, 9.0-9.1 minutes linear gradient 100-5% A, 9.1-10.0 minutes 5% A) to provide the title compound (0.0041 g, 16%). ¹H NMR (400 MHz, pyridine-d₅) δ ppm 7.67-7.58 (m, 2H), 7.51 (s, 1H), 7.36-7.26 (m, 2H), 7.13-7.01 (m, 4H), 6.89 (d, J=15.4 Hz, 2H), 3.85 (dd, J=6.2, 5.0 Hz, 1H), 3.69 (s, 3H), 3.63 (s, 2H), 3.43 (ddd, J=11.9, 8.7, 3.2 Hz, 1H), 3.23 (ddd, J=11.6, 7.1, 3.3 Hz, 2H), 2.89-2.69 (m, 4H), 2.14 (dtd, J=13.5, 6.8, 3.1 Hz, 2H), 1.94-1.82 (m, 2H). MS m/z: 531.1 [M+H]⁺.

### Example 52

### 2-(2-chloro-4-phenoxyphenyl)-7-[4-(prop-2-enoyl)piperazin-1-yl]-4,5,6,7-tetrahydro-2H-pyrazolo[4,3-b]pyridine-3-carboxamide

### Step 52.1

### (2E)-2-[2-(2-chloro-4-phenoxyphenyl)hydrazinylidene]-3-oxobutanenitrile

Step 52.1 was prepared according to the procedure for Step K.1, substituting 2-chloro-4-phenoxyaniline for 4-phenoxyaniline.

### Step 52.2

### ethyl 3-acetyl-4-amino-1-(2-chloro-4-phenoxyphenyl)-1H-pyrazole-5-carboxylate

Step 52.2 was prepared according to the procedure for Step K.2, substituting (2E)-2-[2-(2-chloro-4-phenoxyphenyl)hydrazinylidene]-3-oxobutanenitrile (Step 52.1) for (2E)-3-oxo-2-[2-(4-phenoxyphenyl)hydrazinylidene]butanenitrile. MS (ESI) m/z: 400 [M+H]⁺.

### Step 52.3

### ethyl 2-(2-chloro-4-phenoxyphenyl)-7-hydroxy-2H-pyrazolo[4,3-b]pyridine-3-carboxylate

Step 52.3 was prepared according to the procedure for Step K.3, substituting ethyl 3-acetyl-4-amino-1-(2-chloro-4-phenoxyphenyl)-1H-pyrazole-5-carboxylate (Step 52.2) for ethyl 3-acetyl-4-amino-1-(4-phenoxyphenyl)-1H-pyrazole-5-carboxylate. MS (ESI) m/z: 408 [M−H]⁻.

### Step 52.4

### ethyl 2-(2-chloro-4-phenoxyphenyl)-4-[(4-methoxyphenyl)methyl]-7-oxo-4,7-dihydro-2H-pyrazolo[4,3-b]pyridine-3-carboxylate

Step 52.4 was prepared according to the procedure for Step K.4, substituting ethyl 2-(2-chloro-4-phenoxyphenyl)-7-hydroxy-2H-pyrazolo[4,3-b]pyridine-3-carboxylate (Step 52.3) for ethyl 7-hydroxy-2-(4-phenoxyphenyl)-2H-pyrazolo[4,3-b]pyridine-3-carboxylate. MS (ESI) m/z: 530.16 [M+H]⁺.

### Step 52.5

### ethyl 2-(2-chloro-4-phenoxyphenyl)-4-[(4-methoxyphenyl)methyl]-7-oxo-4,5,6,7-tetrahydro-2H-pyrazolo[4,3-b]pyridine-3-carboxylate

Step 52.5 was prepared according to the procedure for Step K.5, substituting ethyl 2-(2-chloro-4-phenoxyphenyl)-4-[(4-methoxyphenyl)methyl]-7-oxo-4,7-dihydro-2H-pyrazolo[4,3-b]pyridine-3-carboxylate (Step 52.4) for ethyl 4-[(4-methoxyphenyl)methyl]-7-oxo-2-(4-phenoxyphenyl)-4,7-dihydro-2H-pyrazolo[4,3-b]pyridine-3-carboxylate.

### Step 52.6

### 2-(2-chloro-4-phenoxyphenyl)-4-[(4-methoxyphenyl)methyl]-7-oxo-4,5,6,7-tetrahydro-2H-pyrazolo[4,3-b]pyridine-3-carboxylic acid

A flask was charged with ethyl 2-(2-chloro-4-phenoxyphenyl)-4-[(4-methoxyphenyl)methyl]-7-oxo-4,5,6,7-tetrahydro-2H-pyrazolo[4,3-b]pyridine-3-carboxylate (385 mg, 0.724 mmol, Step 52.5) and lithium hydroxide (52.0 mg, 2.171 mmol) in dimethyl sulfoxide (3 mL) and water (0.5 mL). The reaction mixture was stirred for 16 hours at ambient temperature. The reaction was diluted with water and extracted with ethyl acetate (2×). The combined organic layers were washed with brine, then water, dried over Na₂SO₄ and concentrated under reduced pressure to afford the title compound (365 mg, 100%).

### Step 52.7

### 2-(2-chloro-4-phenoxyphenyl)-4-[(4-methoxyphenyl)methyl]-7-oxo-4,5,6,7-tetrahydro-2H-pyrazolo[4,3-b]pyridine-3-carboxamide

Step 52.7 was prepared according to the procedure for Step K.7, substituting 2-(2-chloro-4-phenoxyphenyl)-4-[(4-methoxyphenyl)methyl]-7-oxo-4,5,6,7-tetrahydro-2H-pyrazolo[4,3-b]pyridine-3-carboxylic acid (Step 52.6) for 4-[(4-methoxyphenyl)methyl]-7-oxo-2-(4-phenoxyphenyl)-4,5,6,7-tetrahydro-2H-pyrazolo[4,3-b]pyridine-3-carboxylic acid.

### Step 52.8

### tert-butyl 4-{3-carbamoyl-2-(2-chloro-4-phenoxyphenyl)-4-[(4-methoxyphenyl)methyl]-4,5,6,7-tetrahydro-2H-pyrazolo[4,3-b]pyridin-7-yl}piperazine-1-carboxylate

Step 52.8 was prepared according to the procedure for Step 43.1, substituting 2-(2-chloro-4-phenoxyphenyl)-4-[(4-methoxyphenyl)methyl]-7-oxo-4,5,6,7-tetrahydro-2H-pyrazolo[4,3-b]pyridine-3-carboxamide (Step 52.7) for 4-[(4-methoxyphenyl)methyl]-7-oxo-2-(4-phenoxyphenyl)-4,5,6,7-tetrahydro-2H-pyrazolo[4,3-b]pyridine-3-carboxamide (Intermediate K) and tert-butyl piperazine-1-carboxylate for (S)-tert-butyl 2-methylpiperazine-1-carboxylate. MS (ESI) m/z: 673.3 [M+H]⁺.

### Step 52.9

### 2-(2-chloro-4-phenoxyphenyl)-7-(piperazin-1-yl)-4,5,6,7-tetrahydro-2H-pyrazolo[4,3-b]pyridine-3-carboxamide

Step 52.9 was prepared according to the procedure for Step 43.2, substituting tert-butyl 4-{3-carbamoyl-2-(2-chloro-4-phenoxyphenyl)-4-[(4-methoxyphenyl)methyl]-4,5,6,7-tetrahydro-2H-pyrazolo[4,3-b]pyridin-7-yl}piperazine-1-carboxylate (Step 52.8) for tert-butyl (2S)-4-{3-carbamoyl-4-[(4-methoxyphenyl)methyl]-2-(4-phenoxyphenyl)-4,5,6,7-tetrahydro-2H-pyrazolo[4,3-b]pyridin-7-yl}-2-methylpiperazine-1-carboxylate. MS (ESI) m/z: 453.0 [M+H]⁺.

### Step 52.10

### 2-(2-chloro-4-phenoxyphenyl)-7-[4-(prop-2-enoyl)piperazin-1-yl]-4,5,6,7-tetrahydro-2H-pyrazolo[4,3-b]pyridine-3-carboxamide

Step 52.10 was prepared according to the procedure for Step 43.3, substituting 2-(2-chloro-4-phenoxyphenyl)-7-(piperazin-1-yl)-4,5,6,7-tetrahydro-2H-pyrazolo[4,3-b]pyridine-3-carboxamide (Step 52.9) for 7-[(3S)-3-methylpiperazin-1-yl]-2-(4-phenoxyphenyl)-4,5,6,7-tetrahydro-2H-pyrazolo[4,3-b]pyridine-3-carboxamide. ¹H NMR (400 MHz, dimethyl sulfoxide-d₆) δ ppm 7.51-7.43 (m, 2H), 7.40 (d, J=8.7 Hz, 1H), 7.24 (tt, J=7.4, 1.1 Hz, 1H), 7.15 (dd, J=7.5, 1.8 Hz, 3H), 6.99 (dd, J=8.7, 2.7 Hz, 1H), 6.78 (dd, J=16.7, 10.4 Hz, 1H), 6.08 (dd, J=16.7, 2.4 Hz, 1H), 5.65 (dd, J=10.4, 2.5 Hz, 1H), 5.17 (d, J=3.8 Hz, 1H), 3.67 (t, J=5.6 Hz, 1H), 3.49 (d, J=22.2 Hz, 4H), 3.27 (s, 1H), 3.11 (d, J=11.6 Hz, 1H), 2.67 (s, 1H), 2.58 (s, 3H), 2.06 (d, J=13.4 Hz, 1H), 1.76 (d, J=6.1 Hz, 1H). MS (ESI) m/z: 507.1 [M+H]⁺.

### Example 53

### 2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide

Example 53 was prepared according to the procedure for Example 43, substituting tert-butyl 4,7-diazaspiro[2.5]octane-4-carboxylate for (S)-tert-butyl 2-methylpiperazine-1-carboxylate in Step 43.1. ¹H NMR (400 MHz, dimethyl sulfoxide-d₆) δ ppm 7.50-7.39 (m, 2H), 7.39-7.28 (m, 2H), 7.21-7.13 (m, 1H), 7.05 (ddd, J=13.3, 7.7, 1.7 Hz, 4H), 6.10 (d, J=16.8 Hz, 1H), 5.68 (d, J=10.4 Hz, 1H), 5.08 (s, 1H), 3.61 (s, 1H), 3.20-3.28 (m, 1H), 3.10 (s, 1H), 2.12-1.94 (m, 1H), 1.70 (s, 1H). MS (ESI) m/z: 499.26 [M+H]⁺.

### Example 54

### 7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide

### Step 54.1

### ethyl 7-[4-(tert-butoxycarbonyl)piperazin-1-yl]-2-{4-[2-(trifluoromethyl)phenoxy]phenyl}-4,5,6,7-tetrahydro-2H-pyrazolo[4,3-b]pyridine-3-carboxylate

Step 54.1 was prepared according to the procedure for Step 78.1, substituting ethyl 7-[4-(tert-butoxycarbonyl)piperazin-1-yl]-2-(4-hydroxyphenyl)-4,5,6,7-tetrahydro-2H-pyrazolo[4,3-b]pyridine-3-carboxylate (Step 57.2) for ethyl (7R)-7-[4-(2-nitrobenzene-1-sulfonyl)piperazin-1-yl]-4,5,6,7-tetrahydro-2H-pyrazolo[4,3-b]pyridine-3-carboxylate and [2-(trifluoromethyl)phenyl]boronic acid for [4-(3-cyclopropylphenoxy)phenyl]boronic acid. MS (ESI) m/z: 615.8 [M+H]⁺.

### Step 54.2

### 7-[4-(tert-butoxycarbonyl)piperazin-1-yl]-2-{4-[2-(trifluoromethyl)phenoxy]phenyl}-4,5,6,7-tetrahydro-2H-pyrazolo[4,3-b]pyridine-3-carboxylic acid

Step 54.2 was prepared according to the procedure for Step 78.2, substituting ethyl 7-[4-(tert-butoxycarbonyl)piperazin-1-yl]-2-{4-[2-(trifluoromethyl)phenoxy]phenyl}-4,5,6,7-tetrahydro-2H-pyrazolo[4,3-b]pyridine-3-carboxylate (Step 54.1) for ethyl (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate. MS (ESI) m/z: 588.0 [M+H]⁺.

### Step 54.3

### tert-butyl 4-(3-carbamoyl-2-{4-[2-(trifluoromethyl)phenoxy]phenyl}-4,5,6,7-tetrahydro-2H-pyrazolo[4,3-b]pyridin-7-yl)piperazine-1-carboxylate

To a solution of 7-[4-(tert-butoxycarbonyl)piperazin-1-yl]-2-{4-[2-(trifluoromethyl)phenoxy]phenyl}-4,5,6,7-tetrahydro-2H-pyrazolo[4,3-b]pyridine-3-carboxylic acid (0.120 g, 0.204 mmol, Step 54.2), ammonium chloride (0.022 g, 0.408 mmol), 1-hydroxybenzotriazole hydrate (0.034 g, 0.225 mmol), N-(3-dimethylaminopropyl)-N-ethylcarbodiimide hydrochloride (0.059 g, 0.306 mmol) in N,N-dimethylformamide (2.0 mL) was added triethylamine (0.085 mL, 0.613 mmol) and the mixture was stirred at ambient temperature. After 23 hours, the reaction mixture was diluted with water and extracted with ethyl acetate (3×). The combined organic layers were washed with brine, dried over Na₂SO₄, filtered, and concentrated under reduced pressure. The crude residue was purified by column chromatography (0% to 100% ethyl acetate in heptanes) to afford the title compound (0.080 g, 67%). MS (ESI) m/z: 587.0 [M+H]⁺.

### Step 54.4

### 7-(piperazin-1-yl)-2-{4-[2-(trifluoromethyl)phenoxy]phenyl}-4,5,6,7-tetrahydro-2H-pyrazolo[4,3-b]pyridine-3-carboxamide

To a solution of tert-butyl 4-(3-carbamoyl-2-{4-[2-(trifluoromethyl)phenoxy]phenyl}-4,5,6,7-tetrahydro-2H-pyrazolo[4,3-b]pyridin-7-yl)piperazine-1-carboxylate (0.080 g, 0.136 mmol, Step 54.3) in dichloromethane (2.7 mL) was added 4 M HCl in 1,4-dioxane (0.512 mL, 2.046 mmol) and the mixture was stirred at ambient temperature. After 3 hours, the reaction mixture was concentrated under reduced pressure, then azeotropically dried from toluene to afford the title compound that was used without purification. MS (ESI) m/z: 487.0 [M+H]⁺.

### Step 54.5

### 7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide

Step 54.5 was prepared according to the procedure for Step 78.5, substituting 7-(piperazin-1-yl)-2-{4-[2-(trifluoromethyl)phenoxy]phenyl}-4,5,6,7-tetrahydro-2H-pyrazolo[4,3-b]pyridine-3-carboxamide (Step 54.4) for (7R)-2-[4-(3-cyclopropylphenoxy)phenyl]-7-(piperazin-1-yl)-4,5,6,7-tetrahydro-2H-pyrazolo[4,3-b]pyridine-3-carboxamide. ¹H NMR (600 MHz, CDCl₃) δ ppm 7.71 (dd, J=7.8, 1.6 Hz, 1H), 7.52 (tdd, J=7.5, 1.8, 0.9 Hz, 1H), 7.47-7.42 (m, 2H), 7.25 (t, J=7.7 Hz, 1H), 7.13-7.10 (m, 2H), 7.03 (d, J=8.3 Hz, 1H), 6.55 (dd, J=16.8, 10.6 Hz, 1H), 6.28 (dd, J=16.8, 1.9 Hz, 1H), 5.67 (dd, J=10.6, 1.9 Hz, 1H), 5.22 (s, 2H), 5.13 (s, 1H), 3.86 (dd, J=6.8, 5.0 Hz, 1H), 3.72 (d, J=69.0 Hz, 2H), 3.58 (s, 2H), 3.38 (dt, J=81.3, 9.7 Hz, 2H), 2.83-2.65 (m, 4H), 2.23-1.93 (m, 2H). MS (ESI) m/z: 541.1 [M+H]⁺.

### Example 55

### 2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide

### Step 55.1

### ethyl 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylate

To a solution of pyridine (0.093 mL, 1.14 mmol), [4-(3,5-difluorophenoxy)phenyl]boronic acid (0.286 g, 1.14 mmol, Intermediate X), and ethyl 7-[4-(tert-butoxycarbonyl)piperazin-1-yl]-4,5,6,7-tetrahydro-2H-pyrazolo[4,3-b]pyridine-3-carboxylate (0.217 g, 0.572 mmol, Intermediate V) in dichloromethane (11 mL) was added copper(II) acetate (0.156 g, 0.858 mmol) at ambient temperature. The reaction mixture was stirred for 16 hours under an atmosphere of air. Saturated aqueous NH₄Cl was added, and the resulting solution was filtered through diatomaceous earth. The filtrate was extracted with dichloromethane (3×). The combined organic layers were concentrated under reduced pressure, and the crude product was purified by silica gel chromatography (0-100% ethyl acetate in heptanes) to provide the title compound (0.168 g, 50%). MS (ESI) m/z: 583.7 [M+H]⁺.

### Step 55.2

### 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylic acid

To a solution of ethyl 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylate (0.168 g, 0.288 mmol, Step 55.1) in 4:4:1 tetrahydrofuran/methanol/water (2.7 mL) was added LiOH (0.069 g, 2.9 mmol). The reaction mixture was warmed to 50° C. and stirred for 2 hours. The reaction mixture was cooled to ambient temperature and acidified with 1 M aqueous HCl. The resulting solution was extracted with ethyl acetate (3×). The combined organic layers were dried over MgSO₄, filtered, and concentrated under reduced pressure to provide the title compound (0.172 g, 100%), which was used without further purification. MS (ESI) m/z: 556.0 [M+H]⁺.

### Step 55.3

### tert-butyl 4-{3-carbamoyl-2-[4-(3,5-difluorophenoxy)phenyl]-4,5,6,7-tetrahydro-2H-pyrazolo[4,3-b]pyridin-7-yl}piperazine-1-carboxylate

To a solution of 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylic acid (0.171 g, 0.308 mmol, Step 55.2), N,N-diisopropylethylamine (0.161 mL, 0.923 mmol, Step 55.2), 1-hydroxybenzotriazole hydrate (0.052 g, 0.34 mmol), and NH₄Cl (0.033 g, 0.62 mmol) in N,N-dimethylformamide (3 mL) was added 1-(3-dimethylaminopropyl)-3-ethylcarbodiimide hydrochloride (0.089 g, 0.462 mmol) at ambient temperature. The reaction mixture was stirred for 1 hour. The reaction mixture was quenched by the addition of saturated aqueous NaHCO₃. The aqueous solution was extracted with ethyl acetate (3×). The combined organic layers were washed with brine (3×) and concentrated under reduced pressure. The crude product was purified by silica gel chromatography (0-10% methanol in dichloromethane) to afford the title compound (0.094 g, 55%). MS (ESI) m/z: 555.1 [M+H]⁺.

### Step 55.4

### 2-[4-(3,5-difluorophenoxy)phenyl]-7-(piperazin-1-yl)-4,5,6,7-tetrahydro-2H-pyrazolo[4,3-b]pyridine-3-carboxamide-2H-pyrazolo[4,3-b]pyridine-3-carboxamide

Step 55.4 was prepared according to the procedure for Step 12.3, substituting tert-butyl 4-{3-carbamoyl-2-[4-(3,5-difluorophenoxy)phenyl]-4,5,6,7-tetrahydro-2H-pyrazolo[4,3-b]pyridin-7-yl}piperazine-1-carboxylate (Step 55.3) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. MS (ESI) m/z: 455.1 [M+H]⁺.

### Step 55.5

### 2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide

To a solution of acrylic acid (0.013 mL, 0.19 mmol), N,N-diisopropylethylamine (0.295 mL, 1.69 mmol), and 2-[4-(3,5-difluorophenoxy)phenyl]-7-(piperazin-1-yl)-4,5,6,7-tetrahydro-2H-pyrazolo[4,3-b]pyridine-3-carboxamide (0.077 g, 0.17 mmol, Step 55.4) in dichloromethane (3 mL) was added 1-propanephosphonic anhydride (0.12 mL, 0.20 mmol, 50% in N,N-dimethylformamide) at ambient temperature. The reaction mixture was stirred for 15 minutes. The reaction mixture was quenched by the addition of methanol. The resulting solution was concentrated under reduced pressure. The crude product was purified by silica gel chromatography (0-20% methanol in dichloromethane) to provide the title compound (0.054 g, 0.11 mmol, 62%). ¹H NMR (400 MHz, dimethyl sulfoxide-d₆) δ ppm 7.40-7.32 (m, 2H), 7.20-7.09 (m, 2H), 6.98 (tt, J=9.3, 2.3 Hz, 1H), 6.81-6.69 (m, 3H), 6.06 (dd, J=16.6, 2.4 Hz, 1H), 5.62 (dd, J=10.5, 2.4 Hz, 1H), 5.13-5.07 (m, 1H), 3.63 (t, J=5.5 Hz, 1H), 3.58-3.35 (m, 5H), 3.27-3.18 (m, 1H), 3.14-3.05 (m, 1H), 2.70-2.49 (m, 3H), 2.09-2.01 (m, 1H), 1.77-1.66 (m, 1H). MS (ESI) m/z: 509.1 [M+H]⁺.

### Example 56

### 2-[4-(4-fluorophenoxy)phenyl]-7-[4-(prop-2-enoyl)piperazin-1-yl]-4,5,6,7-tetrahydro-2H-pyrazolo[4,3-b]pyridine-3-carboxamide

### Step 56.1

### ethyl 7-[4-(tert-butoxycarbonyl)piperazin-1-yl]-2-[4-(4-fluorophenoxy)phenyl]-2H-pyrazolo[4,3-b]pyridine-3-carboxylate

Step 56.1 was prepared according to the procedure for Step 55.1, substituting [4-(4-fluorophenoxy)phenyl]boronic acid for [4-(3,5-difluorophenoxy)phenyl]boronic acid in the presence of 4 Å molecular sieves (0.75 g). MS (APCI) m/z: 548.4 [M+H]⁺.

### Step 56.2

### tert-butyl 4-{3-carbamoyl-2-[4-(4-fluorophenoxy)phenyl]-2H-pyrazolo[4,3-b]pyridin-7-yl}piperazine-1-carboxylate

Ethyl 7-[4-(tert-butoxycarbonyl)piperazin-1-yl]-2-[4-(4-fluorophenoxy)phenyl]-2H-pyrazolo[4,3-b]pyridine-3-carboxylate (0.18 g, 0.321 mmol, Step 56.1) was added to a 20 mL vial, purged with nitrogen for 1 minute, and then dissolved in 7 M ammonia in methanol (3.0 mL, 21.00 mmol). The reaction was heated at 75° C. for 5 hours. The reaction was cooled to ambient temperature, and additional 7 M ammonia in methanol (3.0 mL, 21.00 mmol) was added. The reaction was heated to 65° C. for about 16 hours, then cooled to ambient temperature and concentrated under reduced pressure to afford the title compound. MS (APCI) m/z: 533.4 [M+H]⁺.

### Step 56.3

### tert-butyl 4-{3-carbamoyl-2-[4-(4-fluorophenoxy)phenyl]-4,5,6,7-tetrahydro-2H-pyrazolo[4,3-b]pyridin-7-yl}piperazine-1-carboxylate

Step 56.3 was prepared according to the procedure for Step 36.2, tert-butyl 4-{3-carbamoyl-2-[4-(4-fluorophenoxy)phenyl]-2H-pyrazolo[4,3-b]pyridin-7-yl}piperazine-1-carboxylate (Step 56.2) for tert-butyl 4-{3-carbamoyl-2-[4-(2,4-difluorophenoxy)phenyl]-2H-pyrazolo[4,3-b]pyridin-7-yl}piperazine-1-carboxylate and the reactor pressurized to 60 psi with hydrogen. MS (APCI) m/z: 537.4 [M+H]⁺.

### Step 56.4

### 2-[4-(4-fluorophenoxy)phenyl]-7-(piperazin-1-yl)-4,5,6,7-tetrahydro-2H-pyrazolo[4,3-b]pyridine-3-carboxamide

tert-Butyl 4-{3-carbamoyl-2-[4-(4-fluorophenoxy)phenyl]-4,5,6,7-tetrahydro-2H-pyrazolo[4,3-b]pyridin-7-yl}piperazine-1-carboxylate (0.077 g, 0.143 mmol, Step 56.3) was dissolved in 1,4-dioxane (3.0 mL). 4 M HCl in 1,4-dioxane (0.717 mL, 2.87 mmol) was added dropwise while stirring at ambient temperature. The reaction was stirred for about 23 hours and concentrated under reduced pressure. The residue was dissolved in methanol and stirred with MP-carbonate resin (0.2 g, 0.574 mmol) at ambient temperature for 1 hour. The mixture was filtered, and the filtrate was concentrated under reduced pressure to afford the title compound (0.060 g). MS (APCI) m/z: 437.4 [M+H]⁺.

### Step 56.5

### 2-[4-(4-fluorophenoxy)phenyl]-7-[4-(prop-2-enoyl)piperazin-1-yl]-4,5,6,7-tetrahydro-2H-pyrazolo[4,3-b]pyridine-3-carboxamide

Step 56.5 was prepared according to the procedure for Step 16.8, 2-[4-(4-fluorophenoxy)phenyl]-7-(piperazin-1-yl)-4,5,6,7-tetrahydro-2H-pyrazolo[4,3-b]pyridine-3-carboxamide (Step 56.4) for 2-(4-phenoxyphenyl)-7-(piperidin-4-yl)-4,5,6,7-tetrahydro-2H-pyrazolo[3,4-b]pyrazine-3-carboxamide and cooling the reaction to −78° C. ¹H NMR (500 MHz, dimethyl sulfoxide-d₆) δ ppm 7.38-7.30 (m, 2H), 7.30-7.20 (m, 2H), 7.16-7.08 (m, 2H), 7.07-6.96 (m, 2H), 6.78 (dd, J=16.7, 10.5 Hz, 1H), 6.09 (dd, J=16.7, 2.4 Hz, 1H), 5.66 (dd, J=10.5, 2.4 Hz, 1H), 5.10 (t, J=3.0 Hz, 1H), 3.65 (dd, J=6.2, 4.8 Hz, 1H), 3.45 (q, J=6.9, 6.3 Hz, 4H), 3.25 (ddt, J=8.7, 5.6, 2.9 Hz, 1H), 3.18-3.05 (m, 1H), 2.68 (d, J=8.7 Hz, 1H), 2.62-2.52 (m, 3H), 2.07 (ddt, J=13.6, 6.8, 4.2 Hz, 1H), 1.74 (tq, J=8.0, 4.2, 3.7 Hz, 1H). MS (APCI) m/z: 491.4 [M+H]⁺.

### Example 57

### 2-[4-(3-fluorophenoxy)phenyl]-7-[4-(prop-2-enoyl)piperazin-1-yl]-4,5,6,7-tetrahydro-2H-pyrazolo[4,3-b]pyridine-3-carboxamide

### Step 57.1

### ethyl 7-[4-(tert-butoxycarbonyl)piperazin-1-yl]-2-(4-hydroxyphenyl)-2H-pyrazolo[4,3-b]pyridine-3-carboxylate

To a flask equipped with a stir bar were added [(2-di-tert-butylphosphino-3-methoxy-6-methyl-2′,4′,6′-triisopropyl-1,1′-biphenyl)-2-(2-aminobiphenyl)]palladium(II) methanesulfonate (0.351 g, 0.419 mmol), Cs₂CO₃ (8.18 g, 25.1 mmol), and ethyl 2-(4-bromophenyl)-7-[4-(tert-butoxycarbonyl)piperazin-1-yl]-2H-pyrazolo[4,3-b]pyridine-3-carboxylate (4.44 g, 8.37 mmol). The flask was sealed and purged with N₂ (3×). N,N-Dimethylformamide (100 mL) and water (0.452 mL, 25.1 mmol) were added. The reaction mixture was warmed to 80° C. and stirred for 16 hours. The reaction mixture cooled to ambient temperature and quenched by the addition of saturated aqueous NH₄Cl. The resulting aqueous solution was extracted with ethyl acetate (3×). The combined organic layers were washed with brine (3×) and concentrated under reduced pressure. The crude product was purified by silica gel chromatography (0-100% ethyl acetate in heptanes) to provide the title compound (1.10 g, 2.35 mmol, 28%). MS (ESI) m/z: 468.1 [M+H]⁺.

### Step 57.2

### ethyl 7-[4-(tert-butoxycarbonyl)piperazin-1-yl]-2-(4-hydroxyphenyl)-4,5,6,7-tetrahydro-2H-pyrazolo[4,3-b]pyridine-3-carboxylate

To a Parr reactor were added ethyl 7-[4-(tert-butoxycarbonyl)piperazin-1-yl]-2-(4-hydroxyphenyl)-2H-pyrazolo[4,3-b]pyridine-3-carboxylate (1.10 g, 2.35 mmol, Step 57.1), 10% Pd(OH)₂/C (0.661 g, 2.35 mmol) and 4:1 tetrahydrofuran/methanol (10 mL). The vessel was pressurized with H₂ (100 psi) and warmed to 50° C. The reaction mixture was stirred for 24 hours at ambient temperature. The vessel was cooled to ambient temperature and vented. The reaction mixture was filtered over diatomaceous earth, and the filtrate was concentrated under reduced pressure. The crude product was purified by silica gel chromatography (0-100% ethyl acetate in heptanes) to provide the title compound (0.730 g, 66%). MS m/z: 472.1 [M+H]⁺.

### Step 57.3

### ethyl 7-[4-(tert-butoxycarbonyl)piperazin-1-yl]-2-[4-(3-fluorophenoxy)phenyl]-4,5,6,7-tetrahydro-2H-pyrazolo[4,3-b]pyridine-3-carboxylate

Step 57.3 was prepared according to the procedure for Step 55.1, substituting (3-fluorophenyl)boronic acid for [4-(3,5-difluorophenoxy)phenyl]boronic acid, and ethyl 7-[4-(tert-butoxycarbonyl)piperazin-1-yl]-2-(4-hydroxyphenyl)-4,5,6,7-tetrahydro-2H-pyrazolo[4,3-b]pyridine-3-carboxylate (Step 57.2) for ethyl 7-[4-(tert-butoxycarbonyl)piperazin-1-yl]-4,5,6,7-tetrahydro-2H-pyrazolo[4,3-b]pyridine-3-carboxylate. The crude product was purified by silica gel chromatography (0-100% ethyl acetate in heptanes) to provide the title compound (0.331 g, 55%). MS (ESI) m/z: 566.1 [M+H]⁺.

### Step 57.4

### 7-[4-(tert-butoxycarbonyl)piperazin-1-yl]-2-[4-(3-fluorophenoxy)phenyl]-4,5,6,7-tetrahydro-2H-pyrazolo[4,3-b]pyridine-3-carboxylic acid

Step 57.4 was prepared according to the procedure for Step 55.2, substituting ethyl 7-[4-(tert-butoxycarbonyl)piperazin-1-yl]-2-[4-(3-fluorophenoxy)phenyl]-4,5,6,7-tetrahydro-2H-pyrazolo[4,3-b]pyridine-3-carboxylate (Step 57.3) for ethyl 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylate. MS (ESI) m/z: 538.1 [M+H]⁺.

### Step 57.5

### tert-butyl 4-{3-carbamoyl-2-[4-(3-fluorophenoxy)phenyl]-4,5,6,7-tetrahydro-2H-pyrazolo[4,3-b]pyridin-7-yl}piperazine-1-carboxylate

To a solution of 7-[4-(tert-butoxycarbonyl)piperazin-1-yl]-2-[4-(3-fluorophenoxy)phenyl]-4,5,6,7-tetrahydro-2H-pyrazolo[4,3-b]pyridine-3-carboxylic acid (0.233 g, 0.434 mmol, Step 57.4), N,N-diisopropylethylamine (0.227 mL, 1.30 mmol), 1-hydroxybenzotriazole hydrate (0.073 g, 0.48 mmol), and NH₄Cl (0.046 g, 0.87 mmol) in N,N-dimethylformamide (4 mL) was added (1-(3-dimethylaminopropyl)-3-ethylcarbodiimide hydrochloride) (0.125 g, 0.650 mmol) at ambient temperature. The reaction mixture was stirred for 1 hour, and then quenched by the addition of saturated aqueous NH₄Cl. The aqueous solution was extracted with ethyl acetate (3×). The combined organic layers were washed with brine (3×) and concentrated under reduced pressure. The crude product was purified by silica gel chromatography (0-100% ethyl acetate in heptanes) to afford the title compound (0.122 g, 52%). MS (ESI) m/z: 537.1 [M+H]⁺.

### Step 57.6

### 2-[4-(3-fluorophenoxy)phenyl]-7-(piperazin-1-yl)-4,5,6,7-tetrahydro-2H-pyrazolo[4,3-b]pyridine-3-carboxamide

Step 57.6 was prepared according to the procedure for Step 12.3, substituting tert-butyl 4-{3-carbamoyl-2-[4-(3-fluorophenoxy)phenyl]-4,5,6,7-tetrahydro-2H-pyrazolo[4,3-b]pyridin-7-yl}piperazine-1-carboxylate (Step 57.5) for tert-butyl 4-{3-carbamoyl-2-[4-(3-fluorophenoxy)phenyl]-4,5,6,7-tetrahydro-2H-pyrazolo[4,3-b]pyridin-7-yl}piperidine-1-carboxylate. MS (ESI) m/z: 437.15 [M+H]⁺.

### Step 57.7

### 2-[4-(3-fluorophenoxy)phenyl]-7-[4-(prop-2-enoyl)piperazin-1-yl]-4,5,6,7-tetrahydro-2H-pyrazolo[4,3-b]pyridine-3-carboxamide

To a solution of acrylic acid (0.017 mL, 0.25 mmol), N,N-diisopropylethylamine (0.396 mL, 2.27 mmol), and 2-[4-(3-fluorophenoxy)phenyl]-7-(piperazin-1-yl)-4,5,6,7-tetrahydro-2H-pyrazolo[4,3-b]pyridine-3-carboxamide (0.099 g, 0.23 mmol, Step 57.6) in dichloromethane (2.5 mL) was added 1-propanephosphonic anhydride (0.173 g, 0.272 mmol) at ambient temperature. The reaction mixture was stirred for 15 minutes, and then quenched by the addition of methanol. The resulting solution was concentrated under reduced pressure. The crude product was purified by silica gel chromatography (0-10% methanol in dichloromethane) to provide the title compound (0.079 g, 71%). ¹H NMR (400 MHz, CDCl₃) δ ppm 7.45 (d, J=8.7 Hz, 2H), 7.39-7.29 (m, 1H), 7.13 (d, J=8.8 Hz, 2H), 6.93-6.82 (m, 2H), 6.82-6.74 (m, 1H), 6.56 (dd, J=16.8, 10.5 Hz, 1H), 6.28 (dd, J=16.8, 2.0 Hz, 1H), 5.69 (dd, J=10.5, 1.9 Hz, 1H), 5.41 (s, 2H), 5.13 (s, 1H), 3.99-3.52 (m, 5H), 3.51-3.42 (m, 1H), 3.37-3.28 (m, 1H), 2.97-2.56 (m, 4H), 2.30-2.11 (m, 1H), 2.09-1.89 (m, 1H). MS (ESI) m/z: 491.1 [M+H]⁺.

### Example 58

### (7R)-2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide; and

### Example 59

### (7S)-2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide

The title compounds were obtained by separating the enantiomers of 2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide (Example 55) with preparative supercritical fluid chromatography purification. Preparative supercritical fluid chromatography was performed on a THAR/Waters SFC 80 system running under SuperChrom software control. The preparative supercritical fluid chromatography system was equipped with an 8-way preparative column switcher, CO₂ pump, modifier pump, automated back pressure regulator (ABPR), UV detector, and 6-position fraction collector. The mobile phase was comprised of supercritical CO₂ supplied by a dewar of bone-dry non-certified CO₂ pressurized to 350 psi with a modifier of methanol at a flow rate of 80 g/minute. The column was at ambient temperature and the backpressure regulator was set to maintain 100 bar. The sample was dissolved in methanol at a concentration of 15 mg/mL. The sample was loaded into the modifier stream in 2 mL (30 mg) injections. The mobile phase was held isocratically at 40% methanol:CO₂. Fraction collection was threshold triggered. The instrument was fitted with a ChiralCel® OJ-H column with dimensions 30 mm i.d.×250 mm length with 5 μm particles. The peak that eluted second was concentrated under reduced pressure to provide Example 58: ¹H NMR (500 MHz, CDCl₃) δ ppm 7.52-7.45 (m, 2H), 7.22-7.11 (m, 2H), 6.64-6.52 (m, 4H), 6.29 (dd, J=16.8, 1.9 Hz, 1H), 5.69 (dd, J=10.6, 1.9 Hz, 1H), 5.43 (s, 2H), 5.11 (s, 1H), 4.01-3.53 (m, 5H), 3.51-3.43 (m, 1H), 3.37-3.29 (m, 1H), 3.01-2.58 (m, 4H), 2.26-2.17 (m, 1H), 2.11-1.88 (m, 1H). MS (ESI) m/z: 509.1 [M+H]⁺. The peak that eluted first was concentrated under reduced pressure to provide Example 59: ¹H NMR (400 MHz, dimethyl sulfoxide-d₆) δ ppm 7.40-7.32 (m, 2H), 7.20-7.09 (m, 2H), 6.97 (tt, J=9.3, 2.3 Hz, 1H), 6.80-6.68 (m, 3H), 6.05 (dd, J=16.6, 2.4 Hz, 1H), 5.62 (dd, J=10.5, 2.4 Hz, 1H), 5.13-5.07 (m, 1H), 3.66-3.59 (m, 1H), 3.57-3.36 (m, 4H), 3.28-3.18 (m, 1H), 3.14-3.04 (m, 1H), 2.71-2.49 (m, 4H), 2.06 (s, 1H), 1.76-1.66 (m, 1H). MS (ESI) m/z: 509.1 [M+H]⁺.

### Example 60

### (7R)-7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide; and

### Example 61

### (7S)-7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide

Racemic 7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide (Example 54) was separated by preparative chiral supercritical fluid chromatography (ChiralPakAD-H, 250×30 mm; column temperature 35° C.; Mobile phase: (A) CO₂ and (B) methanol; Gradient B % 35%). Each set of peaks were pooled and re-purified by column chromatography (0 to 5% methanol in dichloromethane) to afford Examples 60 and 61. Example 60: ¹H NMR (600 MHz, CDCl₃) δ ppm 7.71 (dd, J=7.8, 1.6 Hz, 1H), 7.52 (tdd, J=7.5, 1.8, 0.9 Hz, 1H), 7.47-7.42 (m, 2H), 7.25 (t, J=7.7 Hz, 1H), 7.13-7.10 (m, 2H), 7.03 (d, J=8.3 Hz, 1H), 6.55 (dd, J=16.8, 10.6 Hz, 1H), 6.28 (dd, J=16.8, 1.9 Hz, 1H), 5.67 (dd, J=10.6, 1.9 Hz, 1H), 5.22 (s, 2H), 5.13 (s, 1H), 3.86 (dd, J=6.8, 5.0 Hz, 1H), 3.72 (d, J=69.0 Hz, 2H), 3.58 (s, 2H), 3.38 (dt, J=81.3, 9.7 Hz, 2H), 2.83-2.65 (m, 4H), 2.23-1.93 (m, 2H). MS (ESI) m/z: 541.1 [M+H]⁺. [α]D: −45°. Example 61: ¹H NMR (600 MHz, CDCl₃) δ ppm 7.71 (dd, J=7.8, 1.6 Hz, 1H), 7.52 (tdd, J=7.5, 1.8, 0.9 Hz, 1H), 7.47-7.42 (m, 2H), 7.25 (t, J=7.7 Hz, 1H), 7.13-7.10 (m, 2H), 7.03 (d, J=8.3 Hz, 1H), 6.55 (dd, J=16.8, 10.6 Hz, 1H), 6.28 (dd, J=16.8, 1.9 Hz, 1H), 5.67 (dd, J=10.6, 1.9 Hz, 1H), 5.22 (s, 2H), 5.13 (s, 1H), 3.86 (dd, J=6.8, 5.0 Hz, 1H), 3.72 (d, J=69.0 Hz, 2H), 3.58 (s, 2H), 3.38 (dt, J=81.3, 9.7 Hz, 2H), 2.83-2.65 (m, 4H), 2.23-1.93 (m, 2H). MS (ESI) m/z: 541.1 [M+H]⁺. [α]D: +43°.

### Example 62

### 2-[4-(3-fluorophenoxy)phenyl]-7-[1-(prop-2-enoyl)azetidin-3-yl]-4,5,6,7-tetrahydro-2H-pyrazolo[3,4-b]pyrazine-3-carboxamide

### Step 62.1

### tert-butyl 3-[{5-carbamoyl-1-[4-(3-fluorophenoxy)phenyl]-4-nitro-1H-pyrazol-3-yl}(prop-2-en-1-yl)amino]azetidine-1-carboxylate

Step 62.1 was prepared according to the procedure for Step 81.1, substituting [4-(3-fluorophenoxy)phenyl]boronic acid (Intermediate M) for [4-(2,4-difluorophenoxy)phenyl]boronic acid. ¹H NMR (400 MHz, dimethyl sulfoxide-d₆) δ ppm 1.36 (s, 9H) 3.75-3.85 (m, 2H) 3.89-4.08 (m, 5H) 4.25-4.36 (m, 1H) 5.11-5.25 (m, 2H) 5.73-5.89 (m, 1H) 6.91 (br d, J=8.16 Hz, 1H) 6.95-7.10 (m, 2H) 7.21 (d, J=9.04 Hz, 2H) 7.47 (d, J=7.28 Hz, 1H) 7.59 (d, J=9.04 Hz, 2H) 8.22 (s, 1H) 8.46 (s, 1H).

### Step 62.2

### tert-butyl 3-[{5-carbamoyl-1-[4-(3-fluorophenoxy)phenyl]-4-nitro-1H-pyrazol-3-yl}(2-oxoethyl)amino]azetidine-1-carboxylate

Step 62.2 was prepared according to the procedure for Step 81.2, substituting tert-butyl 3-[{5-carbamoyl-1-[4-(3-fluorophenoxy)phenyl]-4-nitro-1H-pyrazol-3-yl}(prop-2-en-1-yl)amino]azetidine-1-carboxylate (Step 62.1) for tert-butyl 3-[{5-carbamoyl-1-[4-(2,4-difluorophenoxy)phenyl]-4-nitro-1H-pyrazol-3-yl}(prop-2-en-1-yl)amino]azetidine-1-carboxylate. MS (ESI) m/z: 499.0 [M-OtButyl+H₂O]⁺.

### Step 62.3

### tert-butyl 3-{3-carbamoyl-2-[4-(3-fluorophenoxy)phenyl]-2,4,5,6-tetrahydro-7H-pyrazolo[3,4-b]pyrazin-7-yl}azetidine-1-carboxylate

Step 62.3 was prepared according to the procedure for Step 81.3, substituting tert-butyl 3-[{5-carbamoyl-1-[4-(3-fluorophenoxy)phenyl]-4-nitro-1H-pyrazol-3-yl}(2-oxoethyl)amino]azetidine-1-carboxylate (Step 62.2) for tert-butyl 3-[{5-carbamoyl-1-[4-(2,4-difluorophenoxy)phenyl]-4-nitro-1H-pyrazol-3-yl}(2-oxoethyl)amino]azetidine-1-carboxylate. MS (ESI) m/z: 509.4 [M+H]⁺.

### Step 62.4

### 7-(azetidin-3-yl)-2-[4-(3-fluorophenoxy)phenyl]-4,5,6,7-tetrahydro-2H-pyrazolo[3,4-b]pyrazine-3-carboxamide-hydrogen chloride (1/3)

Step 62.4 was prepared according to the procedure for Step 81.4, substituting tert-butyl 3-{3-carbamoyl-2-[4-(3-fluorophenoxy)phenyl]-2,4,5,6-tetrahydro-7H-pyrazolo[3,4-b]pyrazin-7-yl}azetidine-1-carboxylate (Step 62.3) for tert-butyl 3-{3-carbamoyl-2-[4-(2,4-difluorophenoxy)phenyl]-2,4,5,6-tetrahydro-7H-pyrazolo[3,4-b]pyrazin-7-yl}azetidine-1-carboxylate. MS (ESI) m/z: 409. [M+H]⁺.

### Step 62.5

### 2-[4-(3-fluorophenoxy)phenyl]-7-[1-(prop-2-enoyl)azetidin-3-yl]-4,5,6,7-tetrahydro-2H-pyrazolo[3,4-b]pyrazine-3-carboxamide

Step 62.5 was prepared according to the procedure for Step 35.4, substituting 7-(azetidin-3-yl)-2-[4-(3-fluorophenoxy)phenyl]-4,5,6,7-tetrahydro-2H-pyrazolo[3,4-b]pyrazine-3-carboxamide-hydrogen chloride (1/3) (Step 62.4) for 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide. ¹H NMR (400 MHz, methanol-d₄) δ ppm 3.28 (br d, J=4.85 Hz, 2H) 3.50 (br d, J=4.63 Hz, 2H) 4.21-4.45 (m, 3H) 4.47-4.65 (m, 2H) 5.73 (dd, J=10.25, 1.65 Hz, 1H) 6.17-6.28 (m, 1H) 6.30-6.43 (m, 1H) 6.71-6.91 (m, 3H) 6.98-7.15 (m, 2H) 7.29-7.45 (m, 3H). MS (ESI) m/z: 463.1 [M+H]⁺.

### Example 63

### 2-[4-(3,4-difluorophenoxy)phenyl]-7-[1-(prop-2-enoyl)azetidin-3-yl]-4,5,6,7-tetrahydro-2H-pyrazolo[3,4-b]pyrazine-3-carboxamide

### Step 63.1

### tert-butyl 3-[{5-carbamoyl-1-[4-(3,4-difluorophenoxy)phenyl]-4-nitro-1H-pyrazol-3-yl}(prop-2-en-1-yl)amino]azetidine-1-carboxylate

Step 63.1 was prepared according to the procedure for Step 81.1, substituting [4-(3,4-difluorophenoxy)phenyl]boronic acid (Intermediate N) for [4-(2,4-difluorophenoxy)phenyl]boronic acid. ¹H NMR (400 MHz, dimethyl sulfoxide-d₆) δ ppm 1.36 (s, 9H) 3.74-3.85 (m, 2H) 3.89-4.09 (m, 5H) 4.30 (br t, J=6.06 Hz, 1H) 5.11-5.24 (m, 2H) 5.81 (br dd, J=16.98, 10.14 Hz, 1H) 6.96 (br d, J=9.26 Hz, 1H) 7.18 (d, J=8.82 Hz, 2H) 7.29-7.40 (m, 1H) 7.51 (d, J=9.92 Hz, 1H) 7.57 (d, J=9.04 Hz, 2H) 8.21 (s, 1H) 8.45 (s, 1H).

### Step 63.2

### tert-butyl 3-[{5-carbamoyl-1-[4-(3,4-difluorophenoxy)phenyl]-4-nitro-1H-pyrazol-3-yl}(2-oxoethyl)amino]azetidine-1-carboxylate

Step 63.2 was prepared according to the procedure for Step 81.2, substituting tert-butyl 3-[{5-carbamoyl-1-[4-(3,4-difluorophenoxy)phenyl]-4-nitro-1H-pyrazol-3-yl}(prop-2-en-1-yl)amino]azetidine-1-carboxylate (Step 63.1) for tert-butyl 3-[{5-carbamoyl-1-[4-(2,4-difluorophenoxy)phenyl]-4-nitro-1H-pyrazol-3-yl}(prop-2-en-1-yl)amino]azetidine-1-carboxylate. MS (ESI) m/z: 517.1 [M-OtButyl+H₂O].

### Step 63.3

### tert-butyl 3-{3-carbamoyl-2-[4-(3,4-difluorophenoxy)phenyl]-2,4,5,6-tetrahydro-7H-pyrazolo[3,4-b]pyrazin-7-yl}azetidine-1-carboxylate

Step 63.3 was prepared according to the procedure for Step 81.3, substituting tert-butyl 3-[{5-carbamoyl-1-[4-(3,4-difluorophenoxy)phenyl]-4-nitro-1H-pyrazol-3-yl}(2-oxoethyl)amino]azetidine-1-carboxylate (Step 63.2) for tert-butyl 3-[{5-carbamoyl-1-[4-(2,4-difluorophenoxy)phenyl]-4-nitro-1H-pyrazol-3-yl}(2-oxoethyl)amino]azetidine-1-carboxylate. MS (ESI) m/z: 527.4 [M+H]⁺.

### Step 63.4

### 7-(azetidin-3-yl)-2-[4-(3,4-difluorophenoxy)phenyl]-4,5,6,7-tetrahydro-2H-pyrazolo[3,4-b]pyrazine-3-carboxamide-hydrogen chloride (1/3)

Step 63.4 was prepared according to the procedure for Step 81.4, substituting tert-butyl 3-{3-carbamoyl-2-[4-(3,4-difluorophenoxy)phenyl]-2,4,5,6-tetrahydro-7H-pyrazolo[3,4-b]pyrazin-7-yl}azetidine-1-carboxylate (Step 63.3) for tert-butyl 3-{3-carbamoyl-2-[4-(2,4-difluorophenoxy)phenyl]-2,4,5,6-tetrahydro-7H-pyrazolo[3,4-b]pyrazin-7-yl}azetidine-1-carboxylate. MS (ESI) m/z: 427.1 [M+H]⁺.

### Step 63.5

### 2-[4-(3,4-difluorophenoxy)phenyl]-7-[1-(prop-2-enoyl)azetidin-3-yl]-4,5,6,7-tetrahydro-2H-pyrazolo[3,4-b]pyrazine-3-carboxamide

Step 63.5 was prepared according to the procedure for Step 35.4, substituting 7-(azetidin-3-yl)-2-[4-(3,4-difluorophenoxy)phenyl]-4,5,6,7-tetrahydro-2H-pyrazolo[3,4-b]pyrazine-3-carboxamide hydrogen chloride (1/3) (Step 63.4) for 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide. ¹H NMR (400 MHz, methanol-d₄) δ ppm 3.28 (br d, J=4.85 Hz, 2H) 3.50 (br s, 2H) 4.20-4.44 (m, 3H) 4.47-4.64 (m, 2H) 5.73 (dd, J=10.14, 1.76 Hz, 1H) 6.18-6.29 (m, 1H) 6.31-6.43 (m, 1H) 6.79-6.88 (m, 1H) 6.92-7.11 (m, 3H) 7.26 (q, J=9.26 Hz, 1H) 7.37 (br d, J=8.60 Hz, 2H). MS (ESI) m/z: 481.1 [M+H]⁺.

### Example 64

### (7R)-2-[4-(3-cyanophenoxy)phenyl]-7-[4-(prop-2-enoyl)piperazin-1-yl]-4,5,6,7-tetrahydro-2H-pyrazolo[4,3-b]pyridine-3-carboxamide

### Step 64.1

### ethyl (7R)-2-[4-(3-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 64.1 was prepared according to the procedure for Step 78.1, substituting (3-cyanophenyl)boronic acid for [4-(3-cyclopropylphenoxy)phenyl]boronic acid. MS (ESI) m/z: 657.9 [M+H]⁺.

### Step 64.2

### (7R)-2-[4-(3-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid

Step 64.2 was prepared according to the procedure for Step 78.2, substituting ethyl (7R)-2-[4-(3-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 64.1) for ethyl (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate. MS (ESI) m/z: 629.9 [M+H]⁺.

### Step 64.3

### (7R)-2-[4-(3-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 64.3 was prepared according to the procedure for Step 78.3, substituting (7R)-2-[4-(3-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid (Step 64.2) for (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid. MS (ESI) m/z: 628.9 [M+H]⁺.

### Step 64.4

### (7R)-2-[4-(3-cyanophenoxy)phenyl]-7-(piperazin-1-yl)-4,5,6,7-tetrahydro-2H-pyrazolo[4,3-b]pyridine-3-carboxamide

Step 64.4 was prepared according to the procedure for Step 78.4, substituting (7R)-2-[4-(3-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 64.3) for (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide. MS (ESI) m/z: 444.2 [M+H]⁺.

### Step 64.5

### (7R)-2-[4-(3-cyanophenoxy)phenyl]-7-[4-(prop-2-enoyl)piperazin-1-yl]-4,5,6,7-tetrahydro-2H-pyrazolo[4,3-b]pyridine-3-carboxamide

Step 64.5 was prepared according to the procedure for Step 78.5, substituting (7R)-2-[4-(3-cyanophenoxy)phenyl]-7-(piperazin-1-yl)-4,5,6,7-tetrahydro-2H-pyrazolo[4,3-b]pyridine-3-carboxamide (Step 64.4) for (7R)-2-[4-(3-cyclopropylphenoxy)phenyl]-7-(piperazin-1-yl)-4,5,6,7-tetrahydro-2H-pyrazolo[4,3-b]pyridine-3-carboxamide. ¹H NMR (400 MHz, dimethyl sulfoxide-d₆) δ ppm 7.66-7.58 (m, 2H), 7.57-7.51 (m, 1H), 7.43-7.31 (m, 3H), 7.15-7.08 (m, 2H), 6.78 (dd, J=16.7, 10.4 Hz, 1H), 6.09 (dd, J=16.7, 2.4 Hz, 1H), 5.66 (dd, J=10.4, 2.5 Hz, 1H), 5.16-5.07 (m, 1H), 3.71-3.62 (m, 1H), 3.50 (d, J=28.9 Hz, 4H), 3.25 (d, J=3.0 Hz, 1H), 3.14 (d, J=15.4 Hz, 1H), 2.68 (s, 1H), 2.60 (s, 3H), 2.09 (s, 1H), 1.75 (td, J=9.5, 4.3 Hz, 1H); MS (ESI) m/z: 498.1 [M+H]⁺.

### Example 65

### (7R)-7-[4-(prop-2-enoyl)piperazin-1-yl]-2-{4-[3-(trifluoromethyl)phenoxy]phenyl}-4,5,6,7-tetrahydro-2H-pyrazolo[4,3-b]pyridine-3-carboxamide

### Step 65.1

### ethyl (7R)-7-[4-(2-nitrobenzene-1-sulfonyl)piperazin-1-yl]-2-{4-[3-(trifluoromethyl)phenoxy]phenyl}-4,5,6,7-tetrahydro-2H-pyrazolo[4,3-b]pyridine-3-carboxylate

Step 65.1 was prepared according to the procedure for Step 78.1, substituting {4-[3-(trifluoromethyl)phenoxy]phenyl}boronic acid for [4-(3-cyclopropylphenoxy)phenyl]boronic acid. MS (ESI) m/z: 700.8 [M+H]⁺.

### Step 65.2

### (7R)-7-[4-(2-nitrobenzene-1-sulfonyl)piperazin-1-yl]-2-{4-[3-(trifluoromethyl)phenoxy]phenyl}-4,5,6,7-tetrahydro-2H-pyrazolo[4,3-b]pyridine-3-carboxylic acid

Step 65.2 was prepared according to the procedure for Step 78.2, substituting ethyl (7R)-7-[4-(2-nitrobenzene-1-sulfonyl)piperazin-1-yl]-2-{4-[3-(trifluoromethyl)phenoxy]phenyl}-4,5,6,7-tetrahydro-2H-pyrazolo[4,3-b]pyridine-3-carboxylate (Step 65.1) for ethyl (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate. MS (ESI) m/z: 672.8 [M+H]⁺.

### Step 65.3

### (7R)-7-[4-(2-nitrobenzene-1-sulfonyl)piperazin-1-yl]-2-{4-[3-(trifluoromethyl)phenoxy]phenyl}-4,5,6,7-tetrahydro-2H-pyrazolo[4,3-b]pyridine-3-carboxamide

Step 65.3 was prepared according to the procedure for Step 78.3, substituting (7R)-7-[4-(2-nitrobenzene-1-sulfonyl)piperazin-1-yl]-2-{4-[3-(trifluoromethyl)phenoxy]phenyl}-4,5,6,7-tetrahydro-2H-pyrazolo[4,3-b]pyridine-3-carboxylic acid (Step 65.2) for (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid. MS (ESI) m/z: 671.8 [M+H]⁺.

### Step 65.4

### (7R)-7-(piperazin-1-yl)-2-{4-[3-(trifluoromethyl)phenoxy]phenyl}-4,5,6,7-tetrahydro-2H-pyrazolo[4,3-b]pyridine-3-carboxamide

Step 65.4 was prepared according to the procedure for Step 78.4, substituting (7R)-7-[4-(2-nitrobenzene-1-sulfonyl)piperazin-1-yl]-2-{4-[3-(trifluoromethyl)phenoxy]phenyl}-4,5,6,7-tetrahydro-2H-pyrazolo[4,3-b]pyridine-3-carboxamide (Step 65.3) for (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide. MS (ESI) m/z: 486.9 [M+H]⁺.

### Step 65.5

### (7R)-7-[4-(prop-2-enoyl)piperazin-1-yl]-2-{4-[3-(trifluoromethyl)phenoxy]phenyl}-4,5,6,7-tetrahydro-2H-pyrazolo[4,3-b]pyridine-3-carboxamide

Step 65.5 was prepared according to the procedure for Step 78.5, substituting (7R)-7-(piperazin-1-yl)-2-{4-[3-(trifluoromethyl)phenoxy]phenyl}-4,5,6,7-tetrahydro-2H-pyrazolo[4,3-b]pyridine-3-carboxamide (Step 65.4) for (7R)-2-[4-(3-cyclopropylphenoxy)phenyl]-7-(piperazin-1-yl)-4,5,6,7-tetrahydro-2H-pyrazolo[4,3-b]pyridine-3-carboxamide. ¹H NMR (500 MHz, dimethyl sulfoxide-d₆) δ ppm 7.65 (t, J=7.9 Hz, 1H), 7.52 (ddt, J=7.8, 1.7, 0.9 Hz, 1H), 7.41-7.33 (m, 4H), 7.16-7.12 (m, 2H), 6.78 (dd, J=16.7, 10.5 Hz, 1H), 6.09 (dd, J=16.7, 2.4 Hz, 1H), 5.66 (dd, J=10.4, 2.4 Hz, 1H), 5.12 (t, J=3.0 Hz, 1H), 3.66 (dd, J=6.2, 4.8 Hz, 1H), 3.61-3.43 (m, 4H), 3.26 (td, J=8.9, 4.4 Hz, 1H), 3.17-3.09 (m, 1H), 2.73-2.54 (m, 4H), 2.12-2.04 (m, 1H), 1.79-1.71 (m, 1H). MS (ESI) m/z: 541.0 [M+H]⁺.

### Example 66

### 2-(4-phenoxyphenyl)-7-[rac-(3aR,6aS)-5-(prop-2-enoyl)hexahydropyrrolo[3,4-c]pyrrol-2(1H)-yl]-4,5,6,7-tetrahydro-2H-pyrazolo[4,3-b]pyridine-3-carboxamide

### Step 66.1

### ethyl 7-[rac-(3aR,6aS)-5-(tert-butoxycarbonyl)hexahydropyrrolo[3,4-c]pyrrol-2(1H)-yl]-2-(4-phenoxyphenyl)-4,5,6,7-tetrahydro-2H-pyrazolo[4,3-b]pyridine-3-carboxylate

To a suspension of ethyl 7-oxo-2-(4-phenoxyphenyl)-4,5,6,7-tetrahydro-2H-pyrazolo[4,3-b]pyridine-3-carboxylate (770 mg, 2.040 mmol) and cis-tert-butyl hexahydropyrrolo[3,4-c]pyrrole-2(1H)-carboxylate (650 mg, 3.06 mmol) in ethanol (102.00 mL) was added zinc(II) chloride solution in tetrahydrofuran (8.16 mL, 4.08 mmol) and the mixture was brought to reflux for 2 hours. Sodium cyanotrihydroborate (385 mg, 6.12 mmol) was added and the mixture was left to stir overnight at reflux. LCMS analysis of the crude reaction mixture showed full conversion to the desired product as well as a product with m z=541. The resulting mixture was quenched with water and ethanol was evaporated. The mixture was diluted with more water and extracted with ethyl acetate. The combined organic extracts were dried over anhydrous magnesium sulfate and concentrated. The crude residue was purified by column chromatography on silica gel using dichloromethane/methanol gradient (0 to 10%) to afford the title compound (628 mg, 53.7%). MS (ESI) m/z: 574.7 [M+H]⁺.

### Step 66.2

### tert-butyl rac-(3aR,6aS)-5-[3-carbamoyl-2-(4-phenoxyphenyl)-4,5,6,7-tetrahydro-2H-pyrazolo[4,3-b]pyridin-7-yl]hexahydropyrrolo[3,4-c]pyrrole-2(1H)-carboxylate

To a solution of ethyl 7-[rac-(3aR,6aS)-5-(tert-butoxycarbonyl)hexahydropyrrolo[3,4-c]pyrrol-2(1H)-yl]-2-(4-phenoxyphenyl)-4,5,6,7-tetrahydro-2H-pyrazolo[4,3-b]pyridine-3-carboxylate (628 mg, 1.095 mmol, Step 66.1) in a mixture of tetrahydrofuran (4379 μL)/methanol (4379 μL)/water (2189 μL) was added lithium hydroxide monohydrate (138 mg, 3.28 mmol), and the resulting mixture was left to stir for several hours. LCMS analysis of the crude reaction mixture indicated full conversion. The reaction mixture was concentrated to afford the corresponding lithium salt. The crude product was suspended in dry N,N-dimethylformamide (70.1 mL) together with ammonia hydrochloride (0.375 g, 7.01 mmol) and 2-(3H-[1,2,3]triazolo[4,5-b]pyridin-3-yl)-1,1,3,3-tetramethylisouronium hexafluorophosphate(V) (1.067 g, 2.81 mmol). N-Ethyl-N-isopropylpropan-2-amine (1.225 mL, 7.01 mmol) was added and the resulting mixture was left to stir at ambient temperature overnight. The mixture was diluted with ethyl acetate and washed with water and brine. The organic layer was dried over magnesium sulfate and concentrated. The crude residue was purified by column chromatography on silica gel using dichloromethane/methanol gradient (0 to 10% methanol) to afford the title compound (270 mg, 45.2%). MS (ESI) m/z: 545.7 [M+H]⁺.

### Step 66.3

### 7-[rac-(3aR,6aS)-hexahydropyrrolo[3,4-c]pyrrol-2(1H)-yl]-2-(4-phenoxyphenyl)-4,5,6,7-tetrahydro-2H-pyrazolo[4,3-b]pyridine-3-carboxamide-hydrogen chloride (1/1)

To a solution of tert-butyl rac-(3aR,6aS)-5-[3-carbamoyl-2-(4-phenoxyphenyl)-4,5,6,7-tetrahydro-2H-pyrazolo[4,3-b]pyridin-7-yl]hexahydropyrrolo[3,4-c]pyrrole-2(1H)-carboxylate (270 mg, 0.496 mmol, Step 66.2) in dry 1,4-dioxane (4957 μL) was added 4 M hydrogen chloride in 1,4-dioxane (1859 μL, 7.44 mmol), and the resulting mixture was left to stir for 1 hour. UPLC analysis showed full conversion. The resulting precipitate was filtered, dissolved in dimethyl sulfoxide and purified on Cis silica using acetonitrile/0.5 μM aqueous HCl gradient (10 to 45% acetonitrile on a 43 g column over 18 minutes) to afford the title compound (145 mg, 60.8%). MS (ESI) m/z: 445.3 [M+H]⁺.

### Step 66.4

### 2-(4-phenoxyphenyl)-7-[rac-(3aR,6aS)-5-(prop-2-enoyl)hexahydropyrrolo[3,4-c]pyrrol-2(1H)-yl]-4,5,6,7-tetrahydro-2H-pyrazolo[4,3-b]pyridine-3-carboxamide

To a solution of 7-[rac-(3aR,6aS)-hexahydropyrrolo[3,4-c]pyrrol-2(1H)-yl]-2-(4-phenoxyphenyl)-4,5,6,7-tetrahydro-2H-pyrazolo[4,3-b]pyridine-3-carboxamide-hydrogen chloride (1/1) (30 mg, 0.062 mmol) and N-ethyl-N-isopropylpropan-2-amine (32.7 μL, 0.187 mmol, Step 66.3) in dry N,N-dimethyl formamide (0.832 mL) was added acryloyl chloride (5.65 mg, 0.062 mmol) as a solution in N,N-dimethyl formamide (0.416 mL) at −78° C. dropwise, and the resulting mixture was left to stir for 2 minutes. The reaction mixture was acidified with several drops of trifluoroacetic acid and purified by preparative HPLC using acetonitrile/0.1% formic acid (10->40% acetonitrile over 21 minutes) to afford the title compound (2.0 mg, 6.30%). MS (ESI) m/z: 499.2 [M+H]⁺.

### Example 67

### (7R)-2-[4-(3-fluorophenoxy)phenyl]-7-[4-(prop-2-enoyl)piperazin-1-yl]-4,5,6,7-tetrahydro-2H-pyrazolo[4,3-b]pyridine-3-carboxamide

The title compound was obtained by separating the enantiomers of 2-[4-(3-fluorophenoxy)phenyl]-7-[4-(prop-2-enoyl)piperazin-1-yl]-4,5,6,7-tetrahydro-2H-pyrazolo[4,3-b]pyridine-3-carboxamide (Example 57) with preparative supercritical fluid chromatography purification. Preparative supercritical fluid chromatography was performed on a Waters SFC 80Q system running under SuperChrom software control. The preparative supercritical fluid chromatography system was equipped with an 8-way preparative column switcher, CO₂ pump, modifier pump, automated back pressure regulator (ABPR), UV detector, and 6-position fraction collector. The mobile phase was comprised of supercritical CO₂ supplied by a dewar of bone-dry non-certified CO₂ pressurized to 350 psi with a modifier of methanol at a flow rate of 70 g/minutes. The column was at ambient temperature and the backpressure regulator was set to maintain 100 bar. The sample was dissolved in methanol at a concentration of 8.2 mg/mL. The sample was loaded into the modifier stream in 0.5 mL (4.1 mg) injections. The mobile phase was held isocratically at 30% methanol:CO₂. Fraction collection was threshold triggered. The instrument was fitted with a CHIRALPAK® AD-H column with dimensions 21 mm i.d.×250 mm length with 5 μm particles. The peak that eluted first was concentrated under reduced pressure to provide the title compound. ¹H NMR (400 MHz, dimethyl sulfoxide-d₆) δ ppm 7.44-7.36 (m, 1H), 7.35-7.29 (m, 2H), 7.10-7.04 (m, 2H), 6.99-6.92 (m, 1H), 6.91-6.82 (m, 2H), 6.73 (dd, J=16.7, 10.5 Hz, 1H), 6.05 (dd, J=16.7, 2.4 Hz, 1H), 5.62 (dd, J=10.5, 2.4 Hz, 1H), 5.13-4.95 (m, 1H), 3.62 (t, J=5.5 Hz, 1H), 3.36 (s, 5H), 3.12-3.02 (m, 1H), 2.71-2.50 (m, 4H), 2.11-1.95 (m, 1H), 1.79-1.62 (m, 1H). MS (ESI) m/z: 491.1 [M+H]⁺.

### Example 68

### (7R)-2-[4-(2-cyclopropylphenoxy)phenyl]-7-[4-(prop-2-enoyl)piperazin-1-yl]-4,5,6,7-tetrahydro-2H-pyrazolo[4,3-b]pyridine-3-carboxamide

### Step 68.1

### ethyl (7R)-2-[4-(2-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 68.1 was prepared according to the procedure for Step 78.1, substituting [4-(2-cyclopropylphenoxy)phenyl]boronic acid (Intermediate Q) for [4-(3-cyclopropylphenoxy)phenyl]boronic acid. MS (ESI) m/z: 672.9 [M+H]⁺.

### Step 68.2

### (7R)-2-[4-(2-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid

Step 68.2 was prepared according to the procedure for Step 78.2, substituting ethyl (7R)-2-[4-(2-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 68.1) for ethyl (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate. MS (ESI) m/z: 645.0 [M+H]⁺.

### Step 68.3

### (7R)-2-[4-(2-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 68.3 was prepared according to the procedure for Step 78.3, substituting (7R)-2-[4-(2-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid (Step 68.2) for (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid. MS (ESI) m/z: 644.0 [M+H]⁺.

### Step 68.4

### (7R)-2-[4-(2-cyclopropylphenoxy)phenyl]-7-(piperazin-1-yl)-4,5,6,7-tetrahydro-2H-pyrazolo[4,3-b]pyridine-3-carboxamide

Step 68.4 was prepared according to the procedure for Step 78.4, substituting (7R)-2-[4-(2-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 68.3) for (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide. MS (ESI) m/z: 459.1 [M+H]⁺.

### Step 68.5

### (7R)-2-[4-(2-cyclopropylphenoxy)phenyl]-7-[4-(prop-2-enoyl)piperazin-1-yl]-4,5,6,7-tetrahydro-2H-pyrazolo[4,3-b]pyridine-3-carboxamide

Step 68.5 was prepared according to the procedure for Step 78.5, substituting (7R)-2-[4-(2-cyclopropylphenoxy)phenyl]-7-(piperazin-1-yl)-4,5,6,7-tetrahydro-2H-pyrazolo[4,3-b]pyridine-3-carboxamide (Step 68.4) for (7R)-2-[4-(3-cyclopropylphenoxy)phenyl]-7-(piperazin-1-yl)-4,5,6,7-tetrahydro-2H-pyrazolo[4,3-b]pyridine-3-carboxamide to afford the title compound. ¹H NMR (400 MHz, dimethyl sulfoxide-d₆) δ ppm 7.32-7.27 (m, 2H), 7.20 (td, J=7.6, 1.7 Hz, 1H), 7.13 (td, J=7.5, 1.5 Hz, 1H), 6.99 (ddd, J=13.4, 7.8, 1.6 Hz, 2H), 6.96-6.90 (m, 2H), 6.77 (dd, J=16.6, 10.5 Hz, 1H), 6.08 (dd, J=16.6, 2.5 Hz, 1H), 5.65 (dd, J=10.4, 2.4 Hz, 1H), 5.07 (t, J=3.0 Hz, 1H), 3.64 (t, J=5.4 Hz, 1H), 3.59-3.38 (m, 4H), 3.30-3.21 (m, 1H), 3.16-3.07 (m, 1H), 2.72-2.52 (m, 4H), 2.12-1.97 (m, 2H), 1.80-1.67 (m, 1H), 0.92-0.85 (m, 2H), 0.72-0.65 (m, 2H). MS (ESI) m/z: 513.1 [M+H]⁺.

### Example 69

### (7R)-2-[4-(2-cyanophenoxy)phenyl]-7-[4-(prop-2-enoyl)piperazin-1-yl]-4,5,6,7-tetrahydro-2H-pyrazolo[4,3-b]pyridine-3-carboxamide

### Step 69.1

### ethyl (7R)-2-[4-(2-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 69.1 was prepared according to the procedure for Step 55.1, substituting [4-(2-cyanophenoxy)phenyl]boronic acid (Intermediate H) for [4-(3,5-difluorophenoxy)phenyl]boronic acid, and ethyl (7R)-7-[4-(2-nitrobenzene-1-sulfonyl)piperazin-1-yl]-4,5,6,7-tetrahydro-2H-pyrazolo[4,3-b]pyridine-3-carboxylate (Intermediate R) for ethyl 7-[4-(tert-butoxycarbonyl)piperazin-1-yl]-4,5,6,7-tetrahydro-2H-pyrazolo[4,3-b]pyridine-3-carboxylate. ¹H NMR (500 MHz, dimethyl sulfoxide-d₆) δ ppm 8.03-7.96 (m, 2H), 7.98-7.89 (m, 2H), 7.86 (td, J=7.6, 1.3 Hz, 1H), 7.76-7.69 (m, 1H), 7.48-7.41 (m, 2H), 7.34 (td, J=7.6, 1.0 Hz, 1H), 7.23-7.17 (m, 2H), 7.06 (dd, J=8.5, 0.9 Hz, 1H), 5.56-5.51 (m, 1H), 4.23-4.10 (m, 2H), 3.75-3.70 (m, 1H), 3.31-3.24 (m, 1H), 3.24-3.12 (m, 5H), 2.78-2.64 (m, 4H), 2.09-2.04 (m, 1H), 1.81-1.72 (m, 1H), 1.19 (t, J=7.1 Hz, 3H).

### Step 69.2

### (7R)-2-[4-(2-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid

Step 69.2 was prepared according to the procedure for Step 55.2, substituting ethyl (7R)-2-[4-(2-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 69.1) for ethyl 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylate. MS (ESI) m/z: 629.9 [M+H]⁺.

### Step 69.3

### (7R)-2-[4-(2-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 69.3 was prepared according to the procedure for Step 55.3, substituting (7R)-2-[4-(2-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid (Step 69.2) for 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylic acid. MS (ESI) m/z: 628.9 [M+H]⁺.

### Step 69.4

### (7R)-2-[4-(2-cyanophenoxy)phenyl]-7-(piperazin-1-yl)-4,5,6,7-tetrahydro-2H-pyrazolo[4,3-b]pyridine-3-carboxamide

To a solution of decane-1-thiol (0.046 mL, 0.22 mmol) and (7R)-2-[4-(2-cyanophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (0.068 g, 0.11 mmol, Step 69.3) in tetrahydrofuran (0.7 mL) was added sodium 2-methylpropan-2-olate (0.097 mL, 0.19 mmol) at 0° C. The reaction mixture was warmed to ambient temperature and stirred for 1 hour. The reaction mixture was quenched by the addition of saturated aqueous NH₄Cl, and the resulting aqueous solution was extracted with ethyl acetate (3×). The combined organic layers were concentrated under reduced pressure to provide the title compound (0.026 g, 0.058 mmol, 54%), which was used without further purification. MS (ESI) m/z: 444.1 [M+H]⁺.

### Step 69.5

### (7R)-2-[4-(2-cyanophenoxy)phenyl]-7-[4-(prop-2-enoyl)piperazin-1-yl]-4,5,6,7-tetrahydro-2H-pyrazolo[4,3-b]pyridine-3-carboxamide

Step 69.5 was prepared according to the procedure for Step 12.4, substituting (7R)-2-[4-(2-cyanophenoxy)phenyl]-7-(piperazin-1-yl)-4,5,6,7-tetrahydro-2H-pyrazolo[4,3-b]pyridine-3-carboxamide for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (500 MHz, CDCl₃) δ ppm 7.70 (dd, J=7.8, 1.7 Hz, 1H), 7.60-7.54 (m, 1H), 7.52-7.46 (m, 2H), 7.23 (td, J=7.6, 1.0 Hz, 1H), 7.20-7.15 (m, 2H), 7.04 (dd, J=8.5, 1.0 Hz, 1H), 6.56 (dd, J=16.8, 10.6 Hz, 1H), 6.28 (dd, J=16.9, 1.9 Hz, 1H), 5.68 (dd, J=10.6, 2.0 Hz, 1H), 5.48 (s, 2H), 5.11 (s, 1H), 3.97-3.56 (m, 5H), 3.51-3.42 (m, 1H), 3.38-3.26 (m, 1H), 2.92-2.61 (m, 4H), 2.27-2.12 (m, 1H), 2.07-1.90 (m, 1H). MS (ESI) m/z: 498.1 [M+H]⁺.

### Example 70

### (7R)-2-[4-(2,4-difluorophenoxy)phenyl]-7-[4-(prop-2-enoyl)piperazin-1-yl]-4,5,6,7-tetrahydro-2H-pyrazolo[4,3-b]pyridine-3-carboxamide

### Step 70.1

### ethyl (7R)-2-[4-(2,4-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 70.1 was prepared according to the procedure for Step 55.1, substituting [4-(2,4-difluorophenoxy)phenyl]boronic acid (Intermediate I) for [4-(3,5-difluorophenoxy)phenyl]boronic acid and ethyl (7R)-7-[4-(2-nitrobenzene-1-sulfonyl)piperazin-1-yl]-4,5,6,7-tetrahydro-2H-pyrazolo[4,3-b]pyridine-3-carboxylate (Intermediate R) for ethyl 7-[4-(tert-butoxycarbonyl)piperazin-1-yl]-4,5,6,7-tetrahydro-2H-pyrazolo[4,3-b]pyridine-3-carboxylate. MS (ESI) m/z: 668.83 [M+H]⁺.

### Step 70.2

### (7R)-2-[4-(2,4-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

(7R)-2-[4-(2,4-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid was prepared according to the procedure for Step 55.2, substituting ethyl (7R)-2-[4-(2,4-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 70.1) for ethyl 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylate. The title compound was prepared according to the procedure for Step 55.3, substituting (7R)-2-[4-(2,4-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid for 7-[4-(tert-butoxycarbonyl)piperazin-1-yl]-2-[4-(3,5-difluorophenoxy)phenyl]-4,5,6,7-tetrahydro-2H-pyrazolo[4,3-b]pyridine-3-carboxylic acid. MS (ESI) m/z: 640.88 [M+H]⁺.

### Step 70.3

### (7R)-2-[4-(2,4-difluorophenoxy)phenyl]-7-(piperazin-1-yl)-4,5,6,7-tetrahydro-2H-pyrazolo[4,3-b]pyridine-3-carboxamide

Step 70.3 was prepared according to the procedure for Step 74.4, substituting (7R)-2-[4-(2,4-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 70.2) for (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide. MS (ESI) m/z: 454.95 [M+H]⁺.

### Step 70.4

### (7R)-2-[4-(2,4-difluorophenoxy)phenyl]-7-[4-(prop-2-enoyl)piperazin-1-yl]-4,5,6,7-tetrahydro-2H-pyrazolo[4,3-b]pyridine-3-carboxamide

Step 70.4 was prepared according to the procedure for Step 12.4, substituting (7R)-2-[4-(2,4-difluorophenoxy)phenyl]-7-(piperazin-1-yl)-4,5,6,7-tetrahydro-2H-pyrazolo[4,3-b]pyridine-3-carboxamide (Step 70.3) for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (500 MHz, dimethyl sulfoxide-d₆) δ ppm 7.52 (ddd, J=11.0, 8.8, 3.0 Hz, 1H), 7.39-7.29 (m, 3H), 7.21-7.13 (m, 1H), 7.04-6.97 (m, 2H), 6.79 (dd, J=16.7, 10.5 Hz, 1H), 6.10 (dd, J=16.7, 2.4 Hz, 1H), 5.67 (dd, J=10.4, 2.4 Hz, 1H), 5.12-5.09 (m, 1H), 3.65 (dd, J=6.2, 4.8 Hz, 1H), 3.61-3.22 (m, 5H), 3.16-3.09 (m, 1H), 2.79-2.55 (m, 4H), 2.15-2.00 (m, 1H), 1.79-1.70 (m, 1H). MS (ESI) m/z: 509.1 [M+H]⁺.

### Example 71

### (7R)-2-[4-(4-fluorophenoxy)phenyl]-7-[4-(prop-2-enoyl)piperazin-1-yl]-4,5,6,7-tetrahydro-2H-pyrazolo[4,3-b]pyridine-3-carboxamide

### Step 71.1

### ethyl (7R)-2-[4-(4-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 71.1 was prepared according to the procedure for the Step 77.1, substituting [4-(4-fluorophenoxy)phenyl]boronic acid for [4-(2,3-difluorophenoxy)phenyl]boronic acid. ¹H NMR (600 MHz, CDCl₃) δ ppm 8.02-7.86 (m, 1H), 7.71-7.62 (m, 2H), 7.62-7.54 (m, 1H), 7.35-7.29 (m, 2H), 7.09-6.99 (m, 4H), 6.99-6.95 (m, 2H), 4.68 (d, J=2.0 Hz, 1H), 4.22 (qd, J=7.1, 1.2 Hz, 2H), 3.86 (dd, J=6.6, 5.0 Hz, 1H), 3.51-3.40 (m, 1H), 3.31 (ddt, J=13.5, 9.0, 4.5 Hz, 5H), 2.79 (q, J=4.8 Hz, 4H), 2.15 (dtd, J=14.0, 7.2, 3.1 Hz, 1H), 1.95 (dddd, J=13.4, 8.2, 5.0, 3.3 Hz, 1H), 1.23 (t, J=7.1 Hz, 3H). MS (APCI) m/z: 651.3 [M+H]⁺.

### Step 71.2

### (7R)-2-[4-(4-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid

Step 71.2 was prepared according to the procedure for the Step 77.2, substituting ethyl (7R)-2-[4-(4-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 71.1) for ethyl (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate. MS (APCI) m/z: 623.4 [M+H]⁺.

### Step 71.3

### (7R)-2-[4-(4-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 71.3 was prepared according to the procedure for the Step 77.3, substituting (7R)-2-[4-(4-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid (Step 71.2) for (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid. MS (APCI) m/z: 622.3 [M+H]⁺.

### Step 71.4

### (7R)-2-[4-(4-fluorophenoxy)phenyl]-7-(piperazin-1-yl)-4,5,6,7-tetrahydro-2H-pyrazolo[4,3-b]pyridine-3-carboxamide

Step 71.4 was prepared according to the procedure for Step 77.4, substituting (7R)-2-[4-(4-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 71.3) for (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide. MS (APCI) m/z: 437.4 [M+H]⁺.

### Step 71.5

### (7R)-2-[4-(4-fluorophenoxy)phenyl]-7-[4-(prop-2-enoyl)piperazin-1-yl]-4,5,6,7-tetrahydro-2H-pyrazolo[4,3-b]pyridine-3-carboxamide

Step 71.5 was prepared according to the procedure for the Step 18.4, substituting (7R)-2-[4-(4-fluorophenoxy)phenyl]-7-(piperazin-1-yl)-4,5,6,7-tetrahydro-2H-pyrazolo[4,3-b]pyridine-3-carboxamide (Step 71.4) for 7-[(2S,5R)-2,5-dimethylpiperazin-1-yl]-2-(4-phenoxyphenyl)-4,5,6,7-tetrahydro-2H-pyrazolo[4,3-b]pyridine-3-carboxamide to afford the title compound. ¹H NMR (500 MHz, dimethyl sulfoxide-d₆, 90° C.) δ ppm 7.37-7.29 (m, 2H), 7.29-7.19 (m, 2H), 7.15-7.07 (m, 2H), 7.05-6.95 (m, 2H), 6.78 (dd, J=16.7, 10.5 Hz, 1H), 6.09 (dd, J=16.7, 2.4 Hz, 1H), 5.66 (dd, J=10.5, 2.4 Hz, 1H), 5.10 (t, J=3.0 Hz, 1H), 3.65 (dd, J=6.2, 4.8 Hz, 1H), 3.45 (q, J=6.9, 6.3 Hz, 4H), 3.25 (ddt, J=8.7, 5.6, 2.9 Hz, 1H), 3.21-3.05 (m, 1H), 2.76-2.52 (m, 5H), 2.07 (ddt, J=13.6, 6.8, 4.2 Hz, 1H), 1.74 (tq, J=8.0, 4.2, 3.7 Hz, 1H). MS (APCI) m/z: 491.4 [M+H]⁺.

### Example 72

### (7S)-2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide; and

### Example 73

### (7R)-2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide

The racemate, 2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide (0.6 g, Example 45) was separated by preparative supercritical fluid chromatography (Chiralpak® AD-H, 250×21 mm i.d. 5 um; Column temperature 40° C.; Mobile phase: (A) for CO₂ and (B) for methanol (0.1% NH₃.H₂O; Gradient: 30 B %-100%; Flow rate: 70 g/minutes monitoring at 220 nm) to afford Example 72 (0.097 g, 22.0%). ¹H NMR (400 MHz, dimethyl sulfoxide-d₆) δ ppm 7.47-7.38 (m, 2H), 7.36-7.29 (m, 2H), 7.20-7.13 (m, 1H), 7.09-6.97 (m, 4H), 6.33 (dd, J=16.9, 10.2 Hz, 1H), 6.07 (ddd, J=17.0, 6.1, 2.4 Hz, 1H), 5.59 (dd, J=10.2, 2.3 Hz, 1H), 5.07 (d, J=12.9 Hz, 1H), 4.54 (d, J=7.8 Hz, 1H), 4.25 (d, J=7.1 Hz, 1H), 3.81 (dt, J=26.7, 5.8 Hz, 1H), 3.32-3.17 (m, 2H), 3.10 (d, J=10.1 Hz, 1H), 3.06-2.85 (m, 2H), 2.38 (q, J=6.6 Hz, 1H), 2.01 (td, J=14.0, 13.2, 6.5 Hz, 1H), 1.86 (dd, J=17.8, 7.7 Hz, 1H), 1.82-1.65 (m, 1H). MS (APCI) m/z: 485.4 [M+H]⁺; and Example 73 (0.097 g, 22.0%). ¹H NMR (400 MHz, dimethyl sulfoxide-d₆) 7.48-7.37 (m, 2H), 7.37-7.27 (m, 2H), 7.17 (ddt, J=7.4, 6.2, 1.3 Hz, 1H), 7.08-7.00 (m, 4H), 6.38-6.25 (m, 1H), 6.14-6.03 (m, 1H), 5.59 (dd, J=10.3, 2.3 Hz, 1H), 5.07 (dt, J=16.7, 2.9 Hz, 1H), 4.66-4.50 (m, 1H), 4.27-4.22 (m, 1H), 3.81 (ddd, J=34.6, 6.7, 4.9 Hz, 1H), 3.31-3.08 (m, 4H), 3.05-2.86 (m, 2H), 2.43-2.31 (m, 1H), 2.10-1.94 (m, 1H), 1.86 (dd, J=22.6, 7.7 Hz, 1H), 1.72 (dddd, J=17.4, 11.9, 8.2, 3.0 Hz, 1H). MS (APCI) m/z: 485.4 [M+H]⁺.

### Example 74

### (7R)-2-[4-(2,5-difluorophenoxy)phenyl]-7-[4-(prop-2-enoyl)piperazin-1-yl]-4,5,6,7-tetrahydro-2H-pyrazolo[4,3-b]pyridine-3-carboxamide

### Step 74.1

### ethyl (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

To a solution of pyridine (0.174 mL, 2.15 mmol), [4-(2,5-difluorophenoxy)phenyl]boronic acid (0.538 g, 2.15 mmol, Intermediate G), and ethyl (7R)-7-[4-(2-nitrobenzene-1-sulfonyl)piperazin-1-yl]-4,5,6,7-tetrahydro-2H-pyrazolo[4,3-b]pyridine-3-carboxylate (0.500 g, 1.08 mmol, Intermediate R) in dichloromethane (11 mL) was added copper(II) acetate (0.293 g, 1.62 mmol) at ambient temperature. The reaction mixture was stirred for 16 hours under an atmosphere of air. Saturated aqueous NH₄Cl was added, and the resulting solution was filtered through diatomaceous earth. The filtrate was extracted with dichloromethane (3×). The combined organic layers were concentrated under reduced pressure, and the crude product was purified by silica gel chromatography (0-10% methanol in dichloromethane) to provide the title compound (0.239 g, 33%). MS (ESI) m/z: 668.9 [M+H]⁺.

### Step 74.2

### (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid

To a solution of ethyl (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (0.237 g, 0.354 mmol, Step 74.1) in 4:4:1 tetrahydrofuran/methanol/water (3 mL) was added LiOH (0.085 g, 3.54 mmol). The reaction mixture was warmed to 50° C. and stirred for 2 hours. The reaction mixture was cooled to ambient temperature and acidified with 1 M aqueous HCl. The resulting solution was extracted with ethyl acetate (3×). The combined organic layers were dried over MgSO₄, filtered, and concentrated under reduced pressure to provide the title compound (0.179 g, 79%). MS (ESI) m/z: 640.9 [M+H]⁺.

### Step 74.3

### (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

To a solution of (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid (0.179 g, 0.279 mmol, Step 74.2), N,N-diisopropylethylamine (0.000146 mL, 0.838 mmol), and NH₄Cl (0.045 g, 0.84 mmol) in N,N-dimethylformamide (3 mL) was added 1-[bis(dimethylamino)methylene]-1H-1,2,3-triazolo[4,5-b]pyridinium 3-oxid hexafluorophosphate (0.159 g, 0.419 mmol) at ambient temperature. The reaction mixture was stirred for 1 hour, and then the reaction mixture was quenched with saturated aqueous NaHCO₃. The aqueous solution was extracted with ethyl acetate (3×). The combined organic layers were washed with brine (3×) and concentrated under reduced pressure. The crude product was purified by silica gel chromatography (0-10% methanol in dichloromethane) to afford the title compound (0.115 g, 64%). MS (ESI) m/z: 639.9 [M+H]⁺.

### Step 74.4

### (7R)-2-[4-(2,5-difluorophenoxy)phenyl]-7-(piperazin-1-yl)-4,5,6,7-tetrahydro-2H-pyrazolo[4,3-b]pyridine-3-carboxamide

To a solution of decane-1-thiol (0.076 mL, 0.360 mmol) and (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (0.115 g, 0.180 mmol, Step 74.3) in tetrahydrofuran (2 mL) was added sodium 2-methylpropan-2-olate (0.162 mL, 0.324 mmol) at 0° C. The reaction mixture was warmed to ambient temperature and stirred for 1 hour, and the reaction mixture was quenched by the addition of saturated aqueous NH₄Cl. The resulting aqueous solution was extracted with ethyl acetate (3×). The combined organic layers were concentrated under reduced pressure to provide the title compound (0.067 g, 82%). MS (ESI) m/z: 455.0 [M+H]⁺.

### Step 74.5

### (7R)-2-[4-(2,5-difluorophenoxy)phenyl]-7-[4-(prop-2-enoyl)piperazin-1-yl]-4,5,6,7-tetrahydro-2H-pyrazolo[4,3-b]pyridine-3-carboxamide

Step 74.5 was prepared according to the procedure for Step 12.4, substituting (7R)-2-[4-(2,5-difluorophenoxy)phenyl]-7-(piperazin-1-yl)-4,5,6,7-tetrahydro-2H-pyrazolo[4,3-b]pyridine-3-carboxamide (Step 74.4) for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (500 MHz, CDCl₃) δ ppm 7.48-7.42 (m, 2H), 7.27-7.18 (m, 1H), 7.18-7.13 (m, 2H), 7.00-6.84 (m, 2H), 6.51 (dd, J=16.8, 10.5 Hz, 1H), 6.34 (dd, J=16.8, 1.7 Hz, 1H), 5.81 (dd, J=10.5, 1.7 Hz, 1H), 4.69 (t, J=6.6 Hz, 1H), 4.17-3.61 (m, 5H), 3.63-3.35 (m, 2H), 3.11-2.22 (m, 6H). MS (ESI) m/z: 509.0 [M+H]⁺.

### Example 75

### 2-[4-(2-fluorophenoxy)phenyl]-7-[1-(prop-2-enoyl)azetidin-3-yl]-4,5,6,7-tetrahydro-2H-pyrazolo[3,4-b]pyrazine-3-carboxamide

### Step 75.1

### tert-butyl 3-[{5-carbamoyl-1-[4-(2-fluorophenoxy)phenyl]-4-nitro-1H-pyrazol-3-yl}(prop-2-en-1-yl)amino]azetidine-1-carboxylate

Step 75.1 was prepared according to the procedure for Step 81.1, substituting [4-(2-fluorophenoxy)phenyl]boronic acid for [4-(2,4-difluorophenoxy)phenyl]boronic acid (Intermediate I). ¹H NMR (400 MHz, dimethyl sulfoxide-d₆) δ ppm 1.36 (s, 9H) 3.79 (br dd, J=8.62, 5.81 Hz, 2H) 3.88-4.04 (m, 4H) 4.30 (br t, J=6.05 Hz, 1H) 5.09-5.27 (m, 2H) 5.71-5.89 (m, 1H) 7.11 (d, J=8.93 Hz, 2H) 7.24-7.36 (m, 3H) 7.43 (br dd, J=11.19, 1.41 Hz, 1H) 7.55 (d, J=8.93 Hz, 2H) 8.18 (s, 1H) 8.43 (s, 1H).

### Step 75.2

### tert-butyl 3-[{5-carbamoyl-1-[4-(2-fluorophenoxy)phenyl]-4-nitro-1H-pyrazol-3-yl}(2-oxoethyl)amino]azetidine-1-carboxylate

Step 75.2 was prepared according to the procedure for Step 81.2, substituting tert-butyl 3-[{5-carbamoyl-1-[4-(2-fluorophenoxy)phenyl]-4-nitro-1H-pyrazol-3-yl}(prop-2-en-1-yl)amino]azetidine-1-carboxylate (Step 75.1) for tert-butyl 3-[{5-carbamoyl-1-[4-(2,4-difluorophenoxy)phenyl]-4-nitro-1H-pyrazol-3-yl}(prop-2-en-1-yl)amino]azetidine-1-carboxylate. MS (ESI) m/z: 499.1 [M-OtButyl+H₂O]⁺.

### Step 75.3

### tert-butyl 3-{3-carbamoyl-2-[4-(2-fluorophenoxy)phenyl]-2,4,5,6-tetrahydro-7H-pyrazolo[3,4-b]pyrazin-7-yl}azetidine-1-carboxylate

Step 75.3 was prepared according to the procedure for Step 81.3, substituting tert-butyl 3-[{5-carbamoyl-1-[4-(2-fluorophenoxy)phenyl]-4-nitro-1H-pyrazol-3-yl}(2-oxoethyl)amino]azetidine-1-carboxylate (Step 75.2) for tert-butyl 3-[{5-carbamoyl-1-[4-(2,4-difluorophenoxy)phenyl]-4-nitro-1H-pyrazol-3-yl}(2-oxoethyl)amino]azetidine-1-carboxylate. ¹H NMR (400 MHz, dimethyl sulfoxide-d₆) δ ppm 1.37 (br s, 9H) 3.08 (br s, 2H) 3.29 (br d, J=2.87 Hz, 2H) 3.99 (br dd, J=5.40, 1.43 Hz, 2H) 4.03-4.12 (m, 2H) 4.15-4.27 (m, 1H) 5.07 (br s, 1H) 6.95 (br d, J=8.82 Hz, 2H) 7.20-7.26 (m, 4H) 7.35-7.46 (m, 2H).

### Step 75.4

### 7-(azetidin-3-yl)-2-[4-(2-fluorophenoxy)phenyl]-4,5,6,7-tetrahydro-2H-pyrazolo[3,4-b]pyrazine-3-carboxamide-hydrogen chloride (1/3)-pyrazolo[3,4-b]pyrazine-3-carboxamide hydrochloride salt

Step 75.4 was prepared according to the procedure for Step 81.4, substituting tert-butyl 3-{3-carbamoyl-2-[4-(2-fluorophenoxy)phenyl]-2,4,5,6-tetrahydro-7H-pyrazolo[3,4-b]pyrazin-7-yl}azetidine-1-carboxylate (Step 75.3) for tert-butyl 3-{3-carbamoyl-2-[4-(2,4-difluorophenoxy)phenyl]-2,4,5,6-tetrahydro-7H-pyrazolo[3,4-b]pyrazin-7-yl}azetidine-1-carboxylate. MS (ESI) m/z: 409.3 [M+H]⁺.

### Step 75.5

### 2-[4-(2-fluorophenoxy)phenyl]-7-[1-(prop-2-enoyl)azetidin-3-yl]-4,5,6,7-tetrahydro-2H-pyrazolo[3,4-b]pyrazine-3-carboxamide

Step 75.5 was prepared according to the procedure for Step 35.4, substituting 7-(azetidin-3-yl)-2-[4-(2-fluorophenoxy)phenyl]-4,5,6,7-tetrahydro-2H-pyrazolo[3,4-b]pyrazine-3-carboxamide-hydrogen chloride (1/3) (Step 75.4) for 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide. ¹H NMR (400 MHz, methanol-d₄) δ ppm 3.33-3.44 (m, 2H) 3.47-3.67 (m, 2H) 4.20-4.64 (m, 5H) 5.72 (dd, J=10.36, 1.76 Hz, 1H) 6.17-6.27 (m, 1H) 6.33 (br d, J=10.36 Hz, 1H) 6.99 (br d, J=7.50 Hz, 2H) 7.12-7.30 (m, 4H) 7.31-7.43 (m, 2H). MS (ESI) m/z: 463.2 [M+H]+.

### Example 76

### 2-[4-(2,3-difluorophenoxy)phenyl]-7-[1-(prop-2-enoyl)azetidin-3-yl]-4,5,6,7-tetrahydro-2H-pyrazolo[3,4-b]pyrazine-3-carboxamide

### Step 76.1

### tert-butyl 3-[{5-carbamoyl-1-[4-(2,3-difluorophenoxy)phenyl]-4-nitro-1H-pyrazol-3-yl}(prop-2-en-1-yl)amino]azetidine-1-carboxylate

Step 76.1 was prepared according to the procedure for Step 81.1, substituting [4-(2,3-difluorophenoxy)phenyl]boronic acid (Intermediate 0) for [4-(2,4-difluorophenoxy)phenyl]boronic acid. ¹H NMR (400 MHz, dimethyl sulfoxide-d₆) δ ppm 1.36 (s, 9H) 3.80 (br dd, J=8.68, 5.62 Hz, 2H) 3.89-4.03 (m, 4H) 4.24-4.37 (m, 1H) 5.08-5.27 (m, 2H) 5.81 (br dd, J=16.93, 10.33 Hz, 1H) 7.10 (br t, J=7.58 Hz, 1H) 7.17-7.41 (m, 4H) 7.58 (d, J=8.93 Hz, 2H) 8.20 (s, 1H) 8.44 (s, 1H).

### Step 76.2

### tert-butyl 3-[{5-carbamoyl-1-[4-(2,3-difluorophenoxy)phenyl]-4-nitro-1H-pyrazol-3-yl}(2-oxoethyl)amino]azetidine-1-carboxylate

Step 76.2 was prepared according to the procedure for Step 81.2, substituting tert-butyl 3-[{5-carbamoyl-1-[4-(2,3-difluorophenoxy)phenyl]-4-nitro-1H-pyrazol-3-yl}(prop-2-en-1-yl)amino]azetidine-1-carboxylate (Step 76.1) for tert-butyl 3-[{5-carbamoyl-1-[4-(2,4-difluorophenoxy)phenyl]-4-nitro-1H-pyrazol-3-yl}(prop-2-en-1-yl)amino]azetidine-1-carboxylate. MS (ESI) m/z: 517.1 [M-OtButyl+H₂O]+.

### Step 76.3

### tert-butyl 3-{3-carbamoyl-2-[4-(2,3-difluorophenoxy)phenyl]-2,4,5,6-tetrahydro-7H-pyrazolo[3,4-b]pyrazin-7-yl}azetidine-1-carboxylate

Step 76.3 was prepared according to the procedure for Step 81.3, substituting tert-butyl 3-[{5-carbamoyl-1-[4-(2,3-difluorophenoxy)phenyl]-4-nitro-1H-pyrazol-3-yl}(2-oxoethyl)amino]azetidine-1-carboxylate (Step 76.2) for tert-butyl 3-[{5-carbamoyl-1-[4-(2,4-difluorophenoxy)phenyl]-4-nitro-1H-pyrazol-3-yl}(2-oxoethyl)amino]azetidine-1-carboxylate. MS (ESI) m/z: 527.4 [M+H]⁺.

### Step 76.4

### 7-(azetidin-3-yl)-2-[4-(2,3-difluorophenoxy)phenyl]-4,5,6,7-tetrahydro-2H-pyrazolo[3,4-b]pyrazine-3-carboxamide-hydrogen chloride (1/3)

Step 76.4 was prepared according to the procedure for Step 81.4, substituting tert-butyl 3-{3-carbamoyl-2-[4-(2,3-difluorophenoxy)phenyl]-2,4,5,6-tetrahydro-7H-pyrazolo[3,4-b]pyrazin-7-yl}azetidine-1-carboxylate (Step 76.3) for tert-butyl 3-{3-carbamoyl-2-[4-(2,4-difluorophenoxy)phenyl]-2,4,5,6-tetrahydro-7H-pyrazolo[3,4-b]pyrazin-7-yl}azetidine-1-carboxylate. MS (ESI) m/z: 427.3 [M+H]⁺.

### Step 76.5

### 2-[4-(2,3-difluorophenoxy)phenyl]-7-[1-(prop-2-enoyl)azetidin-3-yl]-4,5,6,7-tetrahydro-2H-pyrazolo[3,4-b]pyrazine-3-carboxamide

Step 76.5 was prepared according to the procedure for Step 35.4, substituting 7-(azetidin-3-yl)-2-[4-(2,3-difluorophenoxy)phenyl]-4,5,6,7-tetrahydro-2H-pyrazolo[3,4-b]pyrazine-3-carboxamide hydrogen chloride (1/3) (Step 76.4) for 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide. ¹H NMR (400 MHz, methanol-d₄) 3.32-3.42 (m, 2H) 3.49-3.58 (m, 2H) 4.21-4.46 (m, 3H) 4.47-4.65 (m, 2H) 5.72 (dd, J=10.14, 1.76 Hz, 1H) 6.18-6.27 (m, 1H) 6.30-6.41 (m, 1H) 7.05 (br d, J=8.82 Hz, 5H) 7.38 (br d, J=8.82 Hz, 2H). MS (ESI) m/z: 481.2 [M+H]⁺.

### Example 77

### (7R)-2-[4-(2,3-difluorophenoxy)phenyl]-7-[4-(prop-2-enoyl)piperazin-1-yl]-4,5,6,7-tetrahydro-2H-pyrazolo[4,3-b]pyridine-3-carboxamide

### Step 77.1

### ethyl (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

A mixture of ethyl (7R)-7-[4-(2-nitrobenzene-1-sulfonyl)piperazin-1-yl]-4,5,6,7-tetrahydro-2H-pyrazolo[4,3-b]pyridine-3-carboxylate (0.52 g, 1.119 mmol, Intermediate R), [4-(2,3-difluorophenoxy)phenyl]boronic acid (0.504 g, 2.015 mmol, Intermediate 0), diacetoxycopper (0.224 g, 1.231 mmol), and 4 Å molecular sieves (1.12 g) was stirred in dichloromethane (22.39 mL). Pyridine (0.109 mL, 1.343 mmol) was added dropwise, and the reaction was stirred for 48 hours, open to the air. The reaction mixture was filtered through a short bed (4 cm) of silica gel which was washed with dichloromethane (50 mL) and then 3:1 ethanol/ethyl acetate (100 mL) to elute the product. The eluent was concentrated under reduced pressure to afford a residue which was purified by column chromatography on silica gel (eluted with 0-100% heptane/ethyl acetate) to afford the title compound (0.36 g, 48.1%). MS (APCI) m/z: 669.3 [M+H]⁺.

### Step 77.2

### (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid

To a suspension of ethyl (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (0.36 g, 0.54 mmol, Step 77.1) in tetrahydrofuran (3.23 mL), methanol (1.08 mL) and water (1.08 mL) was added LiOH.H₂O (0.113 g, 2.69 mmol). The reaction mixture was stirred at 60° C. for 6 hours. The reaction was concentrated under reduced pressure, and the aqueous phase was acidified with 1.2 M citric acid solution (2.24 mL, 2.69 mmol) to approximately pH 5 and extracted with ethyl acetate (3×). The combined organic layers were dried over Na₂SO₄ and concentrated under reduced pressure to afford the title compound which used was directly in the next step without purification. MS (APCI) m/z: 641.3 [M+H]⁺.

### Step 77.3

### (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

To a solution of (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid (0.34 g, 0.53 mmol, Step 77.2) in dichloromethane (6.3 mL) was added (1-[bis(dimethylamino)methylene]-1H-1,2,3-triazolo[4,5-b]pyridinium 3-oxid hexafluorophosphate (0.40 g, 1.06 mmol), triethylamine (0.44 mL, 3.18 mmol) and ammonium chloride (0.14 g, 2.65 mmol) in order at 25° C. The resulting mixture was stirred for 6.5 hours. The reaction was diluted with water and extracted with ethyl acetate (3×). The combined organic layers were washed with brine, dried over Na₂SO₄, filtered, and concentrated under reduced pressure. The residue was purified by column chromatography on silica gel (eluted with dichloromethane/acetone 40-60%) to afford the title compound (0.32 g, 93%). MS (APCI) m/z: 640.3 [M+H]⁺.

### Step 77.4

### (7R)-2-[4-(2,3-difluorophenoxy)phenyl]-7-(piperazin-1-yl)-4,5,6,7-tetrahydro-2H-pyrazolo[4,3-b]pyridine-3-carboxamide

To a solution of (7R)-2-[4-(2,3-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (0.316 g, 0.494 mmol, Step 77.3) in tetrahydrofuran (6.18 mL) under nitrogen at 0° C. was added decane-1-thiol (0.125 mL, 0.593 mmol) followed by dropwise addition of 2.0 M sodium tert-butoxide in tetrahydrofuran (0.272 mL, 0.543 mmol). After 15 minutes the reaction was quenched by the addition of 1 M HCl (3.0 mL), and the reaction stirred for 30 minutes at 25° C. The reaction was extracted with tert-butyl methyl ether (3×), made basic with 2 N KOH, and extracted with dichloromethane (3×). The combined organic layer was washed with brine, dried over Na₂SO₄, filtered, and concentrated under reduced pressure to afford the title compound which was used in next step without purification. MS (APCI) m/z: 455.2 [M+H]⁺.

### Step 77.5

### (7R)-2-[4-(2,3-difluorophenoxy)phenyl]-7-[4-(prop-2-enoyl)piperazin-1-yl]-4,5,6,7-tetrahydro-2H-pyrazolo[4,3-b]pyridine-3-carboxamide

Step 77.5 was prepared according to the procedure for the Step 35.4, substituting (7R)-2-[4-(2,3-difluorophenoxy)phenyl]-7-(piperazin-1-yl)-4,5,6,7-tetrahydro-2H-pyrazolo[4,3-b]pyridine-3-carboxamide (Step 77.4) for 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide. ¹H NMR (400 MHz, dimethyl sulfoxide-d₆) δ ppm 7.35-7.17 (m, 4H), 7.11-7.03 (m, 2H), 6.99 (ddt, J=8.6, 7.4, 1.8 Hz, 1H), 6.73 (dd, J=16.7, 10.5 Hz, 1H), 6.05 (dd, J=16.7, 2.4 Hz, 1H), 5.62 (dd, J=10.4, 2.4 Hz, 1H), 5.06 (s, 1H), 3.61 (t, J=5.5 Hz, 1H), 3.46 (d, J=29.1 Hz, 1H), 3.29-3.18 (m, 2H), 3.15-3.01 (m, 1H), 2.72-2.51 (m, 5H), 2.11-1.97 (m, 1H), 1.71 (dt, J=8.6, 4.9 Hz, 1H). MS (APCI) m/z: 509.4 [M+H]⁺.

### Example 78

### (7R)-2-[4-(3-cyclopropylphenoxy)phenyl]-7-[4-(prop-2-enoyl)piperazin-1-yl]-4,5,6,7-tetrahydro-2H-pyrazolo[4,3-b]pyridine-3-carboxamide

### Step 78.1

### ethyl (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

To a solution of ethyl (7R)-7-[4-(2-nitrobenzene-1-sulfonyl)piperazin-1-yl]-4,5,6,7-tetrahydro-2H-pyrazolo[4,3-b]pyridine-3-carboxylate (0.400 g, 0.861 mmol, Intermediate R) in dichloromethane (17 mL) was added Cu(OC(O)CH₃)₂ (0.235 g, 1.292 mmol), pyridine (0.209 mL, 2.58 mmol), [4-(3-cyclopropylphenoxy)phenyl]boronic acid (0.438 g, 1.72 mmol, Intermediate U), and activated 4 Å molecular sieves (1.00 g). The mixture was stirred under air for 24 hours at ambient temperature. The reaction mixture was filtered through a silica plug. The plug was rinsed with ethyl acetate, and the filtrate was concentrated under reduced pressure. The crude residue was purified by column chromatography (0% to 100% ethyl acetate in heptanes) to afford the title compound (0.179 g, 31%). MS (ESI) m/z: 672.9 [M+H]⁺.

### Step 78.2

### (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid

To a solution of ethyl (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (0.179 g, 0.266 mmol, Step 78.1) in tetrahydrofuran (0.800 mL), water (0.270 mL) and methanol (0.270 mL), was added lithium hydroxide monohydrate (0.056 g, 1.33 mmol). The mixture was heated to 50° C. After 100 minutes the reaction mixture was cooled to ambient temperature, acidified with 1 N HCl (1.33 mL, 1.33 mmol) and extracted with ethyl acetate (3×). The combined organic layers were washed with brine, dried over Na₂SO₄, filtered, and concentrated to afford the title compound which was directly for Step 78.3. MS (ESI) m/z: 644.9 [M+H]⁺.

### Step 78.3

### (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

To a solution of (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid (0.172 g, 0.266 mmol, Step 78.2), (1-[bis(dimethylamino)methylene]-1H-1,2,3-triazolo[4,5-b]pyridinium 3-oxid hexafluorophosphate (0.112 g, 0.293 mmol), and ammonium chloride (0.029 g, 0.534 mmol) in N,N-dimethylformamide (2.7 mL), was added N,N-dimethylformamide (2.7 mL) and N,N-diisopropylethylamine (0.140 mL, 0.800 mmol). The mixture was stirred at ambient temperature. After 80 minutes, the reaction mixture was quenched with saturated NaHCO₃, diluted with water, and extracted with ethyl acetate (3×). The combined organic layers were washed with brine, dried over Na₂SO₄, filtered, and concentrated under reduced pressure. The crude residue was purified by column chromatograph (0% to 5% methanol in dichloromethane) to afford the title compound (0.132 g, 77% over 2 steps). MS (ESI) m/z: 643.9 [M+H]⁺.

### Step 78.4

### (7R)-2-[4-(3-cyclopropylphenoxy)phenyl]-7-(piperazin-1-yl)-4,5,6,7-tetrahydro-2H-pyrazolo[4,3-b]pyridine-3-carboxamide

To a solution of (7R)-2-[4-(3-cyclopropylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (0.132 g, 0.205 mmol, Step 78.3) in tetrahydrofuran (1.4 mL) at 0° C. was added decane-1-thiol (0.052 mL, 0.246 mmol), followed by dropwise addition of sodium tert-butoxide (2 M in tetrahydrofuran, 0.113 mL, 0.226 mmoL). After 5 minutes the reaction mixture was acidified with 1 N HCl and extracted with tert-butyl methyl ether (3×). The tert-butyl methyl ether layers were discarded, and the aqueous layer was basified with 2 N KOH and then extracted with dichloromethane (3×). The combined dichloromethane layers were dried over Na₂SO₄, filtered, and concentrated under reduced pressure to afford the title compound, which was directly for Step 78.5. MS (ESI) m/z: 459.1 [M+H]⁺.

### Step 78.5

### (7R)-2-[4-(3-cyclopropylphenoxy)phenyl]-7-[4-(prop-2-enoyl)piperazin-1-yl]-4,5,6,7-tetrahydro-2H-pyrazolo[4,3-b]pyridine-3-carboxamide

To a solution of (7R)-2-[4-(3-cyclopropylphenoxy)phenyl]-7-(piperazin-1-yl)-4,5,6,7-tetrahydro-2H-pyrazolo[4,3-b]pyridine-3-carboxamide (0.92 g, 0.201 mmol, Step 78.4) and N,N-diisopropylethylamine (0.35 mL, 2.01 mmol) in dichloromethane (4 mL) at 0° C. was added acrylic acid (3M in dichloromethane, 0.60 mL, 0.181 mmol) and propylphosphonic anhydride (50% weight in ethyl acetate, 0.143 mL, 0.241 mmol). After 5 minutes, saturated NaHCO₃ (4 mL) was added, and the reaction mixture was transferred to a separatory funnel and extracted with dichloromethane (3×). The combined organic layers were dried over Na₂SO₄, filtered, and concentrated under reduced pressure. The crude residue was purified by column chromatography (0% to 5% methanol in dichloromethane) to afford the title compound (0.045 g, 42% over 2 Steps). ¹H NMR (600 MHz, dimethyl sulfoxide-d₆) δ ppm 7.34-7.30 (m, 2H), 7.27 (td, J=7.7, 0.6 Hz, 1H), 7.02-6.99 (m, 2H), 6.88-6.86 (m, 3H), 6.81-6.75 (m, 3H), 6.09 (dd, J=16.7, 2.4 Hz, 1H), 5.66 (dd, J=10.4, 2.4 Hz, 1H), 5.09 (t, J=3.0 Hz, 1H), 3.65 (dd, J=6.2, 4.8 Hz, 1H), 3.59-3.40 (m, 3H), 3.29-3.22 (m, 1H), 3.11 (ddt, J=11.4, 6.6, 3.1 Hz, 1H), 2.72-2.53 (m, 4H), 2.07 (dtt, J=13.4, 6.6, 2.9 Hz, 1H), 1.92 (tt, J=8.4, 5.1 Hz, 1H), 1.74 (dddd, J=13.5, 8.0, 4.7, 3.3 Hz, 1H), 0.98-0.92 (m, 2H), 0.70-0.65 (m, 2H). MS (ESI) m/z: 513.0 [M+H]⁺.

### Example 79

### (7R)-2-[4-(2-fluorophenoxy)phenyl]-7-[4-(prop-2-enoyl)piperazin-1-yl]-4,5,6,7-tetrahydro-2H-pyrazolo[4,3-b]pyridine-3-carboxamide

### Step 79.1

### ethyl (7R)-2-[4-(2-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 79.1 was prepared according to the procedure for Step 74.1, substituting [4-(2-fluorophenoxy)phenyl]boronic acid for [4-(2,5-difluorophenoxy)phenyl]boronic acid. MS (APCI) m/z: 650.9 [M+H]⁺.

### Step 79.2

### (7R)-2-[4-(2-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid

Step 79.2 was prepared according to the procedure for Step 74.2, substituting ethyl (7R)-2-[4-(2-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 79.1) for ethyl (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate. MS (APCI) m/z: 622.9 [M+H]⁺.

### Step 79.3

### (7R)-2-[4-(2-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 79.3 was prepared according to the procedure for Step 74.3, substituting (7R)-2-[4-(2-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid (Step 79.2) for (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxylic acid. MS (APCI) m/z: 621.9 [M+H]⁺.

### Step 79.4

### (7R)-2-[4-(2-fluorophenoxy)phenyl]-7-(piperazin-1-yl)-4,5,6,7-tetrahydro-2H-pyrazolo[4,3-b]pyridine-3-carboxamide

Step 79.4 was prepared according to the procedure for Step 74.4, substituting (7R)-2-[4-(2-fluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 79.3) for (7R)-2-[4-(2,5-difluorophenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide. MS (APCI) m/z: 437.2 [M+H]⁺.

### Step 79.5

### (7R)-2-[4-(2-fluorophenoxy)phenyl]-7-[4-(prop-2-enoyl)piperazin-1-yl]-4,5,6,7-tetrahydro-2H-pyrazolo[4,3-b]pyridine-3-carboxamide

Step 79.5 was prepared according to the procedure for Step 12.4, substituting (7R)-2-[4-(2-fluorophenoxy)phenyl]-7-(piperazin-1-yl)-4,5,6,7-tetrahydro-2H-pyrazolo[4,3-b]pyridine-3-carboxamide (Step 79.4) for 2-[4-(3-fluorophenoxy)phenyl]-7-(piperidin-4-yl)-4,5,6,7-tetrahydro-2H-pyrazolo[4,3-b]pyridine-3-carboxamide. ¹H NMR (500 MHz, CDCl₃) δ ppm 7.44-7.38 (m, 2H), 7.27-7.13 (m, 4H), 7.11-7.03 (m, 2H), 6.57 (dd, J=16.8, 10.6 Hz, 1H), 6.29 (dd, J=16.8, 1.9 Hz, 1H), 5.69 (dd, J=10.6, 1.9 Hz, 1H), 5.27-5.11 (m, 3H), 3.87 (dd, J=6.7, 5.0 Hz, 1H), 3.83-3.75 (m, 1H), 3.72-3.53 (m, 3H), 3.51-3.41 (m, 1H), 3.39-3.27 (m, 1H), 2.88-2.62 (m, 4H), 2.26-2.12 (m, 1H), 2.05-1.92 (m, 1H). MS (APCI) m/z: 491.0 [M+H]⁺.

### Example 80

### 2-[4-(2,5-difluorophenoxy)phenyl]-7-[1-(prop-2-enoyl)azetidin-3-yl]-4,5,6,7-tetrahydro-2H-pyrazolo[3,4-b]pyrazine-3-carboxamide

### Step 80.1

### tert-butyl 3-[{5-carbamoyl-1-[4-(2,5-difluorophenoxy)phenyl]-4-nitro-1H-pyrazol-3-yl}(prop-2-en-1-yl)amino]azetidine-1-carboxylate

Step 80.1 was prepared according to the procedure for Step 81.1, substituting [4-(2,5-difluorophenoxy)phenyl]boronic acid (Intermediate G), for [4-(2,4-difluorophenoxy)phenyl]boronic acid. MS (ESI) m/z: 515.1 [M-OtButyl+H₂O]⁺.

### Step 80.2

### tert-butyl 3-[{5-carbamoyl-1-[4-(2,5-difluorophenoxy)phenyl]-4-nitro-1H-pyrazol-3-yl}(2-oxoethyl)amino]azetidine-1-carboxylate

Step 80.2 was prepared according to the procedure for Step 81.2, substituting tert-butyl 3-[{5-carbamoyl-1-[4-(2,5-difluorophenoxy)phenyl]-4-nitro-1H-pyrazol-3-yl}(prop-2-en-1-yl)amino]azetidine-1-carboxylate (Step 80.1) for tert-butyl 3-[{5-carbamoyl-1-[4-(2,4-difluorophenoxy)phenyl]-4-nitro-1H-pyrazol-3-yl}(prop-2-en-1-yl)amino]azetidine-1-carboxylate. MS (ESI) m/z: 517.1 [M-OtButyl+H₂O]⁺.

### Step 80.3

### tert-butyl 3-{3-carbamoyl-2-[4-(2,5-difluorophenoxy)phenyl]-2,4,5,6-tetrahydro-7H-pyrazolo[3,4-b]pyrazin-7-yl}azetidine-1-carboxylate

Step 80.3 was prepared according to the procedure for Step 81.3, substituting tert-butyl 3-[{5-carbamoyl-1-[4-(2,5-difluorophenoxy)phenyl]-4-nitro-1H-pyrazol-3-yl}(2-oxoethyl)amino]azetidine-1-carboxylate (Step 80.2) for tert-butyl 3-[{5-carbamoyl-1-[4-(2,4-difluorophenoxy)phenyl]-4-nitro-1H-pyrazol-3-yl}(2-oxoethyl)amino]azetidine-1-carboxylate. MS (ESI) m/z: 527.2 [M+H]⁺.

### Step 80.4

### 7-(azetidin-3-yl)-2-[4-(2,5-difluorophenoxy)phenyl]-4,5,6,7-tetrahydro-2H-pyrazolo[3,4-b]pyrazine-3-carboxamide-hydrogen chloride (1/3)

Step 80.4 was prepared according to the procedure for Step 81.4, substituting tert-butyl 3-{3-carbamoyl-2-[4-(2,5-difluorophenoxy)phenyl]-2,4,5,6-tetrahydro-7H-pyrazolo[3,4-b]pyrazin-7-yl}azetidine-1-carboxylate (Step 80.3) for tert-butyl 3-{3-carbamoyl-2-[4-(2,4-difluorophenoxy)phenyl]-2,4,5,6-tetrahydro-7H-pyrazolo[3,4-b]pyrazin-7-yl}azetidine-1-carboxylate. MS (ESI) m/z: 427.1 [M+H]⁺.

### Step 80.5

### 2-[4-(2,5-difluorophenoxy)phenyl]-7-[1-(prop-2-enoyl)azetidin-3-yl]-4,5,6,7-tetrahydro-2H-pyrazolo[3,4-b]pyrazine-3-carboxamide

Step 80.5 was prepared according to the procedure for Step 35.4, substituting 7-(azetidin-3-yl)-2-[4-(2,5-difluorophenoxy)phenyl]-4,5,6,7-tetrahydro-2H-pyrazolo[3,4-b]pyrazine-3-carboxamide hydrogen chloride (1/3) (Step 80.4) for 7-[(1R,5S)-3,8-diazabicyclo[3.2.1]octan-3-yl]-2-(4-phenoxyphenyl)-4,5,6,7-tetrahydro-2H-pyrazolo[4,3-b]pyridine-3-carboxamide. ¹H NMR (400 MHz, dimethyl sulfoxide-d₆) δ ppm 3.13 (br s, 2H) 3.21-3.28 (m, 2) 4.11 (br s, 2H) 4.33 (br s, 1H) 4.39 (br s, 2H) 5.10 (br s, 1H) 5.65 (br d, J=9.48 Hz, 1H) 6.09 (br d, J=16.54 Hz, 1H) 6.24-6.39 (m, 1H) 7.03 (br s, 4H) 7.25 (br d, J=7.06 Hz, 2H) 7.45 (br s, 1H). MS (ESI) m/z: 481.2 [M+H]⁺.

### Example 81

### 2-[4-(2,4-difluorophenoxy)phenyl]-7-[1-(prop-2-enoyl)azetidin-3-yl]-4,5,6,7-tetrahydro-2H-pyrazolo[3,4-b]pyrazine-3-carboxamide

### Step 81.1

### tert-butyl 3-[{5-carbamoyl-1-[4-(2,4-difluorophenoxy)phenyl]-4-nitro-1H-pyrazol-3-yl}(prop-2-en-1-yl)amino]azetidine-1-carboxylate

To a solution of tert-butyl 3-[(3-carbamoyl-4-nitro-1H-pyrazol-5-yl)(prop-2-en-1-yl)amino]azetidine-1-carboxylate (1 g, 2.73 mmol, Step J.6) in dichloromethane (30 mL) was added pyridine (0.4 mL 5.46 mmol), [4-(2,4-difluorophenoxy)phenyl]boronic acid (1.36 g, 5.46 mmol, Intermediate I), 4 Å molecular sieves (100 mg) and copper (II) acetate (540 mg, 3.00 mmol). The reaction mixture was stirred for 12 hours at 20° C. under oxygen (15 psi). The reaction was quenched with water and extracted with ethyl acetate (3×). The organic phases were combined and washed with brine, dried over Na₂SO₄, and filtered. The filtrate was concentrated under reduced pressure to afford a crude product which was purified by column chromatography on silica gel (1:1 petroleum ether/ethyl acetate) to afford the title compound (1 g, 51.4%). ¹H NMR (400 MHz, dimethyl sulfoxide-d₆) δ ppm 1.36 (s, 9H) 3.73-3.85 (m, 2H) 3.89-4.07 (m, 4H) 4.24-4.36 (m, 1H) 5.09-5.25 (m, 2H) 5.74-5.88 (m, 1H) 7.11 (d, J=9.04 Hz, 2H) 7.15-7.24 (m, 1H) 7.35-7.45 (m, 1H) 7.54 (br d, J=9.04 Hz, 3H) 8.19 (s, 1H) 8.43 (s, 1H).

### Step 81.2

### tert-butyl 3-[{5-carbamoyl-1-[4-(2,4-difluorophenoxy)phenyl]-4-nitro-1H-pyrazol-3-yl}(2-oxoethyl)amino]azetidine-1-carboxylate

To a solution of tert-butyl 3-[{5-carbamoyl-1-[4-(2,4-difluorophenoxy)phenyl]-4-nitro-1H-pyrazol-3-yl}(prop-2-en-1-yl)amino]azetidine-1-carboxylate (1 g, 1.75 mmol, Step 81.1) in tetrahydrofuran (10 mL) and water (5 mL) was added 4-methylmorpholine N-oxide (0.4 g, 3.51 mmol) and osmium tetroxide (0.4 g, 1.57 mmol). The reaction mixture was stirred for 12 hours at 25° C. The reaction was quenched by the addition of sodium bisulfite (1.8 g, 17.53 mmol) in water (10 mL), and the resulting solution was stirred for 10 minutes at 25° C. The mixture was extracted with ethyl acetate (3×20 mL). The combined organic phases were combined, washed with brine (15 mL), dried over Na₂SO₄, and filtered. The filtrate was concentrated under reduced pressure to afford the crude diol. To a solution of the crude diol (1 g) in acetone (10 mL) was added a solution of sodium periodate (0.7 g, 3.31 mmol) in water (1 mL), and the reaction mixture was stirred for 2 hours at 25° C. The mixture was quenched with water and extracted with ethyl acetate (3×). The organic phases were combined, washed with brine, dried over Na₂SO₄, and filtered. The filtrate was concentrated under reduced pressure to afford the title compound (1 g, 84%). ¹H NMR (400 MHz, dimethyl sulfoxide-d₆) δ ppm 1.36 (br s, 9H) 3.83 (br s, 2H) 3.94-4.12 (m, 3H) 4.27 (br s, 1H) 4.45-4.62 (m, 1H) 7.01-7.24 (m, 3H) 7.34-7.46 (m, 1H) 7.54 (br d, J=7.06 Hz, 3H) 8.19 (br s, 1H) 8.43 (br s, 1H) 9.64 (s, 1H).

### Step 81.3

### tert-butyl 3-{3-carbamoyl-2-[4-(2,4-difluorophenoxy)phenyl]-2,4,5,6-tetrahydro-7H-pyrazolo[3,4-b]pyrazin-7-yl}azetidine-1-carboxylate

To a solution of tert-butyl 3-[{5-carbamoyl-1-[4-(2,4-difluorophenoxy)phenyl]-4-nitro-1H-pyrazol-3-yl}(2-oxoethyl)amino]azetidine-1-carboxylate (1 g, 1.39 mmol, Step 81.2) in tetrahydrofuran (10 mL) was added rhodium on carbon (2 g, 1.944 mmol). The reaction mixture was stirred for 12 hours at 25° C. under hydrogen (15 psi). The reaction mixture was filtered and concentrated under reduced pressure to afford the title compound (140 mg, 19.0%). MS (ESI) m/z: 527.4 [M+H]⁺.

### Step 81.4

### 7-(azetidin-3-yl)-2-[4-(2,4-difluorophenoxy)phenyl]-4,5,6,7-tetrahydro-2H-pyrazolo[3,4-b]pyrazine-3-carboxamide-hydrogen chloride (1/3)

To a solution of tert-butyl 3-{3-carbamoyl-2-[4-(2,4-difluorophenoxy)phenyl]-2,4,5,6-tetrahydro-7H-pyrazolo[3,4-b]pyrazin-7-yl}azetidine-1-carboxylate (140 mg, 0.266 mmol, Step 81.3) in ethyl acetate (2 mL) was added 4 N HCl in ethyl acetate (2 mL). The reaction mixture was stirred for 0.5 hours at 25° C. The mixture was concentrated under reduced pressure to afford the title compound (100 mg, 44.1%). MS (ESI) m/z: 427.4 [M+H]⁺.

### Step 81.5

### 2-[4-(2,4-difluorophenoxy)phenyl]-7-[1-(prop-2-enoyl)azetidin-3-yl]-4,5,6,7-tetrahydro-2H-pyrazolo[3,4-b]pyrazine-3-carboxamide

To a solution of acrylic acid (20.2 mg, 0.28 mmol) in dichloromethane (1 mL) was added N,N-diisopropylethylamine (0.12 mL, 0.70 mmol) and 2,4,6-tripropyl-1,3,5,2,4, -trioxatriphosphinane 2,4,6-trioxide (112 mg, 0.35 mmol). The mixture stirred for 1 hour at 0° C. To the reaction mixture was added 7-(azetidin-3-yl)-2-[4-(2,4-difluorophenoxy)phenyl]-4,5,6,7-tetrahydro-2H-pyrazolo[3,4-b]pyrazine-3-carboxamide-hydrogen chloride (1/3) (100 mg, 0.23 mmol, Step 81.4) and N,N-diisopropylethylamine (0.12 mL, 0.70 mmol) in dichloromethane (1 mL). The reaction mixture was stirred for 0.5 hour at 0° C., quenched with water, and extracted with ethyl acetate (3×). The organic phases were combined, washed with brine, dried over Na₂SO₄, and filtered. The filtrate was concentrated under reduced pressure to afford a residue which was purified by preparative HPLC (25-55% acetonitrile in 10 mM ammonium bicarbonate over 8.1 minutes, 55-100% over 0.1 minutes, then 100% for 6 minutes; Column: Waters Xbridge BEH C18 100×25 mm×5 μm particle size; Flow Rate: 25 mL/min; Detection wavelength: 220 nm and 254 nm) to afford the title compound (22 mg, 19.53%). ¹H NMR (400 MHz, methanol-d₄) δ ppm 3.23 (br dd, J=9.37, 4.52 Hz, 2H) 3.44 (br s, 2H) 4.19-4.43 (m, 3H) 4.46-4.53 (m, 1H) 4.54-4.58 (m, 1H) 5.72 (br d, J=10.14 Hz, 1H) 6.17-6.28 (m, 1H) 6.30-6.42 (m, 1H) 6.90-7.04 (m, 3H) 7.09-7.24 (m, 2H) 7.29 (br d, J=8.60 Hz, 2H). MS (ESI) m/z: 481.2 [M+H]⁺.

### Example 82

### (7R)-2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide; and

### Example 83

### (7S)-2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide

The racemate, 2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide (Example 53) was separated by preparative HPLC to afford Example 83. MS (ESI) m/z: 499.2 [M+H]⁺, and Example 82. ¹H NMR (400 MHz, dimethyl sulfoxide-d₆) 7.52-7.37 (m, 2H), 7.37-7.27 (m, 2H), 7.23-7.12 (m, 1H), 7.10-7.00 (m, 4H), 6.10 (d, J=16.9 Hz, 1H), 5.67 (d, J=10.4 Hz, 1H), 5.08 (s, 1H), 3.61 (s, 1H), 3.23 (dd, J=11.4, 8.7 Hz, 1H), 3.10 (dd, J=10.5, 6.3 Hz, 1H), 2.00 (d, J=9.7 Hz, 1H), 1.70 (s, 1H). MS (ESI) m/z: 499.3 [M+H]⁺.

### Example 84

### 7-[4-hydroxy-1-(prop-2-enoyl)piperidin-4-yl]-2-(4-phenoxyphenyl)-4,5,6,7-tetrahydro-2H-pyrazolo[4,3-b]pyridine-3-carboxamide

### Step 84.1

### ethyl 7-[1-(tert-butoxycarbonyl)-4-hydroxypiperidin-4-yl]-2-(4-phenoxyphenyl)-2H-pyrazolo[4,3-b]pyridine-3-carboxylate

To a solution of ethyl 7-[1-(tert-butoxycarbonyl)-1,2,3,6-tetrahydropyridin-4-yl]-2-(4-phenoxyphenyl)-2H-pyrazolo[4,3-b]pyridine-3-carboxylate (0.812 g, 1.50 mmol, Intermediate P) in isopropyl alcohol (5 mL) and dichloromethane (1 mL) was added phenyl silane (0.650 g, 6.01 mmol) and tris(2,2,6,6-tetramethyl-3,5-heptanedionato)manganese(III) (0.272 g, 0.451 mmol). The mixture was stirred under an oxygen atmosphere at ambient temperature for 30 minutes. The reaction mixture was diluted with water and extracted with ethyl acetate (2×10 mL). The combined organic layers were washed with brine, dried over Na₂SO₄, filtered, and concentrated under reduced pressure. The crude residue was purified by column chromatography (0% to 100% ethyl acetate in heptanes) to afford the title compound (0.669 g, 80%). MS (ESI) m/z: 559.3 [M+H]⁺.

### Step 84.2

### ethyl 7-[1-(tert-butoxycarbonyl)-4-hydroxypiperidin-4-yl]-2-(4-phenoxyphenyl)-4,5,6,7-tetrahydro-2H-pyrazolo[4,3-b]pyridine-3-carboxylate

To a solution of ethyl 7-[1-(tert-butoxycarbonyl)-4-hydroxypiperidin-4-yl]-2-(4-phenoxyphenyl)-2H-pyrazolo[4,3-b]pyridine-3-carboxylate (0.195 g, 0.349 mmol, Step 84.1) in methanol (2 mL) and tetrahydrofuran (2 mL) was added 10% Pd(OH)₂/C (0.200 g, 0.349 mmol). The mixture was stirred under a hydrogen atmosphere (60 psi) at 50° C. After 20 hours, the resulting solution was filtered over diatomaceous earth, and the filtrate was concentrated under reduced pressure. The crude residue was purified by column chromatography (0% to 100% ethyl acetate in heptanes) to afford the title compound (0.114 g, 58%). MS (ESI) m/z: 563.3 [M+H]⁺.

### Step 84.3

### tert-butyl 4-[3-carbamoyl-2-(4-phenoxyphenyl)-4,5,6,7-tetrahydro-2H-pyrazolo[4,3-b]pyridin-7-yl]-4-hydroxypiperidine-1-carboxylate

Step 84.3 was prepared according to Step 66.2, substituting ethyl 7-[1-(tert-butoxycarbonyl)-4-hydroxypiperidin-4-yl]-2-(4-phenoxyphenyl)-4,5,6,7-tetrahydro-2H-pyrazolo[4,3-b]pyridine-3-carboxylate (Step 84.2) for ethyl 7-[rac-(3aR,6aS)-5-(tert-butoxycarbonyl)hexahydropyrrolo[3,4-c]pyrrol-2(1H)-yl]-2-(4-phenoxyphenyl)-4,5,6,7-tetrahydro-2H-pyrazolo[4,3-b]pyridine-3-carboxylate. MS (ESI) m/z: 534.3 [M+H]⁺.

### Step 84.4

### 7-(4-hydroxypiperidin-4-yl)-2-(4-phenoxyphenyl)-4,5,6,7-tetrahydro-2H-pyrazolo[4,3-b]pyridine-3-carboxamide

Step 84.4 was prepared according to Step 66.3, substituting tert-butyl 4-[3-carbamoyl-2-(4-phenoxyphenyl)-4,5,6,7-tetrahydro-2H-pyrazolo[4,3-b]pyridin-7-yl]-4-hydroxypiperidine-1-carboxylate (Step 84.3) for rac-(3aR,6aS)-5-[3-carbamoyl-2-(4-phenoxyphenyl)-4,5,6,7-tetrahydro-2H-pyrazolo[4,3-b]pyridin-7-yl]hexahydropyrrolo[3,4-c]pyrrole-2(1H)-carboxylate.

### Step 84.5

### 7-[4-hydroxy-1-(prop-2-enoyl)piperidin-4-yl]-2-(4-phenoxyphenyl)-4,5,6,7-tetrahydro-2H-pyrazolo[4,3-b]pyridine-3-carboxamide

Step 84.5 was prepared according to Step 66.4, substituting 7-(4-hydroxypiperidin-4-yl)-2-(4-phenoxyphenyl)-4,5,6,7-tetrahydro-2H-pyrazolo[4,3-b]pyridine-3-carboxamide (Step 84.4) for 7-[rac-(3aR,6aS)-hexahydropyrrolo[3,4-c]pyrrol-2(1H)-yl]-2-(4-phenoxyphenyl)-4,5,6,7-tetrahydro-2H-pyrazolo[4,3-b]pyridine-3-carboxamide-hydrogen chloride (1/1). ¹H NMR (500 MHz, dimethyl sulfoxide-d₆) δ ppm 7.42-7.35 (m, 3H), 7.25-7.21 (m, 2H), 7.11-7.01 (m, 4H), 6.51 (dd, J=10.3, 10.7 Hz, 1H), 6.25 (dd, J=10.7, 7.8 Hz, 1H), 5.59-5.64 (m, 1H), 4.72 (s, 1H), 4.52-4.43 (m, 2H), 3.46-3.39 (m, 2H), 3.37-3.32 (m, 2H), 3.07 (t, J=8.4 Hz, 2H), 3.02-2.99 (m, 1H), 2.11 (s, 1H), 1.95-1.81 (m, 3H), 1.76 (dd, J=11.2, 11.2 Hz, 1H), 1.67-1.48 (m, 2H). MS (ESI) m/z: 488.2 [M+H]⁺.

### Example 85

### (7R)-7-[4-(prop-2-enoyl)piperazin-1-yl]-2-{4-[4-(trifluoromethoxy)phenoxy]phenyl}-4,5,6,7-tetrahydro-2H-pyrazolo[4,3-b]pyridine-3-carboxamide

### Step 85.1

### ethyl (7R)-7-[4-(2-nitrobenzene-1-sulfonyl)piperazin-1-yl]-2-{4-[4-(trifluoromethoxy)phenoxy]phenyl}-4,5,6,7-tetrahydro-2H-pyrazolo[4,3-b]pyridine-3-carboxylate

Step 85.1 was prepared according to Step 100.1, substituting {4-[4-(trifluoromethoxy)phenoxy]phenyl}boronic acid for {4-[2-(trifluoromethoxy)phenoxy]phenyl}boronic acid to afford the title compound. MS (ESI) m/z: 717.3 [M+H]⁺.

### Step 85.2

### (7R)-7-[4-(2-nitrobenzene-1-sulfonyl)piperazin-1-yl]-2-{4-[4-(trifluoromethoxy)phenoxy]phenyl}-4,5,6,7-tetrahydro-2H-pyrazolo[4,3-b]pyridine-3-carboxamide

Step 85.2 was prepared according to Step 100.2, substituting ethyl (7R)-7-[4-(2-nitrobenzene-1-sulfonyl)piperazin-1-yl]-2-{4-[4-(trifluoromethoxy)phenoxy]phenyl}-4,5,6,7-tetrahydro-2H-pyrazolo[4,3-b]pyridine-3-carboxylate (Step 85.1) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate to afford the title compound. MS (ESI) m/z: 688.6 [M+H]⁺.

### Step 85.3

### (7R)-7-(piperazin-1-yl)-2-{4-[4-(trifluoromethoxy)phenoxy]phenyl}-4,5,6,7-tetrahydro-2H-pyrazolo[4,3-b]pyridine-3-carboxamide

Step 85.3 was prepared according to Step 100.3, substituting (7R)-7-[4-(2-nitrobenzene-1-sulfonyl)piperazin-1-yl]-2-{4-[4-(trifluoromethoxy)phenoxy]phenyl}-4,5,6,7-tetrahydro-2H-pyrazolo[4,3-b]pyridine-3-carboxamide (Step 85.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide to afford the title compound. MS (ESI) m/z: 503.5 [M+H]⁺.

### Step 85.4

### (7R)-7-[4-(prop-2-enoyl)piperazin-1-yl]-2-{4-[4-(trifluoromethoxy)phenoxy]phenyl}-4,5,6,7-tetrahydro-2H-pyrazolo[4,3-b]pyridine-3-carboxamide

Step 85.4 was prepared according to Step 100.4, substituting (7R)-7-(piperazin-1-yl)-2-{4-[4-(trifluoromethoxy)phenoxy]phenyl}-4,5,6,7-tetrahydro-2H-pyrazolo[4,3-b]pyridine-3-carboxamide (Step 85.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide to afford the title compound. ¹H NMR (400 MHz, dimethyl sulfoxide-d₆) δ ppm 7.41 (d, J=8.8 Hz, 2H), 7.36 (d, J=8.9 Hz, 2H), 7.20-7.14 (m, 2H), 7.09 (d, J=8.9 Hz, 2H), 6.78 (dd, J=16.7, 10.5 Hz, 1H), 6.09 (dd, J=16.7, 2.4 Hz, 1H), 5.66 (dd, J=10.4, 2.4 Hz, 1H), 5.11 (s, 1H), 3.65 (t, J=5.5 Hz, 1H), 3.60-3.44 (m, 4H), 3.25 (d, J=8.9 Hz, 1H), 3.17-3.07 (m, 1H), 2.72-2.55 (m, 4H), 2.13-2.02 (m, 1H), 1.80-1.69 (m, 1H). MS (ESI) m/z: 557.2 [M+H]⁺.

### Example 86

### (7R)-2-[4-(2-methylphenoxy)phenyl]-7-[4-(prop-2-enoyl)piperazin-1-yl]-4,5,6,7-tetrahydro-2H-pyrazolo[4,3-b]pyridine-3-carboxamide

### Step 86.1

### ethyl (7R)-2-[4-(2-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 86.1 was prepared according to Step 100.1, substituting [4-(2-methylphenoxy)phenyl]boronic acid for {4-[2-(trifluoromethoxy)phenoxy]phenyl}boronic acid to afford the title compound. MS (ESI) m/z: 647.5 [M+H]⁺.

### Step 86.2

### (7R)-2-[4-(2-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 86.2 was prepared according to Step 100.2, substituting ethyl (7R)-2-[4-(2-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 86.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate to afford the title compound. MS (ESI) m/z: 618.3 [M+H]⁺.

### Step 86.3

### (7R)-2-[4-(2-methylphenoxy)phenyl]-7-(piperazin-1-yl)-4,5,6,7-tetrahydro-2H-pyrazolo[4,3-b]pyridine-3-carboxamide

Step 86.3 was prepared according to Step 100.3, substituting (7R)-2-[4-(2-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 86.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 433.3 [M+H]⁺.

### Step 86.4

### (7R)-2-[4-(2-methylphenoxy)phenyl]-7-[4-(prop-2-enoyl)piperazin-1-yl]-4,5,6,7-tetrahydro-2H-pyrazolo[4,3-b]pyridine-3-carboxamide

Step 86.4 was prepared according to Step 100.4, substituting (7R)-2-[4-(2-methylphenoxy)phenyl]-7-(piperazin-1-yl)-4,5,6,7-tetrahydro-2H-pyrazolo[4,3-b]pyridine-3-carboxamide (Step 86.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide to afford the title compound. ¹H NMR (400 MHz, dimethyl sulfoxide-d₆) δ ppm 7.34 (d, J=7.4 Hz, 1H), 7.29 (d, J=8.8 Hz, 2H), 7.26-7.22 (m, 1H), 7.13 (dd, J=7.3, 7.3 Hz, 1H), 6.97 (d, J=7.9 Hz, 1H), 6.91 (d, J=8.8 Hz, 2H), 6.77 (dd, J=16.7, 10.4 Hz, 1H), 6.09 (dd, J=16.7, 2.3 Hz, 1H), 5.65 (dd, J=10.4, 2.3 Hz, 1H), 5.08 (s, 1H), 3.65-3.62 (m, 1H), 3.57-3.49 (m, 4H), 3.15-3.07 (m, 2H), 2.68-2.59 (m, 4H), 2.20 (s, 3H), 2.10-2.04 (m, 1H), 1.77-1.70 (m, 1H). MS (ESI) m/z: 487.2 [M+H]⁺.

### Example 87

### (7R)-2-[4-(4-methoxyphenoxy)phenyl]-7-[4-(prop-2-enoyl)piperazin-1-yl]-4,5,6,7-tetrahydro-2H-pyrazolo[4,3-b]pyridine-3-carboxamide

### Step 87.1

### ethyl (7R)-2-[4-(4-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 87.1 was prepared according to Step 100.1, substituting [4-(4-methoxyphenoxy)phenyl]boronic acid for {4-[2-(trifluoromethoxy)phenoxy]phenyl}boronic acid. MS (ESI) m/z: 663.4 [M+H]⁺.

### Step 87.2

### (7R)-2-[4-(4-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 87.2 was prepared according to Step 100.2, substituting ethyl (7R)-2-[4-(4-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 87.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 634.5 [M+H]⁺.

### Step 87.3

### (7R)-2-[4-(4-methoxyphenoxy)phenyl]-7-(piperazin-1-yl)-4,5,6,7-tetrahydro-2H-pyrazolo[4,3-b]pyridine-3-carboxamide

Step 87.3 was prepared according to Step 100.3, substituting (7R)-2-[4-(4-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 87.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 449.3 [M+H]⁺.

### Step 87.4

### (7R)-2-[4-(4-methoxyphenoxy)phenyl]-7-[4-(prop-2-enoyl)piperazin-1-yl]-4,5,6,7-tetrahydro-2H-pyrazolo[4,3-b]pyridine-3-carboxamide

Step 87.4 was prepared according to Step 100.4, substituting (7R)-2-[4-(4-methoxyphenoxy)phenyl]-7-(piperazin-1-yl)-4,5,6,7-tetrahydro-2H-pyrazolo[4,3-b]pyridine-3-carboxamide (Step 87.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.29 (d, J=8.9 Hz, 2H), 7.07-6.97 (m, 4H), 6.94 (d, J=8.9 Hz, 2H), 6.77 (dd, J=16.7, 10.5 Hz, 1H), 6.09 (dd, J=16.7, 2.4 Hz, 1H), 5.65 (dd, J=10.4, 2.3 Hz, 1H), 5.08 (s, 1H), 3.76 (s, 3H), 3.64 (t, J=5.3 Hz, 1H), 3.58-3.43 (m, 4H), 3.26-3.22 (m, 1H), 3.14-3.08 (m, 1H), 2.71-2.54 (m, 4H), 2.10-2.02 (m, 1H), 1.77-1.69 (m, 1H). MS (ESI) m/z: 503.2 [M+H]⁺.

### Example 88

### (7R)-2-[4-(3-methylphenoxy)phenyl]-7-[4-(prop-2-enoyl)piperazin-1-yl]-4,5,6,7-tetrahydro-2H-pyrazolo[4,3-b]pyridine-3-carboxamide

### Step 88.1

### ethyl (7R)-2-[4-(3-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 88.1 was prepared according to Step 100.1, substituting [4-(3-methylphenoxy)phenyl]boronic acid for {4-[2-(trifluoromethoxy)phenoxy]phenyl}boronic acid. MS (ESI) m/z: 647.4 [M+H]⁺.

### Step 88.2

### (7R)-2-[4-(3-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 88.2 was prepared according to Step 100.2, substituting ethyl (7R)-2-[4-(3-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 88.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 618.5 [M+H]⁺.

### Step 88.3

### (7R)-2-[4-(3-methylphenoxy)phenyl]-7-(piperazin-1-yl)-4,5,6,7-tetrahydro-2H-pyrazolo[4,3-b]pyridine-3-carboxamide

Step 88.3 was prepared according to Step 100.3, substituting (7R)-2-[4-(3-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 88.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS(ESI) m/z: 433.3 [M+H]⁺.

### Step 88.4

### (7R)-2-[4-(3-methylphenoxy)phenyl]-7-[4-(prop-2-enoyl)piperazin-1-yl]-4,5,6,7-tetrahydro-2H-pyrazolo[4,3-b]pyridine-3-carboxamide

Step 88.4 was prepared according to Step 100.4, substituting (7R)-2-[4-(3-methylphenoxy)phenyl]-7-(piperazin-1-yl)-4,5,6,7-tetrahydro-2H-pyrazolo[4,3-b]pyridine-3-carboxamide (Step 88.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.31-7.27 (m, 2H), 7.25 (d, J=7.8 Hz, 1H), 7.01-6.97 (m, 2H), 6.95 (d, J=7.6 Hz, 1H), 6.86 (s, 1H), 6.82 (dd, J=7.9, 2.4 Hz, 1H), 6.75 (dd, J=16.7, 10.5 Hz, 1H), 6.06 (dd, J=16.7, 2.4 Hz, 1H), 5.63 (dd, J=10.4, 2.4 Hz, 1H), 5.06 (s, 1H), 3.63-3.60 (m, 1H), 3.57-3.46 (m, 4H), 3.12-3.06 (m, 2H), 2.71-2.58 (m, 1H), 2.28 (s, 3H), 2.06-2.02 (m, 1H), 1.73-1.68 (m, 1H). MS (ESI) m/z: 487.2 [M+H]⁺.

### Example 89

### (7R)-7-[4-(prop-2-enoyl)piperazin-1-yl]-2-{4-[3-(trifluoromethoxy)phenoxy]phenyl}-4,5,6,7-tetrahydro-2H-pyrazolo[4,3-b]pyridine-3-carboxamide

### Step 89.1

### ethyl (7R)-7-[4-(2-nitrobenzene-1-sulfonyl)piperazin-1-yl]-2-{4-[3-(trifluoromethoxy)phenoxy]phenyl}-4,5,6,7-tetrahydro-2H-pyrazolo[4,3-b]pyridine-3-carboxylate

Step 89.1 was prepared according to Step 100.1, substituting {4-[3-(trifluoromethoxy)phenoxy]phenyl}boronic acid for {4-[2-(trifluoromethoxy)phenoxy]phenyl}boronic acid. MS (ESI) m/z: 717.5 [M+H]⁺.

### Step 89.2

### (7R)-7-[4-(2-nitrobenzene-1-sulfonyl)piperazin-1-yl]-2-{4-[3-(trifluoromethoxy)phenoxy]phenyl}-4,5,6,7-tetrahydro-2H-pyrazolo[4,3-b]pyridine-3-carboxamide

Step 89.2 was prepared according to Step 100.2, substituting ethyl (7R)-7-[4-(2-nitrobenzene-1-sulfonyl)piperazin-1-yl]-2-{4-[3-(trifluoromethoxy)phenoxy]phenyl}-4,5,6,7-tetrahydro-2H-pyrazolo[4,3-b]pyridine-3-carboxylate (Step 89.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 688.5 [M+H]⁺.

### Step 89.3

### (7R)-7-(piperazin-1-yl)-2-{4-[3-(trifluoromethoxy)phenoxy]phenyl}-4,5,6,7-tetrahydro-2H-pyrazolo[4,3-b]pyridine-3-carboxamide

Step 89.3 was prepared according to Step 100.3, substituting (7R)-7-[4-(2-nitrobenzene-1-sulfonyl)piperazin-1-yl]-2-{4-[3-(trifluoromethoxy)phenoxy]phenyl}-4,5,6,7-tetrahydro-2H-pyrazolo[4,3-b]pyridine-3-carboxamide (Step 89.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 503.3 [M+H]⁺.

### Step 89.4

### (7R)-7-[4-(prop-2-enoyl)piperazin-1-yl]-2-{4-[3-(trifluoromethoxy)phenoxy]phenyl}-4,5,6,7-tetrahydro-2H-pyrazolo[4,3-b]pyridine-3-carboxamide

Step 89.4 was prepared according to Step 100.4, substituting (7R)-7-(piperazin-1-yl)-2-{4-[3-(trifluoromethoxy)phenoxy]phenyl}-4,5,6,7-tetrahydro-2H-pyrazolo[4,3-b]pyridine-3-carboxamide (Step 89.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.57-7.50 (m, 1H), 7.37 (d, J=8.8 Hz, 2H), 7.15 (d, J=7.1 Hz, 1H), 7.13 (d, J=8.9 Hz, 2H), 7.09-7.03 (m, 2H), 6.78 (dd, J=16.6, 10.5 Hz, 1H), 6.09 (dd, J=16.6, 2.4 Hz, 1H), 5.66 (dd, J=10.4, 2.4 Hz, 1H), 5.12 (s, 1H), 3.69-3.63 (m, 1H), 3.58-3.45 (m, 4H), 3.27-3.22 (m, 1H), 3.16-3.09 (m, 1H), 2.69-2.59 (m, 1H), 2.12-2.04 (m, 1H), 1.80-1.70 (m, 1H). MS (ESI) m/z: 557.2 [M+H]⁺.

### Example 90

### (7R)-7-[4-(prop-2-enoyl)piperazin-1-yl]-2-{4-[2-(trifluoromethoxy)phenoxy]phenyl}-4,5,6,7-tetrahydro-2H-pyrazolo[4,3-b]pyridine-3-carboxamide

### Step 90.1

### ethyl (7R)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate

Step 90.1 was prepared according to Step 100.1, substituting ethyl (7R)-7-[4-(2-nitrobenzene-1-sulfonyl)piperazin-1-yl]-4,5,6,7-tetrahydro-2H-pyrazolo[4,3-b]pyridine-3-carboxylate (Intermediate R) for ethyl (7S)-7-[4-(2-nitrobenzene-1-sulfonyl)piperazin-1-yl]-4,5,6,7-tetrahydro-2H-pyrazolo[4,3-b]pyridine-3-carboxylate. MS (ESI) m/z: 717.5 [M+H]⁺.

### Step 90.2

### (7R)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide

Step 90.2 was prepared according to Step 100.2, substituting ethyl (7R)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate (Step 90.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 688.7 [M+H]⁺.

### Step 90.3

### (7R)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide

Step 90.3 was prepared according to Step 100.3, substituting (7R)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide (Step 90.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 503.6 [M+H]⁺.

### Step 90.4

### (7R)-7-[4-(prop-2-enoyl)piperazin-1-yl]-2-{4-[2-(trifluoromethoxy)phenoxy]phenyl}-4,5,6,7-tetrahydro-2H-pyrazolo[4,3-b]pyridine-3-carboxamide

Step 90.4 was prepared according to Step 100.4, substituting (7R)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide (Step 90.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.58-7.52 (m, 1H), 7.43 (ddd, J=8.2, 7.6, 1.6 Hz, 1H), 7.37-7.28 (m, 3H), 7.19 (dd, J=8.2, 1.5 Hz, 1H), 7.06-7.01 (m, 2H), 6.77 (dd, J=16.7, 10.5 Hz, 1H), 6.09 (dd, J=16.7, 2.4 Hz, 1H), 5.65 (dd, J=10.4, 2.4 Hz, 1H), 5.11 (s, 1H), 3.65 (dd, J=5.4, 5.4 Hz, 1H), 3.55-2.99 (m, 2H), 3.25 (d, J=10.5 Hz, 1H), 3.15-3.09 (m, 1H), 2.69-2.48 (m, 4H), 2.08 (ddd, J=13.2, 8.4, 4.2 Hz, 1H), 1.74 (ddd, J=13.2, 7.6, 3.9 Hz, 1H). MS (ESI) m/z: 557.6 [M+H]⁺.

### Example 91

### (7R)-2-[4-(3-methoxyphenoxy)phenyl]-7-[4-(prop-2-enoyl)piperazin-1-yl]-4,5,6,7-tetrahydro-2H-pyrazolo[4,3-b]pyridine-3-carboxamide

### Step 91.1

### ethyl (7R)-2-[4-(3-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 91.1 was prepared according to Step 100.1, substituting [4-(3-methoxyphenoxy)phenyl]boronic acid for {4-[2-(trifluoromethoxy)phenoxy]phenyl}boronic acid. MS (ESI) m/z: 663.0 [M+H]⁺.

### Step 91.2

### (7R)-2-[4-(3-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 91.2 was prepared according to Step 100.2, substituting ethyl (7R)-2-[4-(3-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 91.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 634.2 [M+H]⁺.

### Step 91.3

### (7R)-2-[4-(3-methoxyphenoxy)phenyl]-7-(piperazin-1-yl)-4,5,6,7-tetrahydro-2H-pyrazolo[4,3-b]pyridine-3-carboxamide

Step 91.3 was prepared according to Step 100.3, substituting (7R)-2-[4-(3-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 91.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 449.1 [M+H]⁺.

### Step 91.4

### (7R)-2-[4-(3-methoxyphenoxy)phenyl]-7-[4-(prop-2-enoyl)piperazin-1-yl]-4,5,6,7-tetrahydro-2H-pyrazolo[4,3-b]pyridine-3-carboxamide

Step 91.4 was prepared according to Step 100.4, substituting (7R)-2-[4-(3-methoxyphenoxy)phenyl]-7-(piperazin-1-yl)-4,5,6,7-tetrahydro-2H-pyrazolo[4,3-b]pyridine-3-carboxamide (Step 91.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.33 (d, J=8.8 Hz, 2H), 7.29 (d, J=8.2 Hz, 1H), 7.04 (d, J=8.9 Hz, 2H), 6.82-6.76 (m, 1H), 6.76-6.73 (m, 1H), 6.64 (s, 1H), 6.61-6.58 (m, 1H), 6.09 (dd, J=16.7, 2.4 Hz, 1H), 5.65 (dd, J=10.4, 2.4 Hz, 1H), 5.11 (s, 1H), 3.75 (s, 3H), 3.66-3.63 (m, 1H), 3.59-3.49 (m, 4H), 3.26-3.24 (m, 1H), 3.14-3.10 (m, 1H), 2.68-2.58 (m, 1H), 2.11-2.05 (m, 1H), 1.77-1.71 (m, 1H). MS (ESI) m/z: 503.2 [M+H]⁺.

### Example 92

### (7R)-2-[4-(4-methylphenoxy)phenyl]-7-[4-(prop-2-enoyl)piperazin-1-yl]-4,5,6,7-tetrahydro-2H-pyrazolo[4,3-b]pyridine-3-carboxamide

### Step 92.1

### ethyl (7R)-2-[4-(4-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 92.1 was prepared according to Step 100.1, substituting [4-(4-methylphenoxy)phenyl]boronic acid for {4-[2-(trifluoromethoxy)phenoxy]phenyl}boronic acid. MS (ESI) m/z: 647.2 [M+H]⁺.

### Step 92.2

### (7R)-2-[4-(4-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 92.2 was prepared according to Step 100.2, substituting ethyl (7R)-2-[4-(4-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 92.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 618.8 [M+H]⁺.

### Step 92.3

### (7R)-2-[4-(4-methylphenoxy)phenyl]-7-(piperazin-1-yl)-4,5,6,7-tetrahydro-2H-pyrazolo[4,3-b]pyridine-3-carboxamide

Step 92.3 was prepared according to Step 100.3, substituting (7R)-2-[4-(4-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 92.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 433.1 [M+H]⁺.

### Step 92.4

### (7R)-2-[4-(4-methylphenoxy)phenyl]-7-[4-(prop-2-enoyl)piperazin-1-yl]-4,5,6,7-tetrahydro-2H-pyrazolo[4,3-b]pyridine-3-carboxamide

Step 92.4 was prepared according to Step 100.4, substituting (7R)-2-[4-(4-methylphenoxy)phenyl]-7-(piperazin-1-yl)-4,5,6,7-tetrahydro-2H-pyrazolo[4,3-b]pyridine-3-carboxamide (Step 92.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.33-7.28 (m, 2H), 7.22 (d, J=8.2 Hz, 2H), 7.01-6.95 (m, 4H), 6.77 (dd, J=16.7, 10.5 Hz, 1H), 6.09 (dd, J=16.7, 2.4 Hz, 1H), 5.65 (dd, J=10.4, 2.4 Hz, 1H), 5.09 (s, 1H), 3.66-3.62 (m, 1H), 3.58-3.42 (m, 4H), 3.26-3.07 (m, 2H), 2.71-2.55 (m, 4H), 2.30 (s, 3H), 2.12-2.03 (m, 1H), 1.79-1.68 (m, 1H). MS (ESI) m/z: 487.2 [M+H]⁺.

### Example 93

### (7R)-2-[4-(2-methoxyphenoxy)phenyl]-7-[4-(prop-2-enoyl)piperazin-1-yl]-4,5,6,7-tetrahydro-2H-pyrazolo[4,3-b]pyridine-3-carboxamide

### Step 93.1

### ethyl (7R)-2-[4-(2-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 93.1 was prepared according to Step 100.1, substituting [4-(2-methoxyphenoxy)phenyl]boronic acid for {4-[2-(trifluoromethoxy)phenoxy]phenyl}boronic acid. MS (ESI) m/z: 663.3 [M+H]⁺.

### Step 93.2

### (7R)-2-[4-(2-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 93.2 was prepared according to Step 100.2, substituting ethyl (7R)-2-[4-(2-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 93.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 634.7 [M+H]⁺.

### Step 93.3

### (7R)-2-[4-(2-methoxyphenoxy)phenyl]-7-(piperazin-1-yl)-4,5,6,7-tetrahydro-2H-pyrazolo[4,3-b]pyridine-3-carboxamide

Step 93.3 was prepared according to Step 100.3, substituting (7R)-2-[4-(2-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 93.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 449.5 [M+H]⁺.

### Step 93.4

### (7R)-2-[4-(2-methoxyphenoxy)phenyl]-7-[4-(prop-2-enoyl)piperazin-1-yl]-4,5,6,7-tetrahydro-2H-pyrazolo[4,3-b]pyridine-3-carboxamide

Step 93.4 was prepared according to Step 100.4, substituting (7R)-2-[4-(2-methoxyphenoxy)phenyl]-7-(piperazin-1-yl)-4,5,6,7-tetrahydro-2H-pyrazolo[4,3-b]pyridine-3-carboxamide (Step 93.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.31-7.29 (m, 2H), 7.23-7.15 (m, 2H), 7.09 (d, J=7.8 Hz, 1H), 7.02-6.97 (m, 1H), 6.84 (d, J=8.9 Hz, 2H), 6.77 (dd, J=16.6, 10.5 Hz, 1H), 6.08 (dd, J=16.7, 2.2 Hz, 1H), 5.65 (dd, J=10.5, 2.2 Hz, 1H), 5.06 (s, 1H), 3.76 (s, 3H), 3.62 (t, J=5.0 Hz, 1H), 3.54-3.44 (m, 4H), 3.24 (d, J=8.8 Hz, 1H), 3.13-3.07 (m, 1H), 2.67-2.54 (m, 4H), 2.10-2.03 (m, J=5.0 Hz, 1H), 1.76-1.68 (m, J=5.5 Hz, 1H). MS (ESI) m/z: 503.1 [M+H]⁺.

### Example 94

### (7S)-2-[4-(2-methylphenoxy)phenyl]-7-[4-(prop-2-enoyl)piperazin-1-yl]-4,5,6,7-tetrahydro-2H-pyrazolo[4,3-b]pyridine-3-carboxamide

### Step 94.1

### ethyl (7S)-2-[4-(2-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 94.1 was prepared according to Step 100.1, substituting [4-(2-methylphenoxy)phenyl]boronic acid for {4-[2-(trifluoromethoxy)phenoxy]phenyl}boronic acid. MS (ESI) m/z: 647.5 [M+H]⁺.

### Step 94.2

### (7S)-2-[4-(2-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 94.2 was prepared according to Step 100.2, substituting ethyl (7S)-2-[4-(2-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 94.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 618.4 [M+H]⁺.

### Step 94.3

### (7S)-2-[4-(2-methylphenoxy)phenyl]-7-(piperazin-1-yl)-4,5,6,7-tetrahydro-2H-pyrazolo[4,3-b]pyridine-3-carboxamide

Step 94.3 was prepared according to Step 100.3, substituting (7S)-2-[4-(2-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 94.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 433.4 [M+H]⁺.

### Step 94.4

### (7S)-2-[4-(2-methylphenoxy)phenyl]-7-[4-(prop-2-enoyl)piperazin-1-yl]-4,5,6,7-tetrahydro-2H-pyrazolo[4,3-b]pyridine-3-carboxamide

Step 94.4 was prepared according to Step 100.4, substituting (7S)-2-[4-(2-methylphenoxy)phenyl]-7-(piperazin-1-yl)-4,5,6,7-tetrahydro-2H-pyrazolo[4,3-b]pyridine-3-carboxamide (Step 94.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.34 (d, J=6.9 Hz, 1H), 7.32-7.27 (m, 2H), 7.25 (dd, J=10.8, 4.5 Hz, 1H), 7.15-7.11 (m, 1H), 6.97 (d, J=8.0 Hz, 1H), 6.94-6.88 (m, 2H), 6.77 (dd, J=16.7, 10.4 Hz, 1H), 6.09 (dd, J=16.7, 2.4 Hz, 1H), 5.65 (dd, J=10.4, 2.4 Hz, 1H), 5.08 (s, 1H), 3.64 (t, J=5.4 Hz, 1H), 3.59-3.44 (m, 4H), 3.24 (d, J=9.3 Hz, 1H), 3.11 (dd, J=11.9, 5.6 Hz, 1H), 2.70-2.54 (m, 4H), 2.20 (s, 3H), 2.08 (ddd, J=13.9, 9.4, 5.1 Hz, 1H), 1.78-1.68 (m, 1H). MS (ESI) m/z: 487.4 [M+H]⁺.

### Example 95

### (7S)-2-[4-(3-methylphenoxy)phenyl]-7-[4-(prop-2-enoyl)piperazin-1-yl]-4,5,6,7-tetrahydro-2H-pyrazolo[4,3-b]pyridine-3-carboxamide

### Step 95.1

### ethyl (7S)-2-[4-(3-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 95.1 was prepared according to Step 100.1, substituting [4-(3-methylphenoxy)phenyl]boronic acid for {4-[2-(trifluoromethoxy)phenoxy]phenyl}boronic acid. MS (ESI) m/z: 647.4 [M+H]⁺.

### Step 95.2

### (7S)-2-[4-(3-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 95.2 was prepared according to Step 100.2, substituting ethyl (7S)-2-[4-(3-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 95.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 618.3 [M+H]⁺.

### Step 95.3

### (7S)-2-[4-(3-methylphenoxy)phenyl]-7-(piperazin-1-yl)-4,5,6,7-tetrahydro-2H-pyrazolo[4,3-b]pyridine-3-carboxamide

Step 95.3 was prepared according to Step 100.3, substituting (7S)-2-[4-(3-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 95.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 433.4 [M+H]⁺.

### Step 95.4

### (7S)-2-[4-(3-methylphenoxy)phenyl]-7-[4-(prop-2-enoyl)piperazin-1-yl]-4,5,6,7-tetrahydro-2H-pyrazolo[4,3-b]pyridine-3-carboxamide

Step 95.4 was prepared according to Step 100.4, substituting (7S)-2-[4-(3-methylphenoxy)phenyl]-7-(piperazin-1-yl)-4,5,6,7-tetrahydro-2H-pyrazolo[4,3-b]pyridine-3-carboxamide (Step 95.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.34-7.30 (m, 2H), 7.28 (d, J=7.8 Hz, 1H), 7.07-7.00 (m, 2H), 6.98 (d, J=7.6 Hz, 1H), 6.89 (s, 1H), 6.85 (d, J=8.0 Hz, 1H), 6.78 (dd, J=16.7, 10.4 Hz, 1H), 6.09 (dd, J=16.7, 2.4 Hz, 1H), 5.65 (dd, J=10.4, 2.4 Hz, 1H), 5.09 (s, 1H), 3.64 (t, J=5.4 Hz, 1H), 3.50 (ddd, J=22.0, 14.1, 3.0 Hz, 4H), 3.25 (d, J=9.9 Hz, 1H), 3.11 (dd, J=11.3, 4.1 Hz, 1H), 2.66-2.55 (m, 4H), 2.30 (s, 3H), 2.12-2.02 (m, 1H), 1.74 (4d, J=10.1, 4.7, 4.7 Hz, 1H). MS (ESI) m/z: 487.3 [M+H]⁺.

### Example 96

### (7S)-2-[4-(4-methylphenoxy)phenyl]-7-[4-(prop-2-enoyl)piperazin-1-yl]-4,5,6,7-tetrahydro-2H-pyrazolo[4,3-b]pyridine-3-carboxamide

### Step 96.1

### ethyl (7S)-2-[4-(4-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 96.1 was prepared according to Step 100.1, substituting [4-(4-methylphenoxy)phenyl]boronic acid for {4-[2-(trifluoromethoxy)phenoxy]phenyl}boronic acid. MS (ESI) m/z: 647.5 [M+H]⁺.

### Step 96.2

### (7S)-2-[4-(4-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 96.2 was prepared according to Step 100.2, substituting ethyl (7S)-2-[4-(4-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 96.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 618.2 [M+H]⁺.

### Step 96.3

### (7S)-2-[4-(4-methylphenoxy)phenyl]-7-(piperazin-1-yl)-4,5,6,7-tetrahydro-2H-pyrazolo[4,3-b]pyridine-3-carboxamide

Step 96.3 was prepared according to Step 100.3, substituting (7S)-2-[4-(4-methylphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 96.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 433.1 [M+H]⁺.

### Step 96.4

### (7S)-2-[4-(4-methylphenoxy)phenyl]-7-[4-(prop-2-enoyl)piperazin-1-yl]-4,5,6,7-tetrahydro-2H-pyrazolo[4,3-b]pyridine-3-carboxamide

Step 96.4 was prepared according to Step 100.4, substituting (7S)-2-[4-(4-methylphenoxy)phenyl]-7-(piperazin-1-yl)-4,5,6,7-tetrahydro-2H-pyrazolo[4,3-b]pyridine-3-carboxamide (Step 96.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.34-7.27 (m, 2H), 7.22 (d, J=8.1 Hz, 2H), 7.05-6.92 (m, 4H), 6.77 (dd, J=16.7, 10.5 Hz, 1H), 6.09 (dd, J=16.7, 2.4 Hz, 1H), 5.65 (dd, J=10.4, 2.4 Hz, 1H), 5.09 (s, 1H), 3.66-3.62 (m, 1H), 3.53 (s, 4H), 3.26 (s, 1H), 3.13-3.08 (m, 1H), 2.68-2.59 (m, 4H), 2.30 (s, 3H), 2.11-2.03 (m, 1H), 1.73 (dd, J=9.3, 6.2 Hz, 1H). MS (ESI) m/z: 487.3 [M+H]⁺.

### Example 97

### (7S)-2-[4-(2-methoxyphenoxy)phenyl]-7-[4-(prop-2-enoyl)piperazin-1-yl]-4,5,6,7-tetrahydro-2H-pyrazolo[4,3-b]pyridine-3-carboxamide

### Step 97.1

### ethyl (7S)-2-[4-(2-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 97.1 was prepared according to Step 100.1, substituting [4-(2-methoxyphenoxy)phenyl]boronic acid for {4-[2-(trifluoromethoxy)phenoxy]phenyl}boronic acid. MS (ESI) m/z: 663.2 [M+H]⁺.

### Step 97.2

### (7S)-2-[4-(2-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 97.2 was prepared according to Step 100.2, substituting ethyl (7S)-2-[4-(2-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 97.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 634.5 [M+H]⁺.

### Step 97.3

### (7S)-2-[4-(2-methoxyphenoxy)phenyl]-7-(piperazin-1-yl)-4,5,6,7-tetrahydro-2H-pyrazolo[4,3-b]pyridine-3-carboxamide

Step 97.3 was prepared according to Step 100.3, substituting (7S)-2-[4-(2-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 97.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 449.1 [M+H]⁺.

### Step 97.4

### (7S)-2-[4-(2-methoxyphenoxy)phenyl]-7-[4-(prop-2-enoyl)piperazin-1-yl]-4,5,6,7-tetrahydro-2H-pyrazolo[4,3-b]pyridine-3-carboxamide

Step 97.4 was prepared according to Step 100.4, substituting (7S)-2-[4-(2-methoxyphenoxy)phenyl]-7-(piperazin-1-yl)-4,5,6,7-tetrahydro-2H-pyrazolo[4,3-b]pyridine-3-carboxamide (Step 97.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.35-7.13 (m, 4H), 7.09 (dd, J=7.9, 1.5 Hz, 1H), 7.02-6.97 (m, 1H), 6.88-6.79 (m, 2H), 6.79-6.69 (m, 1H), 6.08 (dd, J=16.7, 2.4 Hz, 1H), 5.65 (dd, J=10.4, 2.4 Hz, 1H), 5.07 (s, 1H), 3.76 (s, 3H), 3.62 (t, J=5.4 Hz, 1H), 3.54-3.43 (m, 4H), 3.24 (d, J=10.9 Hz, 1H), 3.11 (s, 1H), 2.69-2.51 (m, 4H), 2.11-2.02 (m, 1H), 1.77-1.68 (m, 1H). MS (ESI) m/z: 503.3 [M+H]⁺.

### Example 98

### (7S)-2-[4-(3-methoxyphenoxy)phenyl]-7-[4-(prop-2-enoyl)piperazin-1-yl]-4,5,6,7-tetrahydro-2H-pyrazolo[4,3-b]pyridine-3-carboxamide

### Step 98.1

### ethyl (7S)-2-[4-(3-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 98.1 was prepared according to Step 100.1, substituting [4-(3-methoxyphenoxy)phenyl]boronic acid for {4-[2-(trifluoromethoxy)phenoxy]phenyl}boronic acid. MS (ESI) m/z: 663.3 [M+H]⁺.

### Step 98.2

### (7S)-2-[4-(3-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 98.2 was prepared according to Step 100.2, substituting ethyl (7S)-2-[4-(3-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 98.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 634.5 [M+H]⁺.

### Step 98.3

### (7S)-2-[4-(3-methoxyphenoxy)phenyl]-7-(piperazin-1-yl)-4,5,6,7-tetrahydro-2H-pyrazolo[4,3-b]pyridine-3-carboxamide

Step 98.3 was prepared according to Step 100.3, substituting (7S)-2-[4-(3-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 98.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 449.4 [M+H]⁺.

### Step 98.4

### (7S)-2-[4-(3-methoxyphenoxy)phenyl]-7-[4-(prop-2-enoyl)piperazin-1-yl]-4,5,6,7-tetrahydro-2H-pyrazolo[4,3-b]pyridine-3-carboxamide

Step 98.4 was prepared according to Step 100.4, substituting (7S)-2-[4-(3-methoxyphenoxy)phenyl]-7-(piperazin-1-yl)-4,5,6,7-tetrahydro-2H-pyrazolo[4,3-b]pyridine-3-carboxamide (Step 98.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.35-7.31 (m, 2H), 7.29 (d, J=8.2 Hz, 1H), 7.07-7.03 (m, 2H), 6.81-6.72 (m, 2H), 6.64 (t, J=2.3 Hz, 1H), 6.60 (ddd, J=8.1, 2.3, 0.7 Hz, 1H), 6.09 (dd, J=16.7, 2.4 Hz, 1H), 5.65 (dd, J=10.4, 2.4 Hz, 1H), 5.10 (s, 1H), 3.65 (t, J=5.4 Hz, 1H), 3.52 (t, J=21.2 Hz, 4H), 3.37-3.24 (m, 4H), 3.12 (s, 1H), 2.68-2.48 (m, 4H), 2.08 (dd, J=6.7, 4.6 Hz, 1H), 1.74 (dd, J=8.8, 5.6 Hz, 1H). MS (ESI) m/z: 503.3 [M+H]⁺.

### Example 99

### (7S)-2-[4-(4-methoxyphenoxy)phenyl]-7-[4-(prop-2-enoyl)piperazin-1-yl]-4,5,6,7-tetrahydro-2H-pyrazolo[4,3-b]pyridine-3-carboxamide

### Step 99.1

### ethyl (7S)-2-[4-(4-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate

Step 99.1 was prepared according to Step 100.1, substituting [4-(4-methoxyphenoxy)phenyl]boronic acid for {4-[2-(trifluoromethoxy)phenoxy]phenyl}boronic acid. MS (ESI) m/z: 663.2 [M+H]⁺.

### Step 99.2

### (7S)-2-[4-(4-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide

Step 99.2 was prepared according to Step 100.2, substituting ethyl (7S)-2-[4-(4-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxylate (Step 99.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 634.7 [M+H]⁺.

### Step 99.3

### (7S)-2-[4-(4-methoxyphenoxy)phenyl]-7-(piperazin-1-yl)-4,5,6,7-tetrahydro-2H-pyrazolo[4,3-b]pyridine-3-carboxamide

Step 99.3 was prepared according to Step 100.3, substituting (7S)-2-[4-(4-methoxyphenoxy)phenyl]-7-[4-(2-nitrobenzene-1-sulfonyl)piperazin-1-yl]-4,5,6,7-tetrahydro-2H-pyrazolo[4,3-b]pyridine-3-carboxamide (Step 99.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 449.5 [M+H]⁺.

### Step 99.4

### (7S)-2-[4-(4-methoxyphenoxy)phenyl]-7-[4-(prop-2-enoyl)piperazin-1-yl]-4,5,6,7-tetrahydro-2H-pyrazolo[4,3-b]pyridine-3-carboxamide

Step 99.4 was prepared according to Step 100.4, substituting (7S)-2-[4-(4-methoxyphenoxy)phenyl]-7-(piperazin-1-yl)-4,5,6,7-tetrahydro-2H-pyrazolo[4,3-b]pyridine-3-carboxamide (Step 99.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.32-7.25 (m, 2H), 7.08-7.02 (m, 2H), 7.02-6.89 (m, 4H), 6.81-6.74 (m, 1H), 6.09 (dd, J=16.7, 2.4 Hz, 1H), 5.65 (dd, J=10.4, 2.4 Hz, 1H), 5.08 (s, 1H), 3.76 (s, 3H), 3.65-3.62 (m, 1H), 3.53 (s, 3H), 3.23 (s, 1H), 3.11 (dd, J=11.3, 5.9 Hz, 1H), 2.68-2.49 (m, 4H), 2.11-2.03 (m, 1H), 1.77-1.69 (m, 1H). MS (ESI) m/z: 503.4 [M+H]⁺.

### Example 100

### (7S)-7-[4-(prop-2-enoyl)piperazin-1-yl]-2-{4-[2-(trifluoromethoxy)phenoxy]phenyl}-4,5,6,7-tetrahydro-2H-pyrazolo[4,3-b]pyridine-3-carboxamide

### Step 100.1

### ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate

To a solution of ethyl (7S)-7-[4-(2-nitrobenzene-1-sulfonyl)piperazin-1-yl]-4,5,6,7-tetrahydro-2H-pyrazolo[4,3-b]pyridine-3-carboxylate (1.2 g, 2.58 mmol, Intermediate L) in dichloromethane (50 mL) was added {4-[2-(trifluoromethoxy)phenoxy]phenyl}boronic acid (1.16 g, 3.88 mmol), Cu(OC(O)CH₃)₂ (258 mg, 1.42 mmol) and pyridine (1.05 mL, 12.9 mmol) at ambient temperature. The reaction mixture was stirred under an 02 atmosphere for 15 hours. The reaction was diluted with ammonium hydroxide and filtered over diatomaceous earth. The filtrate was extracted with dichloromethane (3×). The combined organic layers were washed with brine, dried over Na₂SO₄, filtered, and concentrated under reduced pressure. The crude residue was purified by column chromatography (0% to 70% ethyl acetate in heptanes) to afford the title compound (1.13 g, 61%). MS (ESI) m/z: 717.2 [M+H]⁺.

### Step 100.2

### (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide

To a solution of ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate (1.10 g, 1.54 mmol, Step 100.1) in methanol (30 mL) was added NaOH (614 mg, 15.4 mmol) in water (1 mL) at ambient temperature. The reaction mixture was heated to 40° C. and stirred for 16 hours. The reaction mixture was cooled to 0° C., and 4 M aqueous HCl was added until pH 6 achieved. The mixture was extracted with dichloromethane (3×100 mL). The combined organic layers were washed with brine, dried over Na₂SO₄, filtered, and concentrated under reduced pressure to afford a residue (966 mg). To a solution of the residue (910 mg) in dichloromethane (30 mL) was added (1-[bis(dimethylamino)methylene]-1H-1,2,3-triazolo[4,5-b]pyridinium 3-oxid hexafluorophosphate (754 mg, 1.982 mmol), N,N-diisopropylethylamine (1.15 mL, 6.61 mmol) and ammonia hydrochloride (141 mg, 2.64 mmol) at ambient temperature, and the resulting mixture was stirred for 30 minutes. The reaction mixture was diluted with water and extracted with dichloromethane (3×). The combined organic layers were washed with brine, dried over Na₂SO₄, filtered, and concentrated under reduced pressure. The crude residue was purified by column chromatography (0% to 100% ethyl acetate in heptanes) to afford the title compound (905 mg, 85%). MS (ESI) m/z: 688.6 [M+H]⁺.

### Step 100.3

### (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide

To a solution of (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide (810 mg, 1.18 mmol, Step 100.2) in tetrahydrofuran (30 mL) was added 4-bromobenzenethiol (334 mg, 1.767 mmol) and Cs₂CO₃ (959 mg, 2.94 mmol) at ambient temperature, and the reaction mixture stirred for 1 hour. The reaction mixture was diluted with water and extracted with ethyl acetate (3×). The combined organic layers were washed with brine, dried over Na₂SO₄, filtered, and concentrated under reduced pressure. The crude residue was purified by column chromatography (0% to 100% ethyl acetate in heptanes) to afford the title compound (330 mg, 56%). MS (ESI) m/z: 503.5 [M+H]⁺.

### Step 100.4

### (7S)-7-[4-(prop-2-enoyl)piperazin-1-yl]-2-{4-[2-(trifluoromethoxy)phenoxy]phenyl}-4,5,6,7-tetrahydro-2H-pyrazolo[4,3-b]pyridine-3-carboxamide

To a solution of acrylic acid (43.0 mg, 0.597 mmol), (1-[bis(dimethylamino)methylene]-1H-1,2,3-triazolo[4,5-b]pyridinium 3-oxid hexafluorophosphate (341 mg, 0.896 mmol) and N,N-diisopropylethylamine (386 mg, 2.99 mmol) in dichloromethane (30 mL) was added (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide (300 mg, 0.597 mmol, Step 100.3) at ambient temperature, and the reaction mixture stirred for 10 minutes. The reaction mixture was diluted with water and extracted with dichloromethane (3×). The combined organic layers were washed with brine, dried over Na₂SO₄, filtered, and concentrated under reduced pressure. The crude residue was purified by reverse phase HPLC (20% to 100% acetonitrile in 0.1% HCO₂H) column to afford the title compound (94.5 mg, 28%). ¹H NMR (400 MHz, dimethyl sulfoxide-d₆) δ ppm 7.58-7.52 (m, 1H), 7.43 (ddd, J=8.2, 7.6, 1.6 Hz, 1H), 7.37-7.28 (m, 3H), 7.19 (dd, J=8.2, 1.5 Hz, 1H), 7.06-7.01 (m, 2H), 6.77 (dd, J=16.7, 10.5 Hz, 1H), 6.09 (dd, J=16.7, 2.4 Hz, 1H), 5.65 (dd, J=10.4, 2.4 Hz, 1H), 5.11 (s, 1H), 3.65 (dd, J=5.4, 5.4 Hz, 1H), 3.55-2.99 (m, 2H), 3.25 (d, J=10.5 Hz, 1H), 3.15-3.09 (m, 1H), 2.64 (ddd, J=12.7, 12.7, 10.4 Hz, 4H), 2.08 (ddd, J=13.2, 8.4, 4.2 Hz, 1H), 1.74 (ddd, J=13.2, 7.6, 3.9 Hz, 1H). MS (ESI) m/z: 557.6 [M+H]⁺.

### Example 101

### (7S)-7-[4-(prop-2-enoyl)piperazin-1-yl]-2-{4-[3-(trifluoromethoxy)phenoxy]phenyl}-4,5,6,7-tetrahydro-2H-pyrazolo[4,3-b]pyridine-3-carboxamide

### Step 101.1

### ethyl (7S)-7-[4-(2-nitrobenzene-1-sulfonyl)piperazin-1-yl]-2-{4-[3-(trifluoromethoxy)phenoxy]phenyl}-4,5,6,7-tetrahydro-2H-pyrazolo[4,3-b]pyridine-3-carboxylate

Step 101.1 was prepared according to Step 100.1, substituting {4-[3-(trifluoromethoxy)phenoxy]phenyl}boronic acid for {4-[2-(trifluoromethoxy)phenoxy]phenyl}boronic acid. MS (ESI) m/z: 717.3 [M+H]⁺.

### Step 101.2

### (7S)-7-[4-(2-nitrobenzene-1-sulfonyl)piperazin-1-yl]-2-{4-[3-(trifluoromethoxy)phenoxy]phenyl}-4,5,6,7-tetrahydro-2H-pyrazolo[4,3-b]pyridine-3-carboxamide

Step 101.2 was prepared according to Step 100.2, substituting ethyl (7S)-7-[4-(2-nitrobenzene-1-sulfonyl)piperazin-1-yl]-2-{4-[3-(trifluoromethoxy)phenoxy]phenyl}-4,5,6,7-tetrahydro-2H-pyrazolo[4,3-b]pyridine-3-carboxylate (Step 101.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 688.6 [M+H]⁺.

### Step 101.3

### (7S)-7-(piperazin-1-yl)-2-{4-[3-(trifluoromethoxy)phenoxy]phenyl}-4,5,6,7-tetrahydro-2H-pyrazolo[4,3-b]pyridine-3-carboxamide

Step 101.3 was prepared according to Step 100.3, substituting (7S)-7-[4-(2-nitrobenzene-1-sulfonyl)piperazin-1-yl]-2-{4-[3-(trifluoromethoxy)phenoxy]phenyl}-4,5,6,7-tetrahydro-2H-pyrazolo[4,3-b]pyridine-3-carboxamide (Step 101.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 503.5 [M+H]⁺.

### Step 101.4

### (7S)-7-[4-(prop-2-enoyl)piperazin-1-yl]-2-{4-[3-(trifluoromethoxy)phenoxy]phenyl}-4,5,6,7-tetrahydro-2H-pyrazolo[4,3-b]pyridine-3-carboxamide

Step 101.4 was prepared according to Step 100.4, substituting (7S)-7-(piperazin-1-yl)-2-{4-[3-(trifluoromethoxy)phenoxy]phenyl}-4,5,6,7-tetrahydro-2H-pyrazolo[4,3-b]pyridine-3-carboxamide (Step 101.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.53 (dd, J=10.8, 6.4 Hz, 1H), 7.40-7.34 (m, 2H), 7.17-7.10 (m, 3H), 7.08-7.04 (m, 2H), 6.78 (dd, J=16.7, 10.4 Hz, 1H), 6.09 (dd, J=16.7, 2.4 Hz, 1H), 5.66 (dd, J=10.4, 2.4 Hz, 1H), 5.12 (s, 1H), 3.66 (t, J=5.5 Hz, 1H), 3.55-3.45 (m, 4H), 3.26 (s, 1H), 3.15-3.09 (m, 1H), 2.68-2.58 (m, 4H), 2.08 (dt, J=10.6, 6.4 Hz, 1H), 1.74 (ddd, J=13.4, 7.9, 4.0 Hz, 1H). MS (ESI) m/z: 557.3 [M+H]⁺.

### Example 102

### (7S)-7-[4-(prop-2-enoyl)piperazin-1-yl]-2-{4-[4-(trifluoromethoxy)phenoxy]phenyl}-4,5,6,7-tetrahydro-2H-pyrazolo[4,3-b]pyridine-3-carboxamide

### Step 102.1

### ethyl (7S)-7-[4-(2-nitrobenzene-1-sulfonyl)piperazin-1-yl]-2-{4-[4-(trifluoromethoxy)phenoxy]phenyl}-4,5,6,7-tetrahydro-2H-pyrazolo[4,3-b]pyridine-3-carboxylate

Step 102.1 was prepared according to Step 100.1, substituting {4-[4-(trifluoromethoxy)phenoxy]phenyl}boronic acid for {4-[2-(trifluoromethoxy)phenoxy]phenyl}boronic acid. MS (ESI) m/z: 717.4 [M+H]⁺.

### Step 102.2

### (7S)-7-[4-(2-nitrobenzene-1-sulfonyl)piperazin-1-yl]-2-{4-[4-(trifluoromethoxy)phenoxy]phenyl}-4,5,6,7-tetrahydro-2H-pyrazolo[4,3-b]pyridine-3-carboxamide

Step 102.2 was prepared according to Step 100.2, substituting ethyl (7S)-7-[4-(2-nitrobenzene-1-sulfonyl)piperazin-1-yl]-2-{4-[4-(trifluoromethoxy)phenoxy]phenyl}-4,5,6,7-tetrahydro-2H-pyrazolo[4,3-b]pyridine-3-carboxylate (Step 102.1) for ethyl (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxylate. MS (ESI) m/z: 688.4 [M+H]⁺.

### Step 102.3

### (7S)-7-(piperazin-1-yl)-2-{4-[4-(trifluoromethoxy)phenoxy]phenyl}-4,5,6,7-tetrahydro-2H-pyrazolo[4,3-b]pyridine-3-carboxamide

Step 102.3 was prepared according to Step 100.3, substituting (7S)-7-[4-(2-nitrobenzene-1-sulfonyl)piperazin-1-yl]-2-{4-[4-(trifluoromethoxy)phenoxy]phenyl}-4,5,6,7-tetrahydro-2H-pyrazolo[4,3-b]pyridine-3-carboxamide (Step 102.2) for (7S)-7-[4-(2-nitrobenzene-1-sulfonyl)piperazin-1-yl]-2-{4-[2-(trifluoromethoxy)phenoxy]phenyl}-4,5,6,7-tetrahydro-2H-pyrazolo[4,3-b]pyridine-3-carboxamide. MS (ESI) m/z: 503.2 [M+H]⁺.

### Step 102.4

### (7S)-7-[4-(prop-2-enoyl)piperazin-1-yl]-2-{4-[4-(trifluoromethoxy)phenoxy]phenyl}-4,5,6,7-tetrahydro-2H-pyrazolo[4,3-b]pyridine-3-carboxamide

Step 102.4 was prepared according to Step 100.4, substituting (7S)-7-(piperazin-1-yl)-2-{4-[4-(trifluoromethoxy)phenoxy]phenyl}-4,5,6,7-tetrahydro-2H-pyrazolo[4,3-b]pyridine-3-carboxamide (Step 102.3) for (7S)-7-(piperazin-1-yl)-2-{4-[2-(trifluoromethoxy)phenoxy]phenyl}-4,5,6,7-tetrahydro-2H-pyrazolo[4,3-b]pyridine-3-carboxamide. ¹H NMR (400 MHz, dimethyl sulfoxide-d₆) δ ppm 7.41 (d, J=8.8 Hz, 2H), 7.36 (d, J=8.8 Hz, 2H), 7.19-7.13 (m, 2H), 7.09 (d, J=8.8 Hz, 2H), 6.78 (dd, J=16.7, 10.4 Hz, 1H), 6.09 (dd, J=16.7, 2.4 Hz, 1H), 5.66 (dd, J=10.4, 2.3 Hz, 1H), 5.11 (s, 1H), 3.65 (t, J=5.4 Hz, 1H), 3.55-3.44 (m, 4H), 3.25 (d, J=8.9 Hz, 1H), 3.12 (s, 1H), 2.68-2.57 (m, 4H), 2.07 (d, J=3.9 Hz, 1H), 1.78-1.70 (m, 1H). MS (ESI) m/z: 557.3 [M+H]⁺.

### Determination of Biological Activity

### BTK and Other Kinase-Compound Screening

Kinase activity, unless otherwise indicated, was measured in vitro using an electrophoretic mobility shift assay (MSA). The phosphorylation of a peptide substrate by BTK and other kinases was measured. The kinase reactions were assembled in a total volume of 25 μL per well in 384 well plates. The following was added to each well: compound buffer (or control); enzyme buffer; and substrate buffer, as further described below.

Specifically the following was added: (1) compound buffer or control: 5 μL of 5× compound buffer [(5× compound buffer comprised of: IX Master Buffer, X μM test compound in 5% dimethyl sulfoxide; (2× Master Buffer comprised of 200 mM HEPES, pH 7.5, 0.2% BSA, and 0.02% Triton X-100)]; and (2) enzyme buffer: 10 μL of 2.5× enzyme buffer (1× Master Buffer, 12.5 mM MgCl₂, 2.5 mM DTT, 25 μM sodium orthovanadate, 25 μM beta-glycerophosphate, and 1.25 nM BTK enzyme—or other kinase). (BTK enzyme Nanosyn-293HEK, WT, available from Nanosyn, Santa Clara, Calif.). Enzyme and compound/inhibitor may be pre-incubated. Additionally, the following was added: (3) substrate buffer: 10 μL of 2.5× substrate buffer (1× Master Buffer, 50 μM ATP, and 2.5 μM of the peptide substrate (as described in Table 3; FAM was carboxyfluorescein). Substrate and ATP concentration varied depending on kinase tested. Each plate was incubated at 25° C. for 3 hours (incubation time varied depending on the kinase tested). The reaction was terminated by adding to each well: 45 L of 1.55× stop buffer (1× Master Buffer and 31 mM EDTA). The final reaction mixture was as follows: 100 mM HEPES, pH 7.5; 0.1% BSA; 0.01% Triton X-100; 1 mM DTT; 5 mM MgCl₂; 10 μM sodium orthovanadate; 10 μM beta-glycerophosphate; 50 μM ATP; 1% dimethyl sulfoxide (from compound); 1 μM (peptide substrate from Table 3); and 0.5 nM BTK-enzyme (or other kinase/substrate).

Kinase activities for Examples 50, 51, 62 and 63 were measured in vitro using the following electrophoretic mobility shift assay (MSA). The kinase reactions were assembled in a total volume of 20 μL in 384 well plates. To each well, 20 nL test compound, or control, in 100% dimethyl sulfoxide was spotted using Echo®, a liquid dispensing instrument (Labcyte, Indianapolis, Ind.). To each well was added: 12 μL of 1.67× Full length BTK enzyme in Master Buffer (Master Buffer components: 20 mM HEPES pH 7.4, 10 mM MgCl₂, 1 mM DTT, 0.1 mM sodium orthovanadate, 10 mM beta-glycerophosphate, and 0.0075% w/v Triton x-100). Full length BTK enzyme was 6His-Flag-(TEV)-S[BTK(hu) (2-659)]*BEV, pFastBac*. [Note: this is a description of the exact amino acid sequence of the expressed BTK construct from N- to C-terminus: 6Histidines, Flag tag sequence, TEV cleavage site, Serine, human BTK residues 2-659; and BEV=expressed in baculovirus, pFastBac is the expression vector used].

Enzyme and compound/inhibitor were optionally pre-incubated. To each well was added: 8 μL of 2.5×ATP and substrate in 1× Master Buffer (1× Master Buffer as described above). The plate was incubated at room temperature for 3 hours. The reaction was terminated by adding 30 μL of 1.67× Stop Buffer (Stop Buffer components 10 mM EDTA in water), to each well. The final reaction mixture was as follows: 20 mM HEPES, pH 7.4; 0.0075% Triton X-100; 1 mM DTT; 10 mM MgCl₂; 0.1 mM sodium orthovanadate; 10 mM beta-glycerophosphate; 0.1% dimethyl sulfoxide (from compound); 1 mM ATP; 1 μM (OG488-SEQ ID NO. 2-NH₂) peptide substrate; and 0.6 nM BTK enzyme. OG488 was Oregon Green dye, which was conjugated to the peptide substrate. The assays for the other kinases were conducted essentially as described above, with the modifications in ATP concentrations and their corresponding substrates in the final reaction mixtures as shown in Table 3. The kinase activity for the kinases in Table 3 were measured at 3 hours incubation time, except that in the case of MAP2K1 and MAP2K2 assay, pre-activation of ERK was done for 30 min with 6 μM of ATP, and then the detection of activated ERK in the presence of compounds was done after 60 minutes of incubation.

TABLE 3
Other Kinase ATP concentrations and corresponding substrate

| | ATP | |
|---|---|---|
| Kinase | Concentration (μM) | Peptide Substrate |
| TEC | 50 | SRCtide (FAM-SEQ ID NO: 1-NH₂) |
| BLK | 20 | SRCtide (FAM-SEQ ID NO: 1-NH₂) |
| EGFR | 5 | CSKtide (FAM-SEQ ID NO: 3-NH₂) |
| ERBB4 | 15 | SRCtide (FAM-SEQ ID NO: 1-NH₂) |
| TXK | 100 | SRCtide (FAM-SEQ ID NO: 1-NH₂) |
| BMX | 10 | SRCtide (FAM-SEQ ID NO: 1-NH₂) |
| MAP2K1 | 25 | ERKtide (FAM-SEQ ID NO: 4-NH₂) |
| MAP2K2 | 30 | ERKtide (FAM-SEQ ID NO: 4-NH₂) |

The terminated reactions were analyzed using a 12 channel LABCHIP® 3000 microfluidic detection instrument (available from Caliper Life Sciences, Waltham, Mass.). The enzymatic phosphorylation of the peptide resulted in a change in net charge, which enabled electrophoretic separation of product from substrate peptide. As substrate and product peptides were separated, two peaks of fluorescence were observed. Change in the relative fluorescence intensity of the substrate and product peaks was the parameter measured, reflecting enzyme activity. In the presence of an inhibitor, the ratio between product and substrate was altered. The signal of the product decreased, while the signal of the substrate increased. Capillary electrophoregrams (RDA acquisition files) were analyzed using HTS Well Analyzer software (available from Caliper Life Sciences, Waltham, Mass.).

TABLE 4
Kinase Assay Substrate Sequences

| SEQ ID NO: | Sequence |
|---|---|
| 1 | GEEPLYWSFPAKKK |
| 2 | GAQEEIYAAFFAKK |
| 3 | KKKKEEIYFFFG |
| 4 | IPTSPITTTYFFFKKK |

Activity in each sample was determined as the product to sum ratio (PSR): P/(S+P), where P was the peak height of the product peptide and S was the peak height of the substrate peptide. For each compound, enzyme activity was measured at various concentrations (12 concentrations of compound spaced by 3× dilution intervals). Negative control samples (00 inhibition in the absence of inhibitor) and positive control samples (10000 inhibition, in the presence of 20 mM EDTA) were assembled in replicates of four and were used to calculate 00 inhibition values for each inhibitor at each concentration. Percent inhibition (Pinh) was determined using following equation: Pinh=(PSR0%−PSRinh)/(PSR0%−PSR100%)*100, where PSRinh was the product sum ratio in the presence of inhibitor, PSR0% was the average product sum ratio in the absence of inhibitor and PSR100% was the average product sum ratio in 100%−inhibition control samples.

The IC₅₀ values of inhibitors were determined by 4 parameter sigmoidal dose response model fitting of the inhibition curves (Pinh versus inhibitor concentration) using XLfit® 4 software (available from IDBS, Boston, Mass.).

TABLE 5
BTK Assay Activity

| Example | BTK IC₅₀ (nM) |
|---|---|
| 1 | 0.426 |
| 2 | 4.07 |
| 3 | 0.993 |
| 4 | 0.331 |
| 5 | 10.5 |
| 6 | 1.1 |
| 7 | 11.5 |
| 8 | 4.7 |
| 9 | 13.8 |
| 10 | 3.78 |
| 11 | 2.25 |
| 12 | 0.495 |
| 13 | 1.84 |
| 14 | 37.5 |
| 15 | 0.684 |
| 16 | 0.592 |
| 17 | 2.72 |
| 18 | 678 |
| 19 | 8.61 |
| 20 | 3.17 |
| 21 | 919 |
| 22 | 1.11 |
| 23 | 2.03 |
| 24 | 0.735 |
| 25 | 29.4 |
| 26 | 2.16 |
| 27 | 126 |
| 28 | 4.66 |
| 29 | 24.2 |
| 30 | 1.05 |
| 31 | 88.3 |
| 32 | 18.1 |
| 33 | 24.8 |
| 34 | 12.5 |
| 35 | 0.884 |
| 36 | 14.2 |
| 37 | 2.48 |
| 38 | 3.58 |
| 39 | 5.8 |
| 40 | 48.6 |
| 41 | 15.8 |
| 42 | 11.8 |
| 43 | 1.17 |
| 44 | 15.4 |
| 45 | 2.33 |
| 46 | 76.7 |
| 47 | 819 |
| 48 | 51.1 |
| 49 | 398 |
| 50 | 38.6 |
| 51 | 507 |
| 52 | 33.3 |
| 53 | 1.2 |
| 54 | 1.19 |
| 55 | 7.63 |
| 56 | 14.8 |
| 57 | 1.76 |
| 59 | 2.17 |
| 58 | 77.5 |
| 60 | 1.42 |
| 61 | 27.2 |
| 62 | 6.2 |
| 63 | 20.3 |
| 64 | 5.58 |
| 65 | 0.953 |
| 66 | 65.7 |
| 67 | 0.737 |
| 68 | 3.18 |
| 69 | 2.18 |
| 70 | 7.09 |
| 71 | 22.8 |
| 72 | 7.84 |
| 73 | 15.7 |
| 74 | 1.51 |
| 75 | 1.14 |
| 76 | 0.968 |
| 77 | 0.533 |
| 78 | 0.671 |
| 81 | 5.24 |
| 82 | 0.515 |
| 83 | 66.6 |
| 84 | 1.4 |
| 85 | 107 |
| 86 | 0.706 |
| 87 | 3.32 |
| 88 | 0.797 |
| 89 | 1.75 |
| 90 | 1.29 |
| 91 | 1.66 |
| 92 | 2.42 |
| 93 | 5.09 |
| 94 | 23.5 |
| 95 | 14.1 |
| 96 | 59.2 |
| 97 | 120 |
| 98 | 14.4 |
| 99 | 136 |
| 100 | 46.3 |
| 101 | 64.1 |
| 102 | >1000 |

TABLE 6
Other Kinase Assay Activity

| | Example 6 | Compound A |
|---|---|---|
| Enzyme | IC₅₀ (nM) | IC₅₀ (nM) |
| BTK | 1.1 | 1 |
| BLK | 137 | 5.8 |
| ERBB4 | 757 | 57.5 |
| MAP2K1 | | 41.3 |
| TEC | 14.7 | 0.8 |
| TXK | 222 | 1.9 |
| EGFR | 844 | 200 |
| MAP2K2 | 7800 | 75 |
| BMX | 15.2 | 5 |

### Whole Blood Compound Screening Phosphorylation of Y223 Site of BTK

Healthy human whole blood (WB) was collected from STEMCELL™ Technologies or ALLCELLS® in sodium heparinized tubes. Whole blood was stored at ambient temperature to avoid coagulation. Each compound was tested in 3 separate donors. Compound stock solutions (10 mM) were used to generate dose titration (8 concentrations, starting concentration 10 μM, 1:4 dilution) in appropriate volume of dimethyl sulfoxide (0.2%). Whole blood was incubated with compounds in 96-well plates for 2 hours in tissue culture incubators at 37° C. under a 5% CO₂ atmosphere. The whole blood was lysed in equal volume of lysis buffer (Cell Signaling Technology®, Danvers, Mass.) containing complete protease inhibitor tablet, PhosSTOP™ tablet, sodium orthovanadate and sodium fluoride and phenylmethanesulfonyl fluoride solution. The lysed whole blood was then frozen at −80° C. for overnight.

Phosphorylation of Y223 of BTK was measured using an MSD assay (Meso Scale Diagnostics, LLC, Rockville, Md.). The thawed lysed whole blood was incubated with the capturing antibody rabbit anti-human BTK (D3H5) (Cell Signaling Technology, Danvers, Mass.) at 4° C. overnight. After washing out the unbound samples, the detection antibody biotinylated rabbit anti-pY223-BTK(D9T6H) (Cell Signaling Technology®, Danvers, Mass.) was added to the plates. After washing out the unbound antibodies, SULFO-TAG Streptavidin (Meso Scale Diagnostics, LLC, Rockville, Md.) was used for detection on Meso Sector S600 (Meso Scale Diagnostics, LLC, Rockville, Md.).

The EC₅₀ values were determined using software from Dotmatics. The activity of exemplary compounds is illustrated below.

TABLE 7
Whole Blood Assay Activity

| | WB BTK- |
|---|---|
| | pY223 EC₅₀ |
| Example | (nM) |
| 1 | 42.2 |
| 3 | 40.7 |
| 4 | 35.7 |
| 6 | 24.1 |
| 12 | 32.9 |
| 15 | 55.6 |
| 16 | 18.3 |
| 22 | 38.2 |
| 24 | 92.9 |
| 30 | 45.3 |
| 35 | 128 |
| 43 | 62.5 |
| 44 | 158 |
| 53 | 71.8 |
| 54 | 76.7 |
| 57 | 85.9 |
| 59 | 117 |
| 60 | 83.4 |
| 62 | 7.75 |
| 63 | 19.4 |
| 64 | 75 |
| 65 | 34.2 |
| 66 | 1820 |
| 67 | 18.8 |
| 68 | 96.2 |
| 69 | 29.2 |
| 70 | 239 |
| 71 | 279 |
| 72 | 21.7 |
| 73 | 255 |
| 74 | 56 |
| 75 | 61 |
| 76 | 85.5 |
| 77 | 31.5 |
| 78 | 57.9 |
| 81 | 75.1 |
| 82 | 44.1 |
| 83 | 1730 |
| 84 | 45.6 |
| 85 | 2030 |
| 86 | 315 |
| 87 | 162 |
| 88 | 225 |
| 89 | 525 |
| 90 | 933 |
| 91 | 399 |
| 92 | 608 |
| 93 | 400 |

### CYP3A4 Induction Assay

CYP induction was evaluated in vitro by determining the expression levels of CYP 3A4 mRNA using the qPCR (quantitative polymerase chain reaction) assay. On day one, cryopreserved human hepatocytes were thawed in prewarmed (37° C.) thawing medium (cryopreserved hepatocyte recovery medium, Gibco™ CM 7000; Thermo Fisher Scientific), centrifuged (1000 g for 10 minutes) and resuspended in plating medium (Gibco™ CM3000—Williams Medium E supplemented with hepatocytes plating supplement pak-serum containing; Thermo Fisher Scientific). Cells were counted by trypan blue exclusion using a hemocytometer and adjusted to a cell density of 1.2×10⁶ cells/μL. Thereafter, 0.05 μL of cell suspension was aliquoted per well in a 96-well plate, resulting in 60,000 cells/well. Plates were shaken in the north-south and east-west direction during plating. Plated cells were incubated at 37° C./5% CO₂ in a humidified cell culture incubator for 4-6 hours. During this time, incubation medium (Gibco™ CM4000, cell maintenance supplement pack; Thermo Fisher Scientific) was mixed with Gibco™ Geltrex™ (Thermo Fischer Scientific) at the appropriate Geltrex™ protein concentration. Following recovery, medium was removed from cells and replaced with freshly prepared incubation medium. Cells were incubated overnight in a cell culture incubator (37° C./5% CO₂).

The next day the incubation medium was prepared by combining the hepatocyte maintenance supplement pack with Williams medium E. The medium was warmed to 37° C. 1000× dimethyl sulfoxide compound stocks were prepared to the desired concentrations. 10 mM compound stocks in dimethyl sulfoxide were diluted (1 μL to 1 mL CM4000; final concentration=10 μM). Overlay medium was aspirated and 0.1 μL dose solution added to the desired labeled 96 well plate. Cells were returned to the humidified incubator overnight (37° C./5% CO₂).

On day 3, incubation medium was warmed to 37° C., and the same process for compound preparation repeated as in day 2, i.e. 1000× dimethyl sulfoxide compound stocks were prepared to the desired concentration, diluted (1:1000) in warm incubation medium, and this freshly prepared medium was used to replace treatment medium. Cells were returned to the humidified incubator (37° C./5% CO₂) overnight.

On day 4, RNA isolation was performed. Prior to RNA isolation, cell viability was assessed using presto blue reagent. 10× dilution of PrestoBlue™ reagent (Thermo Fisher Scientific) was prepared by mixing 1.4 mL PrestoBlue™ with 12.6 mL incubation medium. Medium was aspirated from cells, and 100 μL of cell viability medium (10× dilution of PrestoBlue™ reagent) added and incubated for 17 minutes. Cell viability was determined by measuring absorbance using a microplate reader (Spark® 10M Tecan Life Sciences). After this measurement, media was aspirated using a multichannel aspirator and a glass pipet to completely aspirate from each well. Plates were stored or immediately used for mRNA isolation. All surfaces were cleaned using RNase away. 2× Lysis buffer was well mixed after incubation at ambient temperature. Fresh lysis buffer was prepared by mixing 0.125 μL of 0.5 mM DTT with 4.875 μL 2× lysis buffer. 1× lysis buffer containing DTT was then prepared by adding 5 μL dilution buffer to the 2× lysis buffer and mixing thoroughly. Thereafter, cell lysates were prepared by adding 1× lysis buffer to each sample well (96-well plate). Samples were incubated for 3-6 minutes at ambient temperature while shaking (350-400 rpm) to lyse the cells. Cells were scrapped with tips, and cell lysate (80 μL) was transferred to each well of the mRNA Catcher PLUS™ plate (Thermo Fisher Scientific). mRNA Catcher PLUS™ plate containing samples was covered with adhesive plastic film plate cover and incubated at ambient temperature for 45-60 minutes for RNA hybridization.

After hybridization, lysate was aspirated from the cells. Wash buffer (from mRNA Catcher PLUS™ Purification Kit; W15, 100 μL) was added to the wells, and the plate was incubated for 1 minute at ambient temperature. Reverse pipetting was used to avoid air bubbles. Wash buffer was aspirated and washing repeated twice for a total of 3 washes. Following final wash, all remaining wash buffer was thoroughly aspirated.

To elute, elution buffer (80 μL) was added to the wells of the mRNA Catcher PLUS™ plate. The plate was covered with adhesive plastic film, incubated at 68° C. for 5 minutes, and immediately cooled to 4° C. using a thermocycler with heated lids to avoid condensation. The eluted mRNA was transferred from the wells to RNase-free sealable microtiter plates and stored at −80° C. until use.

Following mRNA isolation, reverse transcription (RT) reactions were performed using Tagman™ Reverse Transcription Reagents (ThermoFisher Scientific). RT master mix was prepared by mixing 2.5 μL 10×RT buffer, 5.5 μL 25 mM MgCl₂, 5 μL deoxyNTP mixture, 1.25 μL 50 mM random hexamers, 0.5 μL 2×RNAse inhibitor, 0.625 μL multiscript RTase (50 U/μL) and 0.625 μL water. An aliquot of RT mix (16 μL) and an aliquot of mRNA sample (9 μL) was added to each well of a 96-well plate. The SimpliAmp™ Thermal cycler PCR System VIIA7 from Applied Biosystems was used for reverse transcription under the following conditions: segment 1: 25° C. for 10 minutes; segment 2: 45° C. for 45 minutes; segment 3: 95° C. for 5 minutes; and segment 4: 4° C. hold; with 1 cycle for each segment. Primers used were HS00604506_m1 for CYP3A4 and HS99999905_m1 for hGAPDH (control, glyceraldeyde-3-phosphate dehydrogenase).

Thereafter, Tagman™ qPCR was performed using Applied Biosystems QuantStudio 7 Flex System (Applied Biosystem, Foster City, Calif.). TaqMan™ Fast Advanced Master Mix and Gene expression assay primers and probes were purchased from Applied Biosystems. The PCR reaction mix included diluted cDNA (2 μL) and 18 μL of the Tagman™ qPCR master mix (10 μL TaqMan™ Fast Advanced Master Mix, 1 μL of gene expression primer probe mix, 7 μL nuclease-free water). qPCR thermal cycling conditions were: segment 1: 50° C. hold for 2 minutes; segment 2: 95° C. hold for 20 seconds; segment 3: 40 cycles at 95° C. for 1 second; and segment 4: 40 cycles at 60° C. for 20 seconds.

The fold induction in CYP isoform mRNA caused by treatment with the compounds was determined using the comparative quantitative real-time polymerase chain reaction (qPCR). This method used the comparative CT (ΔΔCT) method for calculating relative quantitation of gene expression. It assigned the control mean equal to 1 and calculated the fold change using the following equation: Fold change=2ΔΔCT, where ΔCT was the difference in threshold cycle between the target and reference genes and ΔΔCT=ΔCT (treated sample)−ΔCT (vehicle). The induction potential of test compounds was also compared with that of a prototypical human CYP450 inducer where the induction potency of the prototypical inducer was expressed as 100%. The prototypical inducer used was rifampicin for CYP3A4.

TABLE 8
CYP3A4 Induction Assay Activity

| Example/ | | CYP3A4 Ind. fold |
|---|---|---|
| Compound | Structure | (%) |
| 6 | 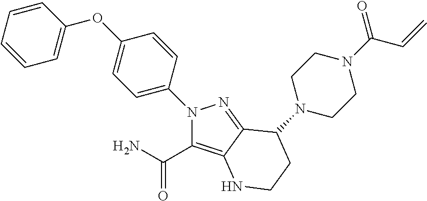 | 0.9 (−1.3) |
| 45 | 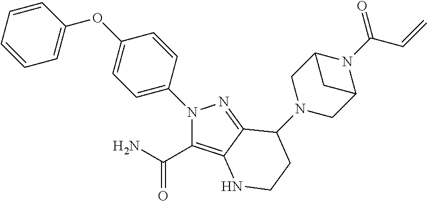 [SMILES: C=CC(=O)N1C2CC1CN(C1CCNc3c1nn(-c1ccc(Oc4ccccc4)cc1)c3C(N)=O)C2] | 25.40 |
| 58 | 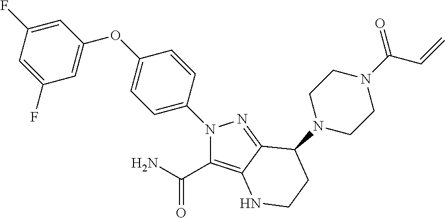 [SMILES: C=CC(=O)N1CCN([C@H]2CCNc3c2nn(-c2ccc(Oc4cc(F)cc(F)c4)cc2)c3C(N)=O)CC1] | 1.58 |
| 59 | 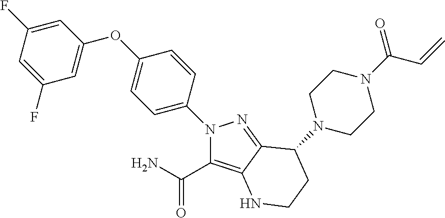 [SMILES: C=CC(=O)N1CCN([C@@H]2CCNc3c2nn(-c2ccc(Oc4cc(F)cc(F)c4)cc2)c3C(N)=O)CC1] | 0.85 |
| Compound A | 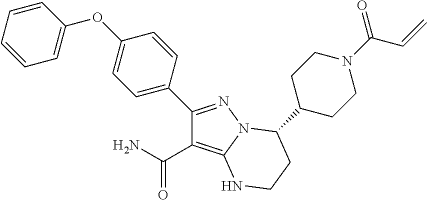 [SMILES: C=CC(=O)N1CCC([C@@H]2CCNc3c(C(N)=O)c(-c4ccc(Oc5ccccc5)cc4)nn32)CC1] | 14.6 (125) |

### Enzyme Involvement in the Metabolism of Compound 6

The evaluation of enzyme involvement in Compound 6 metabolism was carried out in hepatic and recombinant enzyme systems. The percent contribution of cytochrome P450 (all CYPs tested) to Compound 6 metabolism in human hepatocyte suspensions was 38.3%. Of the all CYPs tested (1A2, 2B6, 2C8, 2C9, 2D6, and 3A4) and flavin-containing monooxygenase (FMO) 3 evaluated, CYP3A4 was identified as the major contributing enzyme. Thus, following incubation with a pan-CYP inhibitor cocktail in human hepatocytes, CYPs were found to contribute to approximately 30% of the hepatic metabolism of Compound 6, with additional, non-CYP hepatic enzymes contributing to the remaining clearance (about 70%).

### Human Recombinant CYP and FMO3 Phenotyping

The involvement of CYPs and FMO3 in the metabolism of Compound 6 was evaluated using recombinant human CYP1A2, 2B6, 2C8, 2C9, 2C19, 2D6, 3A4, and FMO3 (CYPs and FMO3 from Corning Life Sciences). Incubations contained 0.5 μM of Compound 6, 100 pmol/mL of each CYP isoform, or 0.5 mg/mL of FMO3 isoform, and 1 mM NADPH cofactor in 50 mM potassium phosphate buffer at pH 7.4. All incubations were pre-warmed at 37° C. for 10 minutes, followed by addition of cofactor to initiate the reaction. Incubations were carried out for 60 minutes at 37° C. At each time point (0, 5, 10, 20, 30 and 60 minutes), an aliquot of sample in the incubation pleases was taken and dispersed into a quench plate containing 3-fold volume of acetonitrile and carbutamide as an internal standard. The samples were centrifuged, and the supernatant was analyzed using LC-MS/MS.

### Chemical Inhibition in Human Hepatocytes

The contribution of CYPs to the metabolism of Compound 6 was evaluated in human hepatocyte suspension (about 500,000 cells/mL) (hepatocytes from BioIVT, Inc.) treated with or without azamulin (CYP3A4 mechanism-based inhibitor) or 1-aminobenzotriazole (ABT)/tienilic acid (a pan-CYP inhibitor regimen) for 30 minutes. The hepatocyte suspension with inhibitors was preincubated with 2× azamulin or 1-ABT/tienilic acid in Williams' media E and then added to 2× Compound 6 in media to give a final concentrations of 2.5 μM azamulin or 0.5 mM 1-ABT/1.5 μM tienilic acid and 0.5 μM Compound 6, respectively.

Final incubation volume was 25 μL. Samples were incubated in 384-well plates at 37° C. with 5% CO₂ and gentle shaking. At each time point (0, 15, 30, 60, 120, and 240 minutes), samples in the incubation plates were quenched with acetonitrile:MeOH (95:5, v:v) with carbutamide as internal standard (quench ration 3:1). The samples were centrifuged, and the supernatant was analyzed using LC-MS/MS.

### Enzyme Kinetic Analysis in Recombinant Human CYP3A4

The incubation mixture contained rCYP3A4 (60 pmol/mL with final protein concentration 0.48 mg/mL) and Compound 6 at varying concentrations (0.8-60 μM) in 50 mM potassium phosphate buffer at pH 7.4. After a 5-minute warm up period, reactions were initiated with addition of 10 mM NADPH solution (1 mM final concentration) and incubated at 37° C. At each time point (0, 5, 10, 20, 30, and 45 minutes), an aliquot was taken and dispensed into a 3-fold volume of acetonitrile with carbutamide as internal standard. The samples were centrifuged, and the supernatant was analyzed by using LC-MS/MS.

### Analytical Methods

Quantitation of the substrate depletion in recombinant CYPs and FMO3 phenotyping, chemical inhibition in hepatocytes, and enzyme kinetics assays were analyzed by monitoring substrate depletion using LC-MS/MS.

Intrinsic Clearance in rCYP and Hepatocytes

The intrinsic clearance of Compound 6 in rCYP and hepatocytes was determined by integrating the parent peak on the chromatograms, and the extent of substrate depletion measured by comparing parent peak area loss over time. The elimination rate constant (k, transformation of % parent remaining versus incubation time: y=a*e^{-(kt)}. Where y is the % parent remaining, t (min) is the time point within the incubation, and a is the % parent remaining at time 0 minute. From this, intrinsic clearance in rCYP (CL_{int, CYP}, expressed in units of μL/min/pmol CYP) and in hepatocytes (CL_{int, hep}, expressed in units of μL/min/million cells were derived using the equations below:

CLint,rCYP=k*1000/CYP content (pmol/mL)

Clint,hep=k*1000/Cell Concentration (million cells/mL)

Based on measured % parent remaining of 85% or greater, the limit quantification of half-life is determined as 240 minutes in recombinant enzyme systems and 960 min in hepatocytes systems.

### Chemical Inhibition

The fraction of metabolism inhibited by the chemical treatment in human hepatocytes was calculated using the equation:

fm,CYP=[Clint,hep(no inhibitor)−Clint,hep(PanCYP or azamulin])/[Clint, hep(no inhibitor)]

### Enzyme Kinetics

Individual kinetic parameters were determined for CYP3A4 using a substrate depletion method over a large range of substrate concentrations. The percentage remaining versus time at each substrate concentration was fitted into a first order decay function to determine initial substrate depletion rate constants (kdep). The Kₘ value was determined by plotting the K_{dep} versus the substrate concentration on a linear-log plot using the equation below:

k_{dep}=k_{dep(S[S]=O)}*(1−[S]/[S]+Kₘ

[S] is the substrate concentration, k_{dep[S]=O)} represents the theoretical maximum depletion rate constant at an infinitesimally low-substrate concentration, and Kₘ is the Michaelis-Menten constant. The Kₘ (μM) is the concentration at the inflection point on the plot equal to the substrate concentration that gives a k_{dep} value that is half the value of k_{dep([S]=O)}. Values of CL_{intCYP (μL/min/pmol CYP)} and Vₘₐₓ (pmol/min/pmol CYP) are calculated from the equations below:

CL_{intCYP}=k_{dep([S]=O)}*1000/[CYP Content (pmol/mL)

Vₘₐₓ=Kₘ*CL_{int,CYP}

TABLE 9
Effect of Chemical Inhibitors on the In vitro Clearance
of Compound 6 in Human Hepatocyte Suspensions

| CLᵢₙₜ, no | CLᵢₙₜ, | CLᵢₙₜ, | | % Total |
|---|---|---|---|---|
| inhibitor¹ | Azamulin¹ | PAN CYP¹ | | CYP |
| (μL/min/ | (μL/min/ | (μL/min/ | % Inhibition | Contribution |
| 10⁶ cells) | 10⁶ cells) | 10⁶ cells) | by azamulin | In Vitro² |
| 13.3 | 9.15 | 8.23 | 31.4 | 38.3 |

¹CLᵢₙₜ, results are the mean of greater than or equal to experiments in singlet.
²Calculated from the PAN CYP inhibitor regimen of 1-ABT and tienilic acid.

All publications and patents mentioned herein are hereby incorporated by reference in their entirety as if each individual publication or patent was specifically and individually indicated to be incorporated by reference.

It is understood that the foregoing detailed description and accompanying examples are merely illustrative and are not to be taken as limitations upon the scope of the present disclosure, which is defined by the appended claims and their equivalents.

What is claimed is:

1. A compound of formula (I): 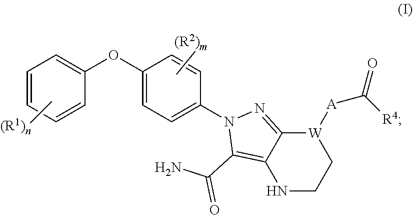
wherein
A is a 4-9 membered heterocycloalkylene substituted with —(R³)ₚ;
W is CH or N;
R¹ is independently selected from the group consisting of halo, C₁-C₄ alkyl, C₃-C₆ cycloalkyl, C₁-C₄ haloalkyl, —CN, —OH, and —OR^{1a};
R^{1a} is selected from the group consisting of C₁-C₄ alkyl and C₁-C₄ haloalkyl;
R² is independently selected from the group consisting of halo, C₁-C₄ alkyl, and OR^{2a};
R^{2a} is selected from the group consisting of C₁-C₄ alkyl and C₁-C₄ haloalkyl;
R³ is independently selected from the group consisting of —OH, C₁-C₄ alkyl, C₁-C₄ hydroxyalkyl, —CH₂CH₂—, and —CH₂CH₂CH₂—;
R⁴ is selected from the group consisting of C₁-C₄ haloalkyl, —CH═CHR^{4a}, and C₂-C₄ alkynyl;
R^{4a} is selected from the group consisting of hydrogen, halo, C₁-C₄ alkyl, —OR^{4b}, —CO₂R^{4b}, and —CO₂NH₂; wherein the R^{4a} C₁-C₄ alkyl may optionally be substituted with —OR⁴, or —NR^{4c}R^{4d};
R^{4b}, R^{4c}, and R^{4d} are each independently C₁-C₄ alkyl;
m is 0, 1, 2, or 3;
n is 0, 1, 2, or 3; and
p is 0, 1, 2, or 3; or a pharmaceutically acceptable salt thereof.

2. The compound of claim 1, wherein A is a nitrogen-containing heterocycloalkylene, or a pharmaceutically acceptable salt thereof.

3. The compound of claim 2, wherein A is a 6-membered nitrogen-containing heterocycloalkylene, or a pharmaceutically acceptable salt thereof.

4. The compound of claim 3, wherein A is piperazinediyl, or a pharmaceutically acceptable salt thereof.

5. The compound of claim 3, wherein A is piperidinediyl, or a pharmaceutically acceptable salt thereof.

6. The compound of claims 1-5, wherein W is CH, or a pharmaceutically acceptable salt thereof.

7. The compound of claims 1-6, wherein W is N, or a pharmaceutically acceptable salt thereof.

8. The compound of claims 1-2, wherein A is selected from the group consisting of: 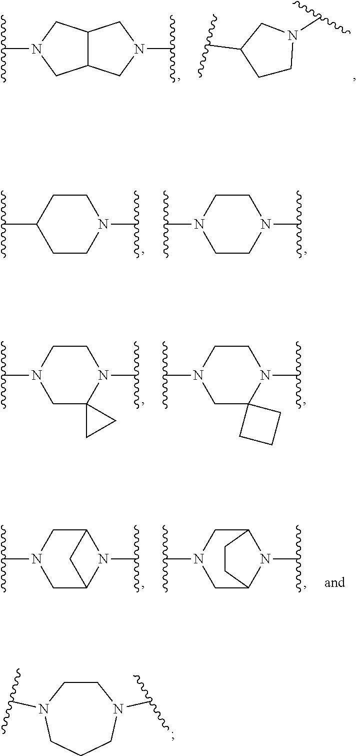
or a pharmaceutically acceptable salt thereof.

9. The compound of claim 1, according to formula (II): 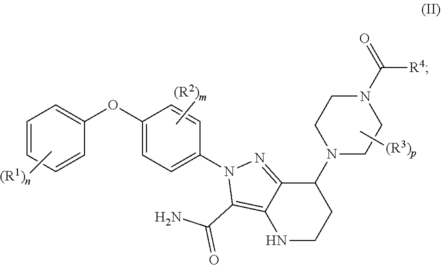
wherein
R¹ is independently selected from the group consisting of halo, C₁-C₄ alkyl, C₃-C₆ cycloalkyl, C₁-C₄ haloalkyl, —CN, —OH, and —OR^{1a};
R^{1a} is selected from the group consisting of C₁-C₄ alkyl and C₁-C₄ haloalkyl;
R² is independently selected from the group consisting of halo, C₁-C₄ alkyl, and OR^{2a};
R^{2a} is selected from the group consisting of C₁-C₄ alkyl and C₁-C₄ haloalkyl;
R³ is independently selected from the group consisting of —OH, C₁-C₄ alkyl, C₁-C₄ hydroxyalkyl, —CH₂CH₂—, and —CH₂CH₂CH₂—;
R⁴ is selected from the group consisting of C₁-C₄ haloalkyl, —CH═CHR^{4a}, and C₂-C₄ alkynyl;
R^{4a} is selected from the group consisting of hydrogen, halo, C₁-C₄ alkyl, —OR^{4b}, —CO₂R^{4b} and —CO₂NH₂; wherein the R^{4a} C₁-C₄ alkyl may optionally be substituted with —OR⁴, or —NR^{4c}R^{4d};
R^{4b}, R^{4c}, and R^{4d} are each independently C₁-C₄ alkyl;
m is 0, 1, 2, or 3;
n is 0, 1, 2, or 3; and
p is 0, 1, 2, or 3; or a pharmaceutically acceptable salt thereof.

10. The compound of claim 9, wherein R⁴ is selected from the group consisting of —CH═CHR^{4a} and C₂-C₄ alkynyl, or a pharmaceutically acceptable salt thereof.

11. The compound of claims 9-10, wherein m is 0, or a pharmaceutically acceptable salt thereof.

12. The compound of claims 9-11, wherein p is 0, or a pharmaceutically acceptable salt thereof.

13. The compound of claims 9-11, wherein R³ is methyl, or a pharmaceutically acceptable salt thereof.

14. The compound of claims 9-13, wherein R¹ is independently selected from the group consisting of F, —CN, —OH, methyl, cyclopropyl, trifluoromethyl, methoxy, and trifluoromethoxy, or a pharmaceutically acceptable salt thereof.

15. The compound of claims 9-13, wherein n is 0, or a pharmaceutically acceptable salt thereof.

16. The compound of claims 9-15, wherein R⁴ is selected from the group consisting of —CH═CH₂ and —C≡CCH₃, or a pharmaceutically acceptable salt thereof.

17. The compound of claim 9, which is: 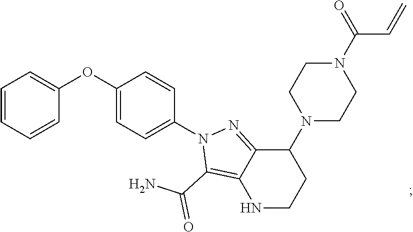 [SMILES: C=CC(=O)N1CCN(C2CCNc3c2nn(-c2ccc(Oc4ccccc4)cc2)c3C(N)=O)CC1]
or a pharmaceutically acceptable salt thereof.

18. The compound of claim 9, which is 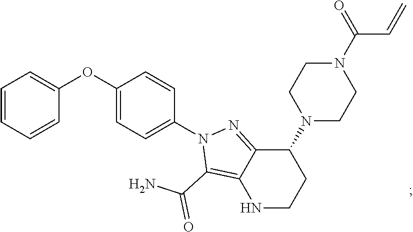 [SMILES: C=CC(=O)N1CCN([C@@H]2CCNc3c2nn(-c2ccc(Oc4ccccc4)cc2)c3C(N)=O)CC1]
or a pharmaceutically acceptable salt thereof.

19. The compound of claim 18, which is:

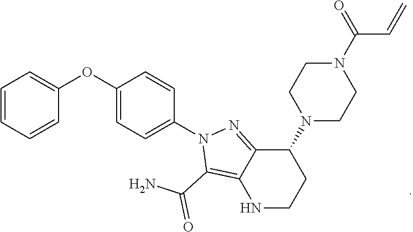 [SMILES: C=CC(=O)N1CCN([C@@H]2CCNc3c2nn(-c2ccc(Oc4ccccc4)cc2)c3C(N)=O)CC1]

20. The compound of claim 18, which is the pharmaceutically acceptable salt of:

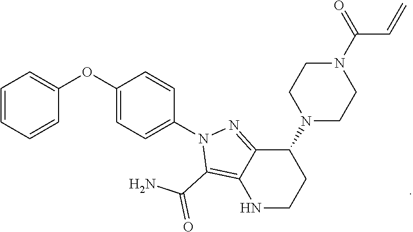 [SMILES: C=CC(=O)N1CCN([C@@H]2CCNc3c2nn(-c2ccc(Oc4ccccc4)cc2)c3C(N)=O)CC1]

21. The compound of claim 1, selected from the group consisting of:
2-(4-phenoxyphenyl)-7-[1-(prop-2-enoyl)piperidin-4-yl]-4,5,6,7-tetrahydro-2H-pyrazolo[4,3-b]pyridine-3-carboxamide;
7-[1-(but-2-ynoyl)piperidin-4-yl]-2-(4-phenoxyphenyl)-4,5,6,7-tetrahydro-2H-pyrazolo[4,3-b]pyridine-3-carboxamide;
2-(4-phenoxyphenyl)-7-[4-(prop-2-enoyl)piperazin-1-yl]-4,5,6,7-tetrahydro-2H-pyrazolo[4,3-b]pyridine-3-carboxamide;
(7R)-2-(4-phenoxyphenyl)-7-[1-(prop-2-enoyl)piperidin-4-yl]-4,5,6,7-tetrahydro-2H-pyrazolo[4,3-b]pyridine-3-carboxamide;
(7S)-2-(4-phenoxyphenyl)-7-[1-(prop-2-enoyl)piperidin-4-yl]-4,5,6,7-tetrahydro-2H-pyrazolo[4,3-b]pyridine-3-carboxamide;
(7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide;
(7S)-2-(4-phenoxyphenyl)-7-[4-(prop-2-enoyl)piperazin-1-yl]-4,5,6,7-tetrahydro-2H-pyrazolo[4,3-b]pyridine-3-carboxamide;
(7SR)-2-(4-phenoxyphenyl)-7-[(3RS)-1-(prop-2-enoyl)piperidin-3-yl]-4,5,6,7-tetrahydro-2H-pyrazolo[4,3-b]pyridine-3-carboxamide;
(7RS)-2-(4-phenoxyphenyl)-7-[(3RS)-1-(prop-2-enoyl)piperidin-3-yl]-4,5,6,7-tetrahydro-2H-pyrazolo[4,3-b]pyridine-3-carboxamide;
2-(4-phenoxyphenyl)-7-[1-(prop-2-enoyl)pyrrolidin-3-yl]-4,5,6,7-tetrahydro-2H-pyrazolo[4,3-b]pyridine-3-carboxamide;
2-[4-(4-fluorophenoxy)phenyl]-7-[1-(prop-2-enoyl)piperidin-4-yl]-4,5,6,7-tetrahydro-2H-pyrazolo[4,3-b]pyridine-3-carboxamide;
2-[4-(3-fluorophenoxy)phenyl]-7-[1-(prop-2-enoyl)piperidin-4-yl]-4,5,6,7-tetrahydro-2H-pyrazolo[4,3-b]pyridine-3-carboxamide;
2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide;
2-(2-methoxy-4-phenoxyphenyl)-7-[1-(prop-2-enoyl)piperidin-4-yl]-4,5,6,7-tetrahydro-2H-pyrazolo[4,3-b]pyridine-3-carboxamide;
2-[4-(3-methoxyphenoxy)phenyl]-7-[1-(prop-2-enoyl)piperidin-4-yl]-4,5,6,7-tetrahydro-2H-pyrazolo[4,3-b]pyridine-3-carboxamide;
2-(4-phenoxyphenyl)-7-[1-(prop-2-enoyl)piperidin-4-yl]-4,5,6,7-tetrahydro-2H-pyrazolo[3,4-b]pyrazine-3-carboxamide;
2-[4-(4-methoxyphenoxy)phenyl]-7-[1-(prop-2-enoyl)piperidin-4-yl]-4,5,6,7-tetrahydro-2H-pyrazolo[4,3-b]pyridine-3-carboxamide;
7-[(2S,5R)-2,5-dimethyl-4-(prop-2-enoyl)piperazin-1-yl]-2-(4-phenoxyphenyl)-4,5,6,7-tetrahydro-2H-pyrazolo[4,3-b]pyridine-3-carboxamide;
7-[1-(but-2-ynoyl)piperidin-4-yl]-2-(4-phenoxyphenyl)-4,5,6,7-tetrahydro-2H-pyrazolo[3,4-b]pyrazine-3-carboxamide;
2-(4-phenoxyphenyl)-7-[(3R)-3-(propan-2-yl)-4-(prop-2-enoyl)piperazin-1-yl]-4,5,6,7-tetrahydro-2H-pyrazolo[4,3-b]pyridine-3-carboxamide;
7-[(2R,5S)-2,5-dimethyl-4-(prop-2-enoyl)piperazin-1-yl]-2-(4-phenoxyphenyl)-4,5,6,7-tetrahydro-2H-pyrazolo[4,3-b]pyridine-3-carboxamide;
2-(4-phenoxyphenyl)-7-[1-(prop-2-enoyl)azetidin-3-yl]-4,5,6,7-tetrahydro-2H-pyrazolo[3,4-b]pyrazine-3-carboxamide;
7-[1-(prop-2-enoyl)piperidin-4-yl]-2-{4-[3-(trifluoromethyl)phenoxy]phenyl}-4,5,6,7-tetrahydro-2H-pyrazolo[4,3-b]pyridine-3-carboxamide;
2-[4-(3-methylphenoxy)phenyl]-7-[1-(prop-2-enoyl)piperidin-4-yl]-4,5,6,7-tetrahydro-2H-pyrazolo[4,3-b]pyridine-3-carboxamide;
2-(4-phenoxyphenyl)-7-[4-(prop-2-enoyl)-1,4-diazepan-1-yl]-4,5,6,7-tetrahydro-2H-pyrazolo[4,3-b]pyridine-3-carboxamide;
2-(4-phenoxyphenyl)-7-[5-(prop-2-enoyl)-5,8-diazaspiro[3.5]nonan-8-yl]-4,5,6,7-tetrahydro-2H-pyrazolo[4,3-b]pyridine-3-carboxamide;
7-[4-(but-2-ynoyl)piperazin-1-yl]-2-(4-phenoxyphenyl)-4,5,6,7-tetrahydro-2H-pyrazolo[4,3-b]pyridine-3-carboxamide;
2-[4-(4-chlorophenoxy)phenyl]-7-[1-(prop-2-enoyl)piperidin-4-yl]-4,5,6,7-tetrahydro-2H-pyrazolo[4,3-b]pyridine-3-carboxamide;
(7S)-2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide;
(7R)-2-[4-(2,4-difluorophenoxy)phenyl]-7-[1-(prop-2-enoyl)piperidin-4-yl]-4,5,6,7-tetrahydro-2H-pyrazolo[4,3-b]pyridine-3-carboxamide;
(7S)-7-[(2S)-2-methyl-4-(prop-2-enoyl)piperazin-1-yl]-2-(4-phenoxyphenyl)-4,5,6,7-tetrahydro-2H-pyrazolo[4,3-b]pyridine-3-carboxamide;
(7R)-7-[(2S)-2-methyl-4-(prop-2-enoyl)piperazin-1-yl]-2-(4-phenoxyphenyl)-4,5,6,7-tetrahydro-2H-pyrazolo[4,3-b]pyridine-3-carboxamide;
2-(4-phenoxyphenyl)-7-[(1R,4R)-5-(prop-2-enoyl)-2,5-diazabicyclo[2.2.1]heptan-2-yl]-4,5,6,7-tetrahydro-2H-pyrazolo[4,3-b]pyridine-3-carboxamide;
2-(4-phenoxyphenyl)-7-[(1S,4S)-5-(prop-2-enoyl)-2,5-diazabicyclo[2.2.1]heptan-2-yl]-4,5,6,7-tetrahydro-2H-pyrazolo[4,3-b]pyridine-3-carboxamide;
2-(4-phenoxyphenyl)-7-[(1R,5S)-8-(prop-2-enoyl)-3,8-diazabicyclo[3.2.1]octan-3-yl]-4,5,6,7-tetrahydro-2H-pyrazolo[4,3-b]pyridine-3-carboxamide;
2-[4-(2,4-difluorophenoxy)phenyl]-7-[4-(prop-2-enoyl)piperazin-1-yl]-4,5,6,7-tetrahydro-2H-pyrazolo[4,3-b]pyridine-3-carboxamide;
2-(2-fluoro-4-phenoxyphenyl)-7-[1-(prop-2-enoyl)piperidin-4-yl]-4,5,6,7-tetrahydro-2H-pyrazolo[4,3-b]pyridine-3-carboxamide;
2-[4-(2-fluorophenoxy)phenyl]-7-[4-(prop-2-enoyl)piperazin-1-yl]-4,5,6,7-tetrahydro-2H-pyrazolo[4,3-b]pyridine-3-carboxamide;
(7S)-7-[4-(but-2-ynoyl)piperazin-1-yl]-2-(4-phenoxyphenyl)-4,5,6,7-tetrahydro-2H-pyrazolo[4,3-b]pyridine-3-carboxamide;
(7R)-7-[4-(but-2-ynoyl)piperazin-1-yl]-2-(4-phenoxyphenyl)-4,5,6,7-tetrahydro-2H-pyrazolo[4,3-b]pyridine-3-carboxamide;
2-(4-phenoxyphenyl)-7-[3-(prop-2-enoyl)-3,6-diazabicyclo[3.1.1]heptan-6-yl]-4,5,6,7-tetrahydro-2H-pyrazolo[4,3-b]pyridine-3-carboxamide;
7-[(3R)-3-methyl-4-(prop-2-enoyl)piperazin-1-yl]-2-(4-phenoxyphenyl)-4,5,6,7-tetrahydro-2H-pyrazolo[4,3-b]pyridine-3-carboxamide;
7-[(3S)-3-methyl-4-(prop-2-enoyl)piperazin-1-yl]-2-(4-phenoxyphenyl)-4,5,6,7-tetrahydro-2H-pyrazolo[4,3-b]pyridine-3-carboxamide;
(7R)-2-[4-(4-hydroxyphenoxy)phenyl]-7-[4-(prop-2-enoyl)piperazin-1-yl]-4,5,6,7-tetrahydro-2H-pyrazolo[4,3-b]pyridine-3-carboxamide;
2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide;
(7R)-7-{4-[(2E)-4-(dimethylamino)but-2-enoyl]piperazin-1-yl}-2-(4-phenoxyphenyl)-4,5,6,7-tetrahydro-2H-pyrazolo[4,3-b]pyridine-3-carboxamide;
(7R)-7-{4-[(2E)-but-2-enoyl]piperazin-1-yl}-2-(4-phenoxyphenyl)-4,5,6,7-tetrahydro-2H-pyrazolo[4,3-b]pyridine-3-carboxamide;
(7R)-7-{4-[(2E)-4-amino-4-oxobut-2-enoyl]piperazin-1-yl}-2-(4-phenoxyphenyl)-4,5,6,7-tetrahydro-2H-pyrazolo[4,3-b]pyridine-3-carboxamide;
7-[4-(fluoroacetyl)piperazin-1-yl]-2-(4-phenoxyphenyl)-4,5,6,7-tetrahydro-2H-pyrazolo[4,3-b]pyridine-3-carboxamide;
(7R)-7-{4-[(2E)-3-ethoxyprop-2-enoyl]piperazin-1-yl}-2-(4-phenoxyphenyl)-4,5,6,7-tetrahydro-2H-pyrazolo[4,3-b]pyridine-3-carboxamide;
methyl (2E)-4-{4-[3-carbamoyl-2-(4-phenoxyphenyl)-4,5,6,7-tetrahydro-2H-pyrazolo[4,3-b]pyridin-7-yl]piperazin-1-yl}-4-oxobut-2-enoate;
2-(2-chloro-4-phenoxyphenyl)-7-[4-(prop-2-enoyl)piperazin-1-yl]-4,5,6,7-tetrahydro-2H-pyrazolo[4,3-b]pyridine-3-carboxamide;
2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide;
7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide;
2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide;
2-[4-(4-fluorophenoxy)phenyl]-7-[4-(prop-2-enoyl)piperazin-1-yl]-4,5,6,7-tetrahydro-2H-pyrazolo[4,3-b]pyridine-3-carboxamide;
2-[4-(3-fluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(3,5-difluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide;
(7S)-7-[4-(prop-2-enoyl)piperazin-1-yl]-2-{4-[2-(trifluoromethyl)phenoxy]phenyl}-4,5,6,7-tetrahydro-2H-pyrazolo[4,3-b]pyridine-3-carboxamide;
2-[4-(3-fluorophenoxy)phenyl]-7-[1-(prop-2-enoyl)azetidin-3-yl]-4,5,6,7-tetrahydro-2H-pyrazolo[3,4-b]pyrazine-3-carboxamide;
2-[4-(3,4-difluorophenoxy)phenyl]-7-[1-(prop-2-enoyl)azetidin-3-yl]-4,5,6,7-tetrahydro-2H-pyrazolo[3,4-b]pyrazine-3-carboxamide;
(7R)-2-[4-(3-cyanophenoxy)phenyl]-7-[4-(prop-2-enoyl)piperazin-1-yl]-4,5,6,7-tetrahydro-2H-pyrazolo[4,3-b]pyridine-3-carboxamide;
(7R)-7-[4-(prop-2-enoyl)piperazin-1-yl]-2-{4-[3-(trifluoromethyl)phenoxy]phenyl}-4,5,6,7-tetrahydro-2H-pyrazolo[4,3-b]pyridine-3-carboxamide;
2-(4-phenoxyphenyl)-7-[rac-(3aR,6aS)-5-(prop-2-enoyl)hexahydropyrrolo[3,4-c]pyrrol-2(1H)-yl]-4,5,6,7-tetrahydro-2H-pyrazolo[4,3-b]pyridine-3-carboxamide;
(7R)-2-[4-(3-fluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2-cyclopropylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2-cyanophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2,4-difluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(4-fluorophenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide;
(7R)-2-(4-phenoxyphenyl)-7-[6-(prop-2-enoyl)-3,6-diazabicyclo[3.1.1]heptan-3-yl]-4,5,6,7-tetrahydro-2H-pyrazolo[4,3-b]pyridine-3-carboxamide;
(7R)-2-[4-(2,5-difluorophenoxy)phenyl]-7-[4-(prop-2-enoyl)piperazin-1-yl]-4,5,6,7-tetrahydro-2H-pyrazolo[4,3-b]pyridine-3-carboxamide;
2-[4-(2-fluorophenoxy)phenyl]-7-[1-(prop-2-enoyl)azetidin-3-yl]-4,5,6,7-tetrahydro-2H-pyrazolo[3,4-b]pyrazine-3-carboxamide;
2-[4-(2,3-difluorophenoxy)phenyl]-7-[1-(prop-2-enoyl)azetidin-3-yl]-4,5,6,7-tetrahydro-2H-pyrazolo[3,4-b]pyrazine-3-carboxamide;
(7R)-2-[4-(2,3-difluorophenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(3-cyclopropylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2-fluorophenoxy)phenyl]-7-[4-(prop-2-enoyl)piperazin-1-yl]-4,5,6,7-tetrahydro-2H-pyrazolo[4,3-b]pyridine-3-carboxamide;
2-[4-(2,5-difluorophenoxy)phenyl]-7-[1-(prop-2-enoyl)azetidin-3-yl]-4,5,6,7-tetrahydro-2H-pyrazolo[3,4-b]pyrazine-3-carboxamide;
2-[4-(2,4-difluorophenoxy)phenyl]-7-[1-(prop-2-enoyl)azetidin-3-yl]-4,5,6,7-tetrahydro-2H-pyrazolo[3,4-b]pyrazine-3-carboxamide;
(7R)-2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide;
(7S)-2-(4-phenoxyphenyl)-7-[4-(prop-2-enoyl)-4,7-diazaspiro[2.5]octan-7-yl]-4,5,6,7-tetrahydro-2H-pyrazolo[4,3-b]pyridine-3-carboxamide;
7-[4-hydroxy-1-(prop-2-enoyl)piperidin-4-yl]-2-(4-phenoxyphenyl)-4,5,6,7-tetrahydro-2H-pyrazolo[4,3-b]pyridine-3-carboxamide;
(7R)-7-[4-(prop-2-enoyl)piperazin-1-yl]-2-{4-[4-(trifluoromethoxy)phenoxy]phenyl}-4,5,6,7-tetrahydro-2H-pyrazolo[4,3-b]pyridine-3-carboxamide;
(7R)-2-[4-(2-methylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(4-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(3-methylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-7-[4-(prop-2-enoyl)piperazin-1-yl]-2-{4-[3-(trifluoromethoxy)phenoxy]phenyl}-4,5,6,7-tetrahydro-2H-pyrazolo[4,3-b]pyridine-3-carboxamide;
(7R)-7-[4-(prop-2-enoyl)piperazin-1-yl]-2-{4-[2-(trifluoromethoxy)phenoxy]phenyl}-4,5,6,7-tetrahydro-2H-pyrazolo[4,3-b]pyridine-3-carboxamide;
(7R)-2-[4-(3-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(4-methylphenoxy)phenyl]-7-[4-(prop-2-enoyl)piperazin-1-yl]-4,5,6,7-tetrahydro-2H-pyrazolo[4,3-b]pyridine-3-carboxamide;
(7R)-2-[4-(2-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(2-methylphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(3-methylphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(4-methylphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(2-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(3-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7S)-2-[4-(4-methoxyphenoxy)phenyl]-7-[4-(prop-2-enoyl)piperazin-1-yl]-4,5,6,7-tetrahydro-2H-pyrazolo[4,3-b]pyridine-3-carboxamide;
(7S)-7-[4-(prop-2-enoyl)piperazin-1-yl]-2-{4-[2-(trifluoromethoxy)phenoxy]phenyl}-4,5,6,7-tetrahydro-2H-pyrazolo[4,3-b]pyridine-3-carboxamide;
(7S)-7-[4-(prop-2-enoyl)piperazin-1-yl]-2-{4-[3-(trifluoromethoxy)phenoxy]phenyl}-4,5,6,7-tetrahydro-2H-pyrazolo[4,3-b]pyridine-3-carboxamide; and
(7S)-7-[4-(prop-2-enoyl)piperazin-1-yl]-2-{4-[4-(trifluoromethoxy)phenoxy]phenyl}-4,5,6,7-tetrahydro-2H-pyrazolo[4,3-b]pyridine-3-carboxamide;
or a pharmaceutically acceptable salt thereof.

22. The compound of claim 21, which is 2-(4-phenoxyphenyl)-7-[4-(prop-2-enoyl)piperazin-1-yl]-4,5,6,7-tetrahydro-2H-pyrazolo[4,3-b]pyridine-3-carboxamide.

23. The compound of claim 21, which is (7R)-2-(4-phenoxyphenyl)-7-[4-(prop-2-enoyl)piperazin-1-yl]-4,5,6,7-tetrahydro-2H-pyrazolo[4,3-b]pyridine-3-carboxamide, or a pharmaceutically acceptable salt thereof.

24. A method of treating CLL and/or SLL comprising administering to a subject in need thereof a therapeutically effective amount of a compound of formula (I).

25. A pharmaceutical composition comprising a therapeutically effective amount of a compound of formula (I), or a pharmaceutically acceptable salt thereof, in combination with a pharmaceutically acceptable carrier.


SEQUENCE LISTING (4 sequences)

SEQ ID NO: 1 - protein, 14 aa, Artificial Sequence
  Synthetic Polypeptide
GEEPLYWSFPAKKK

SEQ ID NO: 2 - protein, 14 aa, Artificial Sequence
  Synthetic Polypeptide
GAQEEIYAAFFAKK

SEQ ID NO: 3 - protein, 12 aa, Artificial Sequence
  Synthetic Polypeptide
KKKKEEIYFFFG

SEQ ID NO: 4 - protein, 16 aa, Artificial Sequence
  Synthetic Polypeptide
IPTSPITTTYFFFKKK